(19) United States
(12) Patent Application Publication
(10) Pub. No.: US 2023/0109393 A1
(43) Pub. Date: Apr. 6, 2023

(54) DESIGNER PEPTIDES AND PROTEINS FOR THE DETECTION, PREVENTION AND TREATMENT OF CORONAVIRUS DISEASE, 2019 (COVID-19)

(71) Applicant: United Biomedical, Inc., Hauppauge, NY (US)

(72) Inventors: Chang Yi Wang, Cold Spring Harbor, NY (US); Feng Lin, Hauppauge, NY (US); Shuang Ding, Hauppauge, NY (US); Wen-Jiun Peng, Hukou Township (TW)

(21) Appl. No.: 17/801,055

(22) PCT Filed: Feb. 19, 2021
(86) PCT No.: PCT/US2021/018855

§ 371 Date: Aug. 19, 2022

Related U.S. Application Data
(60) Provisional application No. 63/118,596, filed on Nov. 25, 2020.
(60) Provisional application No. 63/027,290, filed on May 19, 2020.
(60) Provisional application No. 62/990,382, filed on Mar. 16, 2020.
(60) Provisional application No. 62/978,596, filed on Feb. 19, 2020.

(51) Int. Cl. C07K 14/005; C07K 16/10; A61K 39/215

(52) U.S. Cl. CPC C07K 14/005; C07K 16/10; A61K 39/215; A61K 2039/543

(57) ABSTRACT

The present disclosure is directed to a relief system for the effective detection, prevention, and treatment of COVID-19, including (1) serological diagnostic assays for the detection of viral infection and epidemiological surveillance, (2) high-precision, site-directed peptide immunogen constructs for the prevention of infection by SARS-CoV-2, (3) receptor-based antiviral therapies for the treatment of the disease in infected patients, and (4) designer protein vaccine containing S1-RBD-sFc. The disclosed relief system utilizes amino acid sequences from SARS-CoV-2 proteins as well as human receptors for the design and manufacture of optimal SARS-CoV-2 antigenic peptides, peptide immunogen constructs, CHO-derived protein immunogen constructs, long-acting CHO-derived ACE2 proteins, and formulations thereof, as diagnostics, vaccines, and antiviral therapies for the detection, prevention, and treatment of COVID-19.

The present application is a PCT International Application that claims the benefit of U.S. Provisional Application Ser. No. 62/978,596, filed Feb. 19, 2020, U.S. Provisional Application Ser. No. 62/990,382, filed Mar. 16, 2020, U.S. Provisional Application Ser. No. 63/027,290, tiled May 19, 2020, U.S. Provisional Application Ser. No. 63/118,596, tiled Nov. 25, 2020, all of which are hereby incorporated herein by reference in their entireties.

### FIELD OF THE INVENTION

The present disclosure relates to a Coronavirus Disease, 2019 (COVID-19) relief system for the detection, prevention, and treatment of COVID-19, caused by the virus SARS-CoV-2. The disclosed relief system utilizes viral and host-receptor amino acid sequences for the manufacture of optimal SARS-CoV-2 antigenic peptides, peptide immunogen constructs, CHO-derived protein immunogen constructs, long-acting CHO-derived ACE2 proteins, and formulations thereof, as diagnostics, vaccines, and antiviral therapies for the detection, prevention, and treatment of COVID-19.

### BACKGROUND OF THE INVENTION

In December 2019, a zoonotic coronavirus crossed species to infect human populations for the third time in recent decades. The disease caused by the virus, SARS-CoV-2, has been officially named by the World Health Organization (WHO) as “COVID-19” for Coronavirus Disease, 2019, as the illness was first detected at the end of 2019. The virus SARS-CoV-2 was first identified in Wuhan, China and affected people exposed to a seafood wholesale market where other live animals were also sold. The virus SARS-CoV-2 is transmitted human-to-human and causes a severe respiratory disease similar to outbreaks caused by two other pathogenic human respiratory coronaviruses (i.e., severe acute respiratory syndrome-related coronavirus (SARS-CoV) and Middle East respiratory syndrome coronavirus (MERS-CoV)).

Coronaviruses (family Coronaviridae, order Nidovirales) are large, enveloped, positive-stranded RNA viruses with a typical crown-like appearance (website: en.wikipedia.org/wiki/Coronavirus). Their viral genomes (26 to 32 kb) are some of the largest known among all RNA viruses: Coronaviruses are classified into four subgroups (Alphacoronavirus, Betacoronavirus, Gammacoronavirus, and Deltacoronavirus), initially based on antigenic relationships of the spike (S), envelope (E), membrane (M), and nucleocapsid (N) proteins. The Betacoronavirus subgroup includes HCoV-OC43, HCoV-HKU1, SARS-CoV, MERS-CoV, and SARS-CoV-2. Genetic recombination readily occurs between members of the same and of different subgroups providing opportunity for increased genetic diversity.

Zhu, N., et al., 2020, identified and characterized SARS-CoV-2 and sequenced the viral genome from clinical specimens (bronchoalveolar-lavage fluid) and human airway epithelial cells virus isolates. The sequences were found to have 86,9% nucleotide sequence identity to a previously published bat SARS-like CoV genome (bat-SL-CoVZC45, MG772933.1). Additional articles (Chen, Y., et al., 2020 and Perlman, S., 2020) further characterize the genome structure, replication, and pathogenesis of emerging coronaviruses, including SARS-CoV, MERS-CoV, and SARS-CoV-2. A schematic diagram of the SARS-CoV-2 structure is shown in FIG. 1. The viral surface proteins (S, F, M, and N proteins) are embedded in a lipid bilayer envelope produced by the host cell and the single stranded positive-sense viral RNA is associated with the nucleocapsid protein. Unlike other betacoronaviruses, SARS-CoV-2 does not possess a hemagglutinin esterase glycoprotein.

SARS-CoV-2 can be propagated in the same cells used for growing SARS-CoV and MERS-CoV However, SARS-CoV-2 grows better in primary human airway epithelial cells, whereas both SARS-CoV and MERS-CoV infect intrapulmonary epithelial cells more than cells of the upper airways. In addition, transmission of SARS-CoV and MERS-CoV occurs primarily from patients demonstrating known signs and symptoms of the illness, whereas SARS-CoV-2 can be transmitted from asymptomatic patients or patients with mild or nonspecific signs. These differences likely contribute to the faster and more wide-spread transmission of SARS-CoV-2 compared to SARS-CoV and MERS-CoV.

It has been reported that SARS-CoV-2 uses the cellular receptor hACE2 (human angiotensin-converting enzyme 2) for cell entry, which is the same receptor used by SARS-CoV and different from the CD26 receptor used by MERS-CoV (Zhou, P., et al, 2020 and Lei, C., 2020). Accordingly, it has been suggested that transmission of SARS-CoV-2 is expected only after signs of lower respiratory tract disease have developed.

SARS-CoV mutated over the 2002-2004 epidemic to better bind to its cellular receptor and to optimize replication in human cells, which enhanced its virulence. Adaptation readily occurs because coronaviruses have error-prone RNA-dependent RNA polymerases, making mutations and recombination events frequent. By contrast, MERS has not been found to have mutated significantly to enhance human infectivity since it was detected in 2012. It is likely that SARS-CoV-2 will behave more like SARS-CoV and will further adapt to the human host, with enhanced binding to hACE2.

Following the SARS-CoV and MERS-CoV epidemics, great efforts were devoted to the development of new antiviral agents that target coronavirus proteases, polymerases, MTases, and entry proteins. However, none of them has been shown to be efficacious in clinical trials (Chan, J F W, et al., 2013; Cheng, K W, et al., 2015; Wang, Y., et al., 2015). Plasma and antibodies obtained, from the convalescent patients have been used, out of the emergency situations, to treat patients with severe clinical symptoms (Mair-Jenkins, J., et al., 2015). In addition, various vaccine strategies targeting SARS-CoV and MERS-CoV; such as inactivated viruses, live-attenuated viruses, viral vector-based vaccines, subunit vaccines, recombinant proteins, and DNA vaccines, have been developed but have only been evaluated in animals so far (Graham, R L, et al., 2013; de Wit, E., et al., 2016).

Since there is no effective therapy or vaccine in face of the tragic outbreaks of COVID-19, the best current measures to reduce transmission of the virus, and to avoid unnecessary social panic resulting in huge economic losses, are to control the source of infection through (1) early detection by RT-PCR assays, (2) case reporting and quarantining of those in contact with the confirmed positive individuals with strict adherence to universal precautions in health care settings, (3) supportive treatments, and (4) timely publishing epidemic information. Individuals can also help reduce the transmission of SARS-CoV-2 through good personal hygiene, using a fitted mask, and avoiding crowded places.

There is an urgent need for the development of (a) serological assays for effective and rapid detection and surveillance of SARS-CoV-2, (b) vaccines to prevent non-infected individuals from contracting SARS-CoV-2, and (c) antiviral therapies to effectively treat individuals infected with SARS-CoV-2, in order to control the outbreak and reduce the resulting sufferings, including death.

### References

The following documents that are cited in this application as well as additional references cited therein are hereby incorporated by reference in their entireties as if fully disclosed herein.

1. AHMED, S. F., et al., “Preliminary identification of potential vaccine targets for 2019-nCoV based on SARS-CoV immunological studies.” DOI: 10.1101/2020,02.03.933226 (2020)

2. ARENDSE, L. B., et al., “Novel therapeutic approaches targeting the Renin-Angiotensin system and associated peptides in hypertension and heart failure.” Pharmacol. Rev., 71, 539-570 (2019)

3. BLUMBERG, R. S., et al., “Receptor specific transepithelialus transport of therapeutics.” U.S. Pat. No. 6,030,613 (2000), U.S. Pat. No. 6,086,875 (2000), and U.S. Pat. No. 6,485,726 (2002)

4. BLUMBERG, R. S., et al., “Central airway administration for systemic delivery of therapeutics.” WO 03/077834 (2002) and US Patent Publication US2003-0235536A1 (2003)

5. BRAUN, J., et al., “SARS-CoV-2-reactive T cells in healthy donors and patients with COVID-19.” Nature, 587, 270—.274 (2020).

6. CAPON, D. J., et al., “Designing CD4 immunoadhesins for AIDS therapy.” Nature, 337:525 (1989)

7. CAPON, D. J., et al., “Hybrid immunoglobulins.” U.S. Pat. No. 5,116,964 (1992)

8. CHAN, J. F. W., et al,, “Broad-spectrum antivirals for the emerging Middle East respiratory syndrome coronavirus.” J. Infect., 67(6):606- 616 (2013)

9. CHANG, J. C. C., et al., “Adjuvant activity of incomplete Freund's adjuvant.” Advanced Drug Delivery Reviews, 32(3):1.73-186 (1998)

10. CHEN, Y., et al., “Emerging coronaviruses: Genome structure, replication, and pathogenesis.” J Med Virol. DOI: 10.1002/jmv.25681 (2020)

11. CHENG, K. W., et al., “Thiopurine analogs and mycophenolic acid synergistically inhibit the papain-like protease of Middle East respiratory syndrome coronavirus.” Antiviral Res., 115: 9-16 (2015)

12. DE WIT, E., et al,, “SARS and MERS: recent insights into emerging coronaviruses,” Nat Rev. Microbial., 14(8):523-534 (2016)

13. FERRETTI, A. P., et al., “Unbiased Screens Show CD8(+) T Cells of COVID-19 Patients Recognize Shared Epitopes in SARS-CoV-2 that Largely Reside outside the Spike Protein.” Immunity, (2020) doi: 10.1016/j.immuni.2020.10.006.

14. FIELDS, G. B., et al., Chapter 3 in Synthetic Peptides: A User's Guide, ed. Grant, W. H. Freeman & Co., New York, N.Y., p.77 (1992)

15. GOEBL, N. A., et al., “Neonatal 1⁻7^{,}'e Receptor Mediates Internalization of Fc in Transfected Human Endothelial Cells.” Mol. Biol. Cell, 19(12):5490-5505 (2008)

16. GRAHAM, R. L., et al., “A decade after SARS: strategies for controlling emerging coronaviruses.” Nat. Rev. Microbiol., (12):836- 848 (2013)

17. JUNGHANS, R. P., et al., “The protection receptor for IgG catabolism is the beta.2-microglobulin-containing neonatal intestinal transport receptor.” Proc. Natl. Acad. Sci. USA, 93(11):5512-5516 (1996)

18. LE BERT, N., et al., “SARS-CoV-2-specific T cell immunity in cases of COVID-19 and SARS, and uninfected controls.” Nature, 584, 457-462 (2020).

19. LEI, C., et al., “Potent neutralization of 2019 novel coronavirus by recombinant ACE2-Ig.” DOI: 10.1101/2020.02.01.929976 (2020)

20. LIU, H., et al., “Fc Engineering for Developing Therapeutic Bispecific Antibodies and Novel Scaffolds.” Frontiers in Immunology., 8, 38 (2017).

21. LONG. Q.-X., et al., “Clinical and immunological assessment of asymptomatic SARS-CoV-2 infections.” Nat. Med. 26, 1200-1204 (2020).

22. MAIR-JENKINS, J., et al.. “The effectiveness of convalescent plasma and hyperimmune immunoglobulin for the treatment of severe acute respiratory infections of viral etiology: a systematic review and exploratory meta-analysis.” J. Infect. Dis., 211(1):80-90 (2015)

23. NG, O.-W., et al., “Memory T cell responses targeting the SARS coronavirus persist up to 11 years post-infection.” Vaccine, 34, 2008-2014 (2016).

24. OSBORN, B. L., et al., “Pharmacokinetic and pharmacodynamic studies of a human serum albumin-interferon-alpha fusion protein in cynomolgus monkeys,” J. Pharmacol. Exp. Ther., 303(2):540-8 (2002)

25. PERLMAN, S., “Another decade, another coronavirus.” N. Engl. J. Med., DOI: 10.1056/NEJMe2001126 (2020)

26. SHUBIN, Z., et al., “An HIV Envelope gp120-Fc Fusion Protein Elicits Effector Antibody Responses in Rhesus Macaques.” Clin. Vaccine Immunol., 24, (2017).

27. SUI, J., et al. “Potent neutralization of severe acute respiratory syndrome (SARS) coronavirus by a human mAb to S1 protein that blocks receptor association.” Proc. Natl. Adad. Sci. USA, 101, 2536-2541 (2004).

28. WANG, C. Y., et al., “UB-311, a novel UBITh(®) amyloid β peptide vaccine for mild Alzheimer's disease.” Alzheimer's Dement., 3, 262-272 (2017).

29. WANG, C. Y., “Artificial promiscuous T helper cell epitopes as immune stimulators for synthetic peptide immunogens.” PCT Publication No. WO 2020/132275A1 (2020).

30. WANG, Y., et al., “Coronavirus nsp10/nsp16 methyltransferase can be targeted by nsp10-derived peptide in vitro and in vivo to reduce replication and pathogenesis.” J. Virol., 89(16):8416-8427 (2015)

31. WIKIPEDIA, The free encyclopedia, “Coronavirus” available at website: en.wikipedia.org/wiki/Coronavirus (accessed Feb. 17, 2020).

32. WYLLIE, D., et al,, “SARS-CoV-2 responsive T cell numbers are associated with protection from COVID-19: A prospective cohort study in keyworkers.” medRxiv 2020.11.02.20222778 (2020) doi: 10.1101/2020.11.02.20222778.

33. ZHAO, B., et al., “Immunization With Fc-Based Recombinant Epstein-Barr Virus gp350 Elicits Potent Neutralizing Humoral Immune Response in a BALB/c Mice Model.” Front. Immunol., 9, 932 (2018).

34. ZHOU, P., et al., “Discovery of a novel coronavirus associated with the recent pneumonia outbreak in humans and its potential bat origin.” DOI: 10.1101/2020.01.22.914952 (2020)

35. ZHU, N., et al., “A novel coronavirus from patients with pneumonia in China, 2019.” N. Engl. J. Med., DOI: 10.1056/NEJMoa2001017 (2020)

### SUMMARY OF THE INVENTION

The present disclosure is directed to a relief system for the effective detection, prevention, and treatment of COVID-19, including (1) serological diagnostic assays for the detection of viral infection and epidemiological surveillance, (2) high-precision, site-directed peptide immunogen constructs for the prevention of infection by SARS-CoV-2, (3) receptor-based antiviral therapies for the treatment of the disease in infected patients, and (4) designer protein vaccine containing S1-RBD-sFc. The disclosed relief system utilizes amino acid sequences from SARS-CoV-2 proteins as well as human receptors for the design and manufacture of optimal SARS-CoV-2 antigenic peptides, peptide immunogen constructs, CHO-derived protein immunogen constructs, long-acting CHO-derived ACE2 proteins, and formulations thereof, as diagnostics, vaccines, and antiviral therapies for the detection, prevention, and treatment of COVID-19.

More specifically, the present invention relates to a systematic approach to develop (1) serological diagnostic assays employing modified SARS-CoV-2 antigenic peptides derived from the M protein (e.g., SEQ ID NOs: 4 and 5), the N protein (e.g., SEQ ID NOs: 17 and 18, 259, 261, 263, 265, 266, and 270), and the S protein (e.g., SEQ ID NOs: 23, 24, 26-34, 37, 38, 281, 308, 321, 322, 323, 324) for detection of viral infection and epidemiological surveillance or monitoring of serum neutralizing antibodies in an infected and/or vaccinated individual; (2) high precision S-RBD (Receptor Binding Domain from the S protein of SARS-CoV-2, also referred to as S1-RBD) derived B epitope immunogen constructs (SEQ ID NOs: 107-144, 20, 226, 227, 239, 240, 241, 246, 247), SARS-CoV-2 derived CTL epitope peptides (SEQ ID NOs: 145-160), T helper cell (Th) epitope derived from a pathogen protein (e.g., SEQ ID NOs: 49-100), Th epitope peptides derived from SARS-CoV-2 (e.g., SEQ ID NOs: 161-165), (3) CHO-expressed S1-RBD-single chain Fc (s-Fc) fusion proteins (SEQ ID NOs: 235 and 236) and CHO-expressed ACE2-ECD-single chain Fc fusion proteins (extra-cellular domain of ACE2) (SEQ ID NOs: 237 and 238) proteins as antiviral therapies for treatment of COVID-19; and (4) designer protein vaccine containing S1-RBD-sFc (e.g., SEQ ID NOs: 235 and 236); utilizing bioinformatics including SARS-CoV-2 viral and receptor amino acid sequences for the design and manufacture of SARS-CoV-2 antigenic peptides, peptide immunogen constructs, and long acting ACE2 receptor proteins and formulations thereof.

### BRIEF DESCRIPTION OF THE DRAWINGS

FIG. 1. Schematic diagram showing the structure of SARS-CoV-2. The viral surface proteins (spike, envelope, and membrane) are embedded in a lipid bilayer envelope derived from the host cell. Unlike other betacoronaviruses. SARS-CoV-2 does not possess a hemagglutinin esterase glycoprotein. The single stranded positive-sense viral RNA is associated with the nucleocapsid protein.

FIG. 2. A representative design of SARS-CoV-2 S-RBD (i.e., Receptor Binding Domain from the Spike protein) derived B cell epitope peptide immunogen constructs comprising constrained loop A, B, and C, respectively, based on an adapted 3D structure of ACE2 and SARS-CoV binding complex (image acquired through the Protein Data Bank (PDB) entry: 2AJF).

FIG. 3. Alignment of M protein sequences from SARS-CoV-2, SARS-CoV, and MERS-CoV An asterisk (*) represents an identical amino acid for the position, a colon (:) represents conserved substitution, a period (.) represents semi-conserved substitution, and an underline (_) represents an antigenic peptide.

FIG. 4. Alignment of N protein sequences from SARS-CoV-2, SARS-CoV and MERS-CoV An asterisk (*) represents identical amino acid for the position, a colon (:) represents conserved substitution, a period (.) represents semi-conserved substitution, an underline (_)represents an antigenic peptide, a dashed line (--) represents a CM epitope, and a dotted line ( . . . ) represents a Th epitope.

FIGS. 5A-5C. Alignment of S protein sequences from SARS-CoV-2, SARS-CoV and MERS-CoV. An asterisk (*) represents identical amino acid for the position, a colon (:) represents conserved substitution, a period (.) represents semi-conserved substitution, an underline (_) represents an antigenic peptide, a dashed line (--) represents a CTL epitope, a dotted line ( . . . ) represents a Th epitope, and a box (□) represents a B cell epitope.

FIGS. 6A-6D. Illustrates the design of a single chain fusion protein according to various embodiments of the present disclosure. FIG. 6A illustrates the structure of a fusion protein comprising an S-RBD at the N-terminus that is covalently linked to a hinge region and Fc fragment (C_{H}2 and C_{H}3 domains) of human IgG. FIG. 6B illustrates a fusion protein comprising an S-RBD from SARS-CoV-2 at the N-terminus that is covalently linked through a linker to a hinge region and Fc fragment (C_{H}2 and C_{H}3 domains) of human IgG. FIG. 6C illustrates a fusion protein comprising an ACE2-ECD (i.e., extra-cellular domain of ACE2) at the N-terminus that is covalently linked to a hinge region and Fc fragment (C_{H}2 and C_{H}3 domains) of human IgG. FIG. 6D illustrates a fusion protein comprising an ACE2-ECD at the N-terminus that is covalently linked through a linker to a hinge region and Fc fragment (C_{H}2 and CH.3 domains) of human IgG.

FIG. 7. Illustrates a map of pZD/S-RBD-sFc plasmid. The pZD/S-RBD-sFc plasmid encodes an S-RBD-sFc fusion protein according to an embodiment of the present invention.

FIG. 8. Illustrates a map of pZD/hACE2-sFc plasmid. The pZD/hACE2-sFc plasmid encodes an ACE2-sFc fusion protein according to an embodiment of the present invention.

FIG. 9. Illustrates the biochemical characterization of a representative purified designer S1-RBD-sFC protein by SDS-PAGE with Coomassie blue staining under non-reducing and reducing conditions.

FIG. 10. illustrates the biochemical characterization of a representative purified designer S1-RBD-His protein by SDS-PAGE with Coomassie blue staining under non-reducing and reducing conditions.

FIG. 11. illustrates the biochemical characterization of a representative purified designer ACE2-ECD-sFC protein by SDS-PAGE with Coomassie blue staining under non-reducing and reducing conditions.

FIG. 12. Illustrates the biochemical characterization of a representative purified designer S1-RBD-His protein by LC mass spectrometry analysis.

FIG. 13. Illustrates the N- and O-glycosylation patterns of a representative purified designer S1-RBD-sFc protein having the sequence of SEQ ID NO: 235.

FIG. 14. Illustrates the biochemical characterization of a representative purified designer S1-RBD-sFc protein by LC mass spectrometry analysis.

FIG. 15. Illustrates the N- and O-glycosylation patterns of a representative purified designer ACE2-ECD-sFc protein having the sequence of SEQ ID NO: 237.

FIG. 16. Illustrates the biochemical characterization of a representative purified designer ACE2-ECD-sFc protein by MALDI-TOF mass spectrometry analysis.

FIG. 17. Illustrates the design and identification of antigenic peptides from SARS-CoV-2 N (Nucleocapsid) protein. A schematic of the full-length N protein is shown at the top and the designer peptide antigens disclosed herein are shown below.

FIG. 18. Illustrates the design and identification of antigenic peptides from SARS-CoV-2 S (Spike) protein. A schematic of the full-length S protein is shown at the top and the designer peptide antigens disclosed herein are shown below.

FIG. 19. Illustrates the design and identification of antigenic peptides from SARS-CoV-2 M (Membrane) protein. A schematic of the full-length M protein is shown at the top and the designer peptide antigens disclosed herein are shown below.

FIG. 20. Illustrates the design and identification of antigenic peptides from SARS-CoV-2 E (Envelope) protein. A schematic of the full-length E protein is shown at the top and the designer peptide antigens disclosed herein are shown below.

FIG. 21. Illustrates the design and identification of antigenic peptides from SARS-CoV-2 ORF9b protein. A schematic of the full-length ORF9b protein is shown at the top and the designer peptide antigens disclosed herein are shown below.

FIG. 22. Illustrates the reactivities with identified antigenic peptides from various regions derived from SARS-CoV-2 N (Nucleocapsid) protein by serum antibodies obtained from representative COVID-19 patients.

FIG. 23. Illustrates the mapping of antigenic regions from SARS-CoV-2 S (Spike) protein by serum antibodies from representative COVID-19 patients.

FIG. 24. Illustrates the sites of four antigenic peptides on the SARS-CoV-2 S (Spike) protein by a 3D structure.

FIG. 25. Illustrates the antigenic regions from SARS-CoV-2 E (Envelope) protein by serum antibodies from representative COVID-19 patients.

FIG. 26. Illustrates of the antigenic regions from SARS-CoV-2 M (Membrane) protein by serum antibodies from representative COVID-19 patients.

FIG. 27. Illustrates of the antigenic regions from SARS-CoV-2 ORF9b protein by serum antibodies from representative COVID-19 patients.

FIG. 28. Illustrates the analytical sensitivity of SARS-CoV-2 ELISA with sera from representative PCR positive COVID-19 patients.

FIG. 29. Illustrates sero-reactivity patters of COVID-19 patient sera detected by ELISA with plates coated with individual antigenic peptides derived from N protein (SEQ ID NOs: 18, 261, and 266), M protein (SEQ ID NO: 5), and S protein (SEQ ID NOs: 38, 281, and 322).

FIG. 30. Illustrates sero-reactivity patters of SARS-CoV-2 ELISA positive, asymptomatic individuals by confirmatory ELISAs with plates coated with individual antigenic peptides derived from N protein (SEQ ID NOs: 18, 261, and 266), M protein (SEQ ID NO: 5), and S protein (SEQ ID NOs: 38, 281, and 322).

FIG. 31. Illustrates the distribution of mean Non-Reactive Control (NRC) values by plate run.

FIG. 32. Illustrates the distribution of OD450nm readings for COVID-19 patients from samples taken less than 10 days after hospitalization, more than 10 days from hospitalization, on the day of discharge, and 14 days after hospital discharge.

FIG. 33. Illustrates the distribution of S/C ratios of samples from COVID-19 patients taken a different, time points and from samples collected from individuals unrelated to SARS-CoV-2 infection.

FIG. 34. Illustrates the binding of HRP conjugated S1-RBD protein to ACE2-ECD-sFc by ELISA.

FIG. 35. Illustrates the inhibition of S1-RBD binding to ACE2-ECD-sFc by ELISA using immune sera generated by S1-RBD immunization.

FIG. 36. Illustrates the assessment of immunogenicity associated with varying forms of designer proteins by ELISA using S1 protein coated plates.

FIGS. 37A-37B. Illustrate the immunogenicity and neutralization assessment of the S1-RBD fusion proteins by ELISA. FIG. 37A provides the immunogenicity assessment by titration of immune sera (3 and 5 WPI) by ELISA using S1 protein coated plates. FIG. 37B provides the neutralization and inhibitory dilution ID₅₀ (Geometric Mean Titer; GMT) in S1 protein binding to ACE2 on ELISA by guinea pigs immune sera at 5 WPI.

FIG. 38. Illustrates immunogenicity assessment by titration of immune sera (3 and 5 WPI) by ELISA using S1 protein coated plates.

FIG. 39. Illustrates assessment of neutralizing antibody titers by an S1-RBD and ACE2 Binding inhibition assay using two separate methods, Method A and Method B.

FIG. 40. Illustrates the assessment of S1-RBD and ACE2 binding inhibition by immune sera (5 WPI) generated by varying forms of designer S1-RBD protein immunogens at different serum dilutions using Method A.

FIG. 41. Illustrates the assessment of S1-RBD and ACE2 binding inhibition by immune sera generated using method (B) varying forms of designer S1-RBD protein immunogens at different serum dilutions.

FIG. 42. Illustrates assessment of S1-RBD and ACE2 binding inhibition by immune sera generated by varying forms of designer S1-RBD protein immunogens through a cell-based blocking assay.

FIG. 43. Illustrates assessment of S1-RBD and ACE2 binding inhibition by immune sera generated by varying forms of designer S1-RBD protein immunogens through a cell-based blocking assay at different serum dilutions.

FIG. 44. Illustrates the assessment of S1-RBD and ACE2 binding inhibition by immune sera (0, 3 and 5 WPI) generated by varying forms of designer S1-RBD protein immunogens through a cell-based blocking assay at different serum.

FIG. 45. Illustrates Phase I clinical trial design for a representative designer vaccine against SARS-CoV-2.

FIG. 46. Illustrates the selection criteria for vaccines from healthy adult volunteers.

FIG. 47. Illustrates the clinical design for a Phase I, open-label study to evaluate the safety, tolerability, and immunogenicity of a designer vaccine against SARS-CoV-2 in healthy adult volunteers.

FIG. 48. Illustrates the clinical activities associated with a Phase I, open-label study to evaluate the safety, tolerability, and immunogenicity of a designer vaccine against SARS-CoV-2 in healthy adult volunteers.

FIG. 49. Illustrates the clinical design for a Phase I, open-label study to evaluate the safety, tolerability, and immunogenicity of a designer vaccine against SARS-CoV-2 in healthy adult volunteers in two stages with four cohorts.

FIG. 50. Illustrates the ACE2-sFc binds to SARS-CoV-2 S1 protein with a high binding affinity.

FIG. 51. Illustrates that ACE2-sFc is able to block S I protein binding to ACE2 coated on ELISA plates.

FIG. 52A-52C. Illustrates the amino acid sequence, structure, and function of S1-RBD-sFc. FIG. 52A provides the sequence of S1-RBD-sFc, and identifies the N-linked glycosylation site (*), the O-linked glycosylation site (+) the Asn-to-His mutation (underlined residue), and the disulfide bonds (connected lines). FIG. 52B summarizes the disulfide bonding in the S1-RBD-sFc fusion protein. FIG. 53C is a graph that shows the binding ability of S1-RBD-sFc to hACE2 by optical density.

FIG. 53. Illustrates the comparative S1-RBD:ACE2 binding inhibition by guinea pig sear and convalescent sera SARS-CoV-2 inhibition rates were evaluated with human serum samples from normal healthy persons (NHP, n=10) and virologically diagnosed COVID-19 patients (n=10) tested at a 1:20 dilution. Pooled immune sera from S1-RBD-sFc vaccinated GP collected at 3 WPI and 5 WPI, were tested at 1:1000 and 1:8000 dilutions, respectively.

FIG. 54. Illustrates the potent neutralization of live SARS-CoV-2 by immune sera. Immune sera collected at 5 WPI from guinea pigs vaccinated at 0 and 3 WPI with S1-RBD-sFc, S1-RBDa-sFc, and S1-RBD-Fc with MONTANIDE™ ISA 50V2 were analyzed. The monolayers of Vero-E6 cells infected with virus-serum mixtures were assessed by immunofluorescence (IFA). Cells were stained with human anti-SARS-CoV-2 N protein antibody and detected with anti-human IgG-488 (light shading). The nuclei were counter stained with DAPI (4′,6-diamidino-2-phenylindole) (dark shading).

FIG. 55. Illustrates neutralization tests on blinded serum samples. Neutralization was assessed with a recombinant SARS-CoV-2 expressing neon green protein ic-SARS-CoV-2-mNG) using the fluorescent signal as a readout for viral replication. The limit of detection of the assay is 1:20 and negative samples were assigned a 1:10 titer. As a positive control, plasma from a convalescent COVID-19 human patient was included. There was a strong correlation (R=0.94) in this assay with the neutralization titers obtained at Academia Sinica.

FIG. 56. A schematic illustrating the components of a multitope protein/peptide vaccine disclosed herein. The vaccine composition contains an S1-RBD-sFc fusion protein for the B cell epitopes, five synthetic Th/CTL peptides for class I and II MHC molecules derived from SARS-CoV-2 S, M, and N proteins, and the UBITh®1a peptide. These components are mixed with CpG1 which binds to the positively (designed) charged peptides by dipolar interactions and also serves as an adjuvant, which is then bound to ADJU-PHOS® adjuvant to constitute the multitope vaccine drug product.

FIGS. 57A-57C. Illustrates the humoral immunogenicity testing in rats. FIG. 57A shows the immunogenicity of a vaccine composition adjuvanted with ISA51/CpG3 (left panel) or ADJU-PHOS®/CpG1 (right panel). Sprague Dawley rats were immunized at weeks 0 and 2 with the vaccine composition (at a dose range of 10-300 μg/dose of S1-RBD-sFc, formulated with synthetic designer peptides and adjuvants). Immune sera at 0, 2, 3, and 4 WPI were assayed for direct binding to S1-RBD protein on ELISA. FIG. 57B (left panel) shows the hACE binding inhibition by antibodies from rats immunized with a vaccine composition adjuvanted with ISA51/CpG3 or ADM-PHOS®/CpG1 from samples taken 4 WPI. FIG. 57B (right panel) shows potent neutralization of live SAM-CoV-2 by rat immune sera expressed as VNT50 for vaccine compositions adjuvanted with ISA51/CpG3 or ADJU-PHOS®/CpG1. FIG. 57C shows the RBD:ACE2 inhibiting titers of sera from rats immunized with varying doses of vaccine compositions in comparison with convalescent COVID-19 patients (left panel) and the potent neutralization of live SARS-CoV-2 expressed as VNT50 (right panel).

FIGS. 58A-58C. Illustrates the cellular immunogenicity testing in rats (ELISpot detection of IFN-γ, IL-2, and IL-4 secreting cells in rats immunized with a vaccine composition. FIG. 58A shows the IFN-γ and IL-4-secreting ELISpot analysis from cells stimulated with Th/CTL peptide pools of rats immunized with vaccine compositions ranging from 1 μg to 100 μg on 0 and 2 WPI. FIG. 58B shows the IL-2 and IL-4-secreting ELISpot analysis from cells stimulated with Th/CTL, peptide pools of rats immunized with vaccine compositions ranging from 1 μg to 100 μg on 0 and 2 WPI. FIG. 58C shows the IL-2 and IL-4 responses from cells stimulated with the individual peptides shown. Cytokine-secreting cells (SC) per million cells was calculated by subtracting the negative control wells. Bars represent the mean SD (n=3). The secretion of IFN-γ or IL-2 was observed to be significantly higher than that of IL-4 in 30 and 100 μg group (*** p<0.005 using Least Square Mean and paired wise comparison) but they were not statistically different in 1 or 3 μg dose groups. Lanes 1, 2, 3, and 4 represent animals immunized with 1, 3, 30, and 100 μg/dose of the vaccine composition, respectively.

FIGS. 59A-59C. Illustrates results from live SARS-CoV-2 challenge testing in hACE-transduced mice after receiving different doses of the disclosed vaccine composition. FIG. 59A is a schematic showing the immunization and challenge schedule. FIG. 59B shows the SARS-CoV-2 titers by RT-PCR (left panel) and TCID50 (right panel) from mice challenged with live virus. FIG. 59C shows stained sections of lungs isolated from mice challenged with live virus.

FIGS. 60A-60C. Illustrates immunogenicity results in rhesus macaques (RM) after receiving different doses of the disclosed vaccine composition. FIG. 60A shows the direct binding of RM immune sera to S1-RBD by ELISA. ELISA-based serum antibody titer (mean Log10 SD) was defined as the highest dilution fold with OD450 value above the cutoff value (* p≤0.05, ** p≤0.01). FIG. 60B shows potent neutralization of live SARS-CoV-2 by RM immune sera. Immune sera collected at Day 42 from RM vaccinated at weeks 0 and 4 were assayed in SARS-CoV-2 infected Vero-E6 cells for cytopathic effect (CPE). FIG. 60C shows IFN-γ ELISpot analysis of RM peripheral blood mononuclear cells (PBMCs) collected at Day 35 and stimulated with a Th/CTL peptide pool (** p≤0.01).

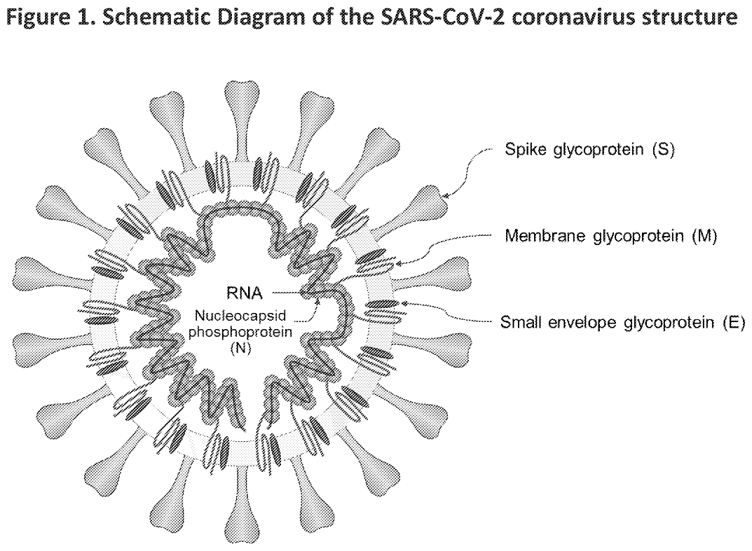

### DETAILED DESCRIPTION OF THE INVENTION

The present disclosure is directed to a relief system for the effective detection, prevention, and treatment of COVID-19, including (1) serological diagnostic assays for the detection of viral infection and epidemiological surveillance, (2) high-precision, site-directed peptide immunogen constructs for the prevention of infection by SARS-CoV-2, (3) receptor-based antiviral therapies for the treatment of the disease in infected patients, and (4) designer protein vaccines containing S1-RBD-sFc protein. The disclosed relief system utilizes amino acid sequences from SARS-CoV-2 proteins as well as human receptors for the design and manufacture of optimal SARS-CoV-2 antigenic peptides, peptide immunogen constructs, CHO-derived protein immunogen constructs. long-acting CHO-derived ACE2 proteins, and formulations thereof, as diagnostics, vaccines, and antiviral therapies for the detection, prevention, and treatment of COVID-19.

Each aspect of the disclosed relief system is discussed in further detail below.

### General

The section headings used herein are for organizational purposes only and are not to be construed as limiting the subject matter described. All references or portions of references cited in this application are expressly incorporated by reference herein in their entirety for any purpose. Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence, the phrase “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples disclosed herein are illustrative only and not intended to be limiting.

The term “SARS-CoV-2”, as used herein, refers to the 2019 novel coronavirus strain that was first identified in Wuhan, China and affected people exposed to a seafood wholesale market where other live animals were also sold. SARS-CoV-2 is also known as the severe acute respiratory syndrome coronavirus 2 (SARS-CoV-2) and is the cause of the coronavirus disease 2019 (COVID-ID).

The term “COVID-19”, as used herein, refers to the human infectious disease caused by the SARS-CoV-2 viral strain. COVID-19 was initially known as SARS-CoV-2 acute respiratory disease. The disease may initially present with few or no symptoms, or may develop into fever, coughing, shortness of breath, pain in the muscles and tiredness. Complications may include pneumonia and acute respiratory distress syndrome.

### A. Serological Diagnostic Assays for the Detection of Viral Infection and Epidemiological Surveillance

### 1. Rationale

The first aspect of the disclosed relief system relates to serological diagnostic assays for the detection of viral infection and epidemiological surveillance.

Detection of antibodies in serum samples from an infected patient at two or more time points is important to demonstrate the seroconversion status upon infection. The collection and analysis of serological data from at risk populations would assist healthcare professionals with constructing a surveillance pyramid to guide the response to the COVID-19 outbreak by SARS-CoV-2. Currently, there is no knowledge about where SARS-CoV-2 falls on the scale of human-to-human transmissibility. Within one month from official announcement of the SARS-CoV-2 outbreak in Wuhan, the virus has been found to be far more transmissible compared to SARS-CoV and MERS-CoV with seemingly lower pathogenicity, thus posing a lower health threat on the individual level. However, the outbreak has resulted in a large-scale spread through super-spreader events and has posed an unprecedented high risk on the population level, which has caused disruption of global public health systems and economic losses.

An aggressive response aimed at tracing and diagnosing infected individuals and monitoring at-risk individuals in order to break the transmission chain of SARS-CoV-2 would require a fast, accurate, and easy-to-perform serological test that detects antibodies to SARS-CoV-2 in a biological sample from individuals. Preferably, such a serological test could be processed using an automated blood screening operation. A fast, accurate, and easy-to-perform serological test for the detection of antibodies to SARS-CoV-2, would be of significant value for the identification, control, and elimination of SARS-CoV-2.

One aspect of the present disclosure is directed to one or more SARS-CoV-2 antigenic peptides, or a fragment(s) thereof, for use in immunoassays assays and/or diagnostic kits as the immunosorbent to detect and diagnose infection by SARS-CoV-2. Immunoassays and/or diagnostic kits containing one or more of antigenic peptides, or fragment(s) thereof, are useful for identifying and detecting antibodies induced by infection or by vaccination. Such tests can be used to screen for the presence of SARS-CoV-2 infection in the clinic, for epidemiological surveillance, and for testing the efficacy of vaccines.

2. Antigenic Peptides for the Detection of Antibodies to M, N, and S proteins of SARS-CoV-2 in Infected Individuals

The disclosed serological diagnostic assays utilize the full-length Membrane (M), Nucleocapsid (N), and Spike (S) proteins of SARS-CoV-2 or fragments thereof. In some embodiments, the diagnostic assays utilize antigenic peptides derived from amino acid sequences from the M, N, and S proteins of SARS-CoV-2. Such antigenic peptides correspond to portions of the amino acid sequences in the M, N, and S proteins that form an epitope for antibody recognition. Preferably, the antigenic peptides are B cell epitopes from SARS-CoV-2 that patients with COVID-19 have produced antibodies against. Such epitopes can be empirically determined using samples from COVID-19 patients known to be infected with SARS-CoV-2. Any immunoassay known in the art (e.g., ELISA, immunodot, immunoblot, etc.) using the antigenic peptides can be used to detect the presence of SARS-CoV-2 antibodies in a biological sample from a subject.

The antigenic peptides can vary in length from about 15 amino acid residues to the full-length amino acid sequence of the M protein (SEQ ID NO: 1), N protein (SEQ ID NO: 6), or S protein (SEQ ID NO: 20). Preferably, the antigenic peptides of the invention are about 20 to about 70 amino acid residues.

Antigenic peptides from the M, N, and S proteins of SARS-CoV-2 using bioinformatics and sequence alignments with the corresponding protein sequences from SARS-CoV. They were initially designed, synthesized, and extensively tested by a large panel of sera from patients with COVID-19 for their ability to be bound by these patient sera. Several antigenic peptides from SARS-CoV-2 were identified using this approach that were considered to have the most significant and consistent antigenicity and binding affinity for the SARS-CoV-2 positive serum panel:

M protein: amino acid residues 1-23 (SEQ ID NO: 4);

N protein: amino acid residues 355-419 (SEQ ID NO: 17, 259, 261, 263, 265, 266, 270); and

S protein: amino acid residues 785-839 (SEQ ID NO: 37, 281, 308, 321, 322, 323, 324).

These three antigenic peptides were further optimized for increased solubility and plate coating efficiency by an addition of three lysine residues (KKK) at their N-terminal ends to produce the optimized antigenic peptides of SEQ ID NOs: 5, 18, and 38, respectively. The optimized antigenic peptides containing the N-terminal lysine tail (SEQ ID NOs: 5, 18, and 38) can be used in serological diagnostic assays individually, or they can be combined in a mixture to produce an optimal antibody capture phase for the detection of antibodies to SARS-CoV-2.

In some embodiments, the serological diagnostic assays and/or diagnostic kits utilize a mixture of optimized antigenic peptides selected from those of SEQ ID NOs: 5, 18, 259. 261, 263, 265, 266, 270, 38, 281, 308, 321, 322, 323, and 324 as the antibody capture phase for the detection of antibodies to SARS-CoV-2. In certain embodiments, antibody binding to the optimized antigenic peptides is detected using ELISA.

### 3. Antigenic Peptides for the Detection of Antibodies in Vaccinated Individuals

In addition to detecting and diagnosing whether a patient has been infected with SARS-CoV-2, it is also important to evaluate the efficacy of patients immunized with a SARS-CoV-2 vaccine, disclosed herein. A serological assay utilizing antigenic peptides used in vaccine compositions can be used to determine the efficacy of immunizations with a vaccine.

B cell cluster antigenic peptides were identified and designed around the receptor binding domain (RBD) (SEQ ID NO: 226) or neutralizing sites from the S protein of SARS-CoV-2 that can be used to detect antibodies produced in vaccinated individuals. A representative number of B cell cluster antigenic peptides from the RBD of the S1 protein are shown in Tables 3, 11, and 13 (e.g., SEQ ID NOs: 23-24, 26-27, 29-34, 226, 227, and 319). Several of these B cell epitope peptides contain cyclic/looped structures created by disulfide bonds between the cysteine residues that allows local constraints for conformation preservation.

In some embodiments, the serological assay for detecting SARS-CoV-2 antibodies produced in infected individuals and vaccinated individuals receiving a S-RBD peptide immunogen construct described herein utilizes the B cell epitope peptide of SEQ ID NO: 26, 38, 226, 227, 281, 315-319, and 322 as the antibody capture phase, In certain embodiments, antibody binding to the B cell epitope peptide is detected using ELISA.

### 4. Two Serological Tests for Detection of Antibodies to SARS-CoV-2

The present disclosure is directed to two serological tests for detection of antibodies to SARS-CoV-2. In one embodiment, the serological test involves a solid phase coated with peptides selected from those of SEQ ID NOs: 5. 18 and 38, 259, 261, 263, 265, 266, 270, 281, 308, 321, 322, 323, and 324 for identification of individuals infected with SARS-CoV-2. In the second test, which can be differentiated from the first test, a solid phase is coated with the peptide of SEQ ID NO: 26, 226, 227 or 319 to assess the titers of neutralizing antibodies. The production and use of diagnostic test kits comprising SARS-CoV-2 peptides (e.g., SEQ ID NOs: 5, 18. and 38, 259, 261. 263, 265, 270, 38, 281, 308, 321, 322, 323, and 324) and (SEQ ID NO: 26, 226, 227 or 319) are within the scope of various exemplary embodiments of the disclosure.

In specific embodiments, the antigenic peptides or B cell epitope peptides are useful for the detection of SARS-CoV-2 antibodies in a biological sample from a patient for the diagnosis of COVID-19. A biological sample includes any bodily fluid or tissue that may contain antibodies. including, but not limited to, blood, serum, plasma, saliva, urine, mucus, fecal matter, tissue extracts, and tissue fluids. The term patient is meant to encompass any mammal such as non-primates (e.g., cow, pig, horse, cat, dog, rat etc.) and primates (e.g., monkey and human), preferably a human.

The antigenic peptides and the B cell epitope peptides of the disclosure can be used in immunoassays to detect the presence of SARS-CoV-2 antibodies in the biological sample from a patient. Any immunoassay known in the art can be used. For example, the biological sample can be contacted with one or more SARS-CoV-2 antigenic or B cell epitope peptides or immunologically functional analogues thereof under conditions conducive to binding. Any binding between the biological sample and the antigenic or B cell epitope peptides or immunologically functional analogues thereof can be measured by methods known in the art. Detection of binding between said biological sample and the SARS-CoV-2 antigenic peptides or immunologically functional analogues thereof indicates the presence of SARS-CoV-2 in the sample. In a more specific embodiment, an ELISA immunoassay can be used to evaluate the presence of SARS-CoV-2 antibodies in a sample. Such ELISA immunoassay comprises the steps of:
i. attaching a peptide, or mixture of peptides, comprising an antigenic peptide (e.g., SEQ ID NOs: 4-5, 17-18, 37-38, 259, 261, 263, 265, 266, 270, 281, 308, 321, 322, 323, and 324) or a B cell epitope peptide (e.g., SEQ ID NOs: 23-24, 26, 27, and 29-34, 226, 227 and 315-319) to a solid support,
ii. exposing the antigenic peptide or B cell epitope peptide attached to the solid support to a biological sample containing antibodies from a patient, under conditions conducive to binding of the antibody to the peptide, and
iii. detecting the presence of antibodies bound to the peptide attached to the solid support.

### 5. Immunologically Functional Homologues and Analogues of the SARS-CoV-2 Peptides

In some embodiments, the antigenic peptides (e.g., SEQ ID NOs: 4-5, 17-18, 37-38, 259, 261, 263, 265, 266. 270, 281, 308, 321, 322, 323, and 324) or B cell epitope peptides (e.g., SEQ ID NOs: 23-24, 26, 27, 29-34, 226, 227, and 315-319) include immunologically functional homologues and/or analogues that have corresponding sequences and conformational elements from mutant and variant strains of SARS-CoV-2.

Homologues and/or analogues of the disclosed SARS-CoV-2 peptides bind to or cross-react with antibodies elicited by SARS-CoV-2 are included in the present disclosure. Analogues, including allelic, species, and induced variants, typically differ from naturally occurring peptides at one, two, or a few positions, often by virtue of conservative substitutions. Analogues typically exhibit at least 75%, 80%, 85%, 90%, or 95% sequence identity with natural peptides. Some analogues also include unnatural amino acids or modifications of N- or C-terminal amino acids at one, two, or a few positions.

Variants that are functional analogues can have a conservative substitution in an amino acid position; a change in overall charge; a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan, and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

In a particular embodiment, the functional analogue has at least 50% identity to the original amino acid sequence. In another embodiment, the functional analogue has at least 80% identity to the original amino acid sequence. In yet another embodiment, the functional analogue has at least 85% identity to the original amino acid sequence. In still another embodiment, the functional analogue has at least 90% identity to the original amino acid sequence.

Homologous SARS-CoV-2 peptides contain sequences that have been modified when compared to the corresponding peptide in some way (e.g., change in sequence or charge, covalent attachment to another moiety, addition of one or more branched structures, and/or multimerization) yet retains substantially the same immunogenicity as the original SARS-CoV-2 peptide.

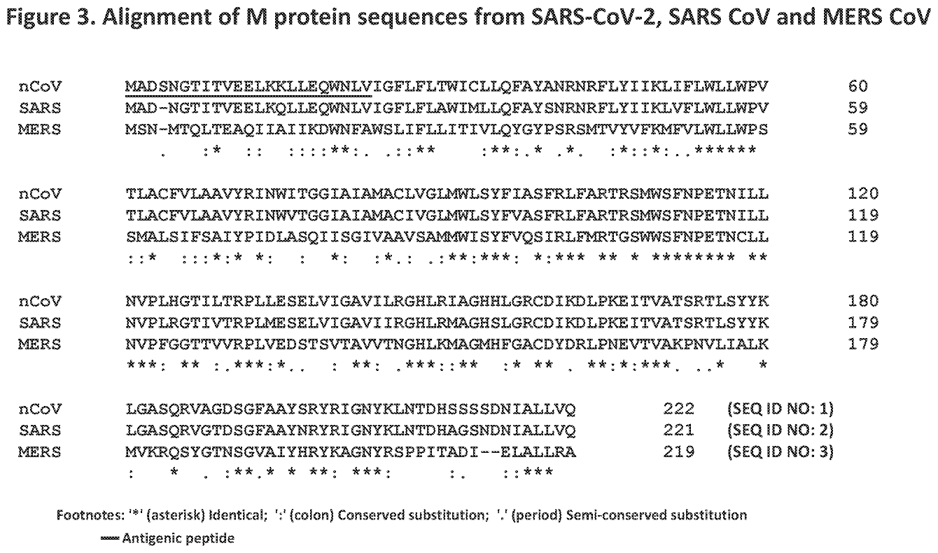

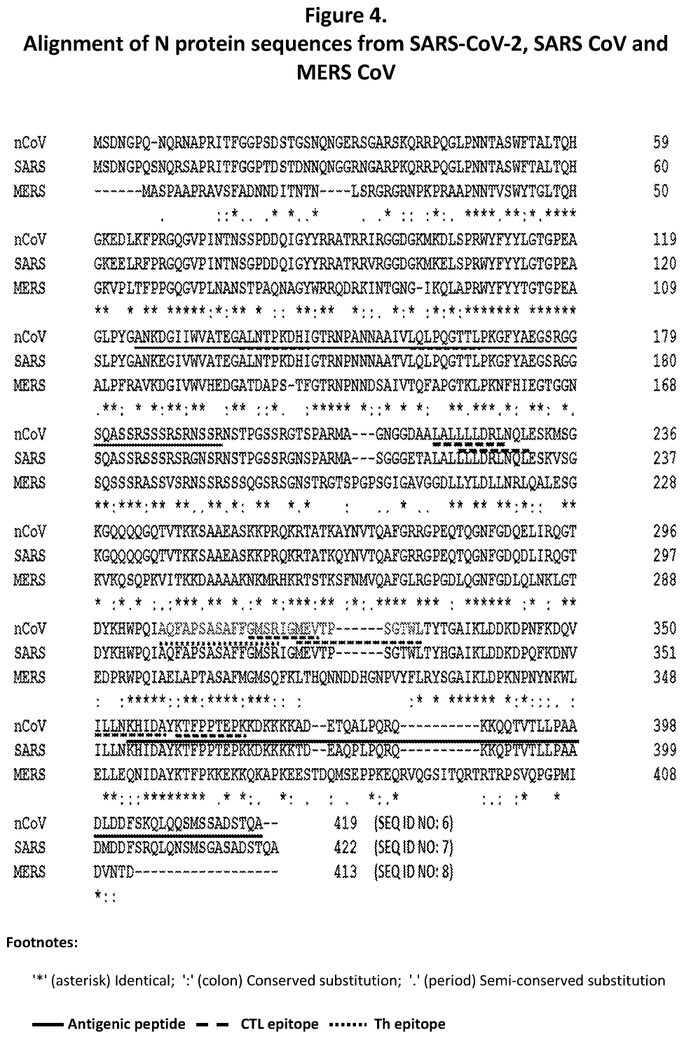

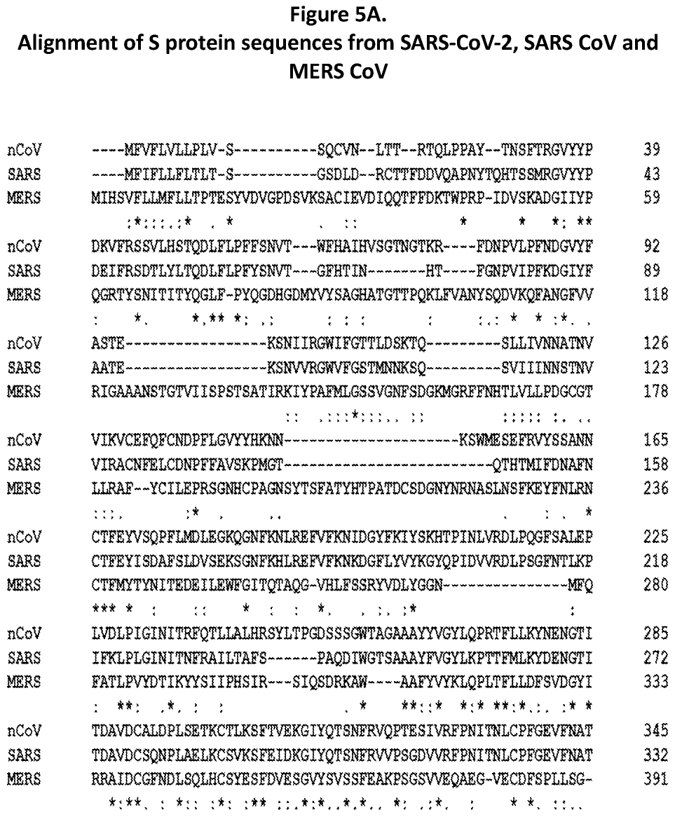

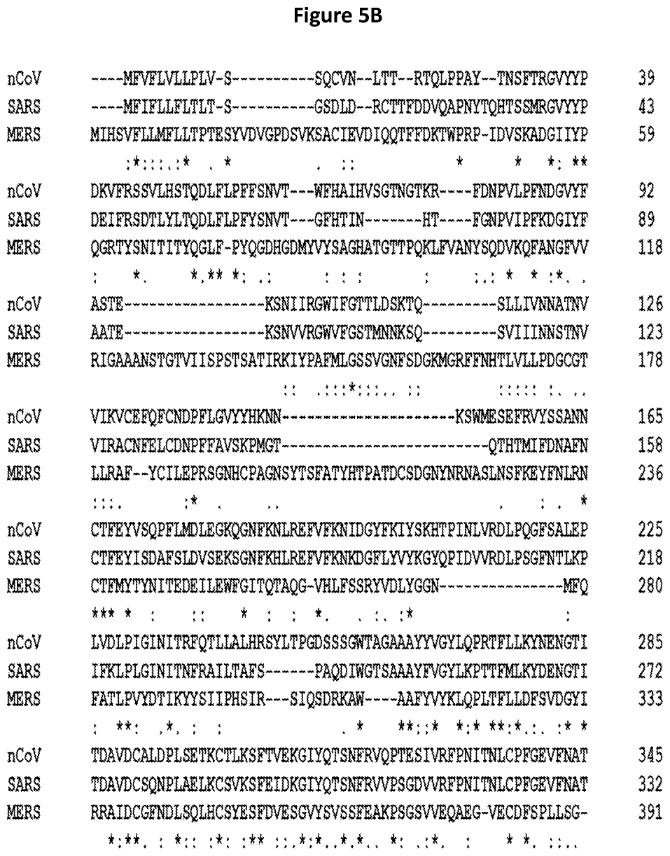

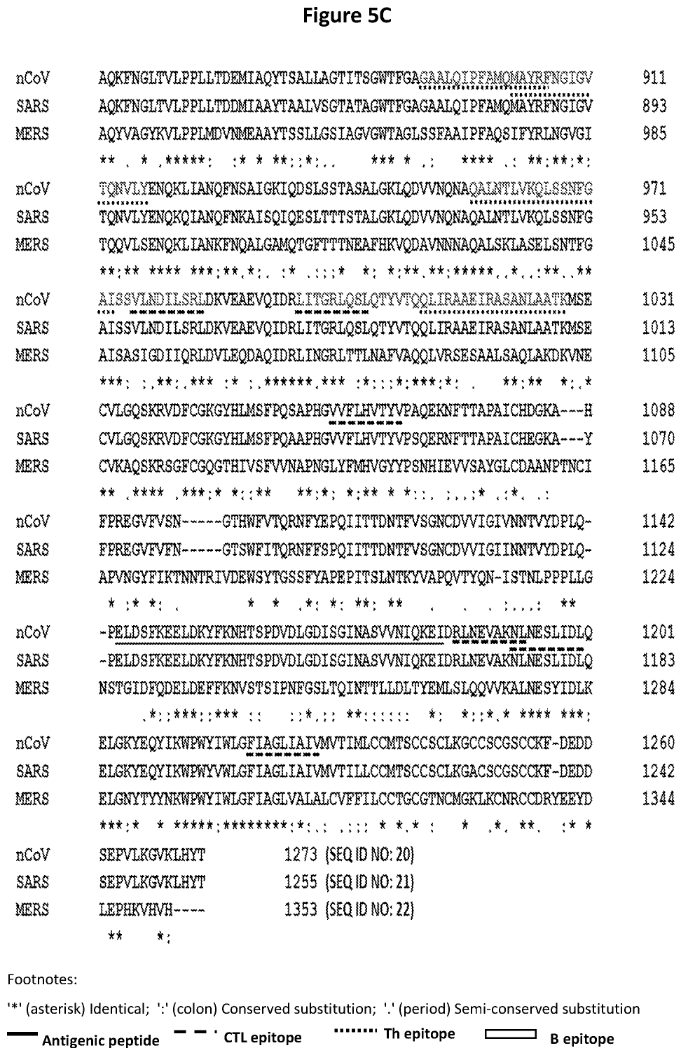

Homologues can be readily identified through sequence alignment programs such as Clustal Omega or protein BLAST analyses. FIGS. 3-5 provide alignments of the amino acid sequences from the coronavirus strains of SARS-CoV-2, SARS CoV, and MERS CoV. These homologous peptides can used individually or can be combined in a mixture to constitute the most optimal antibody capture phase for the detection of antibodies to M, N, and S proteins of SARS-CoV-2 by immunoassay (e.g., ELISA) in biological samples from infected or vaccinated individuals. Homologues of the disclosed peptides are further defined as those peptides derived from the corresponding positions of the amino acid sequences of the variant strains, such as SARS-CoV or MERS-CoV having at least 50% identity to the peptides.

In some embodiments, the variant peptide homologue is derived from amino acid positions of sequences from SARS-CoV or MERS-CoV (e.g., SEQ ID NOs: 2, 3, 721, or 22) that have about >50%, 75%, 80%, 85%, 90%, or 95% sequence identity to SEQ ID NOs: 1, 6, 20 of SARS-CoV-2. In another embodiment, the SARS strain S-RBD peptide homologue (SEQ ID NO: 28) has about 58.6% identity to SEQ ID NO: 26.

A series of synthetic peptides representing antigenic regions of the SARS-CoV-2 M protein (e.g., SEQ ID NOs: 4-5), N protein (e.g., SEQ ID NOs: 17-18, 259, 261, 263, 265, 266, and 270), and S protein (e.g., SEQ ID NOs: 37-38, 281, 308, 321, 322, 323, and 324) and homologues thereof, can be useful, alone or in combination, for the detection of antibodies to SARS-CoV-2 in biological samples from patients for the detection and diagnosis of infection by SARS-CoV-2. In addition, a series of synthetic peptides representing receptor binding domain of the S protein (S-RBD or S1-RBD) of the SARS-CoV-2 (e.g., SEQ ID NO: 26. 226. 227 or 315-319) and homologues thereof, can be useful, alone or in combination, for the detection of neutralizing antibodies to SARS-CoV-2 in biological samples to determine the immunization efficacy of individuals vaccinated with formulations described herein.

### 6. UBI® SARS-CoV-2 ELISA Product

a. Trade Name & Intended Use

The UBI® SARS-CoV-2 ELISA is an Enzyme-Linked Immunosorbent Assay (ELISA) intended for qualitative detection of IgG antibodies to SARS-CoV-2 in human serum and plasma (sodium heparin or dipotassium (K2) EDTA). The UBI® SARS-CoV-2 ELISA is intended for use as an aid in identifying individuals with an adaptive immune response to S AR S-CoV-2, indicating recent or prior infection. At this time, it is unknown for how long antibodies persist following infection and if the presence of antibodies confers protective immunity. The UBI® SARS-CoV-2 ELISA should not be used to diagnose or exclude acute SARS-CoV-2 infection. Testing is limited to laboratories certified under the Clinical Laboratory Improvement Amendments of 1988 (CLIA), 42 U.S.C 263a, that meet requirements to perform high complexity testing.

Results are for the detection of IgG SARS CoV-2 antibodies. IgG antibodies to SARS-CoV-2 are generally detectable in blood several days after initial infection, although the duration of time antibodies are present post-infection is not well characterized. Individuals may have detectable virus present for several weeks following seroconversion.

Laboratories within the United States and its territories are required to report all results to the appropriate public health authorities.

The sensitivity of the UBI® SARS-CoV-2 ELISA early after infection is unknown. Negative results do not preclude acute SARS-CoV-2 infection. If acute infection is suspected, direct testing for SARS-CoV-2 is necessary.

False positive results with the UBI SARS-CoV-2 ELISA may occur due to cross-reactivity from pre-existing antibodies or other possible causes. Due to the risk of false positive results, confirmation of positive results should be considered using a second, different IgG antibody assay.

Samples should only be tested from individuals that are 15 days or more post symptom onset.

The UBI® SARS-CoV-2 ELISA is currently only for use under the Food and Drug Administration's Emergency Use Authorization.

b. Summary and Explanation of the Test

The UBI® SARS-CoV-2 ELISA is an immunoassay that employs synthetic peptides derived from the Matrix (M), Spike (S) and Nucleocapsid (N) proteins of SARS-CoV-2 for the detection of antibodies to SARS- CoV-2 in human sera or plasma. These synthetic peptides, free from cellular or E. coli-derived impurities which the recombinant viral proteins are produced from, bind antibodies specific to highly antigenic segments of SARS-CoV-2 structural M, N and S proteins and constitute the solid phase antigenic immunosorbent. Specimens with absorbance values greater than or equal to the Cutoff Value (i.e., Signal to Cut-off ratio≥1.00) are defined as positive.

c. Chemical and Biological Principles of the Procedure

The UBI® SARS-CoV-2 ELISA employs an immunosorbent bound to the wells of the REACTION MICROPLATE consisting of synthetic peptides that capture antibodies with specificities for highly antigenic segments of the Spike (S), Matrix (M) and Nucleocapsid (N) proteins of SARS-CoV-2.

During the course of the assay, diluted negative controls and specimens are added to the REACTION MICROPLATE wells and incubated. SARS-CoV-2-specific antibodies, if present, will bind to the immunosorbent. After a thorough washing of the REACTION MICROPLATE wells to remove unbound antibodies and other serum/plasma components, a standardized preparation of Horseradish peroxidase-conjugated goat anti-human IgG antibodies specific for human IgG is added to each well. This conjugate preparation is then allowed to react with the captured antibodies. After another thorough washing of the wells to remove unbound horseradish peroxidase-conjugated antibody, a substrate solution containing hydrogen peroxide and 3,3′,5,5′-tetramethylbenzidine (TMB) is added. A blue color develops in proportion to the amount of SARS-CoV-2-specific IgG antibodies present, if any, in most settings. Absorbance of each well is measured within 15 minutes at 450 nm by using a microplate reader such as a VERSAMAX™ by Molecular Devices® or equivalent.

d. Reagent Components and Their Storage Conditions

| UBI ® SARS-CoV-2 ELISA | 192 tests | |
|---|---|---|
| SARS-CoV-2 Reaction Microplates | 192 | wells |
| Each microplate well contains adsorbed SARS-CoV-2 | | |
| synthetic peptides. Store at 2-8° C. sealed with desiccant. | | |
| Non-Reactive Control/Calibrator | 0.2 | ml |
| Inactivated normal human serum containing 0.1% sodium | | |
| azide and 0.02% gentamicin as preservatives. Store at 2-8° C. | | |
| Specimen Diluent (Buffer 1) | 45 | mL |
| Phosphate buffered saline solution containing casein, gelatin, | | |
| and preservatives: 0.1% sodium azide and 0.02% gentamicin. | | |
| Store at 2-8° C. | | |
| Conjugate | 0.5 | mL |
| Horseradish peroxidase-conjugated goat anti-human IgG | | |
| antibodies, with 0.02% gentamicin and 0.05% 4-dimethyl- | | |
| aminoantipyrine. Store at 2-8° C. | | |
| Conjugate Diluent (Buffer II) | 30 | mL |
| Phosphate buffered saline containing surfactant and heat- | | |
| treated normal goat serum, with 0.02% gentamicin as a | 14 | mL |
| preservative. Store at 2-8° C. | | |
| TMB Solution | | |
| 3,3′,5,5′-tetramethylbenzidine (TMB) solution. Store at 2-8° C. | 14 | mL |
| Substrate Diluent | | |
| Citrate buffer containing hydrogen peroxide. Store at 2-8° C. | 25 | mL |
| Stop Solution | | |
| Diluted sulfuric acid solution (1.0M H₂SO₄). Store at 2-30° C. | 150 | mL |
| Wash Buffer Concentrate | | |
| A 25-fold concentrate of phosphate buffered saline with | | |
| surfactant. Store at 2-30° C. | | |
| Dilution Microplates | 192 | wells |
| Blank, yellow microplates for predilution of specimens. | | |
| Store at 2° to 30° C. | | |
| Plate Covers | 6 | sheets |

Clear, plastic adhesive sheets to be used to cover the Reaction Microplate wells during each incubation. Plastic sheets may be cut, before removing the paper backing, whenever less than a full plate of Reaction Microplate wells is being assayed. Alternatively, standard microplate lids may be used.

### Materials Required—Not Provided

| | | |
|---|---|---|
| 1. | Anti-SARS-CoV-2 Positive Control | 0.2 mL |
| | Inactivated human plasma containing SARS-CoV-2 | |
| | IgG antibodies. Store at ≤−20° C. It may be purchased | |
| | separately as Anti-SARS-CoV-2 Positive Control | |
| | (PN 200238) for UBI SARS-CoV-2 ELISA. | |
| 2. | Manual or automatic multi-channel-8 or 12 channel | |
| | pipettors (50 μL to 300 μL) | |
| 3. | Manual or automatic variable pipettors (From 1 μL to | |
| | 200 μL). | |
| 4. | Incubator (37 ± 2° C.). | |
| 5. | Polypropylene or glass containers (25 mL capacity), | |
| | with a cap. | |
| 6. | Sodium hypochlorite solution, 5.25% (liquid | |
| | household bleach). | |
| 7. | A microplate reader capable of transmitting light at a | |
| | wavelength of 450 ± 2 nm. | |
| 8. | Automatic or manual aspiration-wash system capable | |
| | of dispensing and aspirating 250-350 μL | |
| 9. | Pipettor troughs or boats. | |
| 10. | Reagent grade (or better) water. | |
| 11. | Disposable gloves. | |
| 12. | Timer. | |
| 13. | Absorbent tissue. | |
| 14. | Biohazardous waste containers | |
| 15. | Pipettor tips. | |

### Warnings and Precautions

### For In Vitro Diagnostic Research Use

### Currently for Prescription Use Only

### Currently for Emergency Use Authorization Only

1. As of the filing date of this application:
   a. This test has not been FDA cleared or approved but has been authorized for emergency use by FDA under an EUA for use by laboratories certified under the Clinical Laboratory Improvement Amendments of 1988 (CLIA), 42 U.S.C. 263a, that meet requirements to perform high complexity tests.
   b. The emergency use of this test has been authorized only for detecting IgG antibodies against SARS-CoV-2, not for any other viruses or pathogens.
   c. The emergency use of this test is only authorized for the duration of the declaration that circumstances exist justifying the authorization of emergency use of in vitro diagnostic tests for detection and/or diagnosis of COVID-19 under Section 564(b)(1) of the Federal Food, Drug, and Cosmetic Act, 21 U.S.C. § 360bbb-3(b)(1), unless the declaration is terminated or authorization is revoked sooner
2. HANDLE ASSAY SPECIMENS, REACTIVE AND NON-REACTIVE CONTROLS AS IF CAPABLE OF TRANSMITTING AN INFECTIOUS AGENT. Wear disposable gloves throughout the test procedure. Dispose of gloves as biohazardous waste. Wash hands thoroughly afterwards.
3. DO NOT SUBSTITUTE REAGENTS FROM ONE KIT LOT TO ANOTHER, CONJUGATE and REACTION MICROPLATES are matched for optimal performance. Use only the reagents supplied by manufacturer.
4. Do not use kit components beyond their expiration date.
5. The NON-REACTIVE CONTROL/CALIBRATOR should be assayed in triplicate on each plate with each run of specimens. and should be diluted in the same manner as the specimen.
6. Use only reagent grade quality water to dilute the WASH BUFFER CONCENTRATE.
7. Allow all kit reagents and materials to reach room temperature (15 to 30° C.) before use.
8. Do not remove MICROPLATE from the storage bag until needed. Unused strips should be stored at 2 to 8° C. securely sealed in its foil pouch with the desiccant provided.
9. Caution: STOP SOLUTION (1 mol/L H₂SO₄) causes burns. Never add water to this product. In case of contact with eyes, rinse immediately with plenty of water and seek medical advice.
10. Avoid contact of the 1 mol/L SULFURIC ACID (Stop Solution) with any oxidizing agent or metal.
11. Follow the installation, operation, calibration, and maintenance instructions provided by the instrument manufacturers for both microplate reader and automatic microplate washer.
12. Spills should be cleaned thoroughly using either an iodophor disinfectant or sodium hypochlorite solution.
   Iodophor Disinfectant: should be used at a dilution providing at least 100 ppm available iodine. Sodium Hypochlorite:
   a. Non acid-containing spills should be wiped up thoroughly with a 5.25% sodium hypochlorite solution.
   b. Acid-containing spills should be wiped dry. Spill areas should then be wiped with a 525% sodium hypochlorite solution (liquid household bleach).
13.This product contains sodium azide as a preservative. Sodium azide may form lead or copper azides in laboratory plumbing.
   These azides may explode on percussion, such as hammering. To prevent formation of lead or copper azide, thoroughly flush drains with water after disposing of waste solutions. To remove suspected of azide accumulation, the National Institute for Occupational Safety and Health (USA) recommends: (1) siphon liquid from drain trap using a hose, (2) fill with 10% sodium hydroxide solution, (3) allow to stand for 16 hours, and (4) flush well with water.

### Waste Disposal

Dispose of all specimens and materials used to perform the test as if they contain infectious agents. Autoclaving at 121° C. or higher is recommended prior to incineration.

Liquid wastes NOT CONTAINING ACID may be mixed with sodium hypochlorite in volumes such that the final mixture contains 1.0% sodium hypochlorite. Liquid waste containing acid must be neutralized with a proportional amount of base prior to the addition of sodium hypochlorite. Allow at least 30 minutes at room temperatures for decontamination to be completed. The liquid may then be disposed in accordance with local ordinances.

### Specimen Collection and Preparation

1. UBI® SARS-CoV-2 ELISA may be performed on human serum or plasma (anticoagulant sodium heparin or dipotassium EDTA). Specimens containing precipitates or particulate matter may give inconsistent test results. If necessary, specimens should be clarified by centrifugation prior to testing.
2. Specimens must not be heat-inactivated prior to assay.
3. Specimens may be stored at 2-8° C. for up to 48 hours or at ≤−20° C. for up to two months.
4. Specimens may be frozen and thawed once.

### Preparation of Reagents

After removing assay reagents from the refrigerator, al low them to reach room temperature and mix thoroughly by gentle swirling before pipetting.

### Wash Buffer

Prepare and load into plate washer prior to beginning ASSAY PROCEDURE. Dilute 1 volume of WASH BUFFER CONCENTRATE with 24 volumes of reagent grade water. Mix well. Once prepared, diluted WASH SOLUTION is stable for 3 months with occasional mixing. Store at 2 to 30° C. Do not use diluted WASH SOLUTION until it has reached room temperature (15 to 30° C.) if it has been stored in the refrigerator.

### Working Conjugate Solution

Prepare as step 6 of the ASSAY PROCEDURE. Dilute the conjugate 1:100 with the Conjugate Diluent. Refer to the chart below for the correct amount of Working Conjugate Solution to prepare. Mix well to ensure a homogenous solution.

### Working Conjugate Solution Preparation Chart

| Number | Number | Conjugate | Diluent |
|---|---|---|---|
| of Strips | of Tests | (μL) | (mL) |
| 1 to 2 | 8 to 24 | 25 | 2.5 |
| 3 to 6 | 25 to 48 | 50 | 5.0 |
| 7 to 9 | 49 to 72 | 75 | 7.5 |
| 10 to 12 | 73 to 96 | 100 | 10.0 |

### TMB Substrate Solution

Prepare as step 8 of the ASSAY PROCEDURE. Mix the TMB Solution and Substrate Diluent in equal volumes. Refer to the chart below for the correct amount of TMB substrate solution to prepare. USE WITHIN 10 MINUTES OF PREPARATION, PROTECT FROM DIRECT SUNLIGHT.

### TMB Substrate Solution Preparation

| Number of Tests | TMB Buffer (mL) | Substrate Diluent (mL) |
|---|---|---|
| 16 | 1.1 | 1.1 |
| 24 | 1.6 | 1.6 |
| 32 | 2.1 | 2.1 |
| 40 | 2.5 | 2.5 |
| 48 | 2.8 | 2.8 |
| 56 | 3.5 | 3.5 |
| 64 | 3.8 | 3.8 |
| 72 | 4.0 | 4.0 |
| 80 | 4.5 | 4.5 |
| 88 | 5.0 | 5.0 |
| 96 | 5.5 | 5.5 |

All materials should be used at room temperature (15 to 30° C.). Liquid reagents should be thoroughly and gently mixed before use.

### Storage Instructions

1. Store UBI® SARS-CoV-2 ELISA kit and its components at 2 to 8° C. when not in use and use by the kit expiration date.
2. After opening, unused strips of the REACTION MICROPLATES must be stored at 2 to 8° C. securely sealed in foil pouch with the desiccant provided. When kept in the closed pouch at 2 to 8° C., after opening once, the REACTION MICROPLATES are stable for 8 weeks.

### Indications of Instability or Deterioration

1. Changes in the physical appearance of the reagents supplied may indicate deterioration of these materials; do not use reagents which are visibly turbid.
2. The TMB Solution, Substrate Diluent and the prepared SUBSTRATE SOLUTION should be colorless to pale yellow in color for proper performance of the assay. Any other color may indicate deterioration of the TMB Solution and/or Substrate Solution.

### Indications of Instability or Deterioration

The Anti-SARS-CoV-2 Positive Control is treated in the same manner as the test samples and is used to validate the test run. It is recommended that the Positive Control is run in a separate well, concurrently with patient specimens, in each run. The Positive Control absorbance value should be ≥0.5 and the Signal to Cutoff ratio should be >1.0. If either the Positive Control absorbance value or the Signal to Cut-off ratio falls outside the limits, the plate is invalid and the test must be repeated.

The Non-Reactive Control/Calibrator is tested as described in the section Assay Procedure.

Expected results for the Non-Reactive Control/Calibrator are provided in the section Assay Validation.

### Assay Procedure

1. To the DILUTION MICROPLATE:
   A. Dispense 200 μL of SPECEMEN DILUENT (Buffer I) into all wells.
   B. Use well A1 as reagent blank.
   C. Add 10 μL of Non-Reactive Control/Calibrator to wells B1, C1, D1
   D. Add 10 μL of Anti-SARS-CoV-2 Positive Control to the appropriate well.
   E. Add 10 μL of TEST SPECIMEN to the appropriate wells.
2. Ensure that the contents of the wells are thoroughly mixed. Manual mixing with a pipette or gently vibrating the plate is acceptable.
3. Open the foil pouch and remove the REACTION MICROPLATE. When not using the complete REACTION MICROPLATE, remove excess strips from the frame and return them to the storage pouch provided and securely seal. It may be necessary to insert alternate strips, depending on the washing system used.
4. Transfer 100 μL of Reagent Blanks, Non-Reactive Control it Calibrator and Diluted Specimens from each well of the DILUTION MICROPLATE to its corresponding well in the REACTION MICROPLATE.
5. Cover and incubate 60±2 minutes at 37±2° C.
6. Prepare the WORKING CONJUGATE SOLUTION (1:101) as described in PREPARATION OF REAGENTS prior to washing the REACTION MICROPLATES.
7. Wash the MICROPLATE with WASH BUFFER as described in PREPARATION of REAGENTS.
   A. Automatic Microplate Washer—Use six (6) washes with at least 300 μL/well/wash.
   B. Manual Microplate Washer or Pipettor (8 or 12 channel)—wash six (6) times, using at least 300 μL/well/wash. Fill the entire plate, then aspirate in the same order.
8. Make sure that the rest volume is minimal, e.g., by blotting dry by tapping plate onto absorbent paper.
9. Add 100 μL of the prepared WORKING CONJUGATE SOLUTION (1:101) to all wells of the REACTION MICROPLATE. Cover and incubate for 30±1 minute at 37±2° C.
10. Prepare TMB SUBSTRATE SOLUTION during the incubation prior to use according to the PREPARATION OF REAGENTS. Shield the solution from direct light.
11. Repeat the wash procedure as in step 7 and step 8.
12. Add 100 μL of the prepared TMB SUBSTRATE SOLUTION to each well of the REACTION MICROPLATE.
13. Cover and incubate for 15±1 minute at 37±2° C.
14. Add 100 μL of STOP-SOLUTION to each well of the REACTION MICROPLATE. Mix, e.g., by gently tapping or vibrating the plate.
15. Read the absorbance at 450±2 nm with air blank. NOTE: Absorbance should be read within 15 minutes of the addition of the STOP SOLUTION to the REACTION MICROPLATE.

### Assay Validation and Calculation of Results

The presence or absence of antibody specific for SARS-CoV-2 is determined by relating the absorbance of the specimens to the Cutoff Value.

### Assay Validation

For the assay to be valid:
1. The Reagent Blank absorbance values should be less than 0.150. If it is outside the limit, the plate is invalid and the test must be repeated.
2. Individual Non-Reactive Control/Calibrator absorbance values should be less than 0.200 and greater than the Reagent Blank. If one of the three Non-Reactive Control/Calibrator values is outside either of these limits, recalculate the Non-Reactive/Calibrator mean based upon the two acceptable control values. If two or more of the three control values are outside either of the limits (Less than 0.200 and greater than the reagent blank), the plate is invalid and the test must be repeated.
3. The Anti-SARS-CoV-2 Positive Control absorbance value should be ≥0.5 and the Signal to Cutoff ratio should be >1.0. If either the Positive Control absorbance value or the Signal to Cut-off ratio falls outside the limits, the plate is invalid and the test must be repeated.

### Calculation of Results

| 1. | Absorbance of the Reagent Blank (RB) | | |
|---|---|---|---|
| | Example: | Reagent Blank | Absorbance |
| | | Well A1 | 0.044 |
| 2. | Determine the Mean of the NON-REACTIVE CONTROL/ | | |
| | CALIBRATOR (NRC) | | |
| | Example: | NRC | Absorbance |
| | | Well B1 | 0.062 |
| | | Well C1 | 0.066 |
| | | Well D1 | 0.063 |
| | | Total | 0.191 |
| | | Mean | 0.191 ÷ 3 = 0.064 |
| 3. | Calculation of the Cutoff Value: | | |
| | | Cutoff Value | =Mean NRC + 0.2 |
| | Example: | Mean NRC | =0.064 |
| | | Cutoff Value | =0.064 + 0.2 = 0.264 |
| 4. | Calculation of the Signal to Cutoff (S/C) ratio: | | |
| | | S/C ratio | =OD of sample ÷ Cutoff Value |
| | Example: | Sample OD | =0.542 |
| | | Cutoff Value | =0.264 |
| | | S/C ratio | =0.542 / 0.264 = 2.05 |

### Interpretation of Results

1. Specimens with absorbance values less than the Cut-off Value (i.e., Signal to Cutoff ratio <1.00) are negative by the criteria of the UBI® SARS-CoV-2 ELISA and may be considered negative for IgG antibodies to SARS-CoV-2.
2. Specimens with absorbance values greater than or equal to the Cutoff Value (i.e., Signal to Cutoff ratio ≥1.00) are positive by the criteria of the UBI® SARS-CoV-2 ELISA and may be considered positive for antibodies to SARS-CoV-2.

Results of the UBI® SARS-CoV-2 ELISA are interpreted as follows:

| S/C ratio | Result | Interpretation |
|---|---|---|
| <1.00 | Negative | Negative for IgG antibodies to SARS-CoV-2 |
| ≥1.00 | Positive | Positive for IgG antibodies to SARS-CoV-2 |

The magnitude of the measured result above the cutoff is not indicative of the total amount of antibody present in the sample.

### Limitations of the Procedure

1. Use of the UBI SARS CoV-2 ELISA is limited to laboratory personnel who have been trained. Not for home use.
2. The UBI® SARS-CoV-2 ELISA PROCEDURE and the INTERPRETATION OF RESULTS sections must be closely adhered to.
3. Performance has only been established with the specimen types listed in the Intended Use. Other specimen types have not been evaluated and should not be used with this assay.
4. This assay has not been evaluated with fingerstick specimens. This test is not authorized for use with fingerstick whole blood.
5. SARS-CoV-2 antibodies may be below detectable levels in samples collected from patients who have been exhibiting symptoms for less than 15 days. Samples should be collected from individuals that are ≥15 days post symptom onset. Samples should not be tested if collected. from individuals less than 15 days post symptom onset.
6. Assay results should be utilized in conjunction with other clinical and laboratory methods to assist the clinician in making individual patient decisions.
7. Assay results should not be used to diagnose or exclude acute COVID-19 infection or to inform infection status. Direct viral nucleic acid detection or antigen detection methods should be performed if acute infection is suspected.
8. False positive results may occur due to cross-reactivity from pre-existing antibodies or other possible causes.
9. A negative result for an individual subject indicates absence of detectable anti-SARS-CoV-2 antibodies. Negative results do not preclude SARS-CoV-2 infection and should not be used as the sole basis for patient management decisions. The sensitivity of this assay early after infection is unknown.
10. A negative result can occur if the quantity of antibodies for the SARS-CoV-2 virus present in the specimen is below the detection limit of the assay, or the antibodies that are detected are not present during the stage of disease in which a sample is collected.
11. Pedigreed specimens with direct evidence of antibodies to non-SARS-CoV-2 coronavirus (common cold) strains such as HKU1, NL63, OC43, or 229E have not been evaluated with this assay.
12. If the results are inconsistent with clinical evidence, additional testing is suggested to confirm the result.
13. It is not known at this time if the presence of antibodies to SARS-CoV-2 confers immunity to infection.
14. A positive result may not indicate previous SARS-CoV-2 infection. Consider other information including clinical history and local disease prevalence, in assessing the need for a second but different serology test to confirm an immune response.
15. The UBI® SARS-CoV-2 ELISA is authorized for use with a manual assay procedure. Assay performance has not been established for use on automated instrument platforms.
16. Not for the screening of donated blood.

### Conditions of Authorization for the Laboratory

The UBI® SARS-CoV-2 ELISA Letter of Authorization, along with the authorized Fact Sheet for Healthcare Providers, the authorized Fact Sheet for Patients, and authorized labeling are available on the FDA website (Website: www.fda.gov/medical-devices/coronavirus-disease-2019-covid-19-emergency-use-authorizations-medical-devices/vitro-diagnostics-euas).

Authorized laboratories using the UBI® SARS-CoV-2 ELISA must adhere to the Conditions of Authorization indicated in the Letter of Authorization as listed below:
1. Authorized laboratories (“Laboratories certified under the Clinical Laboratory Improvement Amendments of 1988 (CLIA), 42 U.S.C. § 263a, that meet requirements to perform high complexity tests” as “authorized laboratories”) using the UBI® SARS-CoV-2 ELISA must include with test result reports, all authorized Fact Sheets. Under exigent circumstances, other appropriate methods for disseminating these Fact Sheets may be used, which may include mass media.
2. Authorized laboratories must use the UBI® SARS-CoV-2 ELISA as outlined in the authorized labeling. Deviations from the authorized procedures, including the authorized clinical specimen types, authorized control materials, authorized other ancillary reagents and authorized materials required to use the product are not permitted.
3. Authorized laboratories that receive the UBI® SARS-CoV-2 ELISA must notify the relevant public health authorities of their intent to run the assay prior to initiating testing.
4. Authorized laboratories using the UBI® SARS-CoV-2 ELISA must have a process in place for reporting test results to healthcare providers and relevant public health authorities, as appropriate.
5. Authorized laboratories must collect information on the performance of the UBI® SARS-CoV-2 ELISA and report to DMD/OHT7-OIR/OPEQ/CDRH (via email: CDRH EUA-Reporting(at)fda.hhs.gov) and Technical Support (website: www.unitedbiomedical.com/support.html) any suspected occurrence of false positive or false negative results and significant deviations from the established performance characteristics of the assay of which they become aware.
6. All laboratory personnel using the UBI® SARS-CoV-2 ELISA must be appropriately trained in immunoassay techniques and use appropriate laboratory and personal protective equipment when handling this kit and use the UBI® SARS-CoV-2 ELISA in accordance with the authorized labeling. All laboratory personnel using the assay must also be trained in and be familiar with the interpretation of results of the UBIO SARS-CoV-2 ELISA.
7. United Biomedical Inc., authorized distributors, and authorized laboratories using the UBI® SARS-CoV-2 ELISA must ensure that any records associated with this EUA are maintained until otherwise notified by FDA. Such records will be made available to FDA for inspection upon request.

### Performance Evaluation

Performance evaluation studies are described in further detail in Example 11 below.

### 7. Specific Embodiments

(1) A serological diagnostic assay for the detection of viral infection and epidemiological surveillance for COVID-19 comprising an antigenic peptide from the M protein (SEQ ID NO: 1), N protein (SEQ ID NO: 6), and S protein (SEQ ID NO: 20) of SARS-CoV-2.
(2) The serological diagnostic assay of (1), wherein the antigenic peptide comprise an amino acid sequence selected from the group consisting of SEQ ID NOs: 4-5, 17-18, 37-38, 259, 261, 263, 265. 266, 270, 281, 308, 321, 322. 323, and 324 and any combination thereof.
(3) The serological diagnostic assay of (1), wherein the antigenic peptide is selected from the group consisting of SEQ ID NOs: 5, 18, 38, 261. 266, 281. 322 and any combination thereof.
(4) A method for detecting infection by SARS-CoV-2 comprising:
   a) attaching an antigenic peptide selected from the group consisting of SEQ ID NOs: 4-5, 17-18, 23-24, 26, 29-34, 37-38, 259, 261, 263, 265, 266, 270, 281, 308, 321, 322, 323, and 324 and any combination thereof to a solid support,
   b) exposing the antigenic peptide attached to the solid support in (a) to a biological sample containing antibodies from a patient, under conditions conducive to binding of the antibody to the peptide, and
   c) detecting the presence of antibodies bound to the peptide attached to the solid support.
(⁵) The method of (4), wherein the antigenic peptide of (a) is selected from the group consisting of SEQ ID NOs: 5, 18, 38, 261, 266, 281, 322, and any combination thereof.

### B. High-Precision, Site-Directed Peptide Immunogen Constructs for the Prevention of Infection by SARS-CoV-2

The second aspect of the disclosed relief system relates to high-precision, site-directed peptide immunogen constructs for the prevention of infection by SARS-CoV-2.

1. Development of S-RBD peptide immunogen constructs

The present disclosure provides peptide immunogen constructs containing a B cell epitope peptide having about 6 to about 100 amino acids derived from the SARS-CoV-2 receptor binding domain (RBD) of the Spike protein (S-RBD or S1-RBD) (SEQ ID NO: 226) or homologues or variants thereof (e.g., SEQ ID NO: 227). In certain embodiments, the B cell epitope peptide has an amino acid sequence selected from SEQ ID NOs: 23-24, 26-27, 29-34, and 315-319 as shown in Tables 3 and 13.

The B cell epitope can be covalently linked to a heterologous helper cell (Th) epitope derived from a pathogen protein (e.g., SEQ ID NOs: 49-100, as shown in Table 6) directly or through an optional heterologous spacer (e.g., SEQ ID NOs: 101-103 of Table 7). These constructs, containing both designed B cell- and Th-epitopes act together to stimulate the generation of highly specific antibodies that are cross-reactive with S-RBD site (SEQ ID NO: 226) and fragments thereof (e.g., SEQ ID NO: 26).

The phrase “S-RBD peptide immunogen construct” or “S1-RBD peptide immunogen construct” or “peptide immunogen construct”, as used herein, refers to a peptide with more than about 20 amino acids containing (a) a B cell epitope having more than about 6 contiguous amino acid residues from the S-RBD binding site (SEQ ID NOs: 226 or 227), or a variant thereof, such as SEQ ID NOs: 23-24, 26-27, 29-34, and 315-319; (b) a heterologous Th epitope (e.g., SEQ ID NOs: 49-100); and (c) an optional heterologous spacer.

In certain embodiments, the S-RBD peptide immunogen construct can be represented by the formulae:

(Th)ₘ-(A)ₙ-(S-RBD B cell epitope peptide)-X

or

(S-RBD B cell epitope peptide)-(A)ₙ-(Th)ₘ-(Th)ₘ-X

or

(Th)ₘ-(A)ₙ-(S-RBD B cell epitope peptide)-(A)ₙ-(Th)ₘ-X

wherein

Th is a heterologous T helper epitope;

A is a heterologous spacer;

(S-RBD B cell epitope peptide) is a B cell epitope peptide having from 6 to about 35 amino acid residues from S-RBD (SEQ ID NO: 226) or a variant thereof that can elicit antibodies directed against SARS-CoV-2;

X is an α-COOH or α-CONH₂ of an amino acid;

m is from 1 to about 4; and

n is from 0 to about 10.

The S-RBD peptide immunogen constructs of the present disclosure were designed and selected based on a number of rationales, including:
i. the S-RBD B cell epitope peptide can be rendered immunogenic by using a protein carrier or a potent T helper epitope(s);
ii. when the S-RBD B cell epitope peptide is rendered immunogenic and administered to a host, the peptide immunogen construct:
   a. elicits high titer antibodies preferentially directed against the S-RBD B cell epitope(s) and not the protein carrier or helper epitope(s);
   b. generates highly specific antibodies capable of neutralizing SARS-CoV-2; and
   c. generates highly specific antibodies capable of inhibiting the binding of S-RBD to its receptor ACE2.

The disclosed S-RBD peptide immunogen constructs and formulations thereof can effectively function as a pharmaceutical composition or vaccine formulation to prevent and/or treat (COVID-19).

The various components of the disclosed S-RBD peptide immunogen constructs are described in further detail below.

a. B Cell Epitome Peptide from S-RBD

The present disclosure is directed to a novel peptide composition for the generation of high titer antibodies with specificity for the S-RBD site (e.g., SEQ ID NO: 226 or 227) and fragments thereof (e.g., SEQ ID NO: 23-24, 26-27, 29-34, and 315-319). The site-specificity of the peptide immunogen constructs minimizes the generation of antibodies that are directed to irrelevant sites on other regions of S-RBD or irrelevant sites on carrier proteins, thus providing a high safety factor.

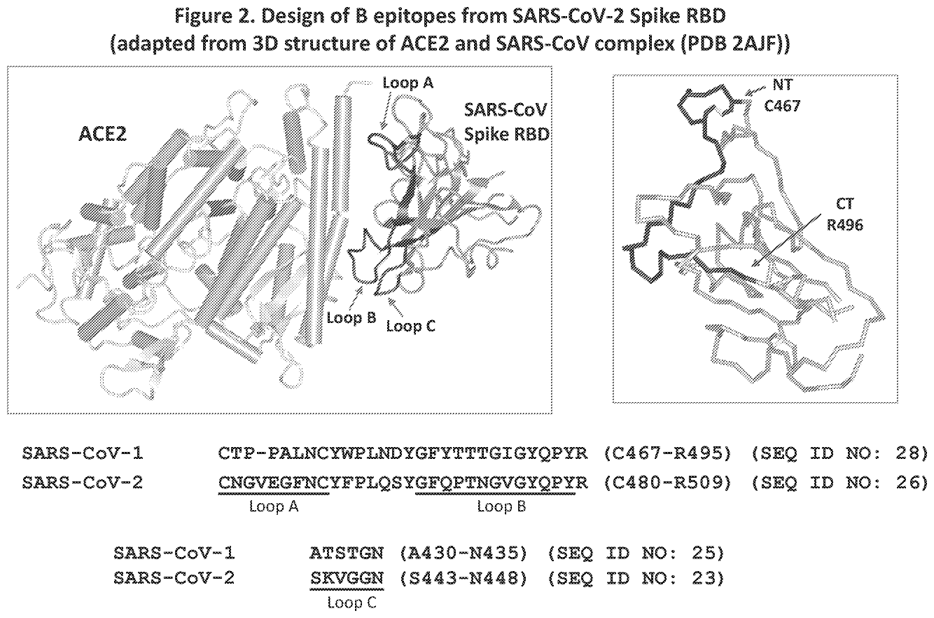

The term “S-RBD” or “S1-RBD”, as used herein, refers to Receptor Binding Domain that contains 200 amino acids and has 8 cysteines forming 4 disulfide bridges between cysteines that binds to its ACE2 receptor (FIG. 2). One aspect of the present disclosure is to prevent and/or treat SARS-CoV-2 infection by active immunization. Thus, the present disclosure is directed to peptide immunogen constructs targeting portions of S-RBD (e.g., SEQ ID NOs: 23-24, 26-27, 29-34, and 315-319) and formulations thereof for elicitation of neutralizing antibodies against SARS-CoV-2 or antibodies that inhibit SARS-CoV-2 binding to the human receptor ACE2.

The B cell epitope portion of the S-RBD peptide immunogen construct can contain between about 6 to about 35 amino acids from the S-RBD site (SEQ ID NO: 226) or a variant thereof. In some embodiments, the B cell epitope peptides have an amino acid sequence selected from SEQ ID NOs: 23-24, 26-27, 29-34, and 315-319, as shown in Tables 3 and 13. The S-RBD B cell epitope peptide of the present disclosure also includes immunologically functional analogues or homologues of S-RBD, including S-RBD sequences from different coronavirus strains, such as SARS-CoV (SEQ ID NO: 21) and MERS-CoV (SEQ ID NO: 22), as shown in Table 3. Functional immunological analogues or homologues of S-RBD B cell epitope peptides include variants that can have substitutions in an amino acid position within the major framework of the protein; a change in overall charge; a covalent attachment to another moiety; or amino acid additions, insertions, or deletions; and/or any combination thereof. In some embodiments, a variant of a sequence from S-RBD includes site directed mutations that replace a natural amino acid residue with a cysteine residue to produce a peptide that can be constrained by a disulfide bond (e.g., SEQ ID NOs: 24, 32, and 34).

Antibodies generated from the peptide immunogen constructs containing B cell epitopes from S-RBD are highly specific and cross-reactive with the full-length S-RBD binding site (e.g., SEQ ID NO: 226) or fragments thereof (e.g., SEQ ID NO: 26). Based on their unique characteristics and properties, antibodies elicited by the disclosed S-RBD peptide immunogen constructs are capable of providing a prophylactic approach to SARS-CoV-2 infection.

b. Heterologous T helper Cell Epitopes (Th Epitopes)

The present disclosure provides peptide immunogen constructs containing a B cell epitope from S-RBD covalently linked to a heterologous T helper cell (Th) epitope directly or through an optional heterologous spacer.

The heterologous Th epitope in the peptide immunogen construct enhances the immunogenicity of the S-RBD B cell epitope peptide, which facilitates the production of specific high titer antibodies directed against the optimized S-RBD B cell epitope peptide screened and selected based on design rationales.

The term “heterologous”, as used herein, refers to an amino acid sequence that is derived from an amino acid sequence that is not part of, or homologous with, the wild-type sequence of S-RBD. Thus, a heterologous Th epitope is a Th epitope derived from an amino acid sequence that is not naturally found in S-RBD (i.e., the Th epitope is not autologous to S-RBD). Since the Th epitope is heterologous to S-RBD, the natural amino acid sequence of S-RBD is not extended in either the N-terminal or C-terminal directions when the heterologous Th epitope is covalently linked to the S-RBD B cell epitope peptide.

The heterologous Th epitope of the present disclosure can be any Th epitope that does not have an amino acid sequence naturally found in S-RBD. The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e., little, if any, of the antibodies generated by the S-RBD peptide immunogen constructs will be directed towards the Th epitope, thus allowing a very focused immune response directed to the targeted B cell epitope peptide of the S-RBD molecule.

Th epitopes of the present disclosure include, but are not limited to, amino acid sequences derived from foreign pathogens, as exemplified in Table 6 (e.g., SEQ ID NOs: 49-100). In certain embodiments, the heterologous Th epitopes employed to enhance the immunogenicity of the S-RBD B cell epitope peptide are derived from natural pathogens EBV BPLF1 (SEQ ID NO: 93), EBV CP (SEQ ID NO: 91), Clostridium Tetani (SEQ ID NOs: 82-87), Cholera Toxin (SEQ ID NO: 81), and Schistosoma mansoni (SEQ ID NO: 100), as well as those idealized artificial Th epitopes derived from Measles Virus Fusion protein (MVF 49-66) and Hepatitis B Surface Antigen (HBsAg 67-79) in the form of either single sequence (e.g., SEQ ID NOs: 49-52, 54-57, 59-60, 62-63, 65-66 for MVF and SEQ ID NOs: 67-71, 73-74, 76-78 for HBsAg) or combinatorial sequences (e.g., SEQ ID NOs: 53, 58, 61, 64 for MvF and SEQ ID NOs: 72 and 75 for HBsAg). The combinatorial idealized artificial Th epitopes contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: SEQ ID NOs: 53, 58, 61, 64, 72, and 75, which are shown in Table 6. Th epitope peptides of the present invention provide broad reactivity and immunogenicity to animals and patients from genetically diverse populations.

c. Heterologous Spacer

The disclosed S-RBD peptide immunogen constructs optionally contain a heterologous spacer that covalently links the S-RBD B cell epitope peptide to the heterologous T helper cell (Th) epitope.

As discussed above, the tern) “heterologous”, refers to an ammo acid sequence that is derived from an amino acid sequence that is not part of, or homologous with, the natural type sequence of S-RBD. Thus, the natural amino acid sequence of S-RBD is not extended in either the N-terminal or C-terminal directions when the heterologous spacer is covalently linked to the S-RBD B cell epitope peptide because the spacer is heterologous to the S-RBD sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the S-RBD peptide immunogen construct. Structurally, the spacer provides a physical separation of the Th epitope from the B cell epitope of the S-RBD fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 103), where Xaa is any amino acid, and preferably aspartic acid.

The spacer can also provide functional features to the S-RBD peptide immunogen construct. For example, the spacer can be designed to change the overall charge of the S-RBD peptide immunogen construct, which can affect the solubility of the peptide immunogen construct. Additionally, changing the overall charge of the S-RBD peptide immunogen construct can affect the ability of the peptide immunogen construct to associate with other compounds and reagents. As discussed in further detail below, the S-RBD peptide immunogen construct can be formed into a stable immunostimulatory complex with a highly charged oligonucleotide, such as CpG oligomers, through electrostatic association. The overall charge of the S-RBD peptide immunogen construct is important for the formation of these stable immunostimulatory complexes.

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally-occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the S-RBD peptide immunogen construct can be covalently linked at either N- or C-terminal end of the Th epitope and the S-RBD B cell epitope peptide. In some embodiments, the spacer is covalently linked to the C-terminal end of the Th epitope and to the N-terminal end of the S-RBD B cell epitope peptide. In other embodiments, the spacer is covalently linked to the C-terminal end of the S-RBD B cell epitope peptide and to the N-terminal end of the Th epitope. In certain embodiments, more than one spacer can be used, for example, when more than one Th epitope is present in the S-RBD peptide immunogen construct. When more than one spacer is used, each spacer can be the same as each other or different. Additionally, when more than one Th epitope is present in the S-RBD peptide immunogen construct, the Th epitopes can be separated with a spacer, which can be the same as, or different from, the spacer used to separate the Th epitope from the S-RBD B cell epitope peptide. There is no limitation in the arrangement of the spacer in relation to the Th epitope or the S-RBD B cell epitope peptide.

In certain embodiments, the heterologous spacer is a naturally occurring amino acid or a non-naturally occurring amino acid. In other embodiments, the spacer contains more than one naturally occurring or non-naturally occurring amino acid. In specific embodiments, the spacer is Lys-, Gly-, Lys-Lys-Lys-, (α, ε-N)Lys, ε-N-Lys-Lys-Lys-Lys (SEQ ID NO: 101), or Lys-Lys-Lys-ε-N-Lys (SEQ ID NO: 102).

d. Specific Embodiments of the S-RBD Peptide Immunogen Constructs

In certain embodiments, the S-RBD peptide immunogen constructs can be represented by the following formulae:

(Th)ₘ-(A)ₙ-(S-RBD B cell epitope peptide)-X

or

(S-RBD B cell epitope peptide)-(A)ₙ-(Th)ₘ-(Th)ₘ-X

or

(Th)ₘ-(A)ₙ-(S-RBD B cell epitope peptide)-(A)ₙ-(Th)ₘ-X

wherein

Th is a heterologous T helper epitope;

A is a heterologous spacer;

(S-RBD B cell epitope peptide) is a B cell epitope peptide having from 6 to 35 amino acid residues from S-RBD (SEQ ID NO: 226 or 227) or a variant thereof that is able to generate antibodies capable of neutralizing SARS-CoV-2 or inhibiting the binding of S-RBD to its receptor ACE2;

X is an α-COOH or α-CONH₂ of an amino acid;

m is from 1 to about 4; and

n is from 0 to about 10.

The B cell epitope peptide can contain between about 6 to about 35 amino acids from portion of the full-length S-RBD polypeptide represented by SEQ ID NO: 226. In some embodiments, the B cell epitope has an amino acid sequence selected from any of SEQ ID NOs: 23-24, 26-27, 29-34, and 315-319, as shown in Tables 3 and 13.

The heterologous Th epitope in the S-RBD peptide immunogen construct has an amino acid sequence selected from any of SEQ ID NOs: 49-100, and combinations thereof, shown in Table 6. in some embodiments, more than one Th epitope is present in the S-RBD peptide immunogen construct.

The optional heterologous spacer is selected from any of Lys-, Gly-, Lys-Lys-Lys-, (α, ε-N)Lys, Pro-Pro-Xaa-Pro-Xaa-Pro (SEQ ID NO: 103), ε-N-Lys-Lys-Lys-Lys (SEQ ID NO: 101), Lys-Lys-Lys-ε-N-Lys (SEQ ID NO: 102), and any combination thereof, where Xaa is any amino acid, but preferably aspartic acid. In specific embodiments, the heterologous spacer is ε-N-Lys-Lys-Lys-Lys (SEQ ID NO: 101) or Lys-Lys-Lys-ε-N-Lys (SEQ ID NO: 102).

In certain embodiments, the S-RBD peptide immunogen construct has an amino acid sequence selected from any of SEQ ID NOs: 107-144 as shown in Table 8.

The S-RBD peptide immunogen constructs comprising Th epitopes are produced simultaneously in a single solid-phase peptide synthesis in tandem with the S-RBD fragment. Th epitopes also include immunological analogues of Th epitopes, immunological Th analogues include immune-enhancing analogues, cross-reactive analogues, and segments of any of these Th epitopes that are sufficient to enhance or stimulate an immune response to the S-RBD B cell epitope peptide.

The Th epitope in the S-RBD peptide immunogen construct can be covalently linked at either N- or C-terminal end of the S-RBD B cell epitope peptide. In some embodiments, the Th epitope is covalently linked to the N-terminal end of the S-RBD B cell epitope peptide. In other embodiments, the Th epitope is covalently linked to the C-terminal end of the S-RBD B cell epitope peptide. In certain embodiments, more than one Th epitope is covalently linked to the S-RBD B cell epitope peptide. When more than one Th epitope is linked to the S-RBD B cell epitope peptide, each Th epitope can have the same amino acid sequence or different amino acid sequences. In addition, when more than one Th epitope is linked to the S-RBD B cell epitope peptide, the Th epitopes can be arranged in any order. For example, the Th epitopes can be consecutively linked to the N-terminal end of the S-RBD B cell epitope peptide, or consecutively linked to the C-terminal end of the S-RBD B cell epitope peptide, or a Th epitope can be covalently linked to the N-terminal end of the S-RBD B cell epitope peptide while a separate Th epitope is covalently linked to the C-terminal end of the S-RBDB cell epitope peptide. There is no limitation in the arrangement of the Th epitopes in relation to the S-RBD B cell epitope peptide.

In some embodiments, the Th epitope is covalently linked to the S-RBD B cell epitope peptide directly. In other embodiments, the Th epitope is covalently linked to the S-RBD fragment through a heterologous spacer.

e. Variants, Homologues, and Functional Analogues

Variants and analogues of the above immunogenic peptide constructs that induce and/or cross-react with antibodies to the preferred S-RBD B cell epitope peptides can also be used. Analogues, including allelic, species, and induced variants, typically differ from naturally occurring peptides at one, two, or a few positions, often by virtue of amino acid substitutions. Analogues typically exhibit at least 75%, 80%, 85%, 90%, or 95% sequence identity with natural peptides. Some analogues also include unnatural amino acids or modifications of N- or C-terminal amino acids at one, two, or a few positions.

Variants that are functional analogues can have a substitution in an amino acid position; a change in overall charge; a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan, and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

In a particular embodiment, the functional analogue has at least 50% identity to the original amino acid sequence. In another embodiment, the functional analogue has at least 80% identity to the original amino acid sequence. In yet another embodiment, the functional analogue has at least 85% identity to the original amino acid sequence. In still another embodiment, the functional analogue has at least 90% identity to the original amino acid sequence.

Functional immunological analogues of the Th epitope peptides are also effective and included as part of the present invention. Functional immunological Th analogues can include conservative substitutions, additions, deletions, and insertions of from one to about five amino acid residues in the Th epitope which do not essentially modify the Th-stimulating function of the Th epitope. The conservative substitutions, additions, and insertions can be accomplished with natural or non-natural amino acids, as described above for the S-RBD B cell epitope peptide. Table 6 identifies another variation of a functional analogue for Th epitope peptide. In particular, SEQ ID NOs: 54 and 55 of MVF1 and MvF2 Th are functional analogues of SEQ ID NOs: 62-64 and 65 MvF4 and MvF5, respectively, in that they differ in the amino acid frame by the deletion (SEQ ID NOs: 54 and 55) or the inclusion (SEQ ID NOs: 62-64 and 65) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 54-64) and from Hepatitis Surface protein HBsAg 1-3 Ths (SEQ ID NOs: 67-76).

### 2 Compositions

The present disclosure also provides compositions comprising the disclosed S-RBD immunogen peptide constructs.

a. Peptide Compositions

Compositions containing the disclosed S-RBD peptide immunogen constructs can be in liquid or solid/lyophilized form. Liquid compositions can include water, buffers, solvents, salts, and/or any other acceptable reagent that does not alter the structural or functional properties of the S-RBD peptide immunogen constructs. Peptide compositions can contain one or more of the disclosed S-RBD peptide immunogen constructs.

b. Pharmaceutical Compositions

The present disclosure is also directed to pharmaceutical compositions containing the disclosed S-RBD peptide immunogen constructs.

Pharmaceutical compositions can contain carriers and/or other additives in a pharmaceutically acceptable delivery system. Accordingly, pharmaceutical compositions can contain a pharmaceutically effective amount of an S-RBD peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the S-RBD peptide immunogen constructs without having any specific antigenic effect itself. Adjuvants used in the pharmaceutical composition can include oils, oil emulsions, aluminum salts, calcium salts, immune stimulating complexes, bacterial and viral derivatives, virosomes, carbohydrates, cytokines, polymeric microparticles. In certain embodiments, the adjuvant can be selected from alum (potassium aluminum phosphate), aluminum phosphate (e.g. ADJU-PHOS®), aluminum hydroxide (e.g. ALHYDROGEL®), calcium phosphate, incomplete Freund's adjuvant (IFA), Freund's complete adjuvant, MF59, adjuvant 65, Lipovant, ISCOM, liposyn, saponin, squalene, L121, ENTULSIGEN®, EmulsIL-6n®, monophosphoryl lipid A (MPL), Quil A, QS21, MONTANIDE® ISA 35, ISA 50V, ISA 50V2, ISA 51, ISA 206, ISA 720, liposomes, phospholipids, peptidoglycan, lipopolysaccahrides (LPS), ASO1, ASO2, ASO3, ASO4, AF03, lipophilic phospholipid (lipid A), gamma inulin, algammulin, glucans, dextrans, glucomannans, galactomannans, levans, xylans, dimethyldioctadecylammonium bromide (DDA), as well as the other adjuvants and emulsifiers.

In some embodiments, the pharmaceutical composition contains MONTANEDE™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e., w/o/w) emulsion with EMULSIGEN or EMULSIGEN D as the adjuvant.

Pharmaceutical compositions can also include pharmaceutically acceptable additives or excipients. For example, pharmaceutical compositions can contain antioxidants, binders, buffers, bulking agents, carriers, chelating agents, coloring agents, diluents, disintegrants, emulsifying agents, fillers, gelling agents, pH buffering agents, preservatives, solubilizing agents, stabilizers, and the like.

Pharmaceutical compositions can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the S-RBD peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for subcutaneous, intradermal, or intramuscular administration. Pharmaceutical compositions suitable for other modes of administration can also be prepared, including oral and intranasal applications.

Pharmaceutical compositions can also be formulated in a suitable dosage unit form. In some embodiments, the pharmaceutical composition contains from about 0.1 μg to about 1 mg of the S-RBD peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight, and general health of the subject as is well known in the therapeutic arts.

In some embodiments, the pharmaceutical composition contains more than one S-RBD peptide immunogen construct. A pharmaceutical composition containing a mixture of more than one S-RBD peptide immunogen construct to allow for synergistic enhancement of the immunoefficacy of the constructs. Pharmaceutical compositions containing more than one S-RBD peptide immunogen construct can be more effective in a larger genetic population due to a broad MHC class II coverage thus provide an improved immune response to the S-RBD peptide immunogen constructs.

In some embodiments, the pharmaceutical composition can contain an S-RBD peptide immunogen construct selected from SEQ ID NOs: 107-144 of Table 8, as well as homologues, analogues and/or combinations thereof.

In certain embodiments, S-RBD peptide immunogen constructs (SEQ ID NOs: 126 and 127) with heterologous Th epitopes derived from MvF and HBsAg in a combinatorial form (SEQ ID NOs: 59-61, 67-72) can be mixed in an equimolar ratio for use in a formulation to allow for maximal coverage of a host population having a diverse genetic background.

Furthermore, the antibody response elicited by the S-RBD peptide immunogen constructs (e.g., utilizing UBITh®1; SEQ ID NOs: 107-116) are mostly (>90%) focused on the desired cross-reactivity against the B cell epitope peptide of S-RBD without much, if any, directed to the heterologous Th epitopes employed for immunogenicity enhancement. This is in sharp contrast to the conventional protein such as KLH or other biological protein carriers used for such S-RBD peptide immunogenicity enhancement.

In other embodiments, pharmaceutical compositions comprising a peptide composition of, for example, a mixture of the S-RBD peptide immunogen constructs in contact with mineral salts including Alum gel (ALHYDROGEL) or Aluminum phosphate (ADJUPHOS) as adjuvant to form a suspension formulation was used for administration to hosts.

Pharmaceutical compositions containing an S-RBD peptide immunogen construct can be used to elicit an immune response and produce antibodies in a host upon administration.

c. Pharmaceutical Compositions also Containing Endogenous SARS-CoV-2 Th and CTL Epitope Peptides

Pharmaceutical compositions containing a S-RBD peptide immunogen construct can also include an endogenous SARS-CoV-2 T helper epitope peptide and/or CTL epitope peptide separate from (i.e., not covalently linked to) the peptide immunogen construct. The presence of Th and CTL epitopes in pharmaceutical/vaccine formulations prime the immune response in treated subjects by initiating antigen specific T cell activation, which correlates to protection from SARS-CoV-2 infection. Additionally, formulations that include carefully selected endogenous Th epitopes and/or CTL epitopes presented on proteins from SARS-CoV-2 can produce broad cell mediated immunity, which also makes the formulations effective in treating and protecting subjects having diverse genetic makeups.

Including one or more separate peptides containing endogenous SARS-CoV-2 Th epitopes and/or CTL epitopes in a pharmaceutical composition containing S-RBD peptide immunogen constructs brings the peptides in close contact to each other, which allows the epitopes to be seen and processed by antigen presenting B cells, macrophages, dendritic cells, etc. These cells process the antigens and present them to the surface to be in contact with the B cell for antibody generation and T cells to trigger further T cell responses to help mediate killing of the virus infected cells.

In some embodiments, the pharmaceutical composition contains one or more endogenous SARS-CoV-2 Th epitope peptide separate from the S-RBD peptide immunogen construct. In certain embodiments, the endogenous SARS-CoV-2 Th epitope peptide is from the N protein or the S protein of SARS-CoV-2. In specific embodiments, the endogenous SARS-CoV-2 Th epitope peptide is selected from the group consisting of SEQ ID NOs: 13, 39-41, and 44 (Table 5), SEQ ID NOs: 161-165 (Table 8), and any combination thereof. The endogenous SARS-CoV-2 Th epitope peptides of SEQ ID NOs: 161-165 (Table 8) correspond to the sequences of SEQ ID NOs: 39, 40, 44, 41, and 13, respectively, but contain a Lys-Lys-Lys (KKK) tail at the N-terminus. The endogenous Th epitopes of SEQ ID NOs: 161-165 are particularly useful when used in a pharmaceutical composition that has been formulated into an immunostimulatory complex with a CpG oligonucleotide (ODN), because the cationic KKK tail is capable of interacting with the CpG ODN through electrostatic association. The use of endogenous SARS-CoV-2 Th epitopes in the peptide immunogen construct can enhance the immunogenicity of the S-RBD B cell epitope peptide to facilitates the production of specific high titer antibodies, upon infection, directed against the optimized S-RBD B cell epitope peptide screened and selected based on design rationales.

In other embodiments, the pharmaceutical composition contains one or more endogenous SARS-CoV-2 CTL epitope peptide separate from the S-RBD peptide immunogen construct. In certain embodiments, the endogenous SARS-CoV-2 CTL epitope peptide is from the N protein or the S protein of SARS-CoV-2. In specific embodiment, the endogenous SARS-CoV-2 CTL epitope peptide is selected from the group consisting of SEQ ID NOs: 9-12, 14-16, 19, 35-36, 42-43, 45-48 (Table 4), SEQ ID NOs: 145-160 (Table 8), and any combination thereof. The endogenous SARS-CoV-2 CTL epitope peptides of SEQ ID NOs: 145-160 correspond to the sequences of SEQ ID NOs: 45, 42, 46, 36, 48, 43, 47, 35, 12, 11, 10, 14, 19, 9, 16, and 15, respectively, but contain a Lys-Lys-Lys (KKK) tail at the N-terminus. The endogenous CTL epitopes of SEQ ID NOs: 145-160 are particularly useful when used in a pharmaceutical composition that has been formulated into an immunostimulatory complex with a CpG oligonucleotide (ODN), because the cationic KKK tail is capable of interacting with the CpG ODN through electrostatic association. The use of endogenous SARS-CoV-2 CTL epitopes in the peptide immunogen construct can enhance the immunogenicity of the S-RBD B cell epitope peptide to facilitates the production of specific high titer antibodies, upon infection, directed against the optimized S-RBD B cell epitope peptide screened and selected based on design rationales.

In some embodiments, the pharmaceutical composition contains one or more S-RBD peptide immunogen constructs (SEQ ID NOs: 107-144 or any combination thereof) together with one or more separate peptides containing an endogenous SARS-CoV-2 Th epitope peptide (SEQ NOs: 13, 39-41, 44, 161-165, or any combination thereof) and/or an endogenous SARS-CoV-2 CTL epitope peptides (SEQ lID NOs: 9-12, 14-16, 19, 35-36, 42-43, 45-48, 145-160, or any combination thereof).

d. Immunostimulatory Complexes

The present disclosure is also directed to pharmaceutical compositions containing an S-RBD peptide immunogen construct in the form of an immunostimulatory complex with a CpG oligonucleotide. Such immunostimulatory complexes are specifically adapted to act as an adjuvant and/or as a peptide immunogen stabilizer. The immunostimulatory complexes are in the form of a particulate, which can efficiently present the S-RBD peptide immunogen to the cells of the immune system to produce an immune response. The immunostimulatory complexes may be formulated as a suspension for parenteral administration. The immunostimulatory complexes may also be formulated in the form of water in oil (w/o) emulsions, as a suspension in combination with a mineral salt or with an in-situ gelling polymer for the efficient delivery of the S-RBD peptide immunogen construct to the cells of the immune system of a host following parenteral administration.

The stabilized immunostimulatory complex can be formed by complexing an S-RBD peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the S-RBD peptide immunogen construct is designed to contain a cationic portion that is positively charged at a pH in the range of 5.0 to 8.0. The net charge on the cationic portion of the S-RBD peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the S-RBD peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the S-RBD peptide immunogen construct is the heterologous spacer. In certain embodiments, the cationic portion of the S-RBD peptide immunogen construct has a charge of +4 when the spacer sequence is (α, ε-N)Lys, (α,ε-N)-Lys-Lys-Lys-Lys (SEQ ID NO: 101), or Lys-Lys-Lys-ε-N-Lys (SEQ ID NO: 102).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X¹ is selected from the group consisting of A (adenine), G (guanine) and T (thymine); and X² is C (cytosine) or T (thymine). Or the anionic oligonucleotide is represented by the formula: 5′ (X³)₂CG(X⁴)₂ 3′ wherein C and G are unmethylated; and X³ is selected from the group consisting of A, T or G; and X⁴ is C or T. In specific embodiments, the CpG oligonucleotide has the sequence of CpG1: 5′ TCg TCg TTT TgT CgT TTT gTC gTT TTg TCg TT 3′ (fully phosphorothioated) (SEQ ID NO: 104), CpG2: 5′ Phosphate TCg TCg TTT TgT CgT TTT gTC gTT 3′ (fully phosphorothioated) (SEQ ID NO: 105), or CpG3 5′ TCg TCg TTT TgT CgT TTT gTC gTT 3′ (fully phosphorothioated) (SEQ ID NO: 106).

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

The present disclosure is also directed to pharmaceutical compositions, including formulations, for the prevention and/or treatment COVID-19. In some embodiments, pharmaceutical compositions comprising a stabilized immunostimulatory complex, which is formed through mixing a CpG oligomer with a peptide composition containing a mixture of the S-RBD peptide immunogen constructs (e.g., SEQ ID NOs: 107-144) through electrostatic association, to further enhance the immunogenicity of the S-RBD peptide immunogen constructs and elicit antibodies that are cross-reactive with the S-RBD binding site of SEQ ID NOs: 226 or fragments thereof, such as SEQ ID NO: 26.

In yet other embodiments, pharmaceutical compositions contain a mixture of the S-RBD peptide immunogen constructs (e.g., any combination of SEQ ID NOs: 107-144) in the form of a stabilized immunostimulatory complex with CpG oligomers that are, optionally, mixed with mineral salts, including Alum gel (ALHYDROGEL) or Aluminum phosphate (ADJUPHOS) as an adjuvant with high safety factor, to form a suspension formulation for administration to hosts.

### 3. Antibodies

The present disclosure also provides antibodies elicited by the S-RBD peptide immunogen constructs.

The present disclosure provides S-RBD peptide immunogen constructs and formulations thereof, cost effective in manufacturing, and optimal in their design that are capable of eliciting high titer neutralizing antibodies against SARS-CoV-2 and inhibiting the binding of S-RBD to its receptor ACE2 with a high responder rate in immunized hosts. In some embodiments, S-RBD peptide immunogen constructs for eliciting antibodies comprise a hybrid of a S-RBD peptide targeting the S-RBD site that is around SARS-CoV-2 S₄₈₀₋₅₀₉ region (SEQ ID NOs: 26) within the full-length S-RBD (SEQ ID NO: 226) that is linked to a heterologous Th epitope derived from pathogenic proteins such as Measles Virus Fusion (MVF) protein and others (e.g., SEQ ID NOs: 49-100 of Table 6) and/or a SARS-CoV-2 derived endogenous Th epitope (SEQ ID NOs: 13, 39-41, and 44 of Table 5 and 161-165 of Table 8) through an optional heterologous spacer. The B cell epitope and Th epitope peptides of the S-RBD peptide immunogen constructs act together to stimulate the generation of highly specific antibodies cross-reactive with the full-length S-RBD site (SEQ ID NO: 226) or fragments thereof (e.g., SEQ ID NO: 26).

Traditional methods for immunopotentiating a peptide, such as through chemical coupling to a carrier protein, for example, Keyhole Limpet Hemocyanin (KLH) or other carrier proteins such as Diphtheria toxoid (DT) and Tetanus Toxoid (TT) proteins, typically result in the generation of a large amount of antibodies directed against the carrier protein. Thus, a major deficiency of such peptide-carrier protein compositions is that most (>90%) of antibodies generated by the immunogen are the non-functional antibodies directed against the carrier protein KLH, DT or TT, which can lead to epitopic suppression.

Unlike the traditional method for immunopotentiating a peptide, the antibodies generated from the disclosed S-RBD peptide immunogen constructs (e.g., SEQ ID NOs: 107-144) are capable of binding with highly specificity to the full-length S-RBD site (SEQ NO: 226) or fragments thereof (e.g., SEQ ID NO: 26) with little, if any, antibodies directed against the heterologous Th epitope (e.g., SEQ ID NOs: 49-100), the endogenous SARS-CoV-2 Th epitope (SEQ ID NOs: 13, 39-41,44, and 161-165), or the optional heterologous spacer.

### 4. Methods

The present disclosure is also directed to methods for making and using the S-RBD peptide immunogen constructs, compositions, and pharmaceutical compositions.

a. Methods for Manufacturing the S-RBD Peptide Immunogen Construct

The disclosed S-RBD peptide immunogen constructs can be made by chemical synthesis methods well known to the ordinarily skilled artisan (see, e.g., Fields, G. B., et al., 1992). The S-RBD peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of S-RBD peptide immunogen constructs comprising combinatorial library peptides for Th epitopes can be accomplished by providing a mixture of alternative amino acids for coupling at a given variable position.

After complete assembly of the desired S-RBD peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art.

The quality of peptides produced by this chemical process can be controlled and defined and, as a result, reproducibility of S-RBD peptide immunogen constructs, immunogenicity, and yield can be assured. A detailed description of the manufacturing of the S-RBD peptide immunogen construct through solid phase peptide synthesis is provided in Example 1.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs.

Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The S-RBD peptide immunogen constructs can also be made using recombinant DNA technology including nucleic acid molecules, vectors, and/or host cells. As such, nucleic acid molecules encoding the S-RBD peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the S-RBD peptide immunogen construct and immunologically functional analogues thereof. For example, methods can include a step of incubating a host cell containing an expression vector containing a nucleic acid molecule encoding an S-RBD peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

b. Methods for the Manufacturing of Immunostimulatory Complexes

Various exemplary embodiments also encompass methods of producing the immunostimulatory complexes comprising S-RBD peptide immunogen constructs and CpG oligodeoxynucleotide (ODN) molecule. Stabilized immunostimulatory complexes (ISC) are derived from a cationic portion of the S-RBD peptide immunogen construct and a polyanionic CpG ODN molecule. The self-assembling system is driven by electrostatic neutralization of charge. Stoichiometry of the molar charge ratio of cationic portion of the S-RBD peptide immunogen construct to anionic oligomer determines extent of association. The non-covalent electrostatic association of S-RBD peptide immunogen construct and CpG ODN is a completely reproducible process. The peptide/CpG ODN immunostimulatory complex aggregates, which facilitate presentation to the “professional” antigen presenting cells (APC) of the immune system thus further enhancing the immunogenicity of the complexes. These complexes are easily characterized for quality control during manufacturing. The peptide/CpG ISC are well tolerated in vivo. This novel particulate system comprising CpG ODN and S-RBD peptide immunogen constructs is designed to take advantage of the generalized B cell mitogenicity associated with CpG ODN use and to promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen presenting cells (APC). Consequently, such formulations are novel conceptually and offer potential advantages by promoting the stimulation of immune responses by alternative mechanisms.

c. Methods for the Manufacturing of Pharmaceutical Compositions

Various exemplary embodiments also encompass pharmaceutical compositions containing S-RBD peptide immunogen constructs. In certain embodiments, the pharmaceutical compositions employ water in oil emulsions and in suspension with mineral salts.

In order for a pharmaceutical composition to be used by a large population, safety becomes another important factor for consideration. Despite there has been use of water-in-oil emulsions in many clinical trial sAlum remains the major adjuvant for use in formulations due to its safety. Alum or its mineral salts Aluminum phosphate (ADJUPHOS) are, therefore, frequently used as adjuvants in preparation for clinical applications.

Other adjuvants and immunostimulating agents include 3 De-O-acylated monophosphoryl lipid A (MPL) or 3-DMP, polymeric or monomeric amino acids, such as polyglutarnic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alisoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) THERAMIDE™), or other bacterial cell wall components. Oil-in-water emulsions include MF59 (see WO 1990/014837 to Van Nest, G., et al., which is hereby incorporated by reference in its entirety), containing 5% Squalene, 0.5% TWEEN 80, and 0.5% Span 85 (optionally containing various amounts of MTP-PE) formulated into submicron particles using a microfluidizer; SAF, containing 10% Squalene, 0.4% TWEEN 80, 5% pluronic-blocked polymer L121, and thr-MDP, either microfluidized into a submicron emulsion or vortexed to generate a larger particle size emulsion; and the RIBI™ adjuvant system (RAS) (RIBI ImmunoChern, Hamilton, Mont.) containing 2% squalene, 0.2% TWEEN 80, and one or more bacterial cell wall components selected from the group consisting of monophosphoryllipid A (MPL), trehalose dimycolate (TDM), and cell wall skeleton (CWS), preferably MPL+CWS (Detox™). Other adjuvants include Complete Freund's Adjuvant (CFA), Incomplete Freund's Adjuvant (IFA), and cytokines, such as interleukins IL-1, IL-2, and IL-12), macrophage colony stimulating factor (M-CSF), and tumor necrosis factor (TNF-α).

The choice of an adjuvant depends on the stability of the immunogenic formulation containing the adjuvant, the route of administration, the dosing schedule, the efficacy of the adjuvant for the species being immunized, and, in humans, a pharmaceutically acceptable adjuvant is one that has been approved or is approvable for human administration by pertinent regulatory bodies. For example, alum, MPL or Incomplete Freund's adjuvant (Chang, J. C. C., et al., 1998), which is hereby incorporated by reference in its entirety) alone or optionally all combinations thereof are suitable for human administration.

The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers, and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immunostimulating agents (i.e., adjuvants).

The pharmaceutical compositions of the present invention can further include a suitable delivery vehicle. Suitable delivery vehicles include, but are not limited to viruses, bacteria, biodegradable microspheres, microparticles, nanoparticles, liposomes, collagen minipellets, and cochleates.

In some embodiments, the pharmaceutical composition is prepared by combining one or more S-RBD peptide immunogen constructs (SEQ ID NOs: 107-144 or any combination thereof) together with one or more separate peptides containing an endogenous SARS-CoV-2 Th epitope peptides (SEQ ID NOs: 13, 39-41, 44, 161-165, or any combination thereof) and/or an endogenous SARS-CoV-2 CTL epitope peptides (SEQ ID NOs: 9-12, 14-16, 19, 35-36, 42-43, 45-48, 145-160, or any combination thereof) in the form of an immunostimulatory complex containing a CpG ODN.

d. Methods of Using Pharmaceutical Compositions

The present disclosure also includes methods of using pharmaceutical compositions containing S-RBD peptide immunogen constructs.

In certain embodiments, the pharmaceutical compositions containing S-RBD peptide immunogen constructs can be used for the prevention and/or treatment of COVID-19.

In some embodiments, the methods comprise administering a pharmaceutical composition comprising a pharmacologically effective amount of an S-RBD peptide immunogen construct to a host in need thereof. In certain embodiments, the methods comprise administering a pharmaceutical composition comprising a pharmacologically effective amount of an S-RBD peptide immunogen construct to a warm-blooded animal (e.g., humans, macaques, guinea pigs, mice, cat, etc.) to elicit highly specific antibodies cross-reactive with the S-RBD site that is around SARS-CoV-2 S₄₈₀₋₅₀₉ region (SEQ ID NO: 26) within the full-length sequence of S-RBD (SEQ ID NO: 226) or S-RBD sequences from other coronaviruses (e.g., SARS-CoV or MERS-CoV).

In certain embodiments, the pharmaceutical compositions containing S-RBD peptide immunogen constructs can be used to prevent COVID-19 caused by infection by SARS-CoV-2.

e. In Vitro Functional Assays and In Vivo Proof of Concept Studies

Antibodies elicited in immunized hosts by the S-RBD peptide immunogen constructs can be used in in vitro functional assays. These functional assays include, but are not limited to:

(1) in vitro binding to S-RBD site (SEQ ID NO: 26) within S-RBD (SEQ ID NO: 226) by serological assays including ELISA assays;

(2) in vitro inhibition of S-RBD binding to its receptor ACE2;

(3) in vitro neutralization of infection mediated by SARS-CoV-2 of host cells;

(4) in vivo prevention of SARS-CoV-2 mediated infection of vaccinated host in animal models.

### 5. Specific Embodiments

(1) An S-RBD peptide immunogen construct having about 20 or more amino acids, represented by the formulae:

(Th)ₘ-(A)ₙ-(S-RBD B cell epitope peptide)-X

or

(S-RBD B cell epitope peptide)-(A)ₙ-(Th)ₘ-(Th)ₘ-X

or

(Th)ₘ-(A)ₙ-(S-RBD B cell epitope peptide)-(A)ₙ-(Th)ₘ-X

wherein
Th is a heterologous T helper epitope;
A is a heterologous spacer;
(S-RBD B cell epitope peptide) is a B cell epitope peptide haying from 6 to about 35 amino acid residues from S-RBD (SEQ ID NO: 226) or variants thereof;
X is an α-COOH or α-CONH₂ of an amino acid;
m is from 1 to about 4; and
n is from 0 to about 10.
(2) The S-RBD peptide immunogen construct according to (1), wherein the S-RBD B cell epitope peptide forms intra-disulfide bond to allow local constraint of the epitope selected from the group consisting of SEQ ID NOs: 23-24, 26-27, and 29-34.
(3) The S-RBD peptide immunogen construct according to (1), wherein the heterologous helper is selected from the group consisting of SEQ ID NOs: 49-100.
(4) The S-RBD peptide immunogen construct according to (1), wherein the S-RBD B cell epitope peptide is selected from the group consisting of SEQ ID NOs: 23-24, 26-27, 29-34, and 315-319 and the Th epitope is selected from the group consisting of SEQ ID NOs: 49-100.
(5) The S-RBD peptide immunogen construct according to (1), wherein the peptide immunogen construct is selected from the group consisting of SEQ ID NOs: 107-144.
(6) An S-RBD peptide immunogen construct comprising:
a. a B cell epitope comprising from about 6 to about 35 amino acid residues from the S-RBD sequence of SEQ ID NO: 226;
b. a heterologous T helper epitope comprising an amino acid sequence selected from the group consisting of SEQ ID NOs: 49-100 and any combination thereof; and
c. an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 101), Lys-Lys-Lys-ε-N-Lys (SEQ ID NO: 102), and Pro-Pro-Xaa-Pro-Xaa-Pro (SEQ ID NO: 103), and any combination thereof,
   wherein the B cell epitope is covalently linked to the T helper epitope directly or through the optional heterologous spacer.
   (7) The S-RBD peptide immunogen construct of (6), wherein the B cell epitope is selected from the group consisting of SEQ ID NOs: 23-24, 26-27, 29-34, and 315-319.
   (8) The S-RBD peptide immunogen construct of (6), wherein the optional heterologous spacer is (α, ε-N)Lys, ε-N-Lvs-Lys-Lys-Lys (SEQ ID NO: 101). Lvs-Lys-Lys-ε-N-Lys (SEQ ID NO: 102), or Pro-Pro-Xaa-Pro-Xaa-Pro (SEQ ID NO:103), where Xaa is any amino acid.
   (9) The S-RBD peptide immunogen construct of (6), wherein the T helper epitope is covalently linked to the amino- or carboxyl-terminus of the B cell epitope.
   (10) The S-RBD peptide immunogen construct of (6), wherein the T helper epitope is covalently linked to the amino- or carboxyl- of the B cell epitope through the optional heterologous spacer.
   (11) A composition comprising the S-RBD peptide immunogen construct according to (1).
   (12) A pharmaceutical composition comprising:
a. a peptide immunogen construct according to (1); and
b. a pharmaceutically acceptable delivery vehicle and/or adjuvant.
   (13) The pharmaceutical composition of (12), wherein
a. the S-RBD B cell epitope peptide is selected from the group consisting of SEQ ID NOs: 23-24, 26-27, 29-34, and 315-319;
b. the heterologous T helper epitope is selected from the group consisting of SEQ ID NOs: 49-100; and
c. the heterologous spacer is selected from the group consisting of an amino acid, Lys-, Gly-, Lys-Lys-Lys-, (α, ε-N)Lys, ε-N-Lys-Lys-Lys-Lys (SEQ ID NO: 101), Lys-Lys-Lys-ε-N-Lys (SEQ ID NO: 102), and Pro-Pro-Xaa-Pro-Xaa-Pro (SEQ ID NO: 103), and any combination thereof; and
   wherein the S-RBD peptide immunogen construct is mixed with an CpG oligodeoxynucleotide (ODN) to form a stabilized immunostimulatory complex.
   (14) The pharmaceutical composition of (12), wherein
a. the S-RBD peptide immunogen construct is selected from the group consisting of SEQ ID NOs: 107-144; and
   wherein the S-RBD peptide immunogen construct is mixed with an CpG oligodeoxynucleotide (ODN) to form a stabilized immunostimulatory complex.
   (15) The pharmaceutical composition of (14), wherein the pharmaceutical composition further contains a separate peptide containing an endogenous SARS-CoV-2 Th epitope sequence of SEQ ID NOs: 13, 39-41, 44, 161-165, or any combination thereof.
   (16) The pharmaceutical composition of (14), wherein the pharmaceutical composition further contains a separate peptide containing an endogenous SARS-CoV-2 CTL epitope sequence of SEQ ID NOs: 9-12, 14-16, 19, 35-36, 42-43, 45-48, 145-160, or any combination thereof.
   (17) The pharmaceutical composition of (14), wherein the pharmaceutical composition further contains
a. a separate peptide containing an endogenous SARS-CoV-2 Th epitope sequence of SEQ ID NOs: 13, 39-41, 44, 161-165, or any combination thereof; and
b. a separate peptide containing an endogenous SARS-CoV-2 CTL epitope sequence of SEQ ID NOs: 9-12, 14-16, 19, 35-36, 42-43, 45-48, 145-160, or any combination thereof.
   (18) A. method for generating antibodies against S-RBD in an animal comprising administering the pharmaceutical composition according to (12) to the animal.
   (19) A method for generating antibodies against S-RBD in an animal comprising administering the pharmaceutical composition according to (15) to the animal.
   (20) A method for generating antibodies against S-RBD in an animal comprising administering the pharmaceutical composition according to (16) to the animal.
   (21) A method for generating antibodies against S-RBD in an animal comprising administering the pharmaceutical composition according to (17) to the animal.
   (22) An isolated antibody or epitope-binding fragment thereof that specifically binds to the amino acid sequence of SEQ ID NOs: 23-24, 26-27, 29-34, or 226.
   (23) The isolated antibody or epitope-binding fragment thereof according to (22) bound to the S-RBD peptide immunogen construct.
   (24) A composition comprising the isolated antibody or epitope-binding fragment thereof according to (22).
   (25) A method of preventing and/or treating COVID-19 in an animal comprising administering the pharmaceutical composition of (12) to the animal.
   (26) A method of preventing and/or treating COVID-19 in an animal comprising administering the pharmaceutical composition of (15) to the animal.
   (27) A method of preventing and/or treating COVID-19 in an animal comprising administering the pharmaceutical composition of (16) to the animal.
   (28) A method of preventing and/or treating COVID-19 in an animal comprising administering the pharmaceutical composition of (17) to the animal.

### C. Receptor-Based Antiviral Therapies for the Treatment of COVID-19 in Infected Patients

The third aspect of the disclosed relief system relates to receptor-based antiviral therapies for the treatment of COVID-19 in infected patients.

The present disclosure is directed to novel fusion proteins comprising a bioactive molecule and portions of an immunoglobulin molecule. Various aspects of the present disclosure relate to fusion proteins, compositions thereof, and methods for making and using the disclosed fusion proteins. The disclosed fusion proteins are useful for extending the serum half-life of bioactive molecules in an organism.

The following is a detailed description provided to aid those skilled in the art in practicing the present invention. Those of ordinary skill in the art would understand that modifications or variations of the embodiments expressly described herein, which do not depart from the spirit or scope of the information contained herein, are encompassed by the present disclosure. The terminology used in the description is for describing particular embodiments only and is not intended to be limiting of the invention. The section headings used below are for organizational purposes only and are not to be construed as limiting the subject matter described.

### 1. Fusion Protein

As used herein, “fusion protein” or a “fusion polypeptide” is a hybrid protein or polypeptide comprising at least two proteins or peptides linked together in a manner not normally found in nature.

One aspect of the present disclosure is directed to a fusion protein comprising an immunoglobulin (Ig) Fc fragment and a bioactive molecule. The bioactive molecule that is incorporated into the disclosed fusion protein has improved biological properties compared to the same bioactive molecule that is either not-fused or incorporated into a fusion protein described in the prior art (e.g., fusion proteins containing a two chain Fc region). For example, the bioactive molecule incorporated into the disclosed fusion protein has a longer serum half-life compared to its non-fused counterpart. Additionally, the disclosed fusion protein maintains full biological activity of the bioactive molecule without any functional decrease, which is an improvement over the fusion proteins of the prior art that have a decrease in activity due to steric hindrance from a two chain Fc region.

The fusion proteins of the present disclosure provide significant biological advantages to bioactive molecules compared to non-fused bioactive molecules and bioactive molecules incorporated into fusion proteins described in the prior art.

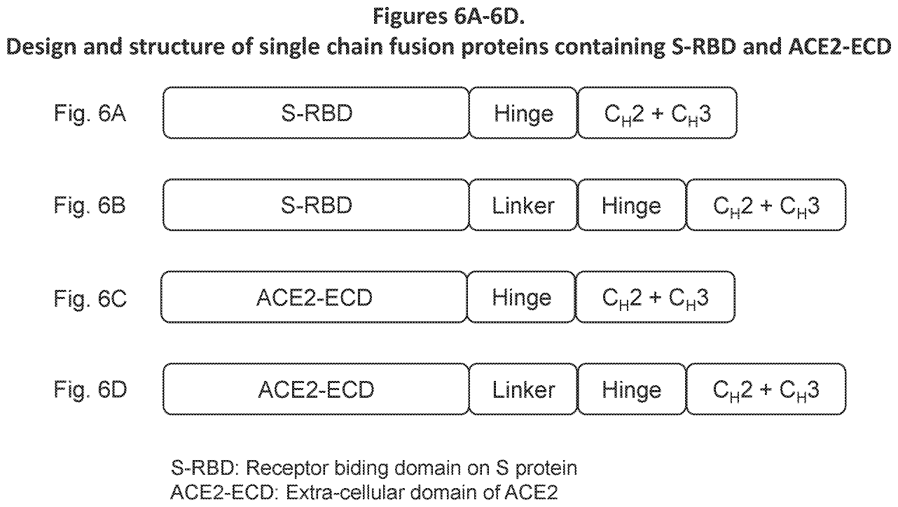

The disclosed fusion protein can have any of the following formulae (also shown in FIGS. 6A-6D):

(B)-(Hinge)-(C_{H}2-C_{H}3)

or

(C_{H}2-C_{H}3)-(Hinge)-(B)

or

(B)-(L)ₘ-(Hinge)-(C_{H}2-C_{H}3)

or

(C_{H}2-C_{H}3)-(Hinge)-(L)ₘ-(B)

wherein

“B” is a bioactive molecule;

“Hinge” is a hinge region of an IgG molecule;

“C_{H}2-CH3” is the C_{H}2 and C_{H}3 constant region domains of an IgG heavy chain;

“L” is an optional linker; and

“m” may be an any integer or 0.

The various portions/fragments of the fusion protein are discussed further below.

a. Fc Region and Fc Fragment

The fusion protein of the present disclosure contains an Fc fragment from an immunoglobulin (Ig) molecule.

As used below, “Fc region” refers to a portion of an immunoglobulin located in the c-terminus of the heavy chain constant region. The Fc region is the portion of the immunoglobulin that interacts with a cell surface receptor (an Fc receptor) and other proteins of the complement system to assist in activating the immune system. In IgG, IgA and IgD isotypes, the Fc region contains two heavy chain domains (C_{H}2 and C_{H}3 domains). In IgM and IgE isotypes, the Fc region contains three heavy chain constant domains (C_{H}2 to C_{H}4 domains). Although the boundaries of the Fc portion may vary, the human IgG heavy chain Fc portion is usually defined to comprise residues C226 or P230 to its carboxyl-terminus, wherein the numbering is according to the EU index.

In certain embodiments, the fusion protein comprises a C_{H}2-C_{H}3 domain, which is an FcRn binding fragment, that can be recycled into circulation again. Fusion proteins having this domain demonstrate an increase in the in vivo half-life of the fusion proteins.

As used herein, “Fc fragment” refers to the portion of the fusion protein that corresponds to an Fc region of an immunoglobulin molecule from any isotype. In some embodiments, the Fc fragment comprises the Fc region of IgG, in specific embodiments, the Fc fragment comprises the full-length region of the Fc region of IgG1. In some embodiments, the Fc fragment refers to the full-length Fc region of an immunoglobulin molecule, as characterized and described in the art. In other embodiments, the Fc fragment includes a portion or fragment of the full-length Fc region, such as a portion of a heavy chain domain (e.g., C_{H}2 domain, C_{H}3 domain, etc.) and/or a hinge region typically found in the Fc region. For example, the Fc fragment of can comprise all or part of the C_{H}2 domain and/or all or part of the C_{H}3 domain. In some embodiments, the Fc fragment includes a functional analogue of the full-length Fc region or portion thereof.

As used herein, “functional analogue” refers to a variant of an amino acid sequence or nucleic acid sequence, which retains substantially the same functional characteristics (binding recognition, binding affinity, etc.) as the original sequence. Examples of functional analogues include sequences that are similar to an original sequence, but contain a conservative substitution in an amino acid position; a change in overall charge; a covalent attachment to another moiety; or small additions, insertions, deletions or conservative substitutions and/or any combination thereof. Functional analogues of the Fc fragment can be synthetically produced by any method known in the art. For example, a functional analogue can be produced by modifying a known amino acid sequence by the addition, deletion, and/or substitution of an amino acid by site-directed mutation. In some embodiments, functional analogues have an amino acid sequence that is at least 50%, 55%, 60%, 65%, 70%, 75%, 80%, 85%, 90%, 92%, 95% 96%, 97%, 98%, or 99% identical to a given sequence. Percent identity between two sequences is determined by standard alignment algorithms such as ClustalOmega when the two sequences are in best alignment according to the alignment algorithm.

The immunoglobulin molecule can be obtained or derived from any animal (e.g., human, cows, goats, swine, mice, rabbits, hamsters, rats, guinea pigs). Additionally, the Fc fragment of the immunoglobulin can be obtained or derived from any isotype (e.g., IgA, IgD, IgE, IgG, or IgM) or subclass within an isotype (IgG1, IgG2, IgG3, and IgG4). In some embodiments, the Fc fragment is obtained or derived from IgG and, in particular embodiments, the Fc fragment is obtained or derived from human IgG, including humanized IgG.

The Fc fragment can be obtained or produced by any method known in the art. For example, the Fc fragment can be isolated and purified from an animal, recombinantly expressed, or synthetically produced. In some embodiments, the Fc fragment is encoded in a nucleic acid. molecule (e.g., DNA or RNA) and isolated from a cell, germ line, cDNA library, or phage library.

The Fc region and/or Fc fragment can include a hinge region found in some immunoglobulin isotypes (IgA, IgD, and IgG). In certain embodiments, the Fc fragment is modified by mutating the hinge region so that it does not contain any Cys and cannot form disulfide bonds. The hinge region is discussed further below.

The Fc fragment of the disclosed fusion protein is preferably a single chain Fc. As used herein, “single chain Fc” (of “sFc”) means that the Fc fragment is modified in such a manner that prevents it from forming a dimer (e.g., by chemical modification or mutation addition, deletion, or substation of an amino acid).

In certain embodiments, the Fc fragment of the fusion protein is derived from human IgG1, which can include the wild-type human IgG1 amino acid sequence or variations thereof. In some embodiments, the Fc fragment of the fusion protein contains an Asn (N) amino acid that serves as an N-glycosylation site at amino acid position 297 of the native human IgG1 molecule (based on the European numbering system for IgG1, as discussed in U.S. Pat. No. 7,501,494), which corresponds to residue 67 in the Fc fragment (SEQ ID NO: 231), shown in Table 11. In other embodiments, the N-glycosylation site in the Fc fragment is removed by mutating the Asn (N) residue with His (H) (SEQ ID NO: 232) or Ala (A) (SEQ ID NO: 233) (Table 11). An Fc fragment containing a variable position at the N-glycosylation site is shown as SEQ ID NO: 234 in Table 11.

In some embodiments, the C_{H}3-C_{H}2 domain of the Fc fragment has an amino acid sequence corresponding to the wild-type sequence (disclosed in SEQ ID NO: 231). In certain embodiments, the C_{H}3-C_{H}2 domain of the Fc fragment has the amino acid sequence of SEQ ID NO: 232, where the N-glycosylation site is removed by mutating the Asn (N) residue with His (H). In certain embodiments, the C_{H}3-C_{H}2 domain of the Fc fragment has the amino acid sequence of SEQ NO: 233, where the N-glycosylation site is removed by mutating the Asn (N) residue with Ala (A).

b. Hinge Region

The disclosed fusion protein can include a hinge region found in some immunoglobulin isotypes (IgA, IgD, and IgG). The hinge region separates the Fc region from the Fab region, and adds flexibility to the molecule, and can link two heavy chains via disulfide bonds. Formation of a dimer, comprising two CH2-CH3 domains, is required for the functions provided by intact Fc regions. Interchain disulfide bonds between cysteines in the wild-type hinge region help hold the two chains of the Fc molecules together to create a functional unit.

In certain embodiments, the hinge region is be derived from IgG, preferably IgG1. The hinge region can be a full-length or a modified (truncated) hinge region.

In specific embodiments, the hinge region contains a modification that prevents the fusion protein from forming a disulfide bond with another fusion protein or an immunoglobulin molecule. In specific embodiments, the hinge region is modified by mutating and/or deleting one or more cysteine amino acids to prevent the formation of a disulfide bond. The N-terminus or C-terminus of the full-length hinge region may be deleted to form a truncated hinge region. In order to avoid. the formation of disulfide bonds, the cysteine (Cys) in the hinge region can be substituted with a non-Cys amino acid or deleted. In specific embodiments, the Cys of hinge region may be substituted with Ser, Gly, Ala, Thr, Leu, Ile, Met or Val. Examples of wild-type and mutated hinge regions from IgG1 to IgG4 include the amino acid sequences shown in Table 9 (SEQ ID NOs: 166-187). Disulfide bonds cannot be formed between two hinge regions that contain mutated sequences. The IgG1 hinge region was modified to accommodate various mutated hinge regions with sequences shown in Table 10 (SEQ ID NOs: 188-225).

c. Linker

The fusion protein may have the bioactive molecule linked to the N-terminus of the Fc fragment. Alternatively, the fusion protein may have the bioactive molecule linked to the C-terminus of the Fc fragment. The linkage is a covalent bond, and preferably a peptide bond.

In the present invention, one or more bioactive molecule may be directly linked to the C-terminus or N-terminus of the Fc fragment. Preferably, the bioactive molecule(s) can be directly linked to the hinge of the Fc fragment.

Additionally, the fusion protein may optionally comprise at least one linker. Thus, the bioactive molecule may not be directly linked to the Fc fragment. The linker may intervene between the bioactive molecule and the Fc fragment. The linker can be linked to the N-terminus of the Fc fragment or the C-terminus of the Fc fragment.

In one embodiment, the linker includes amino acids. The linker may include 1-5 amino acids.

d. Bioactive Molecule

As used herein, the term “biologically active molecule” refers to proteins, or portions of proteins, derived either from proteins of SARS-CoV-2 or host-receptors involved in viral entry into a cell. Examples of biologically active molecules include the spike (S), envelope (E), membrane (M), and nucleocapsid (N) proteins from 2019-CoV, the human receptor ACE2 (hACE2), and/or fragments thereof.

In one embodiment, the biologically active molecule is the S protein of SARS-CoV-2 (SEQ ID NO: 20). In certain embodiments, the biologically active molecule is the receptor binding domain (RBD) of the S protein (S-RBD or S1-RBD) of SARS-CoV-2 (SEQ ID NO: 226), which corresponds to amino acid residues 331-530 of the full-length S protein. In certain embodiments, the cysteine (C) residues at positions 61 and 195 of the S-RBD sequence of SEQ ID NO: 226 are mutated to alanine (A) residues, as shown in SEQ ID NO: 227 (residues 61 and 195 of S-RBD correspond to residues 391 and 525 of the full-length S protein of SEQ 11) NO: 20). The mutated S-RBD sequence is also referred to as S-RBDa in this disclosure. The C61A and C195A mutations in the S-RBD sequence are introduced to avoid a mismatch of disulfide bond formation in the recombinant protein expression.

In another embodiment, the biologically active molecule is the human receptor ACE2 (hACE2) (SEQ ID NO: 228). In certain embodiments, the biologically active molecule is the extracellular domain (ECD) of hACE2 (hACE2_{ECD}) (SEQ ID NO: 229), which corresponds to amino acid residues 1-740 of the full-length hACE2 protein. In some embodiments, the histidine (H) residues at positions 374 and 378 in the hACE2_{ECD} sequence of SEQ ID NO: 229 are mutated to asparagine (N) residues, as shown in SEQ ID NO: 230 (also referred to as ACE2N_{ECD} in this disclosure). The H374N and H378N mutations are introduced to abolish the peptidase activity of hACE2.

### 2. Compositions

In certain embodiments, the present invention relates to compositions, including pharmaceutical compositions, comprising the fusion protein and a pharmaceutically acceptable carrier, adjuvant, and/or other excipients such as diluents, additives, stabilizing agents, preservatives, solubilizing agents, buffers, and the like.

Pharmaceutical compositions can be prepared by mixing the fusion protein with optional pharmaceutically acceptable carriers. Pharmaceutically acceptable carriers include solvents, dispersion media, isotonic agents and the like. Examples of carriers include water, saline solutions or other buffers (such as phosphate, citrate buffers), oil, alcohol, proteins (such as serum albumin, gelatin), carbohydrates (such as monosaccharides, disaccharides, and other carbohydrates including glucose, sucrose, trehalose, mannose, mannitol, sorbitol or dextrins), gel, lipids, liposomes, stabilizers, preservatives, antioxidants including ascorbic acid and methionine, chelating agents such as EDTA; salt forming counter-ions such as sodium; non-ionic surfactants such as TWEEN™, PLURONICS™ or polyethylene glycol (PEG), or combinations thereof.

Pharmaceutical compositions can contain one or more adjuvant that act(s) to accelerate, prolong, or enhance the immune response to the fusion protein without having any specific antigenic effect itself. Adjuvants used in the pharmaceutical composition can include oils, oil emulsions, aluminum salts, calcium salts, immune stimulating complexes, bacterial and viral derivatives, virosomes, carbohydrates, cytokines, polymeric microparticles. In certain embodiments, the adjuvant can be selected from alum (potassium aluminum phosphate), aluminum phosphate (e.g. ADJU-PHOS®), aluminum hydroxide (e.g. ALHYDROGEL®), calcium phosphate, incomplete Freund's adjuvant (IFA), Freund's complete adjuvant, MF59, adjuvant 65, Lipovant, ISCOM, liposyn, saponin, squalene, L121, EMULSIGEN®, EmulsIL-6n®, monophosphoryl lipid A (MPL), Quil A, QS21, MONTANIDE® ISA 35, ISA 50V, ISA 50V2, ISA 51, ISA 206, ISA 720, liposomes, phospholipids, peptidoglycan, lipopolysaccahrides (LPS), ASO1, ASO2, ASO3, ASO4, AF03, lipophilic phospholipid (lipid A), gamma inulin, algammulin, glucans, dextrans, glucomannans, galactomannans, levans, xylans, dimethyldioctadecylammonium bromide (DDA), as well as the other adjuvants and emulsifiers.

In some embodiments, the pharmaceutical composition contains MONTANIDE™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e., w/o/w) emulsion with EMULSIGEN or EMULSIGEN D as the adjuvant.

Pharmaceutical compositions can also include pharmaceutically acceptable additives or excipients. For example, pharmaceutical compositions can contain antioxidants, binders, buffers, bulking agents, carriers, chelating agents, coloring agents, diluents, disintegrants, emulsifying agents, fillers, gelling agents, pH buffering agents, preservatives, solubillizing agents, stabilizers, and the like.

Pharmaceutical compositions can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the S-RBD peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for subcutaneous, intradermal, or intramuscular administration. Pharmaceutical compositions suitable for other modes of administration can also be prepared, including oral and intranasal applications.

Pharmaceutical compositions can also be formulated in a suitable dosage unit form. In some embodiments, the pharmaceutical composition contains from about 0.1 μg to about 1 mg of the fusion protein per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one fusion protein. A pharmaceutical composition containing a mixture of more than one fusion protein to allow for synergistic enhancement of the immunoefficacy of the fusion proteins. Pharmaceutical compositions containing more than one fusion protein can be more effective in a larger genetic population due to a broad MHC class II coverage thus provide an improved immune response to the fusion protein.

The pharmaceutical compositions can also contain more than one active compound. For example, the formulation can contain one or more fusion protein and/or one or more additional beneficial compound(s). The active ingredients can be combined with the carrier in any convenient and practical manner, e.g., by admixture, solution, suspension, emulsification, encapsulation, absorption and the like, and can be made in formulations such as powder (including lyophilized powder), suspensions that are suitable for injections, infusion, or the like. Sustained-release preparations can also be prepared.

In certain embodiments, the pharmaceutical composition contains the fusion protein for human use. The pharmaceutical compositions can be prepared in an appropriate buffer including, but not limited to, citrate, phosphate, Tris, BIS-Tris, etc. at an appropriate pH and can also contain excipients such as sugars (50 mM to 50 mM of sucrose, trehalose, mannitol, or mixtures thereof), surfactants (e.g., 0.025% -0.5% of TWEEN 20 or TWEEN 80), and/or other reagents. The formulation can be prepared to contain various amounts of fusion protein. In general, formulations for administration to a subject contain between about 0.1 μg/mL to about 200 μg/mL. In certain embodiments, the formulations can contain between about 0.5 μg/mL to about 50 μg/mL; between about 1.0 μg/mL to about 50 μg/mL; between about 1 μg/mL to about 25 μg/mL; or between about 10 μg/mL to about 25 μg/mL of fusion protein. In specific embodiments, the formulations contain about 1.0 μg/mL, about 5.0 μg/mL, about 10.0 μg/mL, or about 25.0 μg/mL of fusion protein.

### 3. Methods

Another aspect of the present invention relates to methods for making and using a fusion protein and compositions thereof.

a. Producing the Fusion Protein

In some embodiments, the method for making the fusion protein comprises (i) providing a bioactive molecule and an Fc fragment comprising a hinge region, (ii) modifying the hinge region to prevent it from forming a disulfide bond, and (iii) linking the bioactive molecule directly or indirectly to the sFc through the mutated hinge region to form the fusion protein, hybrid, conjugate, or composition thereof. The present disclosure also provides a method for purifying the fusion protein, comprising (i) providing a fusion protein, and (ii) purifying the fusion protein by Protein A or Protein G-based chromatography media.

The fusion protein may alternatively be expressed by well-known molecular biology techniques. Any standard manual on molecular cloning technology provides detailed protocols to produce the fusion protein of the invention by expression of recombinant DNA and RNA. To construct a gene expressing a fusion protein of this invention, the amino acid sequence is reverse translated into a nucleic acid sequence, preferably using optimized codons for the organism in which the gene will be expressed. Next, a gene encoding the peptide or protein is made, typically by synthesizing overlapping oligonucleotides which encode the fusion protein and necessary regulatory elements. The synthetic gene is assembled and inserted into the desired expression vector. The synthetic nucleic acid sequences encompassed by this invention include those which encode the fusion protein of the invention, and nucleic acid constructs characterized by changes in the non-coding sequences that do not alter the biological activity of the molecule encoded thereby. The synthetic gene is inserted into a suitable cloning vector and recombinants are obtained and characterized. The fusion protein is expressed under conditions appropriate for the selected expression system and host. The fusion protein is purified by an affinity column of Protein A or Protein G (e.g., SOFTMAX®, ACROSEP®, SERA-MAG®, or SEPHAROSE®).

The fusion protein of the present invention can be produced in mammalian cells, lower eukaryotes, or prokaryotes. Examples of mammalian cells include monkey COS cells, CHO cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

The invention also provides a method for producing a single chain Fc (sFc) region of an immunoglobulin G, comprising mutating, substituting, or deleting the Cys in a hinge region of Fc of IgG. In one embodiment, the Cys is substituted with Ser, Gly, The, Ala, Val, Leu, Ile, or Met. In another embodiment, the Cys is deleted. In an additional embodiment, a fragment of the hinge is deleted.

The invention further provides a method for producing a fusion protein comprising: (a) providing a bioactive molecule and an IgG Fc fragment comprising a hinge region, (b) mutating the hinge region by amino acid substitution and/or deletion to form a mutated Fc without disulfide bond formation, and (c) combining the bioactive molecule and the mutated Fc.

b. Using the Fusion Protein

Pharmaceutical compositions containing the fusion proteins can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

The fusion protein of the invention can be administered intravenously, subcutaneously, intra-muscularly, or via any mucosal surface, e.g., orally, sublingually, buccally, sublingually, nasally, rectally, vaginally, or via pulmonary route. In certain embodiments, the pharmaceutical composition is formulated for subcutaneous, intradermal, or intramuscular administration. Pharmaceutical compositions suitable for other modes of administration can also be prepared, including oral and intranasal applications.

The dose of the fusion protein of the invention will vary depending upon the subject and the particular mode of administration. The dosage required will vary according to a number of factors known to those skilled in the art, including, but not limited to, the fusion protein, the species of the subject and the size of the subject. Dosage may range from 0.1 to 100,000 μg/kg body weight. In certain embodiments, the dosage is between about 0.1 μg to about 1 mg of the fusion protein per kg body weight. The fusion protein can be administered in a single dose, in multiple doses throughout a 24-hour period, or by continuous infusion. The fusion protein can be administered continuously or at specific schedule. The effective doses may be extrapolated from dose-response curves obtained from animal models.

### 4. Specific Embodiments

Specific embodiments of the present invention include, but are not limited to, the following:

(1) A fusion protein comprising an Fc fragment of an IgG molecule and a bioactive molecule, wherein the Fc fragment is a single chain Fc (sFc).
(2) The fusion protein according to (1), wherein the Fc fragment comprises a hinge region.
(3) The fusion protein according to (2), wherein the hinge region is mutated and does not form disulfide bonds.
(4) The fusion protein according to (2), wherein the hinge region comprises an amino acid sequence selected from the group consisting of SEQ ID NOs: 166-225.
(5) The fusion protein according to (2), wherein the hinge region comprises an amino acid sequence of SEQ ID NO: 188.
(6) The fusion protein according to (1), wherein the bioactive molecule is the receptor binding domain (RBD) of the S protein (S-RBD) from SARS-CoV-2 of SEQ ID NO: 226 or a mutated form of S-RBD of SEQ ID NO: 227.
(7) The fusion protein according to (1), wherein the bioactive molecule is the extracellular domain (ECD) of human receptor ACE2 (ECD-hACE2) of SEQ ID NO: 228 or a mutated form of ECD-hACE2 of SEQ ID NO: 229.
(8) The fusion protein according to (1), wherein the bioactive molecule is linked to the Fc fragment through a mutated hinge region.
(9) The fusion protein according to (1), wherein the amino acid sequence of the fusion protein is selected from the group consisting of SEQ ID NOs: 235-238.
(10) A pharmaceutical composition comprising the fusion protein according to any one of (1) to (9) and a pharmaceutically acceptable carrier or excipient.
(11) A method for producing a fusion protein comprising:
a) providing a bioactive molecule and an Fc fragment comprising a hinge region,
b) mutating the hinge region by amino acid substitution and/or deletion to form a mutated Fc, and
c) combining the bioactive molecule and the mutated Fc.
   (12) The method according to (11), wherein the hinge region is mutated by substitution and/or deletion of a cysteine residue.
   (13) The method according to (11), wherein the bioactive molecule is combined with the mutated Fc through the hinge region.
   (14) The method according to (11), wherein the bioactive molecule is the receptor binding domain (RBD) of the S protein (S-RBD) from SARS-CoV-2 of SEQ ID NO: 226 or a mutated. form of S-RBD of SEQ ID NO: 227.
   (15) The method according to (11), wherein the bioactive molecule is the extracellular domain (ECD) of human receptor ACE2 (ECD-hACE2) of SEQ ID NO: 228 or a mutated form of ECD-hACE2 of SEQ ID NO: 229.

Additional specific embodiments of the present invention include, but are not limited to the following examples.

### D. A Multitope Protein/Peptice Vaccine Composition for the Prevention of Infection by SARS-CoV-2

The fourth aspect of the disclosed relief system relates to a multitope protein/peptide vaccine composition for the prevention of infection by SARS-CoV-2. The multitope protein/peptide vaccine composition disclosed herein is also referred to as “LTB-612”.

### 1. S1-Receptor-Binding Region-Based Designer Protein

Most of the vaccines currently in clinical trials only target the full-length S protein to induce a neutralizing antibody response. The induction of T cell responses would be limited compared to responses generated by natural multigenic SARS-CoV-2 infections. The S1-RBD region is a critical component of SARS-CoV-2. It is required for cell attachment and represents the principal neutralizing domain of the virus of the highly similar SARS-CoV, providing a margin of safety not achievable with a full-length S antigen and eliminating the possibility of the potentially deadly side effects that led to withdrawal of an otherwise effective inactivated RSV vaccine. Accordingly, the monoclonal antibodies for the treatment of newly diagnosed COVID-19, approved through FDA Emergency Use Authorization (Lilly's neutralizing antibody bamlanivimab, LY-CoV555 and REGN-COV2 antibody cocktail), are all directed to S1-RBD.

Due to the clear advantages of a strong S1-RBD vaccine component, the multitope protein/peptide vaccine composition (UB-612) comprises the S1-receptor-binding region-based designer protein described in Part C above. As described above, S1-RBD-sFc is a recombinant protein made through a fusion of S1-RBD of SARS-CoV-2 to a single chain fragment crystallizable region (sFc) of a human IgG1. Genetic fusion of a vaccine antigen to a Fc fragment has been shown to promote antibody induction and neutralizing activity against HIV gp120 in rhesus macaques or Epstein Barr virus gp350 in BALB/c mice (Shubin, Z., et al., 2017; and Zhao, B., et al., 2018). Moreover, engineered Fc has been used in many therapeutic antibodies as a solution to minimized non-specific binding, increase solubility, yield, thermostability, and in vivo half-life (Liu, H., et al., 2017).

In some embodiments, the vaccine composition contains S1-RBD-sFc fusion protein of SEQ ID NO: 235. The S1-RBD-sFc protein (SEQ ID NO: 235) contains the S1-RBD peptide (SEQ ID NO: 226), which corresponds to amino acid residues 331-530 of the full-length S protein of SARS-CoV-2, fused to the single chain Fc peptide (SEQ ID NO: 232) through a mutated hinge region from IgG (SEQ ID NO: 188).

In some embodiments, the cysteine (C) residues at positions 61 and 195 of the S-RBD sequence of SEQ ID NO: 226 are mutated to alanine (A) residues, as shown in SEQ ID NO: 227 (residues 61 and 195 of S-RBD correspond to residues 391 and 525 of the full-length S protein of SEQ ID NO: 20). The mutated S-RBD sequence is also referred to as S-RBDa in this disclosure. The C61A and C195A mutations in the S-RBD sequence are introduced to avoid a mismatch of disulfide bond formation in the recombinant protein expression. The amino acid sequence of the S-RBDa fused to the single chain Fc peptide (S-RBDa-sFc) is SEQ ID NO: 236.

The amount of the S1-receptor-binding region-based designer protein in the vaccine composition can vary depending on the need or application. The vaccine composition can contain between about 1 μg to about 1,000 μg of the S1-receptor-binding region-based designer protein. In some embodiments, the vaccine composition contains between about 10 μg to about 200 μg of the S1-receptor-binding region-based designer protein.

### 2. Th/CTL Peptides

A neutralizing response against the S protein alone is unlikely to provide lasting protection against SARS-CoV-2 and its emerging variants with mutated B-cell epitopes. A long-lasting cellular response could augment the initial neutralizing response (through memory B cell activation) and provide much greater duration of immunity as antibody titers wane. Recent studies have demonstrated that IgG response to S declined rapidly in >90% of SARS-CoV-2 infected individuals within 2-3 months (Long, Q.-X., et al., 2020). In contrast, memory T cells to SARS have been shown to endure 11-17 years after 2003 SARS outbreak (Ng., O.-W., et al., 2016; and Le Bert, N., et al., 2020). The S protein is a critical antigen for elicitation of humoral immunity which mostly contains CD4+ epitopes (Braun, J., et al., 2020). Other antigens are needed to raise/augment cellular immune responses to clear SARS-CoV-2 infection. The vast majority of reported CD8+ T cell epitopes in SARS-CoV-2 proteins are located in ORF1ab, N, M, and ORF3a regions; only 3 are in S, with only 1 CD8+ epitope being located in the S1-RBD (Ferretti, A. P., et al., 2020). The smaller M and N structural proteins are recognized by T cells of patients who successfully controlled their infection. In a study of nearly 3,000 people in the UK, it was found that individuals with higher numbers of T cells were more protected against SARS-CoV-2 compared to those with low T cell responses, suggesting that T cell immunity may play a critical role in preventing COVID-19 (Wyllie, D., et al., 2020).

To provide immunogens to elicit T cell responses, Th/CTL epitopes from highly conserved sequences derived from S, N, and M proteins of SARS-CoV and SARS-CoV-2 (e.g., Ahmed, S. F., et al., 2020/0 were identified after extensive literature search. These Th/CTL peptides are shown in Tables 4 and 5. Several peptides within these regions were selected and subject to further designs. Each selected peptide contains Th or CTL epitopes with prior validation of MHC I or II binding and exhibits good manufacturability characteristics (optimal length and amenability for high quality synthesis). These rationally designed Th/CTL peptides were further modified by addition of a Lys-Lys-Lys tail to each respective peptide's N-terminus to improve peptide solubility and enrich positive charge for use in vaccine formulation. The designs and sequences of the five final peptides and their respective HLA alleles are shown in Table 32.

To enhance the immune response, a proprietary peptide UBITh®1a (SEQ ID NO: 66) can be added to the peptide mixture of the vaccine composition. UBITh®1a is a proprietary synthetic peptide with an original framework sequence derived from the measles virus fusion protein (MVF). This sequence was further modified to exhibit a palindromic profile within the sequence to allow accommodation of multiple MHC class II binding motifs within this short peptide of 19 amino acids. A Lys-Lys-Lys sequence was added to the N terminus of this artificial Th peptide as well to increase its positive charge thus facilitating the peptide's subsequent binding to the highly negatively charged CpG oligonucleotide molecule to form immunostimulatory complexes through “charge neutralization”. In previous studies, attachment of UBITh®1a to a target “functional B epitope peptide” derived from a self-protein rendered the self-peptide immunogenic, thus breaking immune tolerance (Wang, C. Y., et al, 2017). The Th epitope of UBITh®1 has shown this stimulatory activity whether covalently linked to a target peptide or as a free charged peptide, administered together with other designed target peptides, that are brought together through the “charge neutralization” effect with CpG1, to elicit site-directed B or CTL responses. Such immunostimulatory complexes have been shown to enhance otherwise weak or moderate response of the companion target immunogen (e.g., WO 2020/132275A1). CpG1 is designed to bring the rationally designed immunogens together through “charge neutralization” to allow generation of balanced B cells (induction of neutralizing antibodies) and Th/CTL responses in a vaccinated host. In addition, activation of TLR-9 signaling by CpG is known to promote IgA production and favor Th1 immune response. UBITh®1 peptide is incorporated as one of the Th peptides for its “epitope cluster” nature to further enhance the SARS-CoV-2 derived Th and CTL epitope peptides for their antiviral activities. The amino acid sequence of UBITh®1 is SEQ ID NO: 65 and the sequence of UBITh®1a is SEQ ID NO: 66. The nucleic acid sequence of CpG1 is SEQ ID NO: 104.

In view of the above, the multitope protein/peptide vaccine composition can contain one or more Th/CTL peptides. The Th/CTL peptides can include:
a. peptides derived from the SARS-CoV-2 M protein of SEQ ID NO: 1 (e.g., SEQ ID NO: 361);
b. peptides derived from the SARS-CoV-2 N protein of SEQ ID NO: 6 (e.g., SEQ ID NOs: 9-16, 19, 153-160, 165, 347, 350, 351, and 363);
c. peptides derived from the SARS-Cov-2 S protein of SEQ ID NO: 20 (e.g., SEQ ID NOs: 35-36, 39-48, 145-152, 161-164, 345-346, 348, 362, 364, and 365); and/or
d. artificial Tb epitopes derived from pathogen proteins (e.g., SEQ ID NOs: 49-100).

The vaccine composition can contain one or more of the Th/CTL peptides. In certain embodiments, the vaccine composition contains a mixture of more than one Th/CTL peptides. When present in a mixture, each Th/CTL peptide can be present in any amount or ratio compared to the other peptide or peptides. For example, the Th/CTL peptides can be mixed in equimolar amounts, equal-weight amounts, or the amount of each peptide in the mixture can be different than the amount of the other peptide(s) in the mixture. If more than two Th/CTL peptides are present in the mixture, the amount of the peptides can be the same as or different from any of the other peptides in the mixture.

The amount of Th/CTL peptide(s) present in the vaccine composition can vary depending on the need or application. The vaccine composition can contain a total of between about 0.1 μg to about 100 μg of the Th/CTL peptide(s). In some embodiments, the vaccine composition contains a total of between about 1 μg to about 50 μg of the Th/CTL peptide(s).

In certain embodiments, the vaccine composition contains a mixture of SEQ if) NOs: 345, 346, 347, 348, 361, and 66. These Th/CTL peptides can be mixed in equimolar amounts, equal-weight amounts, or the amount of each peptide in the mixture can be different than the amount of the other peptide(s) in the mixture. In certain embodiments, these Th/CTL peptides are mixed in equal-weight amounts in the vaccine composition.

### 3. Excipients

The vaccine composition can also contain a pharmaceutically acceptable excipient. As used herein, the term “excipient” or “excipients” refers to any component in the vaccine composition that is not (a) the S1-receptor-binding region-based designer protein or (b) the Th/CTL peptide(s). Examples of excipients include carriers, adjuvants, antioxidants, binders, buffers, bulking agents, chelating agents, coloring agents, diluents, disintegrants, emulsifying agents, surfactants, solvents, fillers, gelling agents, pH buffering agents, preservatives, solubilizing agents, stabilizers, and the like. Accordingly, the vaccine composition can contain a pharmaceutically effective amount of an active pharmaceutical ingredient (API), such as the S1-receptor-binding region-based designer protein and/or one or more Th/CTL peptides, together with a pharmaceutically acceptable excipient.

The vaccine composition can contain one or more adjuvants that act to accelerate, prolong, or enhance the immune response to the API without having any specific antigenic effect itself. Adjuvants can include oils, oil emulsions, aluminum salts, calcium salts, immune stimulating complexes, bacterial and viral derivatives, virosomes, carbohydrates, cytokines, polymeric microparticles. In certain embodiments, the adjuvant can be selected from a CpG oligonucleotide, alum (potassium aluminum phosphate), aluminum phosphate (e.g. ADJU-PHOS®), aluminum hydroxide (e.g. ALHYDROGEL®), calcium phosphate, incomplete Freund's adjuvant (IFA), Freund's complete adjuvant, MF59, adjuvant 65, Lipovant, ISCOM, liposyn, saponin, squalene, L121, EMULSIGEN®, EmulsIL-6n®, monophosphoryl lipid A (MPL), Quil A, QS21, MONTANIDE® ISA 35, ISA 50V, ISA 50V2, ISA 51, ISA 206, ISA 720, liposomes, phospholipids, peptidoglycan, lipopolysaccahrides (LPS), ASO1, ASO2, ASO3, ASO4, AF03, lipophilic phospholipid (lipid A), gamma inulin, gammulin, glucans, dextrans, glucomannans, galactomannans, levans, xylans, dimethyldioctadecylammonium bromide (DDA), as well as the other adjuvants and emulsifiers.

In some embodiments, the vaccine composition contains ADJU-PHOS® (aluminum phosphate), MONTANIDE™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e., w/o/w) emulsion with EMULSIGEN or EMULSIGEN D as the adjuvant.

In certain embodiments, the multitope protein/peptide vaccine composition contains ADHJ-PHOS® (aluminum phosphate) as the adjuvant to improve the immune response. Aluminum phosphate serves as a Th2 oriented adjuvant via the nucleotide binding oligometization domain (NOD) like receptor protein 3 (NLRP3) inflammasome pathway. Additionally, it has pro-phagocytic and repository effects with a long record of safety and the ability to improve immune responses to target proteins in many vaccine formulations.

The vaccine composition can contain pH adjusters and/or buffering agents, such as hydrochloric acid, phosphoric acid, citric acid, acetic acid, histidine, histidine HCl.H₂O, lactic acid, tromethamine, gluconic acid, aspartic acid, glutamic acid, tartaric acid, succinic acid, malic acid, fumaric acid, α-ketoglutaric acid, and arginine HCl.

The vaccine composition can contain surfactants and emulsifiers, such as olyoxyethylene sorbitan fatty acid esters (polysorbate, TWEEN®), Polyoxyethylene 15 hydroxy stearate (Macrogol 15 hydroxy stearate, SOLUTOL HS15®), Polyoxyethylene castor oil derivatives (CREMOPHOR® EL, ELP, RH 40), Polyoxyethylene stearates (MYRJ®), Sorbitan fatty acid esters (SPAN®), Polyoxyethylene alkyl ethers (BRIJ®), and Polyoxyethylene nonylphenol ether (NONOXYNOL®).

The vaccine composition can contain carriers, solvents, or osmotic pressure keepers, such as water, alcohols, and saline solutions (e.g., sodium chloride).

The vaccine composition can contain preservatives, such as alkyl/aryl alcohols (e.g., benzyl alcohol, chlorbutanol, 2-ethoxyethanol), amino aryl acid esters (e.g., methyl, ethyl, propyl butyl parabens and combinations), alkyl/aryl acids (e.g., benzoic acid, sorbic acid), biguanides (e.g., chlorhexidine), aromatic ethers (e.g., phenol, 3-cresol, 2-phenoxyethanol), organic mercurials (e.g., thimerosal, phenylmercurate salts).

### 4. Formulations

The vaccine composition can be formulated as immediate release or for sustained release formulations. Additionally, the vaccine composition can be formulated for induction of systemic, or localized mucosal, immunity through immunogen entrapment and co-administration with microparticles. Such delivery systems are readily determined by one of ordinary skill in the art.

The vaccine composition can be prepared as an injectable, either as a liquid solution or suspension. Liquid vehicles containing the vaccine composition can also be prepared prior to injection. The vaccine composition can be administered by any suitable mode of application, for example, i.d., i.v., i.p., i.m., intranasally, orally, subcutaneously, etc. and in any suitable delivery device. In certain embodiments, the vaccine composition is formulated for subcutaneous, intradermal, or intramuscular administration. The vaccine composition can also be prepared for other modes of administration, including oral and intranasal applications.

The vaccine composition can also be formulated in a suitable dosage unit form. In some embodiments, the vaccine composition contains from about 1 μg to about 1,000 μg of the API (e.g., the S1-receptor-binding region-based designer protein and/or one or more of the Th/CTL peptides). Effective doses of the vaccine composition can vary depending upon many different factors, including means of administration, target site, physiological state of the subject, whether the subject is human or an animal, other medications administered, and whether treatment is prophylactic or therapeutic. Usually, the subject is a human, but nonhuman mammals can also be treated. When delivered in multiple doses, the vaccine composition may be conveniently divided into an appropriate amount per dosage unit form. The administered dosage will depend on the age, weight and general health of the subject as is well known in the therapeutic arts.

In some embodiments, the vaccine composition contains a S1-receptor-binding region-based designer protein and one or more Th/CTL peptides in a formulation with additives and/or excipients. In certain embodiments, the vaccine composition contains a S1-receptor-binding region-based designer protein and more than one Th/CTL peptides in a formulation with additives and/or excipients. A vaccine composition containing a mixture of more than one Th/CTL peptides can provide synergistic enhancement of the immunoefficacy of the composition. A vaccine composition containing a S1-receptor-binding region-based designer protein and more than one Th/CTL peptides in a formulation with additives and/or excipients can be more effective in a larger genetic population compared to compositions containing only the designer protein or one Th/CTL peptide, due to a broad MHC class II coverage, thus providing an improved immune response to vaccine composition.

When the vaccine composition contains a S1-receptor-binding region-based designer protein and one or more Th/CTL peptides as the API, the relative amounts of the designer protein and the Th/CTL peptides can be present in any amount or ratio to each other. For example, the designer protein and the Th/CTL peptide(s) can be mixed in equimolar amounts, equal-weight amounts, or the amount of the designer protein and the Th/CTL peptide(s) can be different. In addition, if more than one Th/CTL peptide is present in the composition, the amount of the designer protein and each Th/CTL peptide can be the same as or different from each other. In some embodiments, the molar or weight amount of the designer protein is present in the composition in an amount greater than the Th/CTL peptides. In other embodiments, the molar or weight amount of the designer protein is present in the composition in an amount less than the Th/CTL peptides. The ratio (weight:weight) of the designer protein to Th/CTL peptide(s) can vary depending on the need or application. In some instances, the ratio (w:w) of the designer peptide to Th/CTL peptide(s) can be 10:90, 20:80, 30:70, 40:60, 50:50, 60:40, 70:30, 80:20, or 90:10. In specific embodiments, the ratio (w:w) of the designer peptide to Th/CTL peptide(s) is 95:5, 94:6, 93:7, 92:8, 91:9, 90:10, 89:11, 88:12, 87:13, 86:14, or 85:15. In specific embodiments, the ratio (w:w) of the designer peptide to Th/CTL peptide(s) is 88:12.

In some embodiments, the vaccine composition comprises the S1-receptor-binding region-based designer protein of SEQ ID NO: 235. In other embodiments, the vaccine composition comprises one or more Th/CTL peptides. In some embodiments, the vaccine composition comprises the S1-receptor-binding region-based designer protein of SEQ ID NO: 235 in combination with Th/CTL peptides of SEQ ID NOs: 345. 346, 347, 348, 361, and 66. In certain embodiments, the vaccine composition comprises the S1-receptor-binding region-based designer protein of SEQ ID NO: 235, the Th/CTL peptides of SEQ ID NOs: 345, 346, 347, 348, 361, and 66, together with one or more adjuvant and/or excipient. In various embodiments, the vaccine composition comprises SEQ NO: 235 together with the Th/CTL peptides of SEQ ID NOs: 345, 346, 347, 348, 361, and 66, where the Th/CTL peptides are present in an equal-weight ratio to each other and the ratio (w:w) of SEQ ID NO: 235 to the combined weight of the Th/CTL peptides is 88:12. Specific embodiments of the vaccine composition containing 20 μg/mL, 60 μg/mL, and 200 μg/mL, based on the total weight of the S1-RBD-sFC protein (SEQ ID NO: 235) together with the Th/CTL peptides of SEQ ID NOs: 345, 346, 347, 348, 361, and 66 are provided in Tables 33-35, respectively.

### 5. Antibodies

The present disclosure also provides antibodies elicited by the vaccine composition. The present disclosure provides a vaccine composition comprising a S1-receptor-binding region-based designer protein (e.g., S1-RBD-sFc of SEQ ID NO: 235) and one or more Th/CTL peptides (e.g., SEQ ID NOs: 345, 346, 347, 348, 361, and 66) in a formulation with additives and/or excipients capable of eliciting high titer neutralizing antibodies against SARS-CoV-2 and inhibiting the binding of S-RBD to its receptor ACE2 with a high responder rate in immunized hosts.

Antibodies elicited by the disclosed vaccine composition are also included in the present disclosure. Such antibodies can be isolated and purified using methods known in the field. Isolated and purified antibodies can be included into pharmaceutical compositions or formulations for the use in preventing and/or treating subjects exposed to SARS-CoV-2.

### 6. Methods

The present disclosure is also directed to methods for making and using the vaccine composition and formulations thereof.

a. Methods for Manufacturing the S1-Receptor-Binding Region-Based Designer Protein and Th/CTL Peptides

The disclosed S1-receptor-binding region-based designer protein can be manufactured according to the methods described in Part C(3) above or according to Example 15. In addition, the disclosed Th/CTL peptides can be manufactured according to the methods described in Part B(4) above.

b. Methods for Using the Vaccine Composition

In prophylactic applications, the disclosed multitope protein/peptide vaccine composition can be administered to a subject susceptible to, or at risk of, becoming infected with SARS-CoV-2, the virus that causes COVID-19 to eliminate or reduce the risk, lessen the severity, or delay the onset of the disease.

The amount of the vaccine composition that is adequate to accomplish prophylactic treatment is defined as a prophylactically-effective dose. The disclosed multitope protein/peptide vaccine composition can be administered to a subject in one or more doses to produce a sufficient immune response in order to prevent an infection by SARS-CoV-2. Typically, the immune response is monitored, and repeated dosages are given if the immune response starts to wane.

The vaccine composition can be formulated as immediate release or for sustained release formulations. Additionally, the vaccine composition can be formulated for induction of systemic, or localized mucosal, immunity through immunogen entrapment and co-administration with microparticles. Such delivery systems are readily determined by one of ordinary skill in the art.

The vaccine composition can be prepared as an injectable, either as a liquid solution or suspension. Liquid vehicles containing the vaccine composition can also be prepared prior to injection. The vaccine composition can be administered by any suitable mode of application, for example, i.d., i.v., i.p., i.m., intranasally, orally, subcutaneously, etc. and in any suitable delivery device. In certain embodiments, the vaccine composition is formulated for subcutaneous. intradermal, or intramuscular administration. The vaccine composition can also be prepared for other modes of administration, including oral and intranasal applications.

The dose of the vaccine composition will vary depending upon the subject and the particular mode of administration. The dosage required will vary according to a number of factors known to those skilled in the art, including, but not limited to the species and size of the subject. The dosage may range from 1 μg to 1,000 μg of the combined weight of the designer protein and the Th/CTL peptides. The dosage can between about 1 μg to about 1 mg, between about 10 μg to about 500 μg, between about 20 μg to 200 pig of the combined weight of the designer protein and the Th/CTL peptides. The dosage, as measured by the combined weight of the designer protein and the Th/CTL peptides is about 10 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 250 μg, about 300 μg, about 400 μg, about 500 μg, about 600 μg, about 700 μg, about 800 μg, about 900 μg, about 1,000 μg. The ratio (weight:weight) of the designer protein to Th/CTL peptide(s) can vary depending on the need or application. In some instances, the ratio (w:w) of the designer protein to Th/CTL peptide(s) can be 10:90, 20:80, 30:70, 40:60, 50:50, 60:40, 70:30, 80:20, 90:10, 99:1, or with a fixed amount of the Th/CTL peptides per dose. In specific embodiments, the ratio (w:w) of the designer protein to Th/CTL peptide(s) is 95:5, 94:6, 93:7, 92:8, 91:9, 90:10, 89:11, 88:12, 87:13, 86:14, or 85:15. In specific embodiments, the ratio (w:w) of the designer peptide to Th/CTL peptide(s) is 88:12. In specific embodiments, the vaccine composition contains the components shown in Tables 33-35.

The vaccine composition can be administered in a single dose, in multiple doses over a period of time. The effective doses may be extrapolated from dose-response curves obtained from animal models. In some embodiments, the vaccine composition is provided to a subject in a single administration. In other embodiments, the vaccine composition is provided to a subject in multiple administrations (two or more). When provided in multiple administrations, the duration between administrations can vary depending on the application or need. In some embodiments, a first dose of the vaccine composition is administered to a subject and a second dose is administered about 1 week to about 12 weeks after the first dose. In certain embodiments, the second dose is administered about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after the first administration. In a specific embodiment, the second dose is administered about 4 weeks after the first administration.

A booster dose of the vaccine composition can be administered to a subject following an initial vaccination regimen to increase immunity against SARS-CoV-2. In some embodiments, a booster dose of the vaccine composition is administered to a subject about 6 months to about 10 years after the initial vaccination regimen. In certain embodiments, the booster dose of the vaccine composition is administered about 6 months, about 1 year, about 2 years, about 3 years, about 4 years, about 5 years, about 6 years, about 7 years, about 8 years, about 9 years, or about 10 years after the initial vaccination regimen or after the last booster dose.

### 7. Specific Embodiments

(1) A fusion protein selected from the group consisting of S1-RBD-sFc of SEQ ID NOs: 235, S1-RBDa-sFc of SEQ ID NO: 236, and S1-RBD-Fc of SEQ ID NO: 355.
(2) A COVID-19 vaccine composition comprising
a. the fusion protein according to (1); and
a pharmaceutically acceptable excipient.
   (3) The COVID-19 vaccine composition according to (2), wherein the fusion protein is S1-RBD-sFc of SEQ ID NO: 235.
   (4) The COVID-19 vaccine composition according to (2) further comprising a Th/CTL peptide.
   (5) The COVID-19 vaccine composition according to (4), wherein the Th/CTL peptide is derived from the SARS-CoV-2 M protein of SEQ ID NO: 1, the SARS-CoV-2 N protein of SEQ ID NO: 6, the SARS-Cov-2 S protein of SEQ ID NO: 20, a pathogen protein, or any combination thereof.
   (6) The COVID-19 vaccine composition according to (5), wherein
a. the Th/CTL peptide derived from the SARS-CoV-2 M protein is SEQ ID NO: 361;
b. the Th/CTL peptide derived from the SARS-CoV-2 N protein is selected from the group consisting of SEQ ID NOs: 9-16, 19, 153-160, 165, 347. 350, 351, and 363;
c. the Th/CTL peptide derived from the SARS-CoV-2 S protein is selected from the group consisting of SEQ ID NOs: 35-36, 39-48, 145-152, 161-164, 345-346, 348, 362, 364, and 365;
d. the Th/CTL peptide derived from a pathogen protein is selected from the group consisting of SEQ ID NOs: 49-100.
   (7) The COVID-19 vaccine composition according to 2), further comprising a mixture of Th/CTL peptides of SEQ ID NOs: 345, 346, 347, 348, 361, 66.
   (8) The COVID-19 vaccine composition according to (7), wherein each of the Th/CTL peptides are present in the mixture in equal-weight amounts.
   (9) The COVID-19 vaccine composition according to (8), wherein the ration (w:w) of the S1-RBD-sFc protein to the total weight of the mixture of Th/CTL peptides is 88:12.
   (10) The COVID-19 vaccine composition according to (2), wherein the pharmaceutically acceptable excipient is an adjuvant, buffer, surfactant, emulsifier, pH adjuster, saline solution, preservative, solvent, or any combination thereof.
   (11) The COVID-19 vaccine composition according to (2), wherein the pharmaceutically acceptable excipient is selected from the group consisting of a CpG oligonucleotide, ADJUPHOS (aluminum phosphate), histidine, histidine HCl.H₂O, arginine HCl, TWEEN 80 (polyoxyethylene (20) sorbitan monooleate), hydrochloric acid, sodium chloride, 2-phenoxyethanol, water, and any combination thereof.
   (12) A COVID-19 vaccine composition comprising:
a. a S-RBD-sFc protein of SEQ ID NO: 235;
b. a Th/CTL peptide selected from the group consisting of SEQ ID NOs: 9-16, 19, 35-36, 39-100, 145-165, 345-348, 350, 351, 362-365, and any combination thereof;
c. a pharmaceutically acceptable excipient.
   (13) The COVID-19 vaccine composition according to (12), wherein the Th/CTL peptides in (b) is a mixture of SEQ ID NOs: 345, 346, 347, 348, 361, and 66.
   (14) The COVID-19 vaccine composition according to (12), wherein each of the Th/CTL peptides are present in the mixture in equal-weight amounts.
   (15) The COVID-19 vaccine composition according to (13), wherein the ration (w:w) of the S-RBD-sFc protein to the total weight of the mixture of Th/CTL peptides is 88:12.
   (16) The COVID-19 vaccine composition according to (12), wherein the pharmaceutically acceptable excipient is an adjuvant, buffer, surfactant, emulsifier, pH adjuster, saline solution, preservative, solvent, or any combination thereof.
   (17) The COVID-19 vaccine composition according to (12), wherein the pharmaceutically acceptable excipient is selected from the group consisting of a CpG oligonucleotide, ADJUPHOS (aluminum phosphate), histidine, histidine HCl.H₂O, arginine HCl, TWEEN 80 (polyoxyethylene (20) sorbitan monooleate), hydrochloric acid, sodium chloride, 2-phenoxyethanol, water, and any combination thereof.
   (18) The COVID-19 vaccine composition according to (12), wherein
the Th/CTL peptide is a mixture of SEQ ID NOs: 345, 346, 347, 348, 361, and 66, wherein each peptide is present in the mixture in equal-weight amounts;
the pharmaceutically acceptable excipient is a combination of a CpG1 oligonucleotide, ADJUPHOS (aluminum phosphate), histidine, histidine HCl.H₂O, arginine HCl, TWEEN 80 (polyoxyethylene (20) sorbitan monooleate), hydrochloric acid, sodium chloride, and 2-phenoxyethanol in water.
   (19) The COVID-19 vaccine composition according to (18), wherein
the total amount of the S-RBD-sFc protein of SEQ ID NO: 235 is between about 10 μg to about 200 μg; and
the total amount of the Th/CTL peptides is between about 2 μg to about 25 μg.
   (20) The COVID-19 vaccine composition according to (18), wherein
the total amount of the S-RBD-sFc protein of SEQ ID NO: 235 is between about 17.6 μg; and
the total amount of the Th/CTL peptides is between about 2.4 μg.
   (21) The COVID-19 vaccine composition according to (18), wherein
the total amount of the S-RBD-sFc protein of SEQ ID NO: 235 is between about 52.8 μg; and
the total amount of the Th/CTL peptides is between about 7.2 μg.
   (22) The COVID-19 vaccine composition according to (18), wherein
the total amount of the S-RBD-sFc protein of SEQ ID NO: 235 is between about 176 μg; and
the total amount of the Th/CTL peptides is between about 24 μg.
   (23) A method for preventing COVID-19 in a subject comprising administering a pharmaceutically effective amount of the vaccine composition according to (12) to the subject.
   (24) The method according to (23), wherein the pharmaceutically effective amount of the vaccine composition is administered to the subject in two doses.
   (25) The method according to (24), wherein a first dose of the vaccine composition is administered to the subject and a second dose of the vaccine composition is administered to the subject about 4 weeks after the first dose.
   (26) A method for generating antibodies against SARS-CoV-2 comprising administering a pharmaceutically effective amount of the vaccine composition according to (12) to a subject.
   (27) An isolated antibody or epitope-binding fragment thereof that specifically binds to the S-RBD portion of the SARS-CoV-2 S protein (i.e., SEQ ID NO: 226).
   (28) A composition comprising; the isolated antibody or epitope-binding fragment thereof according to (27).
   (29) A COVID-19 vaccine composition the positing the components in the amounts shown in Table 28.
   (30) A COVID-19 vaccine composition compositing the components in the amounts shown in Table 29.
   (31) A COVID-19 vaccine composition compositing the components in the amounts shown in Table 30.

### 8. Other Specific Embodiments

(1) A fusion protein having an amino acid sequence selected from the group consisting of S1-RBD-sFc (SEQ ID NO: 235, S1-RBDa-sFc (SEQ ID NO: 236), and S1-RBD-Fc (SEQ ID NO: 255).
(2) A composition comprising the fusion protein according to (1).
(3) The composition according to (2) further comprising a SARS-CoV-2 peptide selected from the group consisting of: SEQ ID NOs: 345, 346, 347, 348, 361, and any combination thereof.
(4) The composition according to any one of (2 or 3) further comprising a UBITh®1a peptide (SEQ ID NO: 66).
(5) The composition according to claim 2 further comprising:
a) a SARS-CoV-2 peptide selected from the group consisting of: SEQ ID NOs: 345, 346, 347, 348, 361, and any combination thereof; and
b) a UBITh®1a peptide (SEQ ID NO: 66).
   (6) A composition comprising:
a) the fusion protein according to (1),
b) a mixture of SARS-CoV-2 peptides comprising: SEQ ID NOs: 345, 346, 347, 348, and 361; and
c) a UBITh®1a peptide (SEQ ID NO: 66).
   (7) The composition according to any one of (5 or 6), wherein the fusion protein is S1-RBD-sFc SEQ ID NO: 235)
   (8) The composition according to any one of (5 or 6), wherein the fusion protein is S1-RBDa-sFc (SEQ ID NO: 236).
   (9) The composition according to any one of (5 or 6), wherein the fusion protein is S1-RBD-Fc (SEQ ID NO: 355).
   (10) A composition comprising:
a) a S1-RBD-sFC fusion protein,
b) a mixture of SARS-CoV-2 peptides comprising: SEQ ID NOs: 345, 346, 347, 348, and 361; and
c) a UBITh®1a peptide (SEQ ID NO: 66).
   (11) A SARS-CoV-2 vaccine composition comprising the fusion protein according to (1) and a pharmaceutically acceptable carrier and/or adjuvant.
   (12) The SARS-CoV-2 vaccine composition according to (11) further comprising a SARS-CoV-2 peptide selected from the group consisting of: SEQ ID NOs: 345, 346, 347, 348, 361, and any combination thereof.
   (13) The SARS-CoV-2 vaccine composition according to any one of or 12) further comprising a UBITh®1a peptide (SEQ ID NO: 66).
   (14) The SARS-CoV-2 vaccine composition according to (11) further comprising:
a) a SARS-CoV-2 peptide selected from the group consisting of: SEQ ID NOs: 345, 346. 347, 348, 361, and any combination thereof; and
b) a UBITh®1a peptide (SEQ ID NO: 66).
   (15) The SARS-CoV-2 vaccine composition according to any one of (11 to 14), wherein the pharmaceutically acceptable carrier and/or adjuvant is CpG1 (SEQ ID NO: 104).
   (16) A SARS-CoV-2 vaccine composition comprising:
a) the fusion protein according to (1),
b) a mixture of SARS-CoV-2 peptides comprising: SEQ ID NOs: 345, 346, 347, 348, and 361;
c) a UBITh®1a peptide (SEQ ID NO: 66), and
d) a pharmaceutically acceptable carrier and/or adjuvant.
   (17) The SARS-CoV-2 vaccine composition according to any one of (11 to 16), wherein the fusion protein is S1-RBD-sFc (SEQ ID NO: 235).
   (18) The SARS-CoV-2 vaccine composition according to any one of (11 to 16), wherein the fusion protein is S1-RBDa-sFc (SEQ ID NO: 236.
   (19) The SARS-CoV-2 vaccine composition according to any one of (11 to 16), wherein the fusion protein is S1-RBD-Fc (SEQ ID NO: 355.
   (20) The SARS-CoV-2 vaccine composition according to any one of (11 to 14 or 16 to 19), wherein the pharmaceutically acceptable carrier and/or adjuvant is CpG1 (SEQ ID NO: 104).
   (21) A SARS-CoV-2 vaccine composition comprising:
a) the S1-RBD-sFC fusion protein,
b) a mixture of SARS-CoV-2 peptides comprising: SEQ ID NOs: 345, 346, 347, 348, and 361;
c) a UBITh®1a peptide (SEQ ID NO: 66), and
d) a CpG1 oligonucleotide (SEQ ID NO: 104).
   (22) A method for immunizing a subject against SARS-CoV-2 comprising administering a pharmaceutically effective amount of the SARS-CoV-2 vaccine composition according to any one of (11 to 21) to the subject.
   (23) A method for immunizing a subject against SARS-CoV-2 comprising administering a pharmaceutically effective amount of the SARS-CoV-2 vaccine composition according to (21) to the subject.
   (24) A cell line transfected with a cDNA sequence encoding the fusion protein according to (1).
   (25) The cell line according to claim 24 that is a Chinese Hamster Ovary (CHO) cell line.
   (26) The cell line according to any one of (24 or 25), wherein the fusion protein is S1-RBD-sFc (SEQ ID NO: 235).
   (27) The cell line according to any one of (24 or 25), wherein the fusion protein is S1-RBDa-sFc (SEQ ID NO: 236).
   (28) The cell line according to any one of (24 or 25), wherein the fusion protein is S1-RBD-Fc (SEQ ID NO: 355).
   (29) The cell line according to (24 or 25), wherein the cDNA sequence is selected from the group consisting of S1-RBD-sFc (SEQ ID NO: 246), S1-RBDa-sFc (SEQ ID NO: 247), and S1-RBD-Fc (SEQ ID NO: 357).
   (30) The cell line according to (24 or 25), wherein the cDNA sequence is SEQ ID NO: 246 encoding S1-RBD-sFc.
   (31) The cell line according to (24 or 25), wherein the cDNA sequence is SEQ 11) NO: 247 encoding 51-RBDa-sFc.
   (32) The cell line according to (24 or 25), wherein the cDNA sequence is SEQ ID NO: 357 encoding S1-RBD-Fc.

### EXAMPLE 1

### Synthesis of S-RBD Related Peptides and Preparation of Formulations Thereof

a. Synthesis of S-RBD Related Peptides

Methods for synthesizing SARS-CoV-2 antigenic peptides, endogenous Th and CTL, and S-RBD related peptides that are included in the development of S-RBD peptide immunogen constructs are described. The peptides can be synthesized in small-scale amounts that are useful for serological assays, laboratory pilot studies, and field studies, as well as large-scale (kilogram) amounts, which are useful for industrial/commercial production of pharmaceutical compositions. A large repertoire of S-RBD B cell epitope peptides having sequences with lengths from approximately 6 to 80 amino acids were identified and selected to be the most optimal sequences for peptide immunogen constructs for use in an efficacious S-RBD targeted therapeutic vaccine.

Tables 1 to 3 provide the full-length sequences of SARS-CoV-2 M, N, and S proteins (SEQ ID NOs: 1, 6, and 20, respectively). Tables 1, 3, 11, and 13 also provide the sequences of antigenic peptides derived from SARS-CoV-2 M, N, E, ORF9b, and S proteins (SEQ ID NOs: 4-5, 17-18, 37-38, 4-5, 17-18, 37-38, 226, 227, 250-252, 259, 261, 263, 265, 266, 270, 281. 308, 321, 322, 323, 324, and 328-334) for use as solid phase/immunoadsorbent peptides for use in diagnostic assays for antibody detection. In addition, Tables 3, 11, and 13 provide the sequences of the full-length S-RBD, its fragments or modification thereof (SEQ ID NOs: 226, 227, 23-24, 26-27, 29-34, and 315-319).

Selected S-RBD B cell epitope peptides can be made into S-RBD peptide immunogen constructs by synthetically linking to a carefully designed helper T cell (Th) epitope peptide derived from pathogen proteins, including Measles Virus Fusion protein (MVF), Hepatitis B Surface Antigen protein (HBsAg), influenza, Clostridum tetani, and Epstein-Barr virus (EBV), identified in Table 6 (e.g., SEQ ID NOs: 49-100). The Th epitope peptides can be used either in a single sequence (e.g., SEQ ID NOs: 49-52, 54-57, 59-60, 62-63, 65-66 for MVF and SEQ ID NOs: 67-71, 73-74, 76-78 for HBsAg) or combinatorial library sequences (e.g., SEQ ID NOs: 53, 58, 61, 64 for MvF and SEQ ID NOs: 72 and 75 for HBsAg) to enhance the immunogenicity of their respective S-RBD peptide immunogen constructs. In order to generate memory T cells which would facilitate the recall of B cell or CTL responses of the vaccinated hosts to the SARS-CoV-2, SARS-CoV-2 derived endogenous Th and CTL epitopes are shown in Tables 2, 3, 4, 5, and 8 (SEQ ID NOs: 9-19, 35-48, 345-351) with known MHC binding activities are also designed as synthetic immunogens SEQ ID NOs: 345-351) and synthesized for inclusion in the final SARS-CoV-2 vaccine formulations.

Representative S-RBD peptide immunogen constructs selected from hundreds of peptide constructs are identified in Table 8 (SEQ ID NOs: 107-144). All peptides that can be used for immunogenicity studies or related serological tests for detection and/or measurement of anti-S-RBD antibodies can be synthesized on a small-scale using F-moc chemistry by peptide synthesizers of Applied BioSystems Models 430A, 431 and/or 433. Each peptide can be produced. by an independent synthesis on a solid-phase support, with F-moc protection at the N-terminus and side chain protecting groups of trifunctional amino acids. After synthesis, the peptides can be cleaved from the solid support and side chain protecting groups can be removed with 90% Trifluoroacetic acid (TFA). Synthetic peptide preparations can be evaluated by Matrix-Assisted Laser Desorption/Ionization-Time-Of-Flight (MALDI-TOF) Mass Spectrometry to ensure correct amino acid content. Each synthetic peptide can also be evaluated by Reverse Phase HPLC (RP-HPLC) to confirm the synthesis profile and concentration of the preparation. Despite rigorous control of the synthesis process (including stepwise monitoring the coupling efficiency), peptide analogues might also be produced due to unintended events during elongation cycles, including amino acid insertion, deletion, substitution, and premature termination. Thus, synthesized preparations can typically include multiple peptide analogues along with the targeted peptide.

Despite the inclusion of such unintended peptide analogues, the resulting synthesized peptide preparations will nevertheless be suitable for use in immunological applications including immunodiagnosis (as antibody capture antigens) and pharmaceutical compositions (as peptide immunogens). Typically, such peptide analogues, either intentionally designed or generated through synthetic process as a mixture of byproducts, are frequently as effective as a purified preparation of the desired peptide, as long as a discerning QC procedure is developed to monitor both the manufacturing process and the product evaluation process to guarantee the reproducibility and efficacy of the final product employing these peptides. Large scale peptide syntheses in the multi-hundred to kilo gram quantities can be conducted on a customized automated peptide synthesizer UBI2003 or the like at 15 mmole to 150 mmole scale or larger.

For active ingredients used in the final pharmaceutical composition for clinical trials, S-RBD peptide immunogen constructs can be purified by preparative RP-HPLC under a shallow elution gradient and characterized by MALDI-TOF mass spectrometry, amino acid analysis and ISP-HPLC for purity and identity.

b. Preparation of Compositions Containing S-RBD Peptide Immunogen Constructs

Formulations employing water-in-oil emulsions and in suspension with mineral salts can be prepared. In order for a pharmaceutical composition designed to be used by a large population, safety is another important factor for consideration. Despite the fact that water-in-oil emulsions have been used in humans as pharmaceutical compositions in many clinical trials, Alum remains the major adjuvant for use in pharmaceutical composition due to its safety. Alum or its mineral salts ADJUPHOS (Aluminum phosphate) are therefore frequently used as adjuvants in preparation for clinical applications.

Formulations in study groups can contain all types of designer S-RBD peptide immunogen constructs. A multitude of designer S-RBD peptide immunogen constructs can be carefully evaluated in guinea pigs for their relative immunogenicity against the corresponding S-RBD peptide used as the B cell epitope peptide or the full-length RBD polypeptide (SEQ 11) NOs: 226, 235, 236, and 255). Epitope mapping and serological cross-reactivities can be analyzed among the varying homologous peptides by ELISA assays using plates coated with the evaluated peptides (e.g., SEQ ID NOs: 23-24, 26-27, 29-34.315-319, and 335-344).

The S-RBD peptide immunogen constructs at varying amounts can be prepared in a water-in-oil emulsion with Seppic MONTANIDE™ ISA 51 as the approved oil for human use, or mixed with mineral salts ADJUPHOS (Aluminum phosphate) or ALHYDROGEL (Alum). Compositions can be prepared by dissolving the S-RBD peptide immunogen constructs in water at about 20 to 2,000 μg/mL and formulated with MONTANIDE™ ISA 51 into water-in-oil emulsions (1:1 in volume) or with mineral salts ADJUPHOS or ALHYDROGEL (Alum) (1:1 in volume). The compositions should be kept at room temperature for about 30 min and mixed by vortex for about 10 to 15 seconds prior to immunization. Animals can be immunized with 2 to 3 doses of a specific composition, which are administered at time 0 (prime) and 3 weeks post initial immunization (wpi) (boost), optionally 5 or 6 wpi for a second boost, by intramuscular route. Sera from the immunized animals can then be tested with selected B cell epitope peptide(s) to evaluate the immunogenicity of the various S-RBD peptide immunogen constructs present in the formulation and for the corresponding sera's cross-reactivity with the S-RBD site of SEQ ID NO: 26 or with the full-length S-RBD sequence (SEQ ID NO: 226). The S-RBD peptide immunogen constructs with potent immunogenicity found in the initial screening in guinea pigs can be further tested in in vitro assays for their corresponding sera's functional properties. The selected candidate S-RBD peptide immunogen constructs can then be prepared in water-in-oil emulsion, mineral salts, and alum-based formulations for dosing regimens over a specified period as dictated by the immunization protocols.

Only the most promising S-RBD peptide immunogen constructs will be further assessed extensively prior to being incorporated into final formulations in combination with or without the SARS-CoV-2 Th/CTL peptide constructs for immunogenicity, duration, toxicity and efficacy studies in GLP guided preclinical studies in preparation for submission of an Investigational New Drug application followed by clinical trials in patients with COVID-19.

### EXAMPLE 2

### Serological Assays and Reagents

Serological assays and reagents for evaluating functional immunogenicity of the S-RBD peptide immunogen constructs and formulations thereof are described in detail below.

a. S-RBD or S-RBD B Cell Epitope Peptide-Based ELISA Tests for Immunogenicity and Antibody Specificity Analysis

ELISA assays that can be used to evaluate immune serum samples and/or samples from individuals for the detection of COVID-19 are described below.

The wells of 96-well plates are coated individually for 1 hour at 37° C. with 100 μL of S-RBD (SEQ ID NO: 226) or with S-RBD B cell epitope peptides (e.g., SEQ ID NOs: 23-24, 26-27, and/or 29-34), at 2 μg/mL (unless noted otherwise), in 10 mM NaHCO₃ buffer, pH 9.5 (unless noted otherwise).

The S-RBD or S-RBD B cell epitope peptide-coated wells are incubated with 250 μL of 3% by weight gelatin in PBS at 37° C. for 1 hour to block non-specific protein binding sites, followed by three washes with PBS containing 0.05% by volume TWEEN® 20 and dried. Sera to be analyzed are diluted 1:20 (unless noted otherwise) with PBS containing 20% by volume normal goat serum, 1% by weight gelatin and 0.05% by volume TWEEN® 20. One hundred microliters (100 μL) of the diluted specimens (e.g., serum, plasma) is added to each of the wells and allowed to react for 60 minutes at 37° C. The wells are then washed six times with 0.05% by volume TWEEN® 20 in PBS in order to remove unbound antibodies. Horseradish peroxidase (HRP)-conjugated species (e.g., guinea pig or rat) specific goat polyclonal anti-IgG antibody or Protein A/G are used as a labeled tracer to bind with the antibody/peptide antigen complex formed in positive wells. One hundred microliters (100 μL) of the HRP-labeled detection reagent, at a pre-titered optimal dilution and in 1% by volume normal goat serum with 0.05% by volume TWEEN® 20 in PBS, is added to each well and incubated at 37° C. for another 30 minutes. The wells are washed six times with 0.05% by volume TEEN® 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture is used to detect the peroxidase label by forming a colored product. Reactions are stopped by the addition of 100 μL of 1.0 M H₂SO₄ and absorbance at 450 nm (A₄₅₀) is determined. For the determination of antibody titers of the vaccinated animals that received the various peptide vaccine formulations, or individuals who are being tested for infection with SARS-CoV-2, 10-fold serial dilutions of sera from 1:100 to 1:10,000 or 4-fold serial dilutions of sera from 1:100 to 1:4.19×10⁸ are tested, and the titer of a tested serum, expressed as Logic), is calculated by linear regression analysis of the A₄₅₀ with the cutoff A₄₅₀ set at 0.5.

b. Assessment of Antibody Reactivity Towards Th Peptide by Th Peptide-Based ELISA Tests

The wells of 96-well ELISA plates are coated individually for 1 hour at 37° C. with 100 μL of Th peptide at 2 μg/mL (unless noted otherwise), in 10 mM NaHCO₃ buffer, pH 9.5 (unless noted otherwise) in similar ELISA method and performed as described above. For the determination of antibody titers of the vaccinated animals that received the various formulations containing S-RBD peptide immunogen constructs, 10-fold serial dilutions of sera from 1:100 to 1:10,000 are tested, and the titer of a tested serum, expressed as Log₁₀, is calculated by linear regression analysis of the A₄₅₀ with the cutoff A₄₅₀ set at 0.5.

c. Immunogenicity Evaluation

Preimmune and immune serum samples from animal subjects are collected according to experimental vaccination protocols and heated at 56° C. for 30 minutes to inactivate serum complement factors. Following the administration of the formulations containing the S-RBD peptide immunogen constructs, blood samples can be obtained according to protocols and their immunogenicity against specific target site(s) can be evaluated using the corresponding S-RBD B cell epitope peptide-based. ELISA tests. Serially diluted sera can be tested, and positive titers can be expressed as Log₁₀ of the reciprocal dilution. Immunogenicity of a particular formulation is assessed for its ability to elicit high titer antibody response directed against the desired epitope specificity within the target antigen and high cross-reactivities with the S-RBD polypeptide, while maintaining a low to negligible antibody reactivity towards the helper T cell epitopes employed to provide enhancement of the desired B cell responses.

### EXAMPLE 3

### Peptide Compositions Having a Mixture of Antigenic SARS-CoV-2 Peptides in Assay Formulation Enhances Sensitivity

Although early detection of COVID-19 is done by laboratory criteria such as RT-PCR. assays using molecular probes and by clinical criteria such as elevated body temperature, non-produaive cough, etc., an antibody detection assay that is both sensitive and specific is desirable for serological surveillance.

In developing the disclosed COVID-19 antibody detection assays for serosurveillance and diagnosis, assay specificity is considered a high priority. High specificity is a requisite of an acceptable COVID-19 antibody test so as not to misdiagnose patients for unnecessary isolation, and to avoid the unnecessary implementation of emergency public health measures to contain an outbreak.

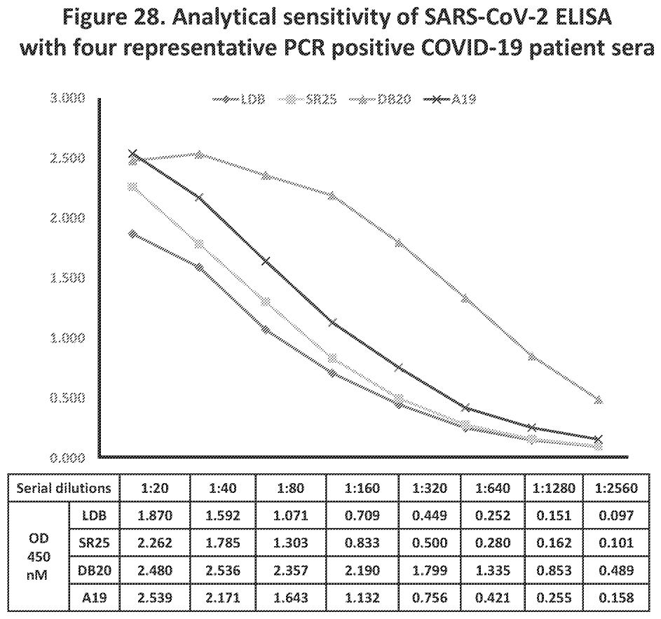

An acceptable immunoassay for serosurveillance and diagnosis must also have high sensitivity. Therefore, mixtures of the corresponding antigenic peptides derived from SARS-CoV-2 M, N, and S proteins, based on previous knowledge of SARS-CoV serology, as peptide homologues (e.g., SEQ ID NOs: 4, 17 and 31), and those designed and identified through extensive serological validation (e.g., SEQ ID NOs: 4, 17, 37, 262, 265, 281, 322, 354) are evaluated as antigens for complimentary sensitivity for antibody detection. In order to enhance the binding capability of selected peptides to ELISA plates, a KKK-lysine tail is added at the N-terminus of each of the selected peptide analogues (e.g., SEQ ID NOs: 5, 18, and 38). Moreover, upon extensive testing, the use of the peptide mixtures should not result in a loss of specificity of the peptide mixtures for the normal sera. Therefore, a mixture of antigenic peptides comprising peptides having the amino acid sequences of SEQ ID NOs: 5, 18, and 38 can be retained for the assay formulations as the solid phase antigen adsorbent. Similarly, a mixture comprising antigenic peptides having the amino acid sequences of SEQ ID NOs: 5, 18, 38, 261, 266, 281, and 322 can be used for the assay formulations as the solid phase antigen adsorbent to have enhanced analytical sensitivity (FIG. 28). These antigenic peptides having amino acid sequences of SEQ ID NOs: 5, 18, 38, 261, 266, 281, 322 can also be formulated individually as the solid phase adsorbent for corresponding component ELISAs each with high specificity, and together they form a confirmatory assay to provide antigenic profiles for an individual shown to be positive for SARS-CoV-2 infection.

### EXAMPLE 4

Evaluation OF COVID-19 Enzyme Immunoassay in Infected, Random Blood Donor, and Other Non-SARS-CoV-2 Infected Populations, in a Large Scale Analysis

a. Sera from Patients Infected with Other Viruses and Normal Sera

Sera obtained prior to 2000 from patients with other viral infections unrelated to COVID-19 are well documented by serological markers. A large panel of sera from normal blood donors was obtained from a Florida Blood bank. The seroprevalence rate for reactivity to SARS-CoV-2 in these sera panels, collected at least three years prior to the report of any known COVID-19 cases were used to evaluate the specificity of the COVID-19 ELISA.

b. Analysis by a Mixed Peptide-Based COVID-19 ELISA for the Detection of SARS-CoV-2

ELISA assays for the detection of SARS-CoV-2 were conducted on 96-well microtiter plates coated with a mixture of SARS-CoV-2 M, N, and S peptides, and with sera diluted 1:20 by the method described below. The wells of 96-well plates were coated separately for 1 hour at 37° C. with 2 μg/mL, of SARS-CoV-2 M, N, and S protein-derived peptide mixture using 100 μL per well in 10mM NaHCO₃ buffer, pH 9.5 unless noted otherwise. The peptide-coated wells were incubated with 250 μL of 3% by weight of gelatin in PBS in 37° C. for 1 hour to block non-specific protein binding sites, followed by three washes with PBS containing 0.05% by volume of TWEEN 20 and dried. Patient sera positive for SARS-CoV-2-reactive antibody by IFA and control sera were used as a positive control through their cross-reactivities with the SARS-CoV-2 peptide coated wells at a 1:20 dilution, unless otherwise noted, with PBS containing 20% by volume normal goat serum, 1% by weight gelatin and 0.05% by volume TWEEN 20. One hundred microliters (100 μL) of the diluted specimens were added to each of the wells and allowed to react for 60 minutes at 37° C. The wells were then washed six times with 0.05% by volume TWEEN 20 in PBS in order to remove unbound antibodies. Horseradish peroxidase-conjugated goat anti-human IgG was used as a labeled tracer to bind with the SARS-CoV-2 antibody/peptide antigen complex formed in positive wells. One hundred microliter (100 μL) of the peroxidase-labeled goat anti-human IgG at a pretitered optimal dilution and in 1% by volume normal goat serum, 0.05% by volume TWEEN 20 in PBS, was added to each well and incubated at 37° C. for another 30 minutes. The wells were washed six times with 0.05% by volume TWEEN 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1.0 M H₂SO₄ and absorbance at 450 nm (A₄₅₀) determined,

c. Criteria for Interpretation

Significant reactivity in the ELISA format, i.e., the cutoff value, was scored by A₄₅₀ absorbances which were greater than the mean A₄₅₀ plus six standard deviations of the distribution of sera from the normal population.

d. Results

The samples from a panel of over 500 normal plasma and serum samples with a presumed zero seroprevalence rate were tested at 1:20 dilutions to assess their respective reactivities in the mixed peptide SARS-CoV-2 ELISA. The normal donor samples gave a mean A₄₅₀ of 0.074±0.0342 (SD), establishing a cutoff value of A₄₅₀ 0.274. The distribution of the Signal to Cutoff (S/C) ratio for the normal sera with the peak S/C ratio having a value of 0.3, with none of the samples showing positive reactivity. Thus, the specificity of this ELISA on the normal samples was 100% at the set cutoff value.

The SARS-CoV-2 ELISA, using peptide homologues with corresponding SARS-CoV-2 derived sequences, are further evaluated for specificity by testing with a large panel of samples from patients with infections unrelated to SARS-CoV-2, such as HIV-1, HIV 2, HCV, HTLV 1/II, and syphilis, and with normal serum samples spiked with interference substances.

Further serological analysis with sera obtained from infected COVID-19 patients from Taiwan, Shanghai, Beijing and WuHan are to be tested to reconfirm the efficacy of the mixed peptide SARS-CoV-2 ELISA. All sera obtained from patients with confirmed COVID-19 and samples shown to have antibody titers against SARS-CoV-2 as detected by IFA, along with serial bleed dates ranging from days 0 to 30 and even longer period are to be tested to assess the seroconversion process and the persistence of such antibodies. Results from these pedigreed seroconversion panels would provide information indicated the earliest detectable levels of anti-SARS-CoV-2 M, N, and S antibodies upon infection and the period throughout for persistence of such antibodies. It is particularly important to conduct large scale serological screening of at-risk individuals including hospital healthcare workers, taxi drivers, airplane stewardesses, and others who are in constant touch with general public to identify those rare super spreaders (<2% found as of this filing date) individuals who are infected by SARS-CoV-2, have high viral load yet remain asymptomatic, to minimize unknown infection to endanger the heath of the general public unintentionally.

In summary, a highly sensitive and specific SARS-CoV-2 antibody detection test in the simple, rapid, and convenient ELISA format was developed for the large-scale application of serosurveillance for COVID-19. The test is based on a solid phase immunosorbent comprising antigenic synthetic peptides corresponding to segments of the SARS-CoV-2 M, N, and S proteins and immunologically functional analogues thereof, branched as well as linear forms, conjugates, and polymers. The immunoassay is suitable for use in combination with molecular probe-based or other virus detection systems. The high specificity of this peptide-based SARS-CoV-2 immunoassay system, provided by the high stringency imposed on the selection of the SARS-CoV-2 antigenic peptides, and the high sensitivity provided by the mixture of peptides having complementary site-specific epitopes results in a test that is appropriate for national epidemiological surveys. Such tests can be used by countries suffering from COVID-19 outbreak or suspecting the presence of COVID-19 for look back epidemiology studies. Also, a highly specific immunoassay can be used to differentiate SARS-CoV-2 infection from diseases caused. by unrelated respiratory viruses and bacteria. An immunoassay of the invention can eliminate the untoward over-reporting of COVID-19, reduce the number of patients in isolation, and reduce other costs associated with emergency measures to contain disease transmission.

### EXAMPLE 5

### Animals Used in Safety, Immunogenicity Toxicity, and Efficacy Studies

a. Guinea Pigs:

Immunogenicity studies can be conducted in mature, naïve, adult male and female Duncan-Hartley guinea pigs (300-350 g/BW). The experiments utilize at least 3 Guinea pigs per group.

Protocols involving Duncan-Hartley guinea pigs (8-12 weeks of age; Covance Research Laboratories, Denver, Pa., USA) are performed under approved IACUC applications at a contracted animal facility under UBI sponsorship.

b. Cynomolgus Macaques:

Immunogenicity and repeated dose toxicity studies in adult male and female monkeys (Macaca fascicularis, approximately 3-4 years of age; JOINN Laboratories, Suzhou, China) are conducted under approved IACUC applications at a contracted animal facility under UBI sponsorship.

### EXAMPLE 6

### Assessment of Functional Properties of Antibodies Elicited by the S-RBD Peptide Immunogen Constructs and Formulations Thereof

Immune sera or purified anti-S-RBD antibodies produced in guinea pigs can be further tested for their ability to (1) bind to S-RBD peptide and polypeptides having the sequences of SEQ ID NOs: 26, 226, and 227; (2) inhibit binding by S-RBD protein to ACE2 receptor in an ELISA assay and an immunofluorescent ACE2 surface expression binding assay; and (3) neutralize in vitro target cell viral replication.

a. Antibody Binding Assay

The aim of this assay is to demonstrate that the immune sera derived from immunized guinea pigs could recognize SARS-CoV-2 Spike (S) protein. Specifically, 1 μg/ml recombinant S proteins is used to coat onto 96-well microliter plates (MaxiSorp NUNC) in 0.1 M carbonate buffer (pH 9.6) at 4° C. overnight. After blocking with 2% BSA, serially diluted antisera are added and incubated at 37° C. for 1 h with shaking, followed by four washes with PBS containing 0.1% TWEEN 20. Bound antisera are detected with Goat Anti-Guinea pig IgG H&L (HRP) (ABcam, ab6908) at 37° C. for 1 h, followed by 4 washes. The substrate, 3,3,5,5-tetramethylbenzidine (TMB), is added into each well and incubated at 37° C. for 20 minutes. The absorbance at 450 nm is measured by an ELISA plate reader (Molecular Device).

b. Antibody Neutralization Assay

The aim of this assay is to demonstrate if antibodies in the immune sera from animals that have been administered with S-RBD peptide immunogen constructs (SEQ ID NOs: 107-144) or S-RBD fusion proteins (S-RBD-sFc and S-RBDa-sFc of SEQ ID NOs: 235 and 236, respectively) have neutralizing or receptor binding inhibition properties in the presence of the ACE2 receptor. Specifically, 1 μg/ml recombinant S protein (SEQ ID NO: 20) or S-RBD protein (SEQ ID NO: 226, 227) is used to coat onto 96-well microtiter plates (MaxiSorp NUNC) in 0.1 M carbonate buffer (pH 9.6) at 4° C. overnight. After blocking with 2% BSA, serially diluted immune sera are co-incubated with hACE2 at 37° C. in S protein or S-RBD polypeptide coated 96 well plate for 1 hour, followed by four washes with PBS containing 0.1% Tween 20. Bound ACE2^{ECD} or ACE2N^{ECD} peptides (SEQ ID NO: 229-230) are detected with Goat-anti-HuACE2 Ab (HRP) (R&D System) at 37° C. for 1 hour, followed by 4 washes. The substrate, 3,3,5,5-tetramethylbenzidine (TMB), is added in to each well and incubated at 37° C. for 20 minutes. The absorbance at 450 nm is measured by an ELISA plate reader (Molecular Device). The signal is in reverse proportion to the neutralization antibody concentration. The neutralization titers would be presented as reciprocal of the serum dilution fold.

c. Cell-Based Neutralization Assay (Flow Cytometry)

The neutralization assay for SARS-CoV-2 S protein binding to ACE2-expressed cells by immune sera directed against S-RBD (S-RBD peptide immunogen constructs, S-RBD-sFc fusion protein, or S-RBDa-sFc fusion protein) is measured by flow cytometry. Briefly, 10⁶ HEK293/ACE2 cells are detached, collected, and washed with HBSS (Sigma-Aldrich). S protein from SARS-CoV-2 is added to the cells to a final concentration of 1 μg/mL in the presence or absence of serial diluted immune sera, followed by incubation at room temperature for 30 min. Cells are washed with HBSS and incubated with anti-SARS-CoV-2 S protein antibody (HRP) at 1/50 dilution at room temperature for an additional 30 min. After washing, cells are fixed with 1% formaldehyde in PBS and analyzed in a FACSCalibur flow cytometer (BD Biosciences) using CellQuest software.

d. Neutralization of SARS-CoV-2 Infection

After immune sera derived from guinea pigs immunized with S-RBD peptide immunogen constructs, S-RBD-sFc fusion protein, or S-RBDa-sFc fusion protein demonstrates effectiveness to neutralize hACE2 in in vitro assays, the immune sera will be tested in a SARS-CoV-2 neutralization assay.

Briefly, Vero E6 cells are plated at 5×10⁴ cells/well in 96-well tissue culture plates and grow overnight. One hundred microliters (100 μL) of 50% tissue-culture infectious dose of SARS-CoV-2 is mixed with an equal volume of diluted guinea pig immune sera and incubated at 37° C. for 1 h. The mixture is added to monolayers of Vero E6 cells. Cytopathic effect (CPE) is recorded on day 3 post-infection. Neutralizing titers representing the dilutions of GP immune sera that completely prevented CPE in 50% of the wells is calculated by Reed-Munch method.

### EXAMPLE 7

Assays Employed in the Development Of ACE2-SFC Fusion Protein as Antiviral Therapies

1. Assays for the hACE2 Protein Drug Development
a. Binding Assay

The following assay is designed to demonstrate that the hACE2 fusion proteins (ACE2-ECD-sFc, ACE2N-ECD-sFc of SEQ ID NOs: 237-238) can be recognized by its natural ligand (the S protein of SARS-CoV-2) in comparison with ACE2-ECD-Fc. Specifically, 1 μg/ml recombinant S protein (Sino Biological) is used to coat 96-well microtiter plates (MaxiSorp NUNC) in 0.1 M carbonate buffer (pH 9.6) at 4° C. overnight. After blocking with 2% BSA, ACE2 protein at a concentration of 0.5 μg/mL is added and incubated at 37° C. for 1 h with shaking, followed by four washes with PBS containing 0.1% TWEEN 20. Bound ACE2 proteins are detected with rabbit anti-human ACE2 polyclonal antibody:HRP (My Biosource, CN: MBS7044727) at 37° C. for 1 h, followed by 4 washes. The substrate, 3,3,5,5-tetramethylbenzidine (TMB), is added into each well and incubated at 37° C. for 20 minutes. The absorbance at 450 nm is measured by an ELISA plate reader (Molecular Device).

b. Blocking Assay

The aim of this assay is to demonstrate if the binding between the S protein and ACE2 can be blocked by the ACE2 fusion proteins (ACE2-ECD-sFc and ACE2N-ECD-sFc of SEQ ID NOs: 237 and 238, respectively) in comparison to ACE2-ECD-Fc. Specifically, 1 μg/ml ACE2 is used. to coat on 96-well microtiter plates (MaxiSorp NUNC) in 0.1 M carbonate buffer (pH 9.6) at 4° C. overnight. After blocking with 2% BSA, serially diluted recombinant ACE2 proteins are co-incubated with SARS-CoV-2 S protein at 37° C. for 1 hour, followed by four washes with PBS containing 0.1% TWEEN 20. Bound S protein is detected with anti-SARS-CoV-2 S antibody (HRP) at 37° C. for 1 hour, followed by 4 washes. The substrate, 3,3,5,5-tetramethylbenzidine (TMB), is added into each well and incubated at 37° C. for 20 minutes. The absorbance at 450 nm is measured by an ELISA plate reader (Molecular Device). The signal is in proportion to the reciprocal of dilution fold of the proteins.

c. Cell-Based Neutralization Assay (Flow Cytometry)

The neutralization of SARS-CoV-2 S protein binding to ACE2-expressed cells by ACE2 fusion proteins (ACE2-ECD-sFc and ACE2N-ECD-sFc of SEQ ID NOs: 237 and 238, respectively) is measured by flow cytometry. Briefly, 10⁶ HEK293/ACE2 cells are detached, collected, and washed with HBSS (Sigma-Aldrich). The SARS-CoV-2 S protein is added to the cells to a final concentration of 1 μg/mL in the presence or absence of serial diluted the ACE2 recombinant proteins, followed by incubation at room temperature for 30 min. Cells are washed with HBSS and incubated with Anti-SARS-CoV-2 S Ab (HRP) at 1/50 dilution at room temperature for an additional 30 min. Ater washing, cells are fixed with 1% formaldehyde in PBS and analyzed in a FACSCalibur flow cytometer (BD Biosciences) using CellQuest software.

d. Affinity Determination by SPR Assay

S-RBD-Fc is immobilized on a CM5 sensor chip as shown in the instruction manual of Capture kit (GE, BR100839) with an SPR instrument (GE, Biacore X100). :For a reaction cycle, a constant level of recombinant protein is initially captured onto the sensor chip. Sequentially, the samples (ACE2-ECD-sFc or ACE2N-ECD-sFc) are flowed at various concentrations in each cycle through the chip for association followed by flowing running buffer through for dissociation. Finally, the chip is regenerated with regeneration buffer for next reaction cycle. For data analysis, the binding patterns (or sensorgrams) from at least five reaction cycles are analyzed with BIAevaluation software to acquire affinity parameters such as KD, Ka and kd.

### EXAMPLE 8

### Design, Plasmid Construction, and Protein Expression of S-RBD Fusion Proteins in Cho Cells

1. Design of the cDNA Sequence

The cDNA sequence of the S protein from SARS-CoV-2 (SEQ ID NO: 239) is optimized for CHO cell expression. This nucleic acid encodes the S protein shown as SEQ ID NO: 20. The receptor binding domain (RBD) of the S protein was identified by aligning with the S protein sequence of SARS-CoV (SEQ ID NO: 21) with the corresponding sequence from SARS-CoV-2 (SEQ ID NO: 20). The S-RBD polypeptide from SARS-CoV-2 (aa331-530) (peptide SEQ (D NO: 226; DNA SEQ ID NO: 240) corresponds with the S-RBD sequence of SARS-CoV, which was proved to be the binding domain binding to hACE2 with high affinity.

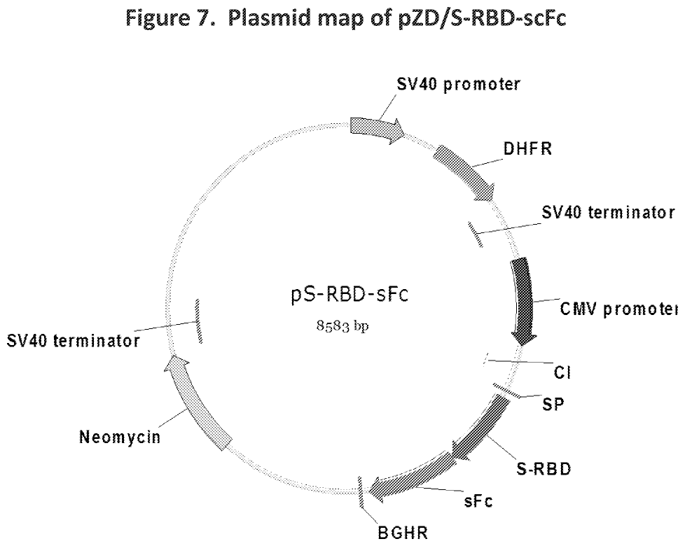

To develop a pharmaceutical composition to protect individuals from COVID-19 infection, the RBD of the S protein is an important target for inducing the antibodies to neutralize SARS-CoV-2 after immunization. To produce the S-RBD-Fc fusion protein (DNA SEQ ID NO: 246), the nucleic acid sequence encoding S-RBD (aa331-530) of SARS-CoV-2 (DNA SEQ ID NO: 240) is fused to the N-terminus of the single chain of the immunoglobulin Fc (DNA SEQ ID NO: 245), as shown in FIG. 6A and the plasmid map shown in FIG. 7. To avoid mismatch of the non-critical disulfide bond formation in the S-RBD fusion protein in CHO expression system, Cys391 replaced by Ala391 and Cys525 replaced by Ala525 in the S-RBD polypeptide (amino acid SEQ ID NO: 227; DNA SEQ ID NO: 241) to produce the S-RBDa-sFc fusion protein (amino acid SEQ ID NO: 236; DNA SEQ ID NO: 247).

To develop the neutralizing intervention by virus inhibition as passive immunization. human angiotensin converting enzyme II (ACE2 accession NP_001358344, amino acid SEQ ID NO: 228; DNA SEQ ID NO: 242), which acts as the receptor of SARS-CoV-2 to mediate virus entrance, is the key target to block the S protein. In a previous study (Sui J., et al. 2004), the binding affinity is 1.70E-9 that corresponds to potent mAb for neutralization. Administration of high dose ACE2 should be safe enough for treatment of coronavirus infected patients since some of the ACE2 clinical trial for hypertension treatment demonstrated the safety profile with very high dose administration (Arendse, L. B. et al. 2019).

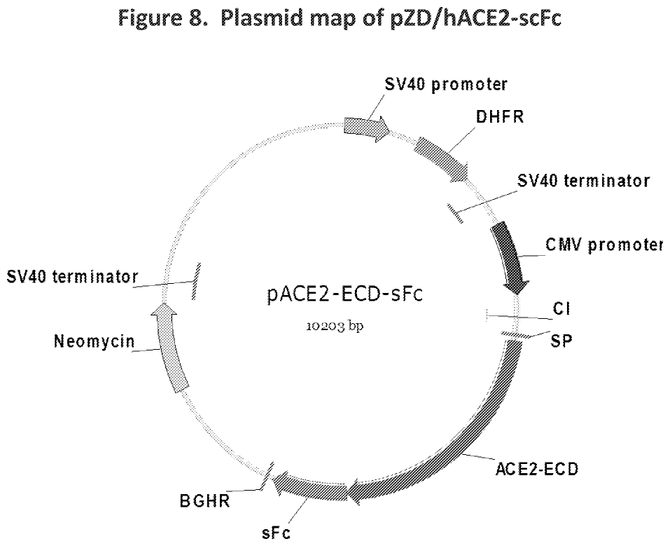

The extra-cellular domain of ACE2 (amino acid SEQ ID NO: 229; DNA SEQ ID NO: 243) is fused with single chain immunoglobulin Fc (amino acid SEQ ID NO: 232; DNA SEQ ID NO: 245) to produce the S-ACE2_{ECD}-Fc fusion protein (DNA SEQ ID NO: 248), as shown in FIG. 6C and the plasmid map shown in FIG. 8. To reduce the safety uncertainty, a fusion protein can be produced that abolishes peptidase activity in the ACE2_{ECD} fusion protein in CHO expression system. Specifically, His374 is replaced by Asn374 and His378 is replaced by Asn378 in zinc binding domain of ACE2 (amino acid SEQ ID NO: 230; DNA SEQ ID NO: 244) to produce the ACE2N_{ECD} fusion protein (amino acid SEQ ID NO: 238; DNA SEQ ID NO: 249). Since no disulfide bonds form in the hinge region, the large protein fusion with sFc would not constrain the binding to S protein to achieve the most potent neutralization effect. The structure of single chain Fc also has the advantage to be purified by protein A binding and elution in purification process. Other disulfide bond forming with Cys345-Cys370, Cys388-Cys441 and Cys489-Cys497 still reserved in the sequence design to maintain the conformation binding to ACE2.

### 2. Plasmid Construction and Protein Expression

a. Plasmid Construction

To express the S-RBD-Fc and S-RBDa-Fc fusion proteins, the cDNA sequences encoding these proteins can be produced in an appropriate cell line. The N-terminus of the cDNA fragment can be added a leader signal sequence for protein secretion, and the C-terminus can be linked to single-chain Fc (sFc) or a His-tag following a thrombin cleavage sequence. The cDNA fragments can be inserted into the pND expression vector, which contains a neomycin-resistance gene for selection and a dhfr gene for gene amplification. The vector and the cDNA fragments are digested with Paci/EcoRV restriction enzymes, and then ligated to yield four expression vectors, pS-RBD, pS-RBD-sFc, pS-RBDa, and pS-RBDa-sFc.

To express the ACE2_{ECD} and ACE2N_{ECD} fusion proteins, the CDNA sequences encoding these proteins can be produced in an appropriate cell line. The C-terminus of the cDNA fragment can be linked to single-chain Fc or a His-tag following a thrombin cleavage sequence. The cDNA fragments can be inserted into pND expression vector to yield four expression vectors, pACE2-ECD, pACE2-ECD-sFc, pACE2N-ECD, pACE2N-ECD-sFc.

b. Host Cell Line

CHO-S™ cell line (Gibco. A1134601) is a stable aneuploid cell line established from the ovary of an adult Chinese hamster. The host cell line CHO-S™ are adapted to serum-free suspension growth and compatible with FREESTYLE™ MAX Reagent for high transfection efficiency. CHO-S cells are cultured in DYNAMIS™ Medium (Gibe° , Cat. A26175-01) supplemented with 8 mM Glutamine supplement (Life Technologies, Cat. 25030081) and anti-dumping agent (Gibco, Cat. 0010057DG).

ExpiCHO-S™ cell line (Gibco, Cat. A29127) is a clonal derivative of the CHO-S cell line. ExpiCHO-S™ cells are adapted to high-density suspension culture in ExpiCHO™ Expression Medium (Gibco, Cat. A29100) without any supplementation. The cells are maintained in a 37° C. incubator with a humidified atmosphere of 8% CO₂.

c. Transient Expression

For transient expression, the expression vectors are individually transfected into ExpiCHO-S cells using EXPIFECTAMINE™ CHO Kit (Gibco, Cat. A29129). On day 1 post-transfection, EXPIFECTAMINE™ CHO Enhancer and first feed is added, and the cells are transferred from a 37° C. incubator with a humidified atmosphere of 8% CO₂ to a 32° C. incubator with a humidified atmosphere of 5% CO₂. Then, the second feed is added on day 5 post-transfection, and the cell culture is harvested after 12-14 days post-transfection. After the cell culture is harvested, the supernatant is clarified by centrifugation and 0.22-μm filtration. The recombinant proteins containing single-chain Fc and His-tag are purified by protein A chromatography (Gibco, Cat. 101006) and Ni-NTA chromatography (Invitrogen, Cat. R90101), respectively.

d. Stable Transfection and Cell Selection

The expression vector is transfected into CHO-S cells using FreeStyle MAX reagent (Gibco, Cat. 16447500) and then incubation with selection DYNAMIS™ medium, containing 8 mM L-Glutamine, anti-clumping agent at 1:100 dilution, puromycin (InvovoGen, Cat. ant-pr-1), and MTX (Sigma, Cat. M8407). After 2 rounds of selection phase, four stable pools (1A, 1B, 2A, 2B) are obtained. Furthermore, the cell clones are plated in semi-solid CloneMedia (Molecular Devices, Cat. K8700) and simultaneously added detection antibody for clone screening and single cell isolation by high throughput system ClonePixTM2 (CP2). The clones picked by CP2 are screened by using a 14-day glucose simple fed-batch culture in DYNAMIS™ Medium with 8 mM Glutamine and anti-clumping agent without selections. After screening, single cell isolation of the clones with high yield are performed by limiting dilution, and the monoclonality is confirmed by imaging using CloneSelect lmager (Molecular Devices).

e. Simple Fed-Batch Culture

A simple ted-batch culture is used to determine the productivity of CHO-S cells expressing the recombinant proteins. CHO-S cells are seeded at 3×10⁵ cells/mL with 30 mL DYNAMIS medium supplemented, 8 mM Glutamine and anti-clumping agent at 1:100 dilution in 125-mL shaker flasks. The cells are incubated in a 37° C. incubator with a humidified atmosphere of 8% CO2. 4 g/L of glucose are added on day 3 and 5, and 6 g/L of glucose are added on day 7. The cultures are collected daily to determine the cell density, viability, and productivity until the cell viability dropped below 50% or day 14 of culture is reached.

f. Accuracy of Gene Transcript

The accuracy of the gene transcription by the CHO-S expressing cells is confirmed by RT-PCR. Briefly, total RNA of the cells is isolated using PURELINK™ RNA Mini Kit (Invitrogen Cat. 12183018A). Then, the first strand cDNA is reverse transcribed from total RNA using Maxima H Minus First Strand cDNA Synthesis Kit (Thermo Cat. K1652). The cDNA of the recombinant proteins is purified and ligated into yT&A Vector (Yeastern Biotech Co., Ltd Cat.YC203). Finally, the cDNA sequence is confirmed by DNA sequencing.

g. Stability of the Expressing Cells

The cells are seeded at 1˜2×10⁵ cells/mL and cultured in a medium without selection reagents for 60 generations. Once the cell density of the cultures reached 1.0×10⁶ cells/mL or more during this period, the cultures are passaged at the cell density at 1˜2×10⁵ cells/mL again. After cultivation for 60 generations, the cell performance and productivity are compared to the cells which had just been thawed from the LMCB using glucose simple fed-batch culture. The criterion of stability of product productivity in cells is titer greater than 70% after cultivation for 60 generations.

### EXAMPLE 9

### Purification and Biochemical Characterization of sFc Fusion Proteins and His-Tagged Proteins

### 1. Purification of sFc Fusion Proteins

All sFc fusion proteins were purified by protein A-sepharose chromatography from the harvested cell culture conditioned medium. The sFc fusion proteins were captured by a Protein A affinity column. After washing and eluting, the pH of protein solution was adjusted to 3.5. The protein solution was then neutralized to pH 6.0 by the addition of 1 M Tris base butler, pH 10.8. The purity of the fusion protein was determined by polyacrylamide gel electrophoresis. The protein concentration was measured according to the UV absorbance at a wavelength of 280 nm.

### 2. His-Tagged Proteins

Conditioned medium was mixed with Ni-NTA resin to purify fusion proteins according to manufacturer's manual. His-tagged proteins were eluted in the elution containing 50 mmol·L—1 NaH₂PO₄, 300 mmol·L—1 NaCl, and 250 mmol·L—1 imidazole, at pH 8.0. The eluted solution was concentrated using Amicon YM-5 and then passed through a Sephadex G-75 column to get rid of impurities and a Sephadex G-25 column to remove salts; then collected protein solution was lyophilized. The purity of the His-Tagged proteins was determined by polyacrylamide gel electrophoresis. The protein concentration was measured according to the UV absorbance at a wavelength of 280 nm.

3. Biochemical Characterization of sFc fusion Proteins and His-Tagged Proteins used for (1) High Precision ELISA for Measurement of Neutralizing Antibodies in SARS-CoV-2 Infected, Recovered, or Vaccinated Individuals, (2) as Immunogens for the Prevention of SARS-CoV-2 Infection, and (3) a Long-Acting Antiviral Protein for Treatment of COVID-19.

S1-RBD-His (SEQ ID NO: 335), S1-RBD-sFc (SEQ ID NO: 235), and ACE2-ECD-sFc (SEQ ID NO: 237) were prepared and purified according to the methods described above for use as (1) reagents in a high precision ELISA for measuring neutralizing antibodies in infected, recovered COVID-19 patients, or in SARS-CoV-2 vaccinated individuals, (2) a representative immunogen in a high precision designer vaccine formulation for prevention of SARS-CoV-2 infection, and (3) as a long acting antiviral protein for treatment of COVID-19.

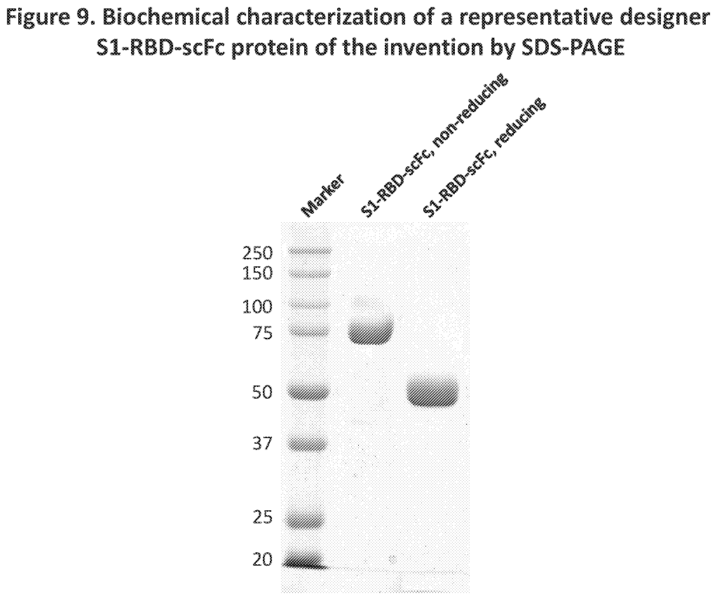

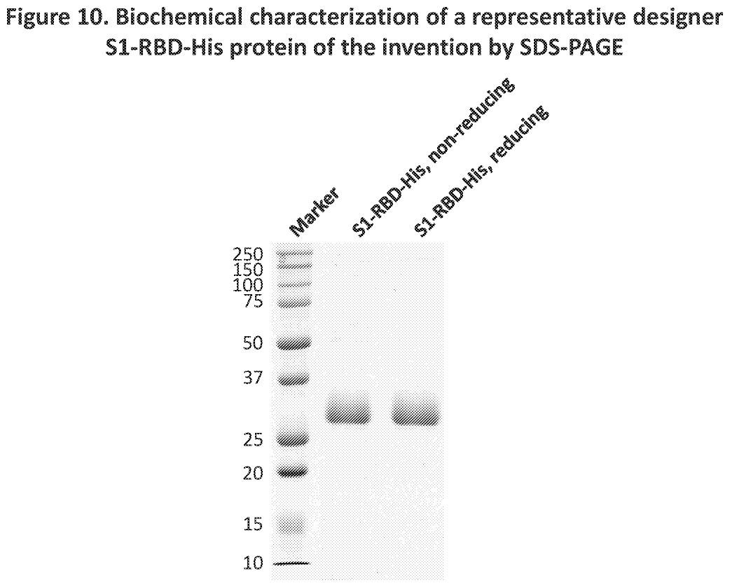

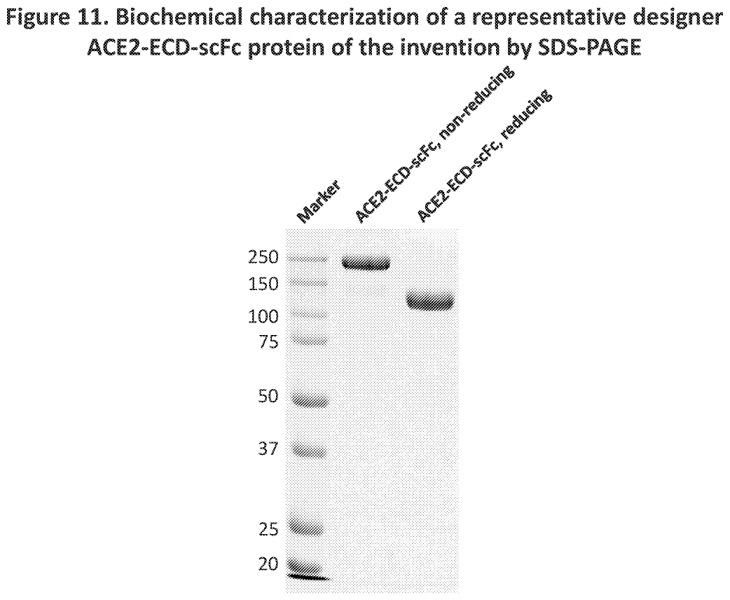

After purification of the sFc fusion proteins and His-tagged proteins, the purity of the proteins was determined by SDS-PAGE using Coomassie blue staining under non-reducing and reducing conditions (FIGS. 9-11.). FIG. 9 is an image showing a highly purified preparation of the S1-RBD-sFc protein under non-reducing conditions (lane 2) and reducing conditions (lane 3). FIG. 10 is an image showing a highly purified preparation of the S1-RBD-His protein under non-reducing conditions (lane 2) and reducing conditions (lane 3). FIG. 11 is an image showing a highly purified preparation of the ACE2-ECD-sFc protein under non-reducing conditions (lane 2) and reducing conditions (lane 3).

The purified proteins were further characterized by mass spectrometry analysis and glycosylation analysis.

a. S1-RBD-His—LC Mass Analysis

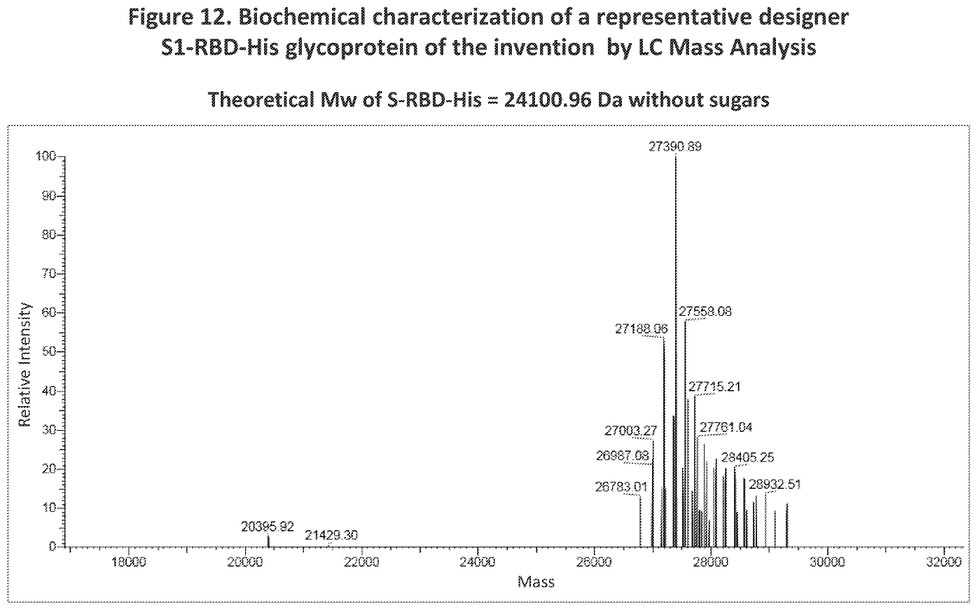

The purified S1-RBD-Efis protein was further characterized by LC mass spectrometry analysis. The theoretical molecular weight of the S1-RBD-His protein, based on its amino acid sequence, is 24,100.96 Da without consideration of any post-translational modifications, including glycosylation. FIG. 12 shows a group of molecular species with molecular weights spanning between 26,783 Da to 28,932 Da were detected, with a major peak at 27,390,89 Da, suggesting that the protein is glycosylated.

b. S-RBD-sFc—LC Mass Analysis and Glyensylation Analysis

i. Glycosylation

Glycoproteins can have two types of glycosylation linkages: N-linked glycosylation and O-linked glycosylation. N-linked glycosylation usually occurs on an asparagine (Asn) residue within a sequence: Asn-Xaa-Ser/Thr, where Xaa is any amino acid residue except Pro, and the carbohydrate moiety attaches on the protein through the NH₂ on the side chain of asparagine. O-linked glycosylation makes use of side chain OH group of a serine or threonine residue.

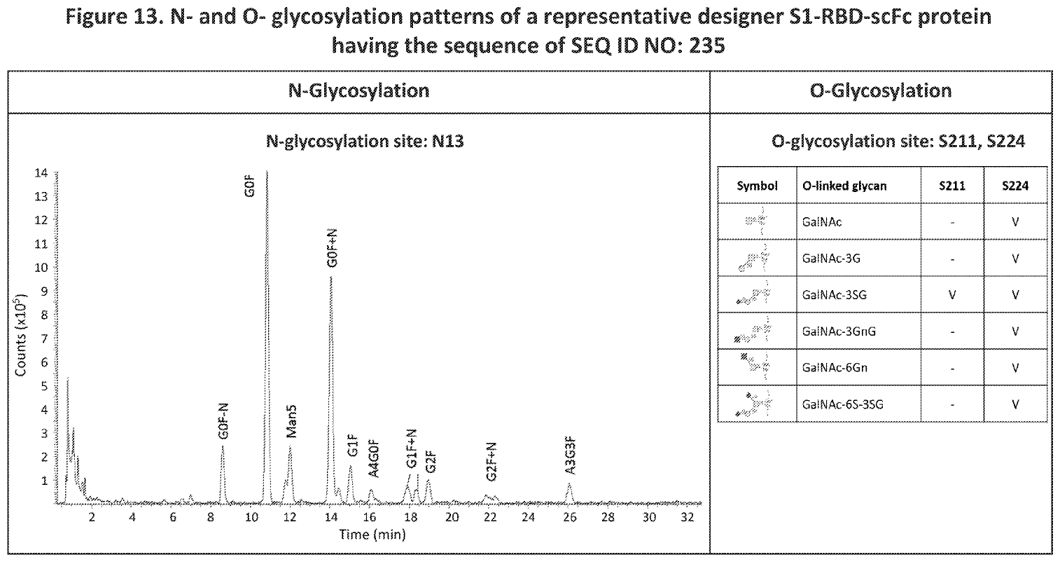

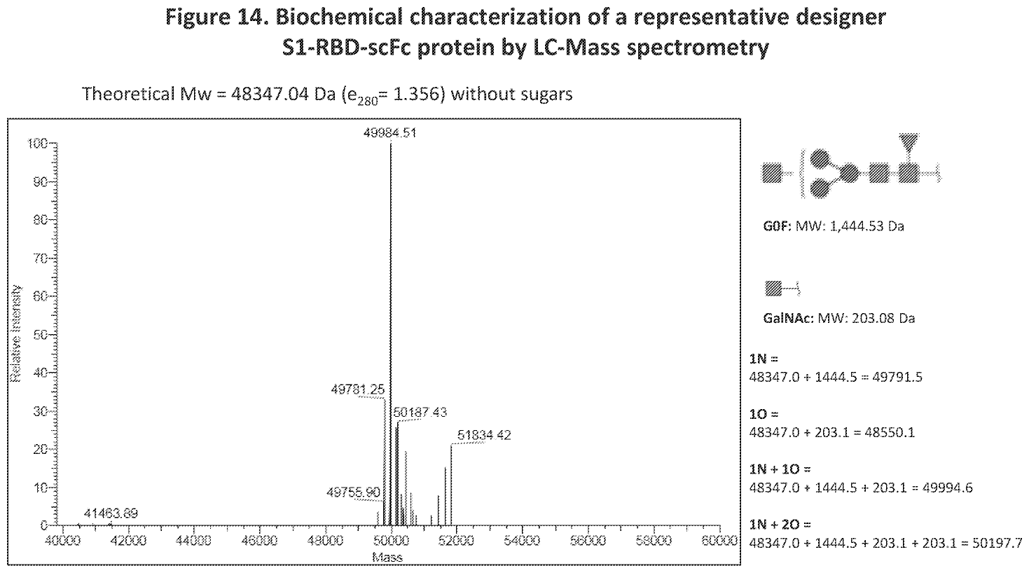

Glycosylation sites of S-RBD-sFc were investigated by trypsin digestion followed by LC-MS and MS/MS (FIGS. 13 and 14). FIG. 13 shows that S-RBD-sFc has one N-linked glycosylation site on the arginine residue at amino acid position 13 (N13) and O-glycosylation sites on the serine residues at amino acid positions 211 (S211) and 224 (S224).

ii. N-Glycosylation

The N-linked glycan structure of S-RBD-sFc was analyzed by mass spectrometry (MS) spectra technology. In brief, PNGase F was used to release N-oligosaccharides from the purified protein. Then the portions of N-linked glycans were further labeled with 2-aminobenzamide (2-AB) to enhance the glycan signals in the mass spectrometry. Finally, conjugated oligosaccharides were investigated by the normal-phase HPLC with fluorescence detector for mapping and by mass spectrometry for structural identification. FIG. 13 shows that 10 N-linked glycans were identified on the S-RBD-sFc protein with the major N-glycans being G0F and G0F+N. The carbohydrate structures of N-linked glycans of S-RBD-sFc are summarized in the Table 14.

iii. O-Glycosylation

The O-linked glycans of S-RBD-sFc were investigated by trypsin digestion followed by mass spectrometry spectra technology. After trypsin digestion, the peaks containing O-linked glycans were collected and their molecular weights were determined by mass spectrometry. FIG. 13 shows that 6 O-linked glycans were identified on the S-RBD-sFc protein. The carbohydrate structures of O-linked glycans of S-RBD-sFc are summarized in the Table15.

iv. LC Mass Spectrometry Analysis

The purified S1-RBD-sFc protein was characterized by LC mass spectrometry analysis. The theoretical molecular weight of the S1-aBD-sFc protein based on its amino acid sequence is 48,347,04 Da. FIG. 14 shows the mass spectrometry profile of the S1-RBD-sFc protein, with a major peak at 49,984,51 Da. The difference between the theoretical molecular weight and the weight observed by LC mass spectrometry is 1,637.47 Da, which suggests that the purified S-RBD-sFc protein contains N- and/or O-glycans, as shown in the figure.

c. ACE2-ECD-sFc—LC Mass Analysis and Glycosylation Analysis

i. Glycosylation

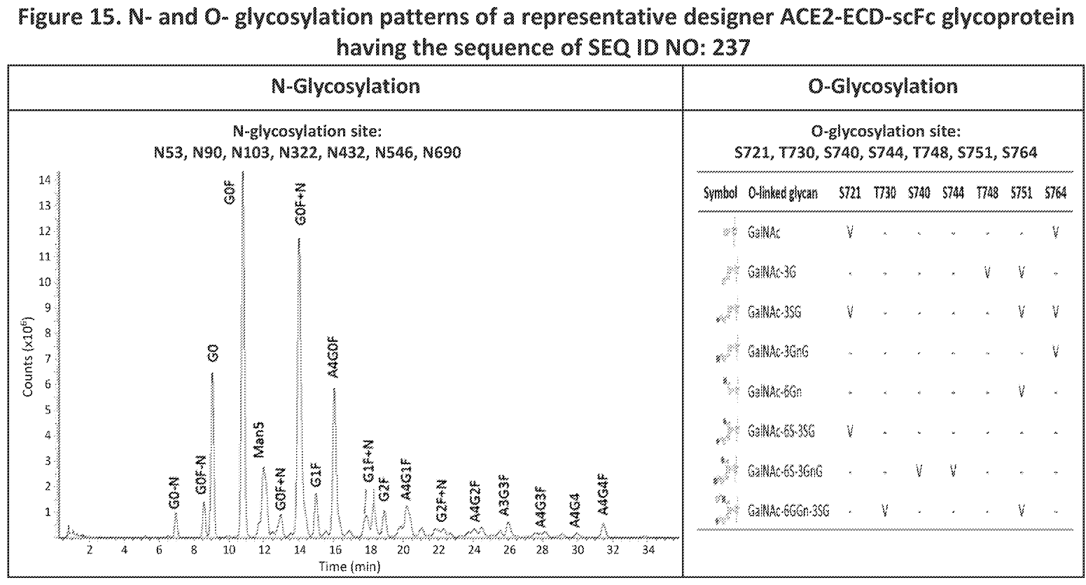

Glycosylation sites of ACE2-ECD-sFc were investigated by trypsin digestion followed by LC-MS and MS/MS. FIG. 15 shows that the ACE2-ECD-sFc protein has seven N-linked glycosylation sites (N53, N90, N103, N322, N432, N546, N690) and seven O-linked glycosylation sites (S721, T730, S740, S744, T748, S751, S764).

ii. N-Glycosylation

The N-linked glycan structure of ACE2-ECD-sFc was analyzed by mass spectrometry (MS) spectra technology. In brief PNGase F was used to release N-oligosaccharides from proteins. Then the portions of N-linked glycans were further labeled with 2-aminobenzamide (2-AB) to enhance the glycan signals in the mass spectrometry. Finally, conjugated oligosaccharides were investigated by the normal-phase HPLC with fluorescence detector for mapping and by mass spectrometry for structural identification. FIG. 15 shows that 17 N-linked glycans were identified on the ACE2-ECD-sFc protein with the major N-glycans being G0F and G0F+N. The carbohydrate structures of N-linked glycans of ACE2-ECD-sFc are summarized in Table 1(.

iii. O-Glycosylation

The O-linked glycan structure of ACE2-ECI)-sFc were investigated by trypsin digestion followed by mass spectrometry spectra technology. After trypsin digestion, the peaks containing O-linked glycans were collected and their molecular weights were determined by mass spectrometry. FIG. 15 shows that 8 O-linked glycans were identified. The carbohydrate structures of the O-linked glycans of ACE2-ECD-sFc are summarized in Table 17.

iv. LC Mass Spectrometry Analysis

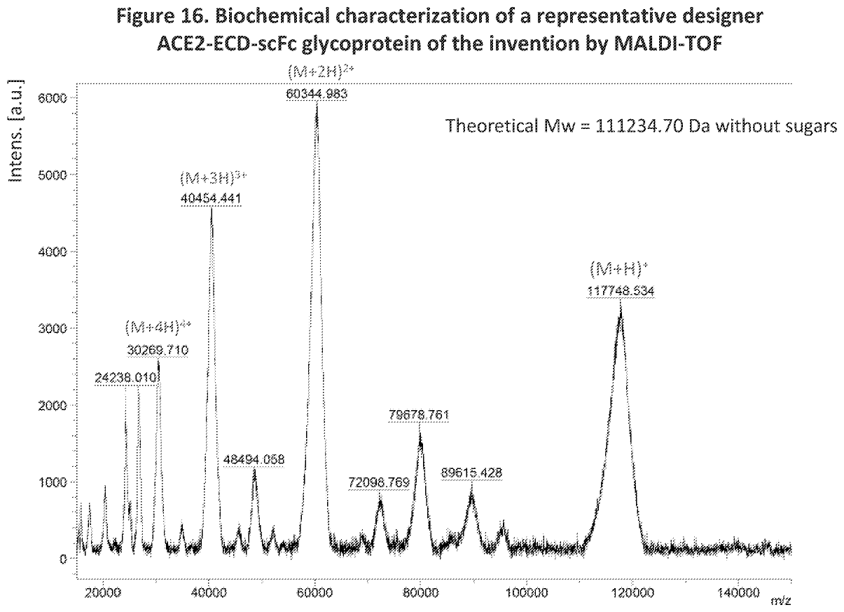

The purified ACE2-ECD-sFc protein was characterized by LC mass spectrometry analysis. The theoretical molecular weight of the ACE2-ECD-sFc protein based on its amino acid sequence is 111,234.70 Da. FIG. 16 shows the mass spectrometry profile of the ACE2-ECD-sFc protein, with a major peak at 117,748,534 Da. The difference between the theoretical molecular weight and the weight Observed by LC mass spectrometry is 1,637.47 Da, which suggests that the purified ACE2-ECD-sFc protein contains N- and/or O-glycans.

d. Sequence and Structure of S1-RBD-sFc

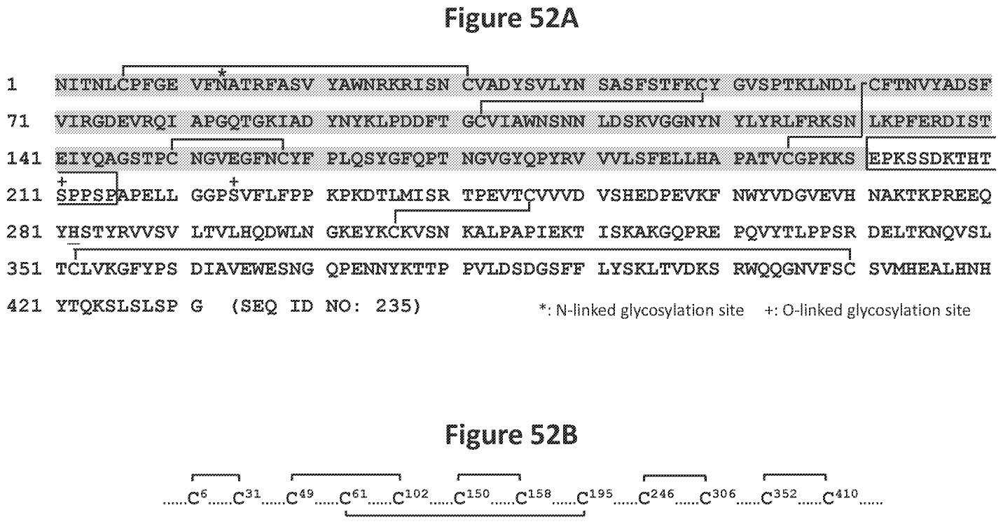

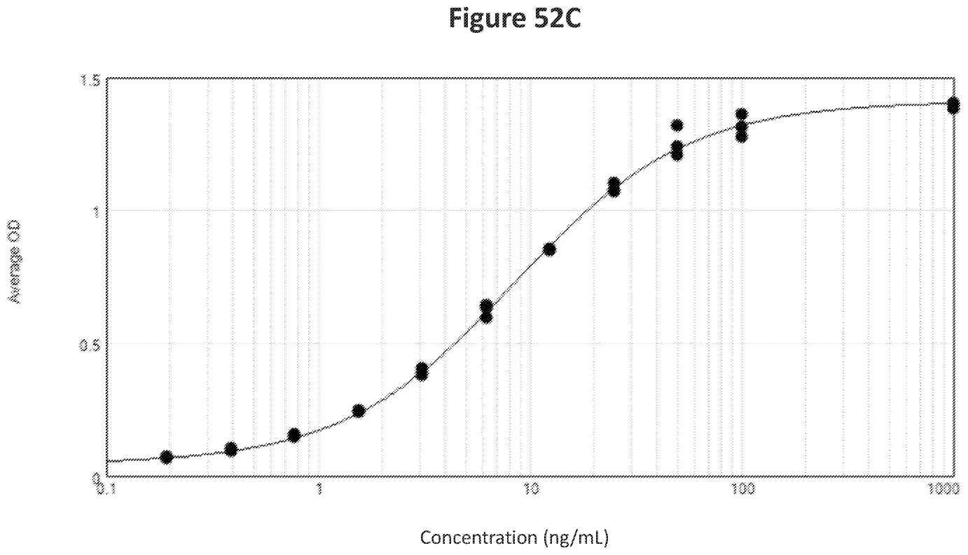

The sequence and structure of S1-RBD-sFc fusion protein (SEQ ID NO: 235) is shown in FIG. 52A. S1-RBD-sFc protein is a glycoprotein consisting of one N-linked glycan (Asn13) and two O-linked glycans (Ser211 and Ser224). The shaded portion (aa1-aa200) represents the S1-RBD portion of SARS-CoV-2 (SEQ ID NO: 226), the boxed portion (a.a.201-aa215) represents the mutated hinge region (SEQ ID NO: 188), and the unshaded/unboxed portion (aa216-aa431) represents the sFc fragment of an IgG1 (SEQ ID NO: 232). The substitution of His297 for Asn297 (EU-index numbering) in single chain Fc of IgG1, (i.e., His282 in SEQ ID NO: 235 shown in FIG. 52A) is indicated by underline. The molecular mass of S1-RBD-sFc protein is about 50 kDa and contains 431 amino acid residues including 12 cysteine residues (Cys6, Cys31,

Cys49, Cys61, Cys102, Cys150, Cys158, Cys195, Cys246, Cys306, Cys352 and Cys410), forming 6 pairs of di sulfide bonds (Cys6-Cys31, Cys49-Cys102, Cys61-Cys195, Cys150-Cys158, Cys246-Cys306 and Cys352- Cys410), which are shown as connecting lines in FIG. 52A. A summary of the disulfide bonding of S1-RBD-sFc is shown in FIG. 52B.

There is one N-glycosylation site Asn13 on the RBD domain and two O-glycosylation sites Ser211 and Ser224 on a sFc fragment. The N-glycosylation site is shown with an asterisk (*) and the two O-glycosylation sites are shown with a plus (+) above the residues shown in FIG. 52A. Glycosylation of an IgG Fc fragment on a conserved asparagine residue, Asn297 (EU-index numbering), is an essential factor for the Fc-mediated effector functions such as complement dependent cytotoxicity (CDC) and antibody-dependent cell-mediated cytotoxicity (ADCC). The Fc fragment in S1-RBD-sFc is designed for purification by protein A affinity chromatography. In addition, the glycosylation site at Asn297 of the heavy chain was removed through mutation to His (N297H-EU numbering, N282H in the S1-RBD-sFc protein) to prevent the depletion of target hACE2 through Fc-mediated effector functions.

e. Binding Activity of S1-RBD-sFc to hACE2

Because the RBD of SARS-CoV-2 binds to hACE2, measurement of binding to hACE2 is a relevant method to demonstrate that S1-RBD-Fc is in a structure representing that of SARS-CoV-2 spike protein. The binding activity of the vaccine was tested in an hACE2 ELISA and was demonstrated to bind hACE2 with an EC50 of 8.477 ng/mL, indicative of high affinity (FIG. 52C).

### EXAMPLE 10

### Design and Identification of Antigenic Peptides from SARS-CoV-2 Nucleocapsid (N), Spike (S), Membrane (M), Envelope (E), and Open Reading Frame 9b (ORF9b) Proteins for use as Immunoadsorbent in Immunoassays

1. Peptide Antigens from the N, S, M, E, and ORF9b proteins

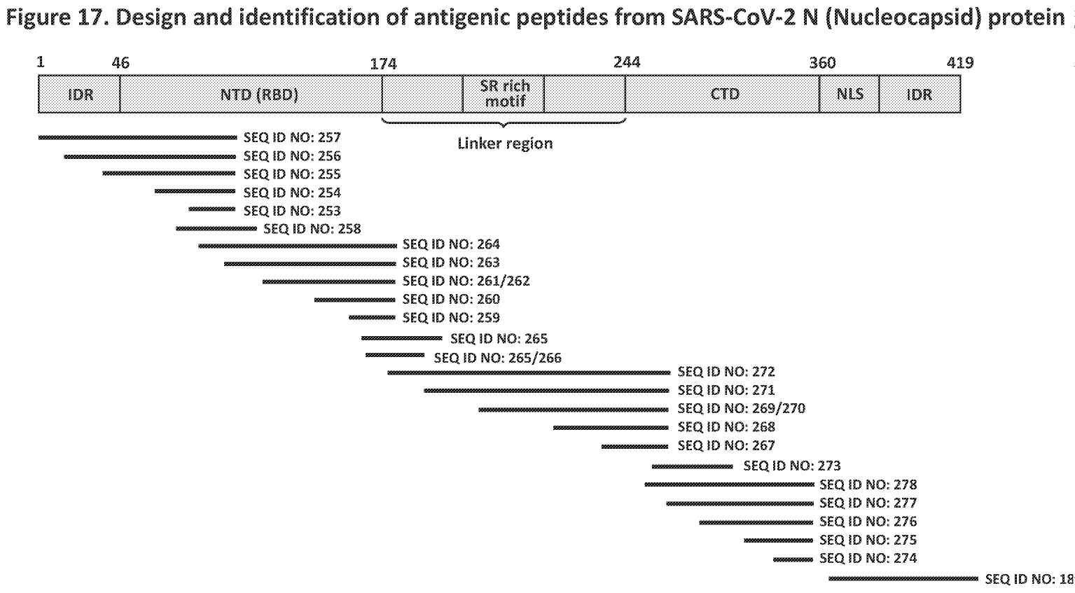

Over 25 carefully designed peptides derived from the SARS-CoV-2 nucleocapsid (N) protein (SEQ ID NO: 6, Table 2) were synthesized for identification of antigenic peptides suitable for use in the preparation of SARS-CoV-2 antigen mixture as immunoadsorbent in various immunoassays for detection of antibodies in infected individuals. The amino acid sequences of the antigenic peptides are shown in Table 13 (SEQ ID NOs: 253 to 278) and the relative position of the peptides within the full-length N protein is shown in FIG. 17.

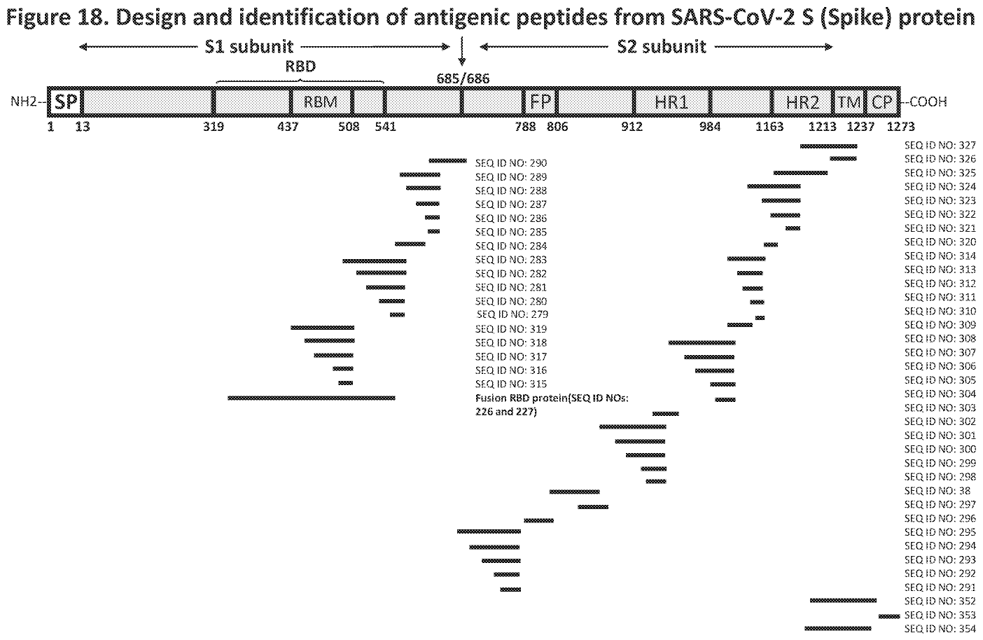

Over 50 carefully designed peptides with sequences derived from the SARS-CoV-2 spike (S) protein (SEQ ID NO: 20, Table 3) were synthesized for identification of antigenic peptides suitable for use in the preparation of SARS-CoV-2 antigen mixture as immunoadsorbent in various immunoassays for detection of antibodies in infected individuals. The amino acid sequences of the antigenic peptides are shown in Table 13 (SEQ ID NOs: 279 to 327) and the relative position of the peptides within the full-length S protein is shown in FIG. 18.

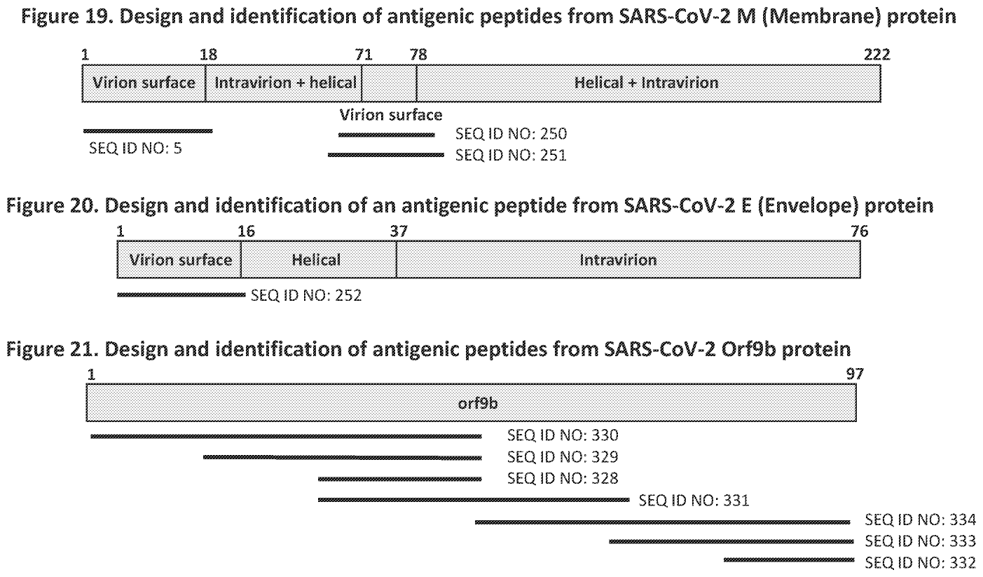

Three carefully designed peptides with sequences derived from the exposed regions of SARS-CoV-2 membrane (M) protein (SEQ ED NO: 1, Table 1) were synthesized for identification of antigenic peptides suitable for use in the preparation of SARS-CoV-2 antigen mixture as immunoadsorbent in various immunoassays for detection of antibodies in infected individuals. The amino acid sequences of the antigenic peptides are shown in Tables 1 and 13 (SEQ ID NOs: 4, 5, 250, and 251) and the relative position of the peptides within the full-length M protein is shown in FIG. 19.

Eight carefully designed peptides with sequences derived from two small SARS-CoV-2 proteins, being the envelope (E) and ORF9b were synthesized for identification of antigenic peptides suitable for use in the preparation of SARS-CoV-2 antigen mixture as immunoadsorbent in various immunoassays for detection of antibodies in infected individuals. The amino acid sequences of the antigenic peptides are shown in Table 13 (SEQ ID NOs: 252 for the E protein and SEQ ID NOs: 328-334 for the ORF9b protein). The relative position of the peptides within the full-length E protein and ORF9b protein is shown in FIGS. 20 and 21, respectively.

### 2. Evaluation of Peptide Antigens as Immunoadsorbent in ELISA

A panel of 10 representative sera from COVID-19 patients, confirmed by both clinical diagnosis and PCR testing, was used for assessment of the relative antigenicity of the peptide antigens.

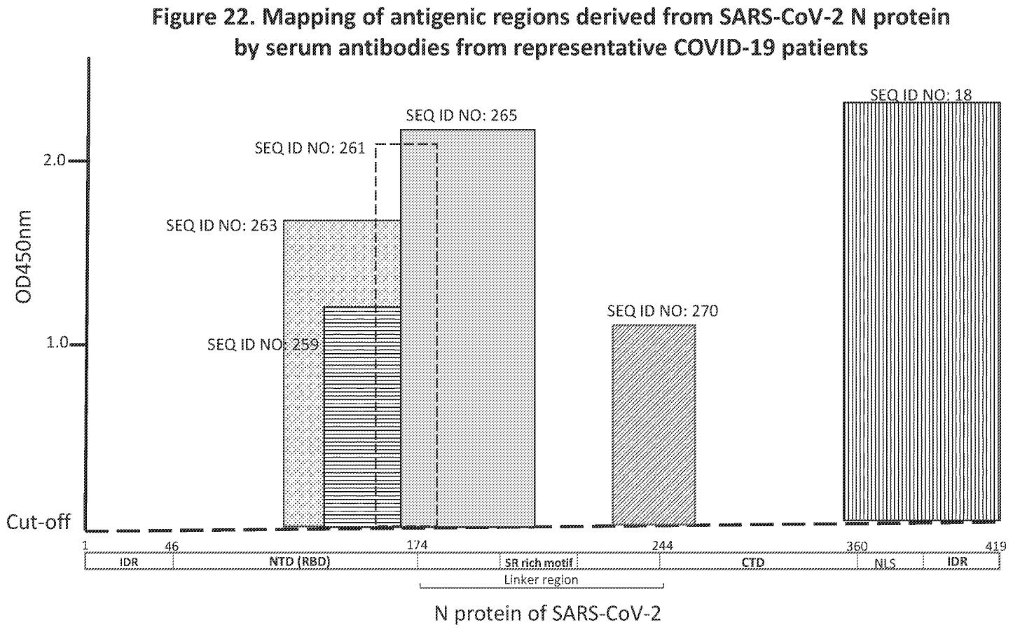

FIG. 22 shows that highly antigenic regions were identified within the N protein that included (a) amino acids 109 to 195 covering part of the N-terminal domain (NTD) and extended to the linker region with SR rich motif (SEQ if) NOs: 259, 261, 263, and 265); (b) amino acids 213 to 266 (SEQ ID NOs: 269 and 270); and (c) amino acids 355-419 (SEQ ID NO: 18) located at the C-terminus covering the NLS and IDR regions.

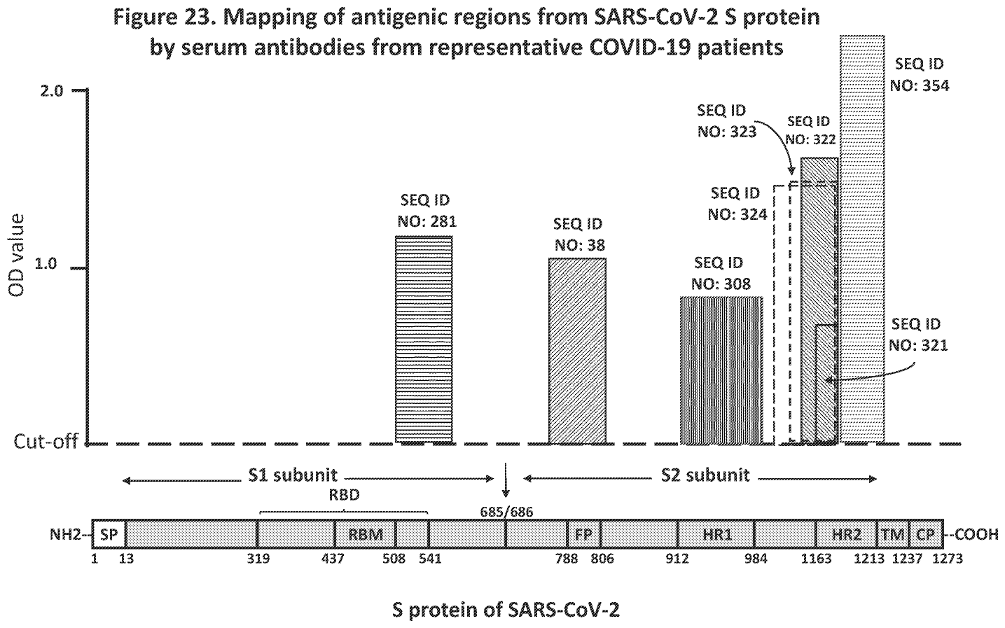

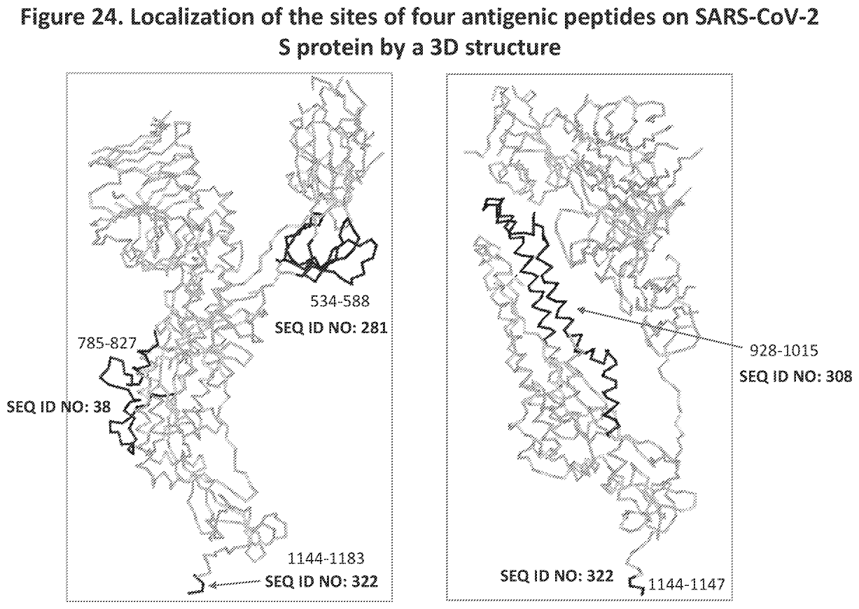

FIG. 23 shows that highly antigenic regions were identified within the S protein that included (a) amino acids 534 to 588 (SEQ NO: 281) covering the region right next to the RBM; (b) amino acids 785 to 839 (SEQ ID NO: 37 and 38) covering the FP region of the S2 subunit; (c) amino acids from 928 to 1015 (SEQ ID NO: 308) covering the HR1 region of the S2 subunit; and (d) amino acids 1104 to 1183 (SEQ ID NOs: 321- 324) covering part of the HR2 region of the S2 subunit. FIG. 24 shows the localization of four antigenic sites (SEQ ID NOs: 38. 281, 308, and 322) in the 3D structure of the S protein. Two antigenic peptides (SEQ ID NOs: 288 and 38) are exposed as globular domains on the surface of the S protein, as shown on the left panel. One antigenic site (SEQ ID NO: 308) is within the elongated helical loop, as shown on the right panel. A fourth antigenic peptide (SEQ ID No: 322) is located around the C-terminal domain is shown in the left and right panel.

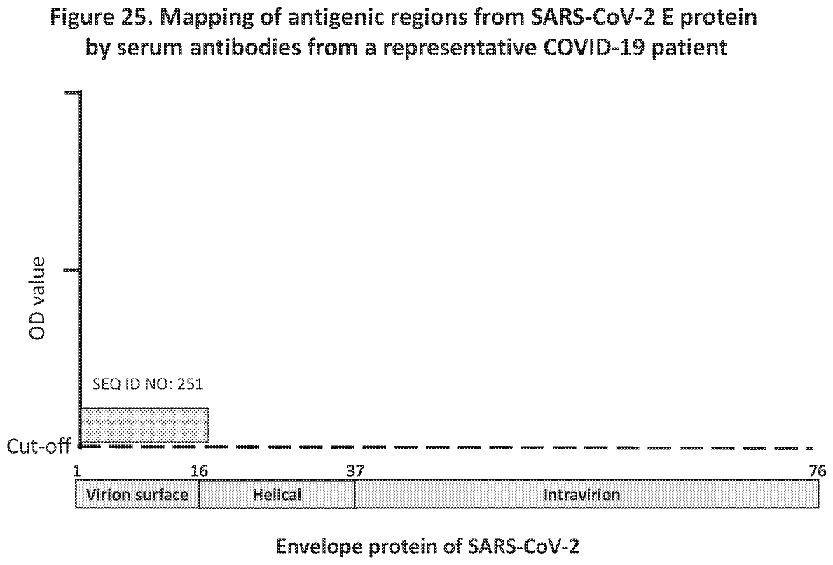

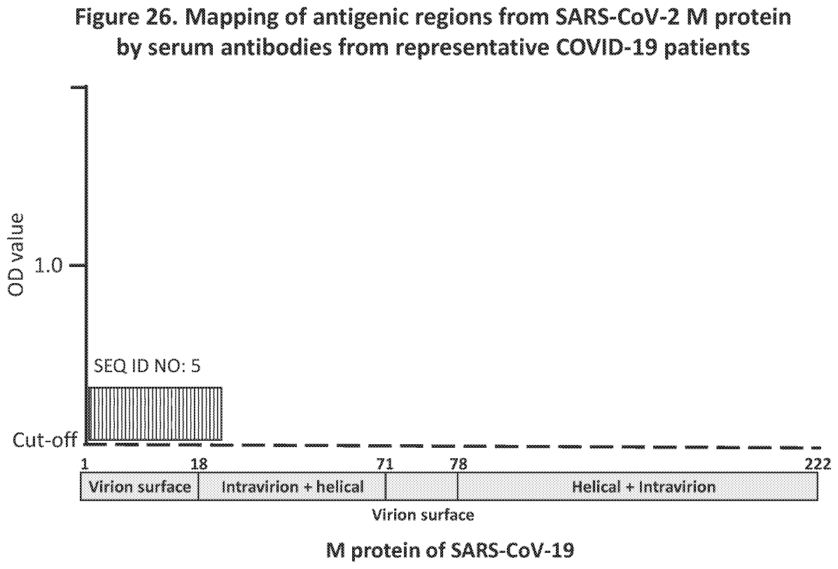

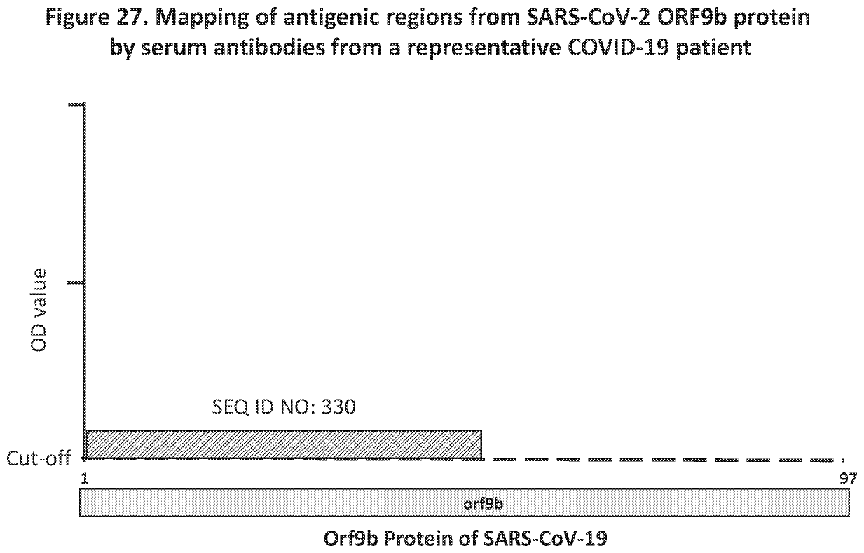

FIGS. 25-27 show that weak antigenic regions were identified from the E protein (SEQ ID NO: 251), M protein (SEQ ID NO: 5), and ORF9b protein (SEQ if) NO: 27), respectively.

Mixtures of antigenic peptides from N, S, and M regions can be formulated as solid phase immunoadsorbent with optimal binding by antibodies from individuals infected by SARS-CoV-2. The mixture of antigenic peptides from the N, S, and M proteins can be used for a sensitive and specific immunoassay for detection of antibodies to SARS-CoV-2 and for sero-surveillance of SARS-CoV-2 infection.

FIG. 28 shows the analytical sensitivity of SARS-CoV-2 ELISA with samples obtained from four representative PCR positive COVID-19 patient sera (LDB, SR25, DB20, and A29). The figure shows high analytical sensitivity, demonstrating positive signals to dilutions as high as 1:640 to as high as >1:2560, by a representative SARS-CoV-2 ELISA formulated with a mixture of antigenic peptides with SEQ ID NOs of 5, 18, 38, 261, 266, 281, and 322 derived from the M, N, and S proteins.

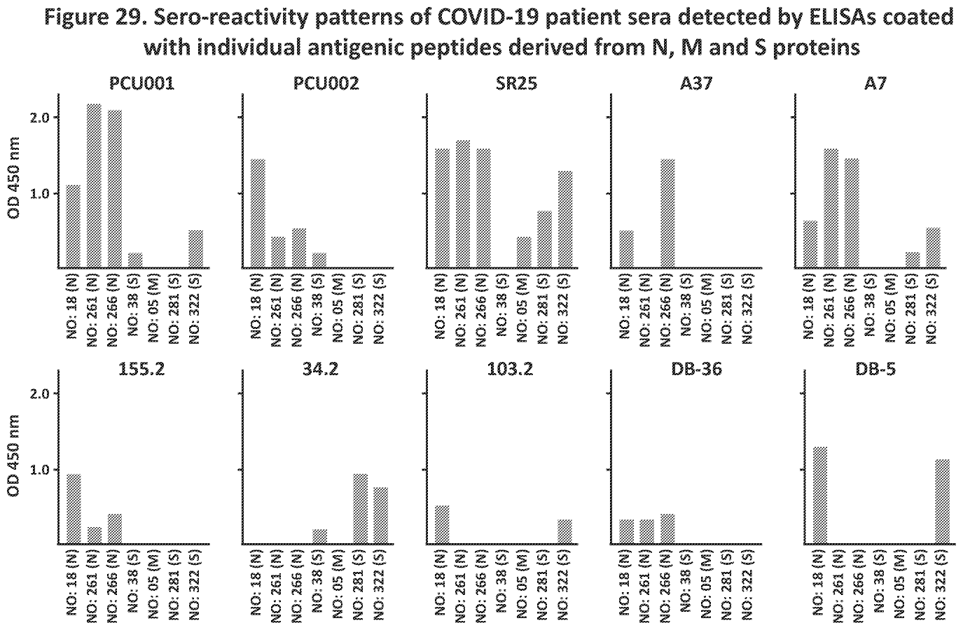

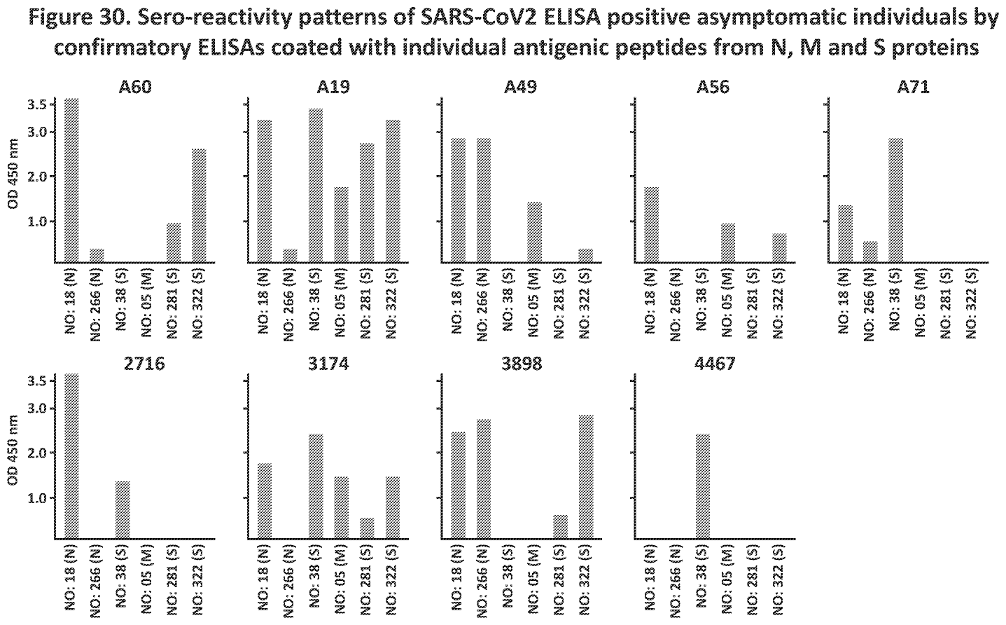

Specific sero-reactivity patterns can be obtained for each patient using individual peptide antigens as immunoadsorbent in ELISA to determine that individual's characteristic antibodies following SARS-CoV-2 infection, as shown in FIGS. 29 and 30. This detailed evaluation of antibodies generated by each individual patient would be in sharp contrast to traditional assays that can only give a simple positive or negative determination with no further confirmatory profiles to assure seropositivity, which frequently could represent a false positive reactivity caused by antibody cross reactivities with protein expressing host cell antigens or other interfering factors.

### EXAMPLE 11

### SARS-CoV-2 ELISA Employs Synthetic Peptide Antigens Derived from SARS-CoV-2 Epitopes for the Detection of Antibodies to SARS-CoV-2 in Human Serum or Plasma

In response to the global pandemic of COVID-19, a blood screening test kit for detection of antibodies against the novel coronavirus SARS-CoV-2 employing SARS-CoV-2 antigenic peptides was developed.

Specimens with absorbance values greater than or equal to the Cutoff Value are defined as “initially reactive”. Initially reactive specimens should be retested in duplicate. Specimens that do not react in either of the duplicate repeat tests are considered “nonreactive” for antibodies to SARS-CoV-2. Initially reactive specimens that are reactive in one or both of the repeat tests are considered “repeatably reactive” for antibodies to SARS-CoV-2.

SARS-CoV-2 ELISA employs an immunosorbent bound to the wells of the reaction microplate consisting of synthetic peptides that capture antibodies with specificities for highly antigenic segments of the Spike (S), Membrane (M) and Nucleocapsid (N) proteins of SARS-CoV-2. During the course of the assay, diluted negative controls and specimens are added to the reaction microplate wells and incubated. SARS-CoV-2-specific antibodies, if present, will bind to the immunosorbent. After a thorough washing of the reaction microplate wells to remove unbound. antibodies and other serum components, a standardized preparation of horseradish peroxidase-conjugated goat anti-human IgG antibodies specific for the Fc portion of human IgG is added to each well. This conjugate preparation is then allowed to react with the captured antibodies. After another thorough washing of the wells to remove unbound horseradish peroxidase-conjugated antibody, a substrate solution containing hydrogen peroxide and 3,3′,5,5′-tetramethylbenzidine (TMB) is added. A blue color develops in proportion to the amount of SARS-CoV-2-specific antibodies present, if any, in most settings, it is appropriate to investigate repeatably reactive specimens by additional immunoassays such as IFA and by more specific tests such as PCR that are capable of identifying antigens for specific gene products of SARS-CoV-2. The lack of detectable reactivities among the U.S. blood donors from serum and plasma samples collected from years before the SARS-CoV-2 pandemic time indicated a specificity for the assay to distinguish SARS-CoV-2 infection from infection by other human coronaviruses. In comparison to other testing, the synthetic antigens of the present disclosure provide advantages of high standardization, freedom from biohazardous reagents, and ease of scale-up production. Moreover, testing by the ELISA format can be readily automated for large-scale screening. The highly specific peptide-based SARS-CoV-2 antibody test is a convenient means to carry out widespread retrospective surveillance. One series of three seroconversion bleeds on days 3, 8, and 10 from a PCR confirmed COVID-19 patient (NTUH, Taiwan) was tested. Day 10 after onset of symptoms was the earliest time point a positive signal with SARS-CoV-2 ELISA was obtained. Several additional seroconversion bleeds were tested with sensitivities of the early period of infection from symptom of onset are reported below in studies 1 and 2.

### 1. Assessment of Assay Specificity and Sensitivity

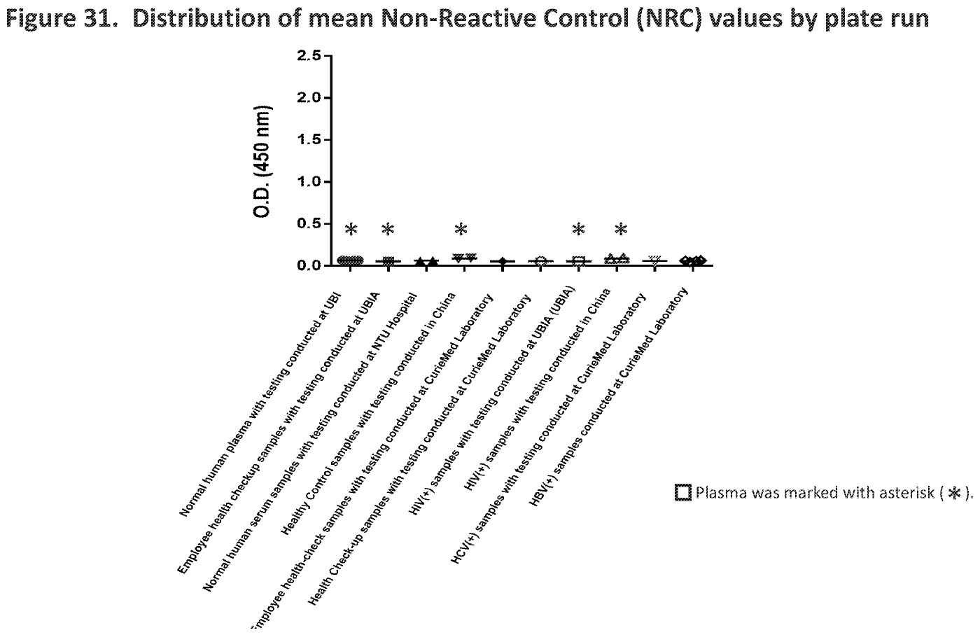

In study 1, the SARS-CoV-2 ELISA was first tested with serum samples/plasma samples collected from (1) those known to have other viral infections unrelated to SARS-CoV-2 (Taiwan and US); and (2) a cohort of employees undergoing routine health-check-ups and from normal human plasma (NHP) collected in 2007. These samples were tested to assess assay specificity using a large number of non-COVID-19 samples (n=922) to establish rationales for determining appropriate cutoff values for the assay. As shown in FIG. 31, the 922 specimens unrelated to SARS-CoV-2 infection all had very low OD readings by the assay.

a. Study 1: Performance Characteristics: Lack of Cross-Reactivity to Other Viral Infections:

Test results for SARS-CoV-2 ELISA obtained with serum samples from patients known to have other viral infections, including samples from patients who are positive for HIV (51 samples), HBV (360 samples), HCV (92 samples) and those having prior Coronavirus infection with strains of NL63 (2 samples) and HKU1 (1 sample), are shown in Table 18. No cross-reactivity was observed in any of these samples, as all of the samples tested with OD readings near that of blanks. Similar near blank OD readings were obtained for all samples from a cohort of employees undergoing routine health-checkups and from normal human plasma (NHP) collected in 2007.

b. Determination of the Cutoff Value of the SARS-CoV-2 ELISA Based on NRC+0.2

The cutoff value of the disclosed SARS-CoV-2 ELISA was set at NRC+0.2 (i.e., the mean of three OD450nm readings of the non-reactive control (NRC) included with the kit for each run of the immunoassay plus 0.2 units) based on the OD readings from 922 samples tested by SARS-CoV-2 ELISA and the rationales discussed below. The cutoff value of NRC+0.2 allows an optimal result that the SARS-CoV-2 ELISA has maximal sensitivity for detection of PCR-positive confirmed COVID-19 patients and a 100% specificity in the general population. Table 19 reports the mean OD450nm readings of NRCs from all the test runs collected for testing of normal human plasma, normal human serum, and serum or plasma samples from individuals with other (i.e., non-SARS-CoV-2) viral infections. The mean values of NRC by plate run were close to the mean of normal human plasma consistently as shown in FIG. 31. When examining the standard deviation (SD) of normal human plasmalserum and serum/plasma samples from individuals with other (i.e., non-SARS-CoV-2) viral infections across testing sites, the standard deviation (SD) ranged from 0.006 to 0.020 (Table 19). Setting the cutoff value to be the mean of NRCs+0.2 units provides a bar higher than the mean of NRCs+4SD (0.020×4=0.080), which allows for 99.99% confidence level (z-value=3.981) for a negative predictive value. Also, a cutoff value of NRC+2 units provides room to establish a grey zone between “Mean NRC+0.12” to “Mean NRC+0.2” for individuals at high risk for SARS-CoV-2 infection (e.g., hospital healthcare workers and public service providers, etc.) who have a higher probability to be on the course of seroconversion into positivity.

c. Study 1: Performance Characteristics: 100% Sensitivity to Detect Seroconversion in all COVID-19 Hospitalized Patients

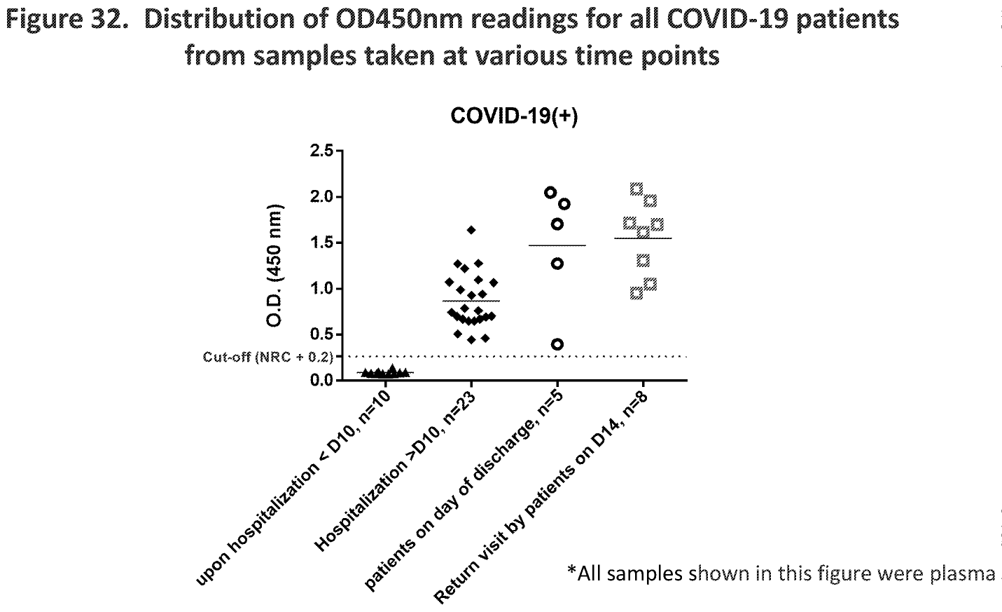

The test results from the SARS-CoV-2 ELISA (serum/plasma) were evaluated based on (1) <10 days post onset of symptoms mostly for samples taken upon enrollment of the patients into the hospital; (2) >10 days post symptom onset for patients during treatment at the hospital, (3) those on the date of hospital discharge, and (4) those upon a revisit of the hospital 14 days after discharge, as shown in Table 20 and FIG. 32.

The results of this Study 1 show that (1) the sensitivity of samples (n=10) collected upon hospital enrollment was 0%, (2) during hospitalization all seroconverted (23 out of 23) into positivity, giving rise to a test sensitivity of 100%, (3) all showing positive reactivity upon the day of hospital discharge (5 out of 5) giving rise to a sensitivity of 100%, and (4) all showing positive reactivity at the return visit to the hospital 14 days after discharge, giving rise to a sensitivity of 100%. The overall sensitivity of the test for study 1 was 78.2% (36/46) (or 37/47=78.7% with one sample taken twice from one patient at a different time point).

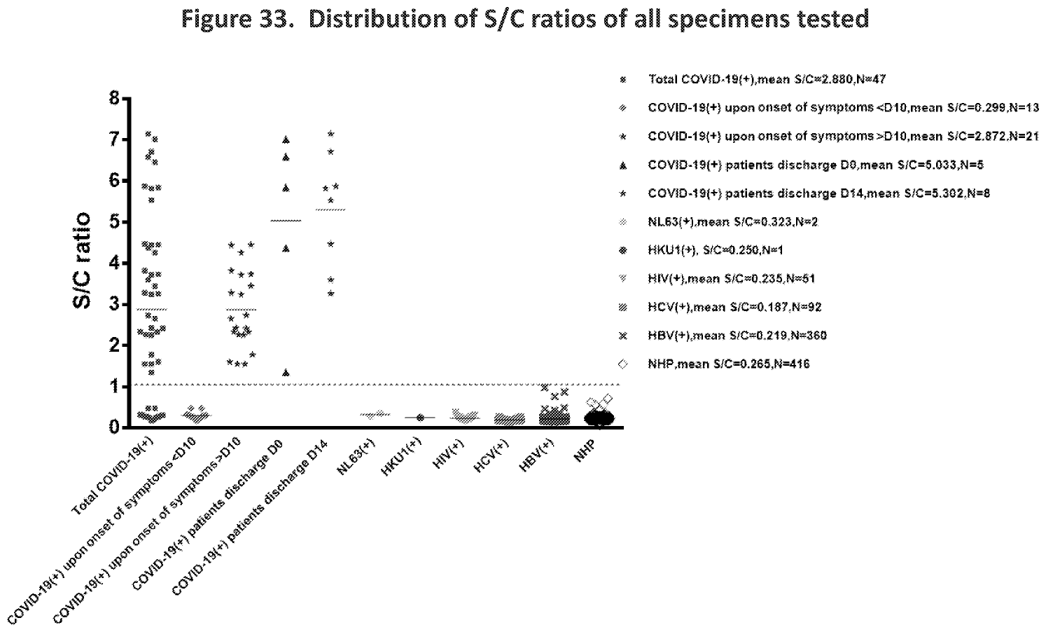

In summary, as shown in FIG. 33, the distribution of S/C ratios, calculated based on the NRC+0.2 cutoff value, was plotted for all samples tested in Study 1. None of the 922 samples collected from individuals unrelated to SARS-CoV-2 infection demonstrated any positive reactivities by this ELISA. Table 21 presented summary results for all samples from those unrelated to SARS-CoV-2 infection and those 46 COVID-19 confirmed patients with samples collected 10 days after onset of symptoms.

The disclosed SARS-CoV-2 ELISA provided an overall specificity of 100% with a sensitivity of 100% for hospitalized COVID-19 patients 10 days after onset of symptoms. An overall sensitivity of 78.2% was obtained when all 46 COVID-19 confirmed patients were factored in, including samples collected from those at the beginning of the onset of symptoms. These positives samples can be further characterized for the antigenic profiles of the SARS-CoV-2 reactive antibodies by other serological assays as described in related Examples for confirmation of the positivity and further assessment of immune status, including the amount of antibodies that can mount neutralizing activities against SARS-CoV-2.

d. Study 2: Performance Characteristics: Sensitivity in Seroconversion of COVID-19 Patients

A total of 37 samples from 17 PCR confirmed and hospitalized COVID-19 patients were tested using the disclosed SARS-CoV-2 ELISA. Detailed information on date of serum collection during treatment as related to onset of symptoms was provided, as shown in Table 22.

The test results from the SARS-CoV-2 ELISA (serum/plasma) were evaluated based on (1) <7 days post hospitalization, (2) 7-14 days post hospitalization, and (3) >14 days post hospitalization, as shown in Table 23. The results show that the relative specificity of samples <7 days post-onset of symptoms was 25%; 7-14 days post onset of symptoms was 63.6%; and >14 days post-hospitalization was 100%. The overall sensitivity of all 37 samples was 81.1% (30/37) and the accuracy for positive predictive value at >14 days post onset of symptoms in this cohort was 100%.

e. Conclusions

The disclosed SARS-CoV-2 ELISA screening assay is a highly sensitive and specific test capable of detecting low levels of antibodies in human serum or plasma. The assay is characterized by:
Capability of detecting SARS-CoV-2 antibodies in human seroconversion sample as early as 2 days post onset of symptoms (one patient with ID No. 11 from Study 2, Table 22) and, in general, 7 to 10 days after onset of symptoms with a positive predictive value of 100% at day 10 and day 14 after onset of symptoms for Study 1 and Study 2, respectively. The overall sensitivity rates for Studies 1 and 2 were 78.2% and 81.1%, respectively.
Specificity of 100% for SARS-CoV-2 from serum/plasma samples collected from normal plasma donors and a cohort of employees undergoing health checkups collected prior to 2020.
No cross-reactivity was found for samples from individuals with other viral infections e.g., HCV; HBV, HIV including other coronavirus, N-63, HKU, collected prior to 2020.

### 2. Special Precautions

The disclosed. SARS-CoV-2 ELISA PROCEDURE and the INTERPRETATION OF RESULTS sections (described above) must be closely adhered to when testing for the presence of antibodies to SARS-CoV-2 in plasma or serum from individual subjects. Because the SARS-CoV-2 ELISA was designed to test individual units of serum or plasma, data regarding its interpretation were derived from testing individual samples. Insufficient data are available to interpret tests performed on other bodily fluids at this time and testing of these specimens is not recommended.

A person whose serum or plasma is found to be positive using the disclosed SARS-CoV-2 ELISA is presumed to have been infected with the virus. Individuals who test positive by the disclosed SARS-CoV-2 ELISA should be tested using other molecular tests (e.g., RT-PCR) to determine if the individual has an active infection that is capable of being transmitted to others. Appropriate counseling and medical evaluation should also be offered. Such an evaluation should be considered an important part of SARS-CoV-2 antibody testing and should include test result confirmation from a freshly drawn sample.

COVID-19 caused by SARS-CoV-2 is a clinical syndrome and its diagnosis can only be established clinically. The disclosed SARS-CoV-2 ELISA testing alone cannot be used to diagnose an active SARS-CoV-2 infection, even if the recommended investigation of reactive specimens confirms the presence of SARS-CoV-2 antibodies. A negative test result at any point in the serologic investigation does not preclude the possibility of exposure to or infection with the SARS-CoV-2 in the future.

### 3. Performance Evaluation of the UBI® SARS-CoV-2 ELISA

a. Cross-Reactivity

The UBI® SARS-CoV-2 ELISA was evaluated in a clinical agreement study (described below) and demonstrated a negative percent agreement of 100% (154/154). In addition, cross-reactivity of non-SARS-CoV-2 specific antibodies were examined using sera with known antibodies against. Respiratory Syncytial viruses (10) and ANA (6). No interference was observed.

b. Clinical Agreement Study

Studies were performed to determine the clinical performance of the UBI® SARS-CoV-2 ELISA assay.

To estimate the positive percent agreement (PPA) between the UBI® SARS-CoV-2 ELISA and the PCR comparator, 100 serum and 5 EDTA plasma specimens were collected from 95 subjects who tested positive for SARS-CoV-2 by a polymerase chain reaction (PCR) method and who also presented with COVID-19 symptoms. Each specimen was tested using the UBI® SARS-CoV-2 ELISA.

To estimate the negative percent agreement (NPA), 62 serum and 92 EDTA plasma specimens were collected from 154 subjects presumed to be negative for SARS-CoV-2. All of the 154 specimens were collected prior to COVID outbreak. Each specimen was tested using the UBI® SARS-CoV-2 ELISA. The results of both groups are presented Tables 24 and 25.

c. Independent Clinical Agreement Validation Study

The UBI® SARS-CoV-2 ELISA was tested on Jun. 17 and Sep. 1, 2020 at the Frederick National Laboratory for Cancer Research (FNLCR) sponsored by the National Cancer Institute (NCI). The test was validated against a panel of previously frozen samples consisting of 58 SARS-CoV-2 antibody-positive serum samples and 97 antibody-negative serum and plasma samples. Each of the 58 antibody-positive samples were confirmed with a nucleic acid amplification test (NAAT) and both IgM and IgG antibodies were confirmed to be present in all 58 samples. The presence of antibodies in the samples was confirmed by several orthogonal methods prior to testing with the UBI SARS-CoV-2 ELISA. The presence of IgM and IgG antibodies specifically was confirmed by one or more comparator methods. Antibody-positive samples were selected at different antibody titers.

All antibody-negative samples were collected prior to 2020 and include: i) Eighty-seven (87) samples selected without regard to clinical status, “Negatives” and ii) Ten (10) samples selected from banked serum from HIV+ patients, “HIV+”. Testing was performed by one operator using one lot of the UBI SARS-CoV-2 ELISA. Confidence intervals for sensitivity and specificity were calculated per a score method described in CLSI EP12-A2 (2008).

For evaluation of cross-reactivity with HIV+, it was evaluated whether an increased false positive rate among antibody-negative samples with HIV was statistically higher than the false positive rate among antibody-negative samples without HIV (for this, a confidence interval for the difference in false positive rates was calculated per a score method described by Altman). Study results and summary statistics are presented in Tables 26 and 27.

The following limitations of this study are noted:
Samples were not randomly selected, and sensitivity and specificity estimates may not be indicative of the real-world performance of the device.
These results are based on serum and plasma samples only and may not be indicative of performance with other sample types, such as whole blood, including finger stick blood.
The number of samples in the panel is a minimally viable sample size that still provides reasonable estimates and confidence intervals for test performance, and the samples used may not be representative of the antibody profile observed in patient populations.
   d. Matrix Equivalency

The matrix equivalency study was conducted with patient-matched serum and plasma samples from five healthy donors. Plasma samples were drawn in vials containing sodium heparin or K2 EDTA as the anticoagulants. The matched samples were negative when tested with the UBI SARS-CoV-2 ELISA. Then the sample pairs were spiked with a sample positive for SARS-CoV-2 IgG to obtain three concentrations, and tested in duplicate. The results showed 100% agreement of positive and negative signal for each matrix, indicative of no effect of matrix-reactivity for the SARS-CoV-2 IgG detection in serum or plasma samples with UBI® SARS-CoV-2 ELISA.

The study demonstrates that the performance of the UBI® SARS-CoV-2 ELISA is equivalent with serum, sodium heparin plasma, and K2 EDTA plasma samples.

e. Class Specificity

Eight serum samples positive for IgG and IgM antibodies to SARS-CoV-2 were tested with the UBI® SARS-CoV-2 ELISA. The samples were then treated with DTT to destroy the IgM antibodies and re-tested with the UBI® SARS-CoV-2 ELISA. Results for all eight samples were positive both before and after DTT treatment, demonstrating class-specific reactivity to human IgG isotypes. The UBI® SARS-CoV-2 ELISA assay demonstrates class-specific reactivity only to human IgG isotypes. No binding interactions were observed to human IgM.

### EXAMPLE 12

### Development of ELISA for the Measurement of Neutralizing Antibodies through Inhibition of S1 Binding to ACE2

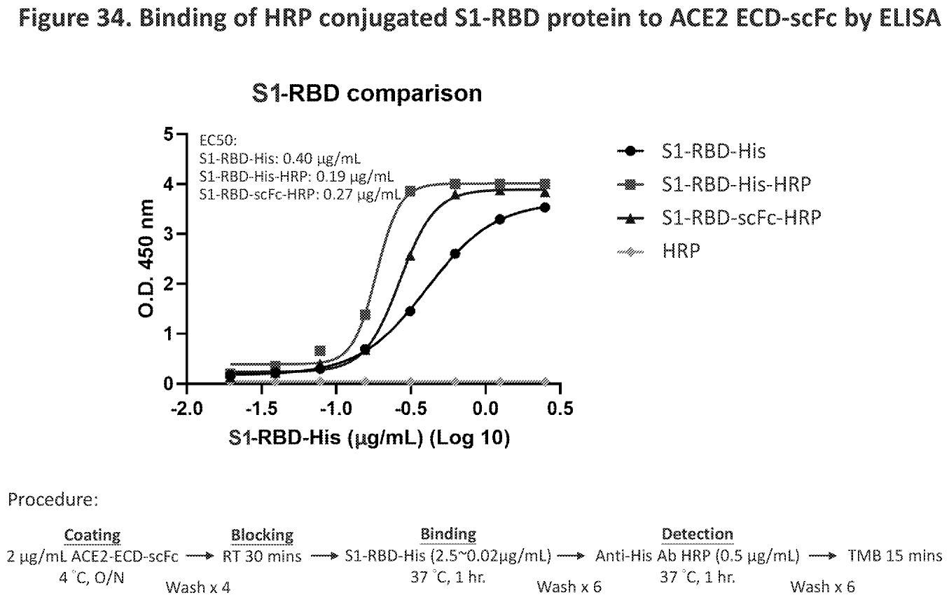

The detailed procedure of an ELISA-based S1-RBD and ACE2 binding assay is illustrated in the bottom portion of FIG. 34. In particular, the ELISA plate was coated with ACE2 ECD-sFc and various S1-RBD proteins were used as a tracer with FIRP alone used as a control tracer. In this study, S1-RBD-His, S1-RBD-His-HRP, S1-RBD-sFc-HRP, and HRP alone were evaluated for their ability to bind to ACE2 ECD-sFc coated on the ELISA plate. FIG. 34 shows that S1-RBD-His, S1-RBD-His-HRP, and S1-RBD-sFc-HRP were able to bind to ACE2 ECD-sFc coated. on the ELISA plate with EC₅₀ values of 0.40 μg/mL, 0.19 μg/mL, and 0.27 μg/mL, respectively. HPR alone was not able to bind to ACE2 ECD-sFc.

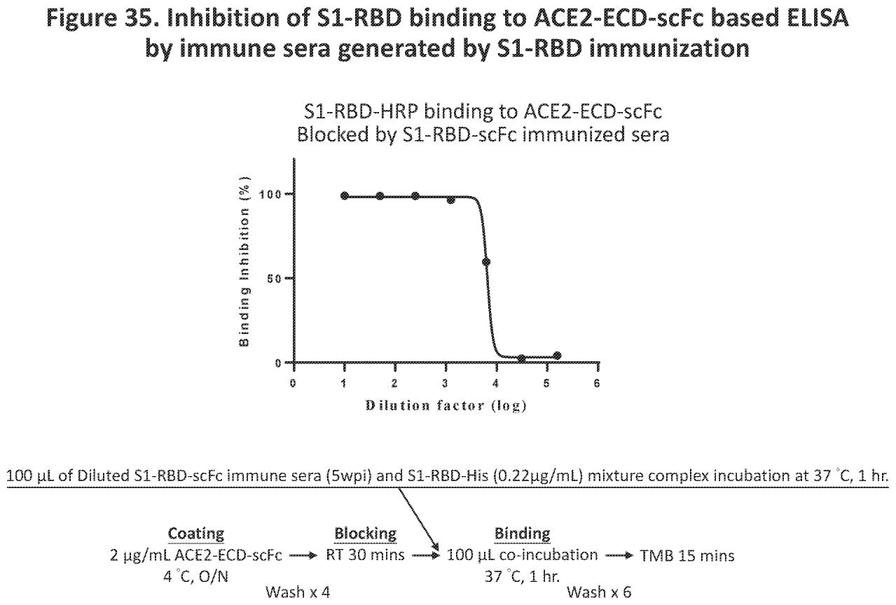

Next, the binding assay described in FIG. 34 was modified in the step prior to the binding step, as shown in the bottom portion of FIG. 35. Specifically, the S1-RBD-His-HRP protein was mixed and incubated with diluted immune sera (5 wpi) containing antibodies directed against S1-RBD-sFc prior to adding the S1-RBD-His-FIRP protein to the ELISA plate coated with ACE2 ECD-sFc. This additional step was added to determine if antibodies raised against S1-RBD-sFc could inhibit the binding of S1-RBD-His-HRP protein to ACE2 ECD-sFc.

FIG. 35 shows a dilution dependent decrease in inhibition of S1-RBD-His-ITRP binding to ACE2 ECD-sFc by immune sera from guinea pigs immunized with S1RBD-sFc ranging from >95% at 1:10 dilution to about <10%, with an EC₅₀ of about 3.5 Log₁₀. The full signal of the binding can be adjusted to allow sensitive detection of the amount of antibodies capable of interfering with, and thus inhibiting, the SL-RBD binding to the ACE2 receptor. A standardized assay can be established for this simplified form of ELISA to measure the extent of serum neutralizing antibodies present in COVID-19 patients, infected and recovered individuals, or individuals receiving S1-RBD comprising vaccines.

Any patient sample found to be positive for antibodies against SARS-CoV-2 by an antibody detection assay can be further tested using this “neutralizing” ELISA to determine if the patient has developed antibodies capable of inhibiting S1-RBD binding to ACE2. Such neutralizing ELISA can be used as a predictor for a patient's ability to prevent re-infection by SARS-CoV-2.

### EXAMPLE 13

### High Precision Designer Vaccine Against SARS-CoV-2 Infection Containing a S1-RBD Fusion Protein

### 1. General Design

An effective immune response against viral infections depends on both humoral and cellular immunity. More specifically, the potential of a high precision designer preventative vaccine would employ designer immunogens, either peptides or proteins, as active pharmaceutical ingredients for (1) induction of neutralizing antibodies through the employment of B cell epitopes on the viral protein that is involved in the binding of the virus to its receptor on the target cell; (2) induction of cellular responses, including primary and memory B cell and CDS⁺ T cell responses, against invading viral antigens through the employment of endogenous Th and CTL epitopes. Such vaccines can be formulated with adjuvants such as ADJUPHOS, MONTANIDE ISA, CpG, etc. and other excipients to enhance the immunogenicity of the high-precision designer immunogens.

A representative designer COVID-19 vaccine employs CHO cell expressed S-RBD-sFc protein (amino acid sequence of SEQ NO: 235 and nucleic acid sequence of SEQ ID NO: 246). This protein was designed and prepared to present the receptor binding domain (RBD) on the SARS CoV-2 Spike (S) protein with the very carbohydrate structure within the RBD to induce high affinity neutralizing antibodies upon immunization. The vaccine can also employ a mixture of designer peptides incorporating endogenous SARS-CoV-2 Th and CTL epitopes capable of promoting host specific Th cell mediated immunity to facilitate the viral-specific primary and memory B cell and CTL responses towards the SARS-CoV-2, for the prevention of SARS-CoV-2 infection. An effective vaccine needs to prime the memory T cells and B cells to allow rapid recall upon viral infection/challenge.

To improve the effectiveness of the disclosed designer immunogens, two representative adjuvant formulations are employed (ADJU-PHOS®/CpG and MONTANIDE™ ISA/CpG) for induction of optimal anti-SARS-CoV-2 immune responses.

ADJUPHOS is generally accepted as an adjuvant for human vaccines. This adjuvant induces a Th2 response by improving the attraction and uptake of designer immunogens by antigen presenting cells (APCs). MONIANIDE™ ISA 51 is an oil which forms an emulsion when mixed with the water phase designer peptide/protein immunogens to elicit potent immune responses to SARS-CoV-2. CpGs Oligonucleotides are TLR9 agonists that improve antigen presentation and the induction of vaccine-specific cellular and humoral responses. In general, the negative charged. CpG molecule is combined with positively charged designer immunogens to form immunostimulatory complexes amenable for antigen presentation to further enhance the immune responses.

The disclosed high precision designer vaccine has the advantage of producing highly specific immune responses compared to weak or inappropriate antibody presentation of vaccines with a more complicated immunogen content employing inactivated viral lysate or other less characterized immunogens. In addition, there are potential pitfalls in COVID-19 vaccine development that are related to a mechanism named antibody-dependent enhancement (ADE). Specifically, ADE is a phenomenon in which binding of a virus to non-neutralizing antibodies enhances its entry into host cells, and sometimes also its replication. This mechanism leads to both increased infectivity and virulence has been observed with mosquito-borne flaviviruses, HIV, and coronaviruses. The disclosed high precision vaccine is designed to avoid vaccine-induced disease enhancement by monitoring the quality and quantity of the antibody responses as they would dictate functional outcomes.

Representative studies discussed below set forth the approach in designing the disclosed high precision SARS-CoV-2 vaccine that can facilitate the elicitation of antibodies that can (1) bind to the CHO-expressed. S1-RBD-sFc protein; (2) inhibit the binding of S1 protein to the ACE2 receptor that is immobilized on a microwell surface or on a cell surface overly expressing ACE2 receptor protein, and (3) neutralize viral mediated cytopathic effect in a cell mediated neutralization assay.

An immunization schedule of the varying forms of S1-RBD-sFc designer proteins (SEQ ID NOs: 235, 236, and 355) in guinea pigs is shown in Table 28 for assessment of antibodies to S protein through a S protein antibody binding assay.

### 2. S1 Protein Antibody Binding Assay (Immunogenicity)

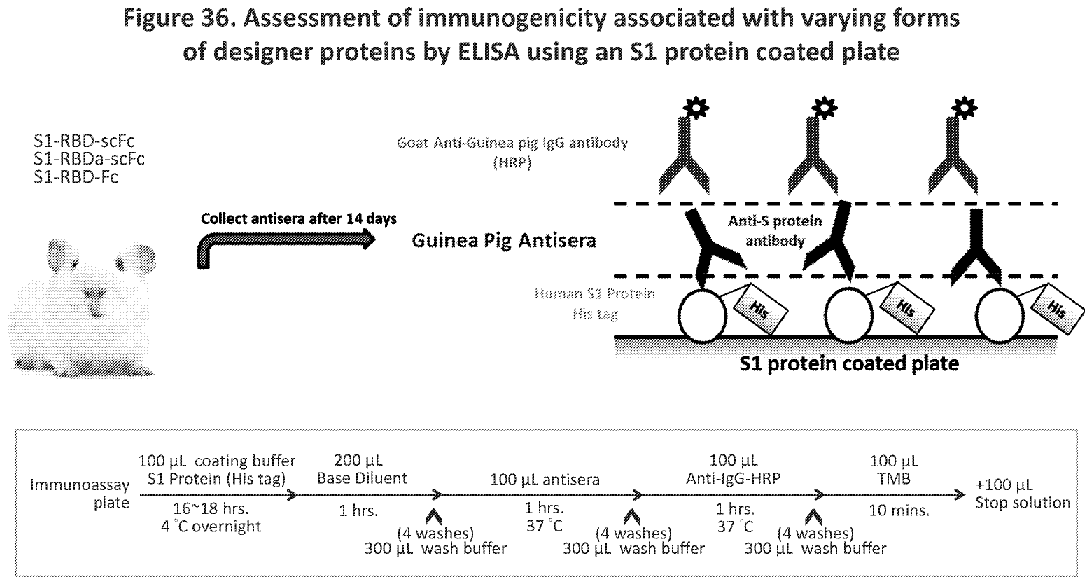

Varying forms of S1-RBD proteins, including S1-RBD-sFc, S1-RBDa-sFc, and S1-RBD-Fc, for each group in the amount of 100μg were mixed with ISA51 to prepare a w/o emulsion. These formulations were immunized into guinea pigs (n=5 per group) intramuscularly using the immunization schedule shown in Table 28. Briefly, guinea pigs were given a primary immunization of 100 μg per dose followed by a boost of 50 μg per dose at 3 weeks with individual serums collected at 0, 3, and 5 weeks post initial immunization (WPI). The collected serum samples were tested for immunogenicity by an S1-coated ELISA with detailed procedure an illustrated in FIG. 36.

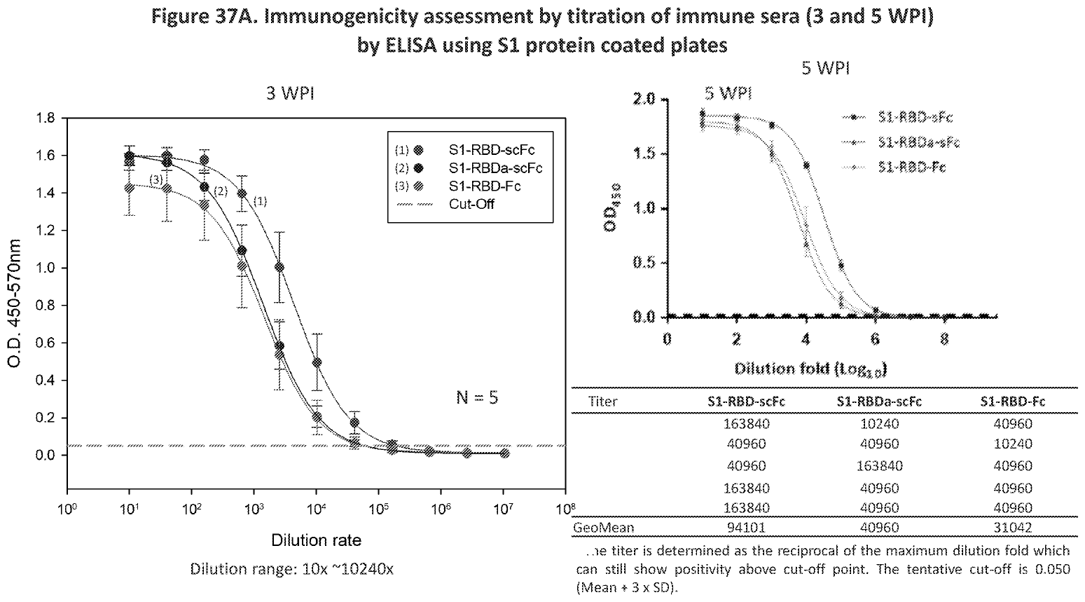

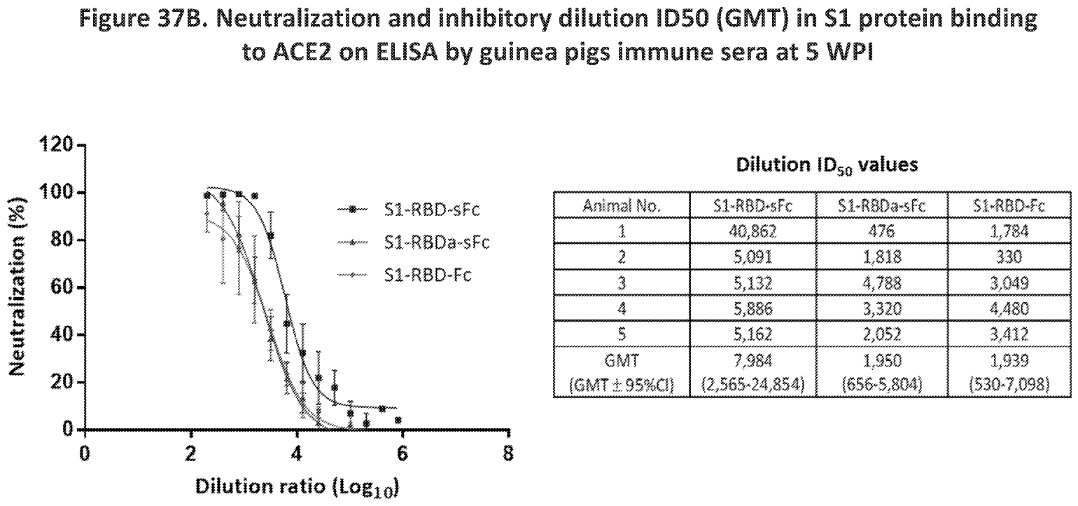

FIG. 37A shows that high titers of S binding antibodies were generated after only a single administration (3 WPI) with GeoMeans of titers being 94,101, 40,960, and 31,042 for S1-RBD-sFc, S1-RBDa-sFc and S1-RBD-Fc, respectively. The titers were determined as the reciprocal of the maximum dilution fold that can still show positivity above the cutoff value, where the cutoff was set as 0.050 OD₄₅₀ reading (Mean+3XSD). These titers indicate that the single chain Fc fusion protein S1-RBD-sFc protein (SEQ ID NO: 235) was the most immunogenic, followed by S-RBDa-sFc (SEQ ID NO: 236), where RBD domain was modified to reduce a Cys-disulfide bond to allow better folding of the domain, and then the double chain Fc fusion protein S-RBD was the least immunogenic. The difference between S1-RBD-sFc and S1-RBDa-sFc at 3 WPI was statistically significant (p≤0.05), indicating that all constructs were highly immunogenic with S1-RBD-sFc apparently holding a slight advantage in terms of binding antibodies responses. At 5 WPI, however, no significant difference was notable for the S1-RBDa-sFc vs. S1-RBD-Tc (p>0.99) and the S1-RBD-sFc vs. S1-RBD-Fc (p=0.20).

FIG. 37B shows the neutralization and inhibitory dilution ID₅₀ (Geometric Mean Titer; GMT) in S1 protein binding to ACE2 on ELISA by guinea pigs immune sera at 5WPI. Serum samples of 5 WPI from each vaccinated animal in the groups were serially diluted and assayed for inhibition activity by an ELISA-based method. The inhibition activity of serum was determined by using the following formula: Inhibitory Activity ={(1−(OD450ₑₓₚ−OD450_{background})/(OD450ₘₐₓ−OD450_{background})}×100%. The resultant inhibition curves (left panel) were expressed as mean±SE. The antibody titer of each animal with inhibition of 50% (right panel) was determined based on the inhibition curve generated by four-parameter logistic regression.

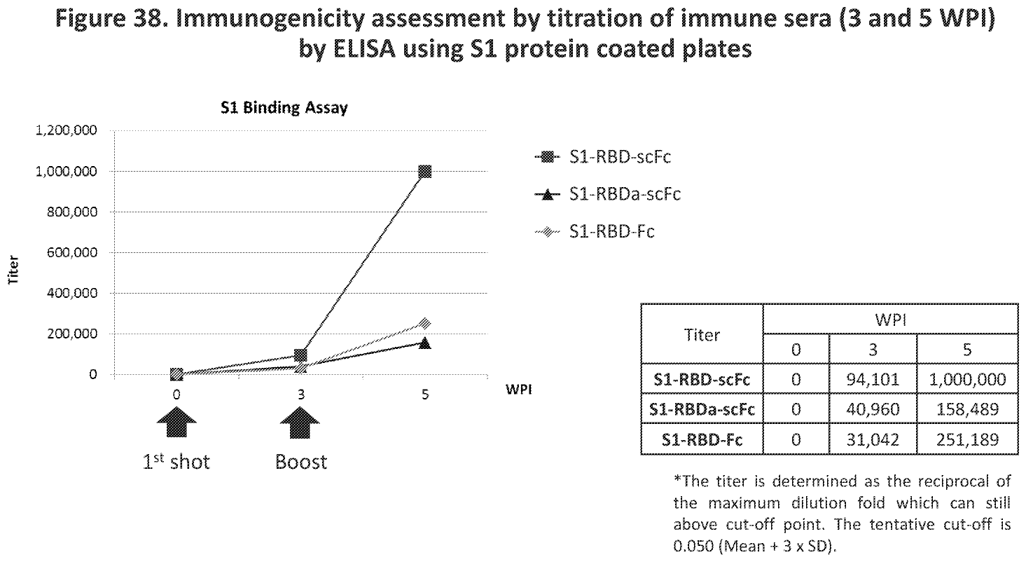

FIG. 38 shows that a minor booster with 50 μg per dose at 3 WPI resulted in an enhancement of antibody titers by 4- to 10-fold for each protein immunogen. Comparing the three designer fusion proteins, S-RBD-sFc fusion protein had a GeoMean S1 binding titer increase of 10⁶ following the booster, a 10-fold increase from the initial immunization.

The functional properties of the antibodies elicited by these three protein immunogens were evaluated for their ability to inhibit the binding of S1-RBD to its surface receptor ACE-2 to prevent entry of the virus into target cells. Two functional assays were established, including (1) an ELISA to assess the direct inhibition of S1-RBD binding to ACE-2 ECD-sFc coated plate by such S1 binding antibodies; and (2) a cell-based S1-RBD-ACE2 binding inhibition assay. These functional assays are described further below.

### 3. ELISA-Based Assays to Determine S1-RBD Binding Inhibition to ACE2

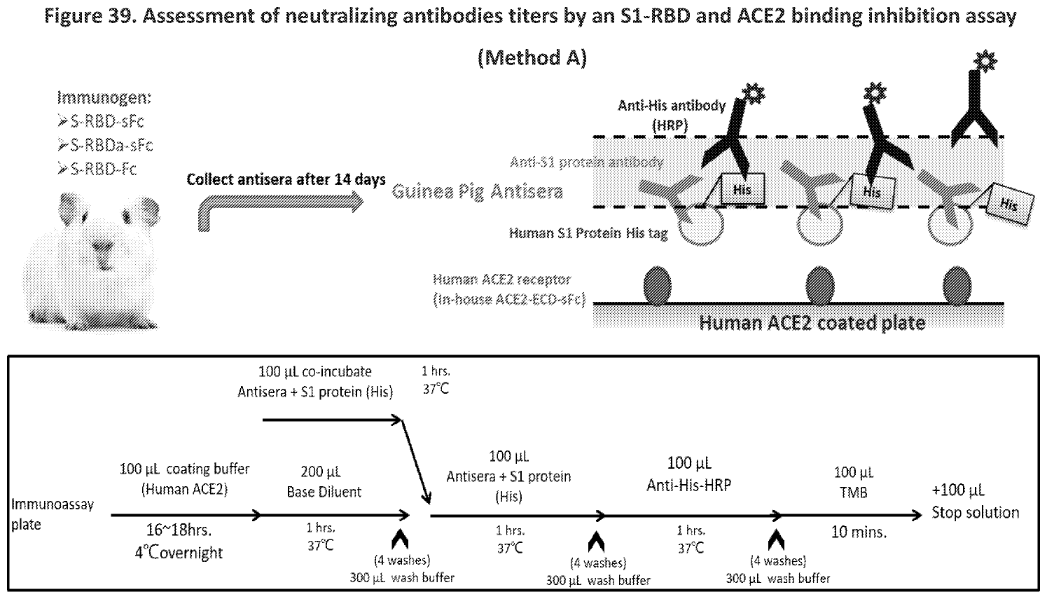

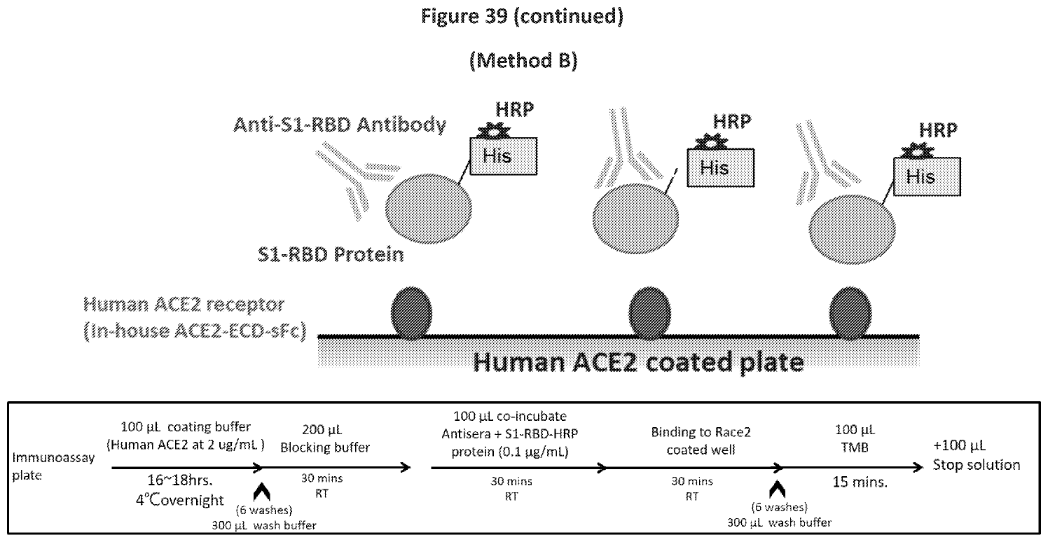

The detailed procedure for two separate ELISA-based S1-RBD/ACE2 binding inhibition assays are illustrated in FIG. 39.

In Method A, the ELISA plates are coated with ACE2 (e.g., ACE2 ECD-sFc) and 100 μL of antisera from an animal immunized with S-RBDa-sFc is mixed and incubated with S1-RBD-His prior to adding the mixture to the ELISA plate. The amount of S1-RBD-His binding/inhibition can be detected using a HRP conjugated anti-His antibody.

In Method B, the ELISA plates are coated with ACE2 (e.g., ACE2 ECD-sFc) and 100 μL of antisera from an animal immunized with S-RBDa-sFc is mixed and incubated with S1-RBD-His-HRP prior to adding the mixture to the ELISA plate. The amount of S1-RBD-His-HRP binding/inhibition can be detected directly.

4. Results from ELISA-Based Assays to Determine S1-RBD binding inhibition to ACE2

The S1-RBD/ACE2 binding inhibition assays of Methods A and B described above were utilized to determine the ability of antibodies against S1-RBD-sFc, S1-RBDa-sFc, and S1-RBD-Fc to inhibit S1-RBD-His binding to ACE2 ECD-sFc by ELISA.

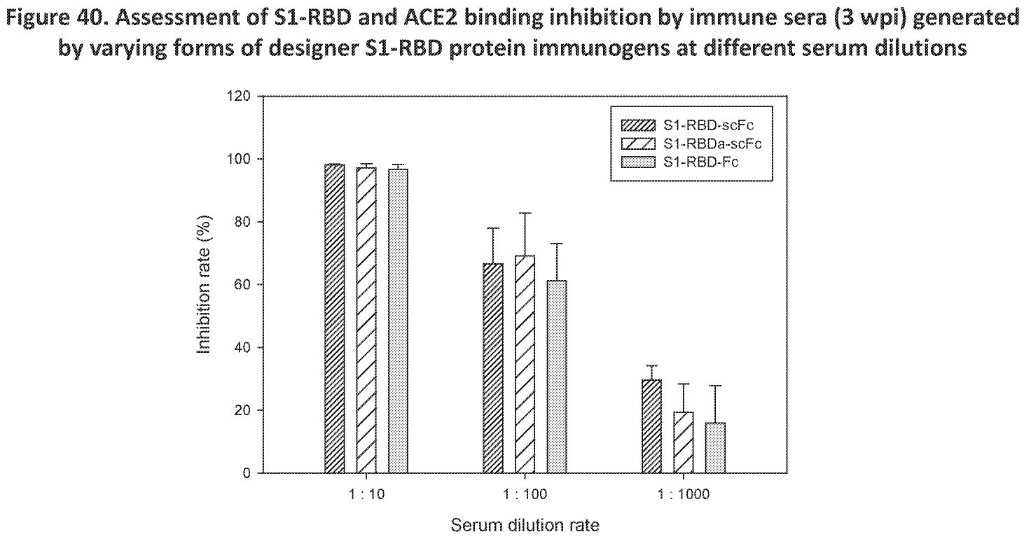

FIG. 40 shows the results obtained using the inhibition assay of Method A. Specifically, FIG. 40 shows that over 95% binding inhibition was observed in this assay with all immune sera collected at 3 wpi after prime dose to guinea pigs immunized with sFc or Fc fusion proteins mixed and incubated with S1-RBD-His protein prior to binding to ACE2 ECD-sFc bound to the ELISA plate, when tested at 1:10 dilution of the sera. A dilution dependent decrease in inhibition of S1-RBD-His to ACE2 ECD-sFc binding was found from >95% at 1:10 dilution of sera, to about 60% inhibition at 1:100 dilution of sera, and about 20% inhibition at 1:1,000 dilution of sera.

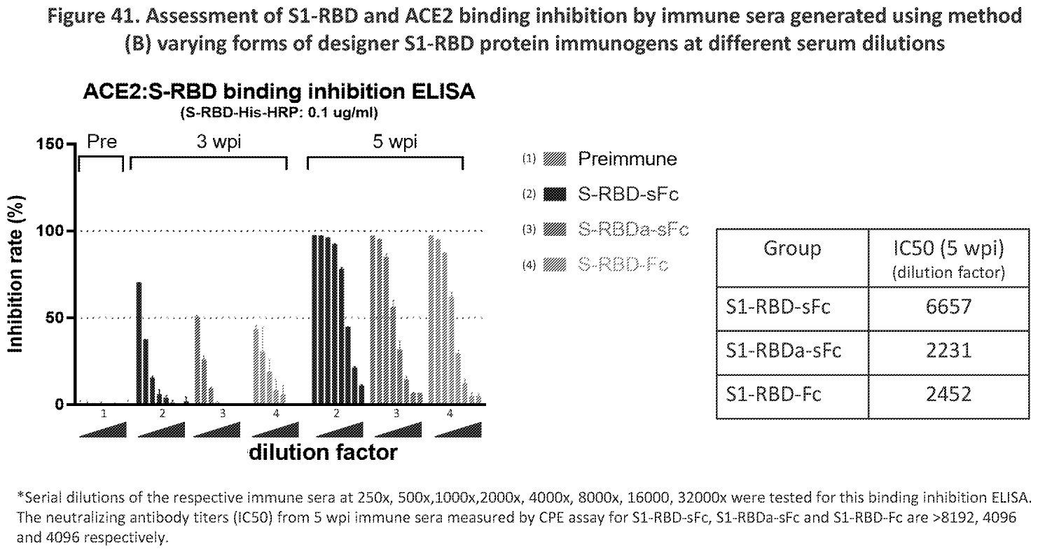

FIG. 41 shows the results obtained using the inhibition assay of Method B. Specifically, FIG. 41 shows that over 95% binding inhibition was observed in this assay with all immune sera collected at 5 wpi after prime and booster doses to guinea pigs immunized with sFc or Fc fusion proteins mixed and incubated with S1-RBD-His-HRP protein prior to binding to ACE2 ECD-sFc bound to the ELISA plate, when tested at 1:250 dilution of the sera. A dilution dependent decrease in inhibition of S1-RBD-His-HRP to ACE2 ECD-sFc binding was found from 1:250 dilution to 1:32,000 dilution.

The differences Observed in the results from Method A (FIG. 40) and Method B (FIG. 41) demonstrate that Method B is more sensitive in detecting binding inhibition compared to Method A.

### 5. Cell-Based Assay to Determine S1-RBD Binding Inhibition to ACE2

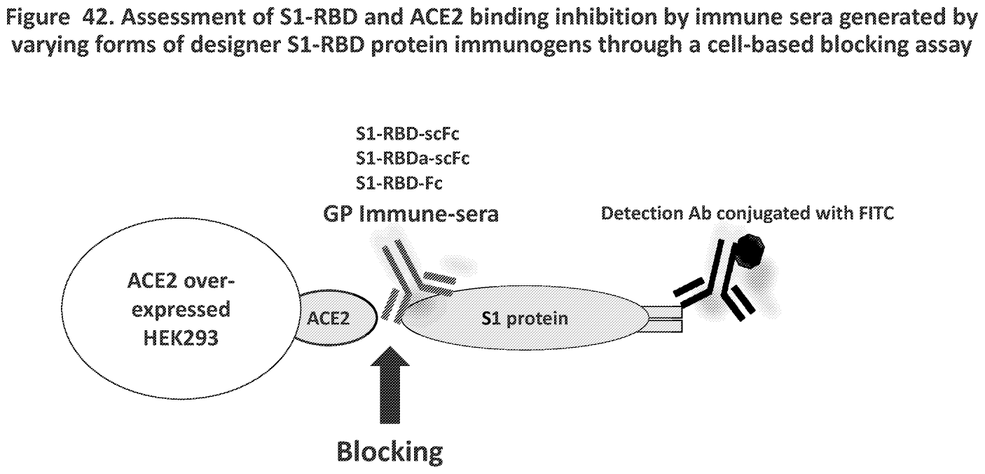

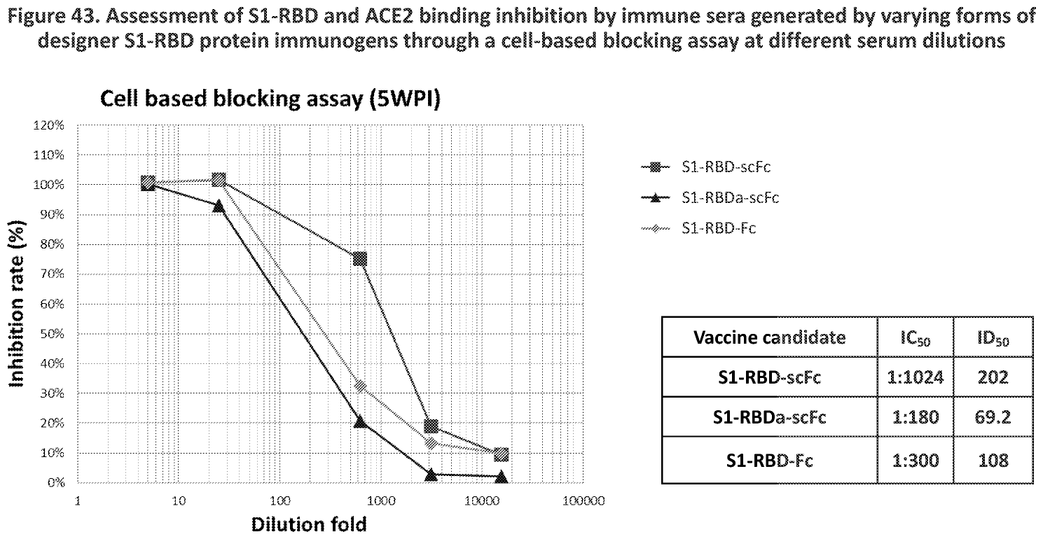

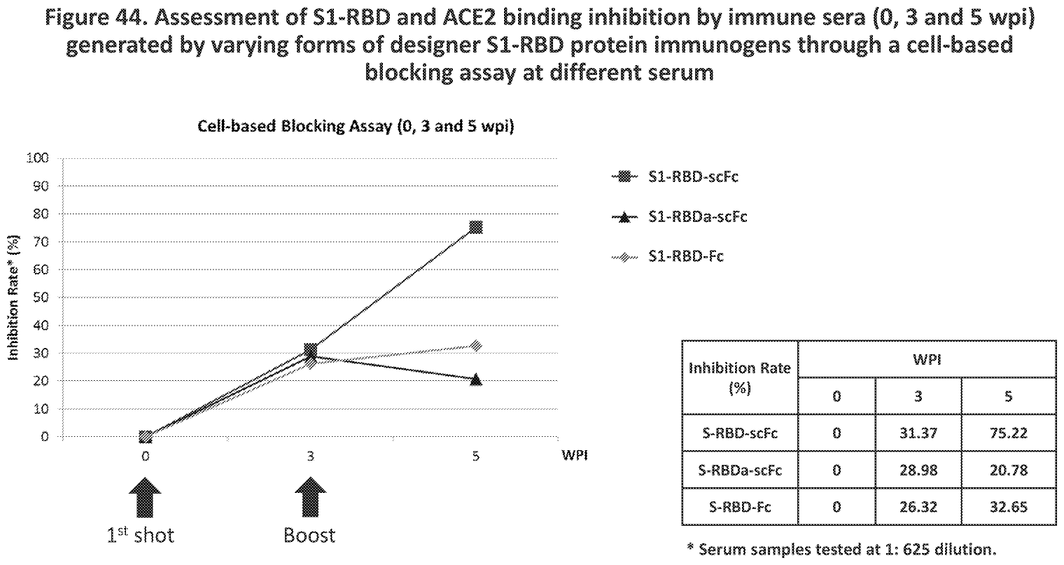

The detailed procedure of a cell-based S1-RBD and ACE2 binding inhibition assay is illustrated in detail in FIG. 42. Specifically, ACE-2 over-expressed HEK293 cells were used as the target cells for such binding. Immune sera obtained from guinea pigs immunized with various forms of fusion proteins of S1-RBD (S1-RBD-sFc, S1-RBDa-sFc, and S-RBD-Fc) were mixed. and incubated with S1-RBD-His protein followed by FITC conjugated detection antibody which is an anti-His-FITC. In this FITC traced ACE2/S1-RBD binding system, the presence of immune sera collected from guinea pigs immunized with varying forms of S-RBD-sFc, S-RBDa-sFc, or S-RBD-Fc were tested for their respective binding inhibition capabilities. As shown in FIG. 43, a dose dependent curve was established for each series of immune sera collected at 5 wpi after prime and booster immunizations for the respective designer protein immunogens from about 100% inhibition down to the range of about 10% inhibition with characteristic IC₅₀ values being at 1:1024, 1:180, and 1:300 for designer protein immunogens of S-RBD-sFc, S-RBDa-Tc, and S-RBD-Fc respectively. The Geometric Mean Titer (GMT) ID₅₀ values for antibodies raised were 202, 69.2, and 108 for designer protein immunogens of S-RBD-sFc, S-RBDa-Fc, and S-RBD-Fc respectively. As shown in FIG. 44, representative plots of the inhibition profiles for all three designer protein immunogens were presented for sera collected at 0, 3, and 5 weeks that were fixed at a 1:625 dilution to assess the relative S1-ACE2 binding inhibition generated by this cell-based blocking assay. This comparative binding inhibition study shows that S-RBD-sFc produced the best functional immunogenicity as exhibited by its high binding inhibition (about 75%) when compared to that of 21 and 33% of inhibition of S-RBDa-sFc (about 21%) and S-RBD-Fc (about 33%).

In view of all of the binding inhibition results, the S-RBD-sFc protein of the present disclosure appears to be the most effective high precision designer immunogen representative of the B cell component for the elicitation of functional antibodies capable of inhibiting S1 and ACE2 binding, a critical pathway for SARS-CoV-2 viral entry.

### 6. In Vitro Neutralization Assay

Serum samples collected from animals immunized with S-RBD-sFc, S-RBDa-Fc, and S-RBD-Fc were inactivated at 56° C. for 0.5 h and serially diluted with cell culture medium in two-fold steps. The diluted sera were mixed with either a CNI strain virus, performed in KeXin laboratory in Beijing or a Taiwan strain virus performed independently in Taipei, suspension of 100 TCID₅₀ in 96-well plates at a ratio of 1:1, followed by 2 hours incubation at 36.5° C. in a 5% CO₂ incubator. Vero cells (1-2×10⁴ cells) were then added to the serum-virus mixture, and the plates were incubated for 5 days at 36.5° C. in a 5% CO₂ incubator. The cytopathic effect (CPE) of each well was recorded under microscope, and the neutralizing titer was calculated by the dilution number of 50% protective condition.

As shown in Table 29 immune sera from guinea pigs after single immunization was collected at 3 wpi and submitted for test by KeXin laboratory in Beijing for this in vitro neutralization test. The pre-bleeds (0 wpi) and other control sera were found to be less than 8 by titer. Immune sera from immunogens with designer protein S-RBD-sFc demonstrated the best titer (1:>256) while the immune sera from S1-RBDa-sFc and S1-RBD-Fc were in the range of 128 and 192, respectively. This in vitro neutralization assay that detects the ability to inhibit virus induced CPE further illustrated the functional efficacy of the tested immune sera to prevent SARS-CoV-2 infection.

Another independent testing for these immune sera was conducted at Nangang, Taipei as shown in Table 29. Immune sera collected from guinea pigs after prime and booster shots with blood collected at 0, 3, and 5 wpi were performed by this CPE based in vitro neutralization assay. In this second site testing, highly reproducible results were obtained for the 0 and 3 wpi immune sera with neutralizing titers measured between 128 and 256, while the titers of the immune sera from these designer proteins were around 4,096 and 8,192, about 15 to 30-fold higher than the immune sera upon single administration. The pre-bleeds and other control sera were found to be less than 8 or 4 depending on the respective laboratory scoring system. Immune sera from constructs with designer protein S1-RBD-sFc demonstrated best titer (1:>256) while the other immune sera were in the range of 128 and 192 as observed in the Beijing laboratory. Thus, at least more than 2-fold in neutralizing titers was found when using the S1-RBD-sFc as the designer immunogen than the other two designer proteins S1-RBD-Fc or S1-RBDa-sFc. The confirmation by this in vitro neutralization assay in two independent laboratories for ability of these designer protein induced antibodies to inhibit virus induced. CPE further illustrated the functional efficacy of these immune sera, thus the utility of these high precision designer proteins as immunogens in vaccine formulations for the prevention of SARS-CoV-2 infection.

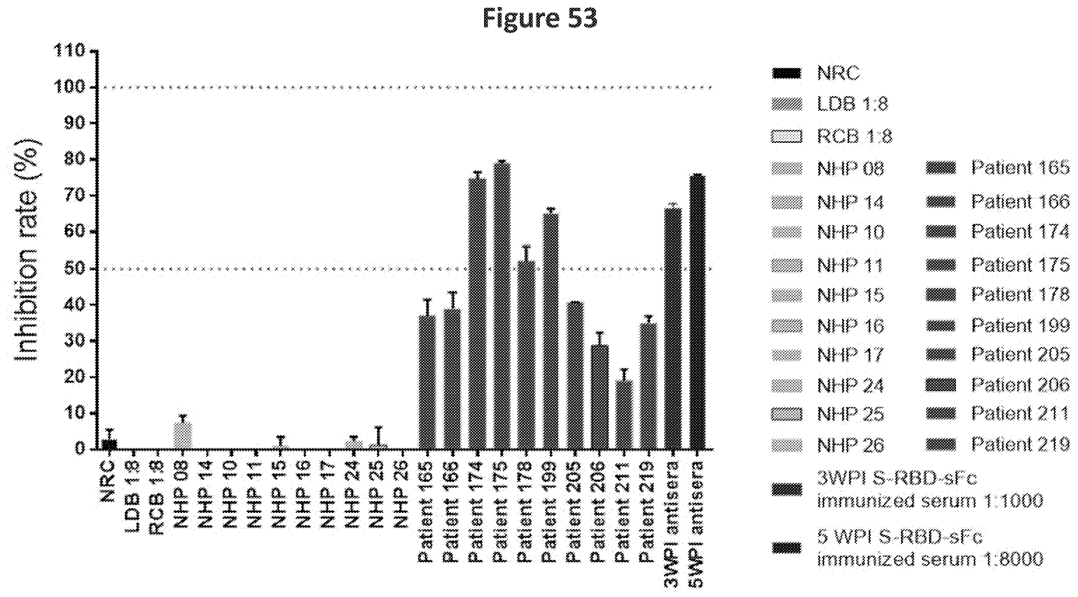

The neutralizing titers in sera from guinea pigs immunized with S1-RBD-sFc were compared against those in convalescent sera of COVID-19 patients. Using the S1-RBD:ACE2 binding inhibition ELISA (also termed as gNeu ELISA), the responses in guinea pigs were compared against those in convalescent sera from Taiwanese COVID-19 patients after discharge from hospitalization. The results, shown in FIG. 53, demonstrated that guinea pig immune sera diluted 1,000-fold (3 WPI) or 8,000-fold (5 WPI) exhibited comparable or higher inhibition of S1-RBD:ACE2 binding than by the convalescent sera of 10 patients diluted at 20-fold, illustrating that the sera of guinea pigs contained ≥50-fold higher antibody titers than human convalescent sera.

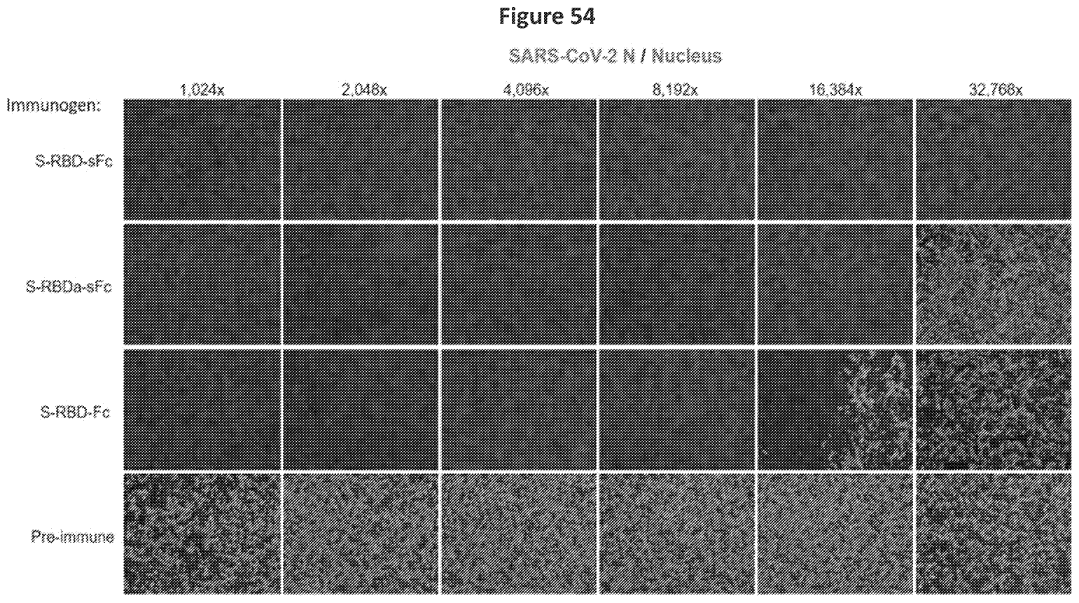

Further confirmation of the neutralizing potency of the antibodies was provided by a separate CPE study with anti-SARS-CoV-2 N protein antibody and immunofluorescent visualization. Again, a complete neutralization of SARS-CoV-2 (VNT100) was observed at a 1:32,768-fold dilution of animal sera in samples from animals immunized with S1-RBD-sFc fusion protein at 5 WPI (FIG. 54). Immune sera collected at 5 WPI from guinea pigs vaccinated at 0 and 3 WPI with S1-RBD-sFc, S1-RBDa-sFc, and S1-RBD-Fc with MONTANIDE™ ISA 50V2 were analyzed. The monolayers of Vero-E6 cells infected with virus-serum mixtures were assessed by immunofluorescence (IFA). Cells were stained with human anti-SARS-CoV-2 N protein antibody and detected with anti-human IgG-488 (light color). The nuclei were counter stained with DAPI (4′,6-diamidino-2-phenylindole) (dark color).

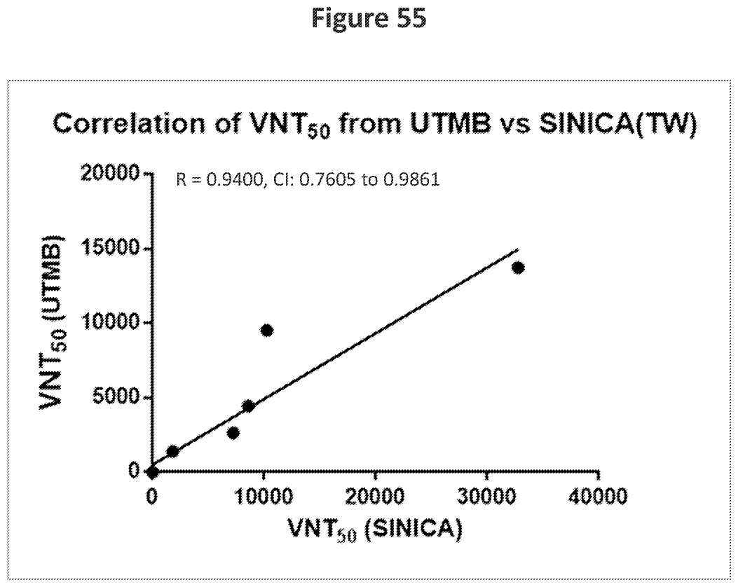

To further verify the neutralizing titers obtained by the CPE assay and IFA, 10 samples (positive and negatives) were blind coded and sent to Dr. Alexander Bukreyev's laboratory at the University of Texas Medical Branch (UTMB) in Galveston, Tex. These were tested in a replicating virus neutralization assay and the VNT50 titer for each sample was calculated. The results showed a strong correlation (r=0.9400) between the two assays performed at UTMB and Academia Sinica (FIG. 55).

In sum, the results from the immunogenicity testing indicated that all three vaccine formulations were immunogenic, with S1-RBD-sFc having clear advantages in terms of S1-RBD binding antibody titer, inhibition of ACE2 binding by SARS-CoV-2 S1-RBD protein, and neutralization of live SARS-CoV-2.

### EXAMPLE 14

### Manufacturing of the Multitope Protein/Peptide Vaccine Composition for the Prevention of Infection by SARS-CoV-2

Different formulations of the vaccine composition were prepared and evaluated in a pre-formulation characterization study to test their suitability for vaccine administration. In a forced degradation study, S-RBD-sFc was shown to be sensitive to heat, light exposure, and agitation but not sensitive to freezing and thawing cycles. The conditions considered sensitive to S-RBD-sFc were used for selecting the appropriate pH and excipients suitable for vaccine administration.

1. pH—Heat and UV Exposure

The isoelectric point (pI) value of S-RBD-sFc is between 7.3 to 8.4 so formulations were prepared with pH ranging from 5.7 to 7.0. In general, as the formulation pH moves away from the isoelectric point (pI), the solutions become clearer because protein solubility increases accordingly.

Size exclusion chromatography was used to determine whether the pH of the formulation had an effect on either heat-induced protein aggregation or UV-induced impurities. In this study, solutions containing S-RBD-sFc with pH ranging from 5.7 to 7.0, using a histidine buffer, were prepared and were either incubated at 35° C. for 24 hours or subjected to UV light for 24 hours. Size exclusion chromatography was used to determine the amount of S-RBD-sFc was present as well as several high molecular weight (HMW) impurities. The results from this study are shown in Table 30. Specifically, the results showed that pH had no obvious effect on heat-induced protein aggregation. The results also showed that, after UV exposure for 24 hours, S-RBD-sFc formed fewer high molecular weight impurities as the pH decreases, particularly from pH 5.7 to 6.4.

Based on this study, the final formulation was selected following the evaluation of prototype formulations at stressed conditions at the target pH of 5.9 using 10 mM histidine and the formulation pH specification limits of pH 5.4 and pH 6.4.

### 2. Surfactant—Agitation

Based on a forced degradation study, S-RBD-sFc was found to be sensitive to agitation stress and prone to form visible particles during agitation. Surfactants are often used to reduce the protein adsorption at the solid-liquid and liquid-air interface, which might lead to protein destabilization. Thus, a study was performed to determine if polysorbate 80 is capable of reducing or preventing precipitation of S-RBD-sFc after agitation.

In this study, three separate solutions containing approximately 2 mg/mL of S-RBD-sFc were agitated at 1,200 RPM at 25° C for 67 hours. The first solution contained 0.03% (w/v) polysorbate 80, the second solution contained 0.06% (w/v) polysorbate 80, and the third solution was a control without any polysorbate 80. In this study, the results showed that 0.06% (w/v) polysorbate 80 efficiently mitigates precipitation of S-RBD-sFc after agitation (data not shown). Therefore, the presence of 0.06% (w/v) polysorbate 80 was determined to improve stability and reduce precipitation of S-RBD-sFc in the formulation.

### 3. Protein Buffers

Additives, such as arginine-HCl, sucrose, and glycerol are frequently used as a protectant in the formulation development of proteins.

In this study, solutions containing S-RBD-sFc together with varying amounts of as arginine-HCl (25 mM to 100 mM), sucrose (25 mM to 100 mM), or glycerol (5% to 15%) were incubated at 50° C. for 1 hour. Size exclusion chromatography was used to determine the amount of S-RBD-sFc was present as well as several high molecular weight (HMW) impurities. The results from this study are shown in Table 30. Specifically, the results indicated that the addition of arginine-HCl, sucrose, or glycerol were able to lower heat-induced aggregation. These results were further confirmed by measuring the turbidity (OD600) of samples incubated at 40° C. for 45 min. Consistent with the size exclusion chromatography results, the addition of arginine-HCl, sucrose, or glycerol efficiently reduced the turbidity of samples (data not shown).

The effect of arginine-HCl, sucrose, or glycerol under UV stress on S-RBD-sFc solutions at pH 5.9 was also evaluated. Size exclusion chromatography results indicated that the addition of arginine-HCl slightly increased light-induced aggregation, but sucrose and glycerol did not have any significant impact on aggregation (Table 30).

### 4. Summary

A summary of the results obtained in the formulation screening studies is provided in Table 31.

### EXAMPLE 15

### Production of the S1-RBD-sFc Protein for use in the Multitope Protein/Peptide Vaccine Composition for the Prevention of Infection by SARS-CoV-2

The fed-batch production development for a small pilot scale batch (15 L) and large-scale batch (100 L) were carried out as described below.

### 1. Pilot Batch (15 L)

a. Fed-Batch Cell Culture Upstream Process

The fed-batch production development at pilot scale was carried out in a 15-L Finesse bioreactor with an initial working volume 9 L. HYPERFORMA™ 15 L bioreactor is a glass vessel bioreactor equipped with HYPERFORMA™ G3Lab Controller and TruFlow gas mass flow controller (MFC). The equipped impeller is a pitched blade impeller, and the sparger is a drilled pipe sparger with 0.8 mm diameter holes for aeration. The 15-L bioreactor parameters were as follows:
a. Medium: DYNAMIS+1 g/kg dextran sulfate+1.17 g/kg glutamine
b. Initial Cell Density: 0.3E6 vc/mL
c. Temperature: 37° C; TS to 32° C. on D5
d. pH: pH 7.0±0.3; base: 1 M Na₂CO₃; acid: CO₂
e. Dissolved Oxygen: Setpoint 50%
f. Feeding Strategy: 83% EX-CELL® ACF CHO Medium+17% EX-CELL® 325 PF CHO Medium supplemented with 50 g/kg glucose and 20 g/kg yeast extract. D3-D7: 3% daily; D8-D12: 4% daily (total feeding ratio: 35% w/w)
g. Glucose Control: D3-D13: add 2 g/kg glucose (stock 300 g/kg) when [Gluc]≤2 g/L
h. Harvest Criteria: Cell viability ≤60% or on D14

In brief, DYNAMIS™ AGT™ Medium (Thermo Fisher Scientific, A2617502) supplemented with L-Glutamine and dextran sulfate was used for both seed train expansion and production process. Bolus nutrient feed to the bioreactor was started on run day 3 (D3). The nutrient feed was formulated by blending 83% EX-CELL® ACF CHO Medium (Merck, C9098) with 17% EX-CELL® 325 PF CHO Medium (Merck, 24340C). Daily monitoring of cell number, cell viability, concentration of the metabolites (glucose, lactate, glutamine, glutamate and ammonia), osmolality, pH, pCO₂ and pO₂ were performed on BioProfile FLEX Analyzer (Nova Biomedical). The harvest criteria were the cell viability below 60% or on production day 14 (D14).

On the day of harvest, the cell culture fluid was clarified by COHC depth filter (Merck, MC0HC05FS1) followed by 0.22 μm capsule filtration. The harvested cell culture fluid (HCCF) was transferred to the Protein Purification Lab for downstream processing immediately.

In this process, the peak VCD was approximately 14E+06 vc/mL on day 7 and the cell viability was able to sustain ≥90% till the end of production. The productivity of S1-RBD-sFc was 1.6 g/L on day 14.

b. Harvest

Millistak+ POD C0HC 0.55 m² and Opticap XL 5 Capsule were applied to harvest materials. The filter was flushed with 100 L/m² of purified water at a flux rate of 600 LMH. The flush rate was 5 L/min and flush time was at least 10 minutes. Blow down was performed to drain off purified water from the POD filter before running filtrate (10 psi for at least 10 minutes). Run harvest cell culture fluid (HCCF) with 500 L/min, which was equal to 54.5 LMH. The first 1.4 L retentate was abandoned and the rest of retentate was collected. During the whole operation, the pressure was monitored and should not exceed 30 psi. The pre-clarification and post-clarification turbidities were 1343 NTU and 12.9 NTU, respectively, and the pre-clarification and post-clarification titers were 1.66 g/L and 1.50 g/L, respectively, Upstream product yields were high (1.5 g/L).

c. Downstream Purification Process Development

Briefly, the harvested cell culture fluid (Hal) was first treated with 1% TWEEN 80 (Merck, 8.17061) and 0,3% TNBP (Merck, 1.00002) and held for 1 hour without agitation at ambient temperature (23±4° C.) for solvent/detergent virus inactivation. The solvent/detergent treated HCCF was purified using a Protein A affinity chromatography column (MabSelectSuRe LX resin, Cytiva Life Sciences, 17-5474-03). The eluate from the Protein A column was neutralized to pH 6.0 immediately by 1 M Tris base solution (Merck, 1.08386). The neutralized protein solution was filtered by two types of depth filter, C0HC (23 cm², Merck Millipore, MC0HC23CL3) and X0SP (23 cm², Merck Millipore, MX0SP23CL3) to remove precipitates and impurities. The clarified protein solution was further purified by a cation exchange chromatography column (NUVIA™ HR-S media, Bio-Rad, 156-0515). The protein concentration was adjusted to 5 mglml, and the protein solution was subjected to viral filtration (PLANOVATM 20N Nano filter, Asahi Kasei, 20NZ-001). The filtrate from the nano filtration was buffer exchanged into formulation buffer by using tangential flow filtration (TANGENX™ SIUS™ PDn TFF Cassette, Repligen, PP030MP1L). After the buffer exchange, TWEEN 80 was then added to the formulated protein solution at a final concentration of 0.06% (w/v) followed by a 0.22 μm filtration, the formulated product was stored at 2-8° C. and protected from light exposure.

d. Process Yields, 15L Pilot Lot

The yield of each step was as follows:
a. Solvent detergent virus inactivation, protein A chromatography, neutralization and depth filtration: 11.30 g (83.1% yield).
b. Cation exchange chromatograph: 10.96 g (96.7% yield).
c. Nano-filtration, formulation by diafiltration and 0.2 μg filtration: 10.50 g (99.7% yield). The overall recovery was 80.3% yield.

### 2. Large Scale Batch (100 L)

A clinical batch of S-RBD-sFc (100 L) was manufactured from the clonal Research Cell Bank. The changes were made only at the drug substance level without changes in final composition. The raw materials and the process parameters were not changed, only the batch size is scaled up. No significant differences are observed between both lots.

The impact of the changes in manufacturing process for S-RBD-sFc drug substance between the pilot batch and the large-scale batch were assessed by a comparability study.

To assess the comparability between drug substance batches from the 15 L scale process and drug substance from the 100 L scale process, the analytical data of release data generated by characterizations and data of forced degradation study were compared and evaluated.

The S-RBD-sFc lots produced by the 15 L scale and 100 L scale manufacturing processes all met release specifications set in the respective specifications. All tested lots showed lot-to-lot consistency with similar levels of size variants and impurity, similar distribution of charge variants and comparable potency.

The results of the characterization study demonstrated comparability and consistency in the protein and carbohydrate structures, post translational modifications, purity/impurity, heterogeneity and biological activity of S-RBD-sFc lots produced by the 15 L scale or 100 L scale manufacturing process. In addition, the forced degradation study showed that the degradation pathways and the sensitivity to specific degradation conditions were similar and comparable for the tested lots manufactured by different process.

Overall, the results demonstrated the comparability of S-RBD-sFc lots between those produced by 15 L scale and 100 L scale with respect to the results obtained from release testing, forced degradation studies and additional characterizations.

### EXAMPLE 16

### A Multitope Protein/Peptide Vaccine Composition for the Prevention of Infection by SARS-CoV-2

The initial immunogenicity assessment in guinea pigs established the humoral immunogenicity of our RBD-based protein and allowed selection of S1-RBD-sFc (SEQ ID NO: 235) as the main immunogenic B cell component for a vaccine against SARS-CoV-2.

The presence of T cell epitopes is important for the induction of B cell memory response against viral antigens. SARS-CoV-2 CTL and Th epitopes, validated by MHC-binding and T cell functional assays, that are conserved between SARS-CoV-2 and SARS-CoV-1 (2003) viruses are employed in the design of the high precision SARS-CoV-2 vaccine against COVID-19. Identification of T cell epitopes on SARS-CoV-1 (2003), determined using MHC-binding assays, were used to determine corresponding T cell epitopes in SARS-CoV-2 (2019) by sequence alignment (see FIGS. 3, 4, and 5A-5C and Table 32). CTL epitopes that are incorporated in the design of the disclosed high precision designer SARS-CoV-2 vaccine were identified in a similar manner. The Th and CTL epitopes that are incorporated in SARS-CoV-2 vaccine design have been validated by MHC Class II binding and T cell stimulation as shown in Table 32. Specific multitope protein/peptide vaccine compositions for the prevention of infection by SARS-CoV-2 containing 20 μg/mL, 60 μg/mL, and 200 μg/mL (combined weight of the S1-RBD-sFc fusion protein and the Th/CTL peptides) are shown in Tables 33 to 35.

### 1. Immunogenicity Study in Rats

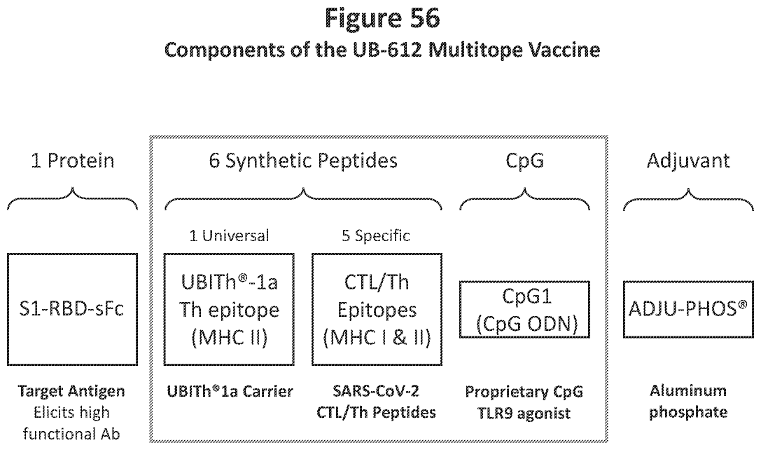

In a set of experiments conducted in rats, a proprietary mixture of Th/CTL peptides (SEQ ID NOs: 345, 346, 348, 348, 361, and 66) were added to the S1-RBS-sFc (SEQ ID NO: 235) B cell component for further assessment of optimal formulations and adjuvants and establishment of the cellular immunity components of the vaccine (e.g., FIG. 56). This vaccine composition was utilized in the following studies.

a. Humoral Immunogenicity Testing in Rats

The guinea pig experiments described in Example 13 were tested with three protein candidates with a single dosing regimen with a prime (100 μg or 200 μg) and a boost (50 μg or100 μg) using ISA 50 as an adjuvant, allowing for a rigorous comparison of the respective candidate constructs. In this set of experiments conducted in rats, varying doses of immunogen and adjuvants were evaluated to allow selection of an optimal adjuvant based on S1-RBD binding antibody titers and balanced Th1/Th2 responses.

The vaccine composition containing the S1-RBD-sFc protein with the Th/CTL peptides were combined the candidate vaccine with two different adjuvant systems, (a) ISA51 combined with CpG3 (SEQ ID NO: 106) and (b) ADJU-PHOS® combined with CpG1 (SEQ ID NO: 104). These vaccine-adjuvant combinations were administered to rats IM on 0 WPI (prime) and 2 WPI (boost) with a wide dose range of 10 to 300 μg per injection. The animals were bled at 0, 2 (i.e., after 1 dose), 3 and 4 WPI (i.e., 1 and 2 weeks after the 2nd dose) for antibody titer analyses.

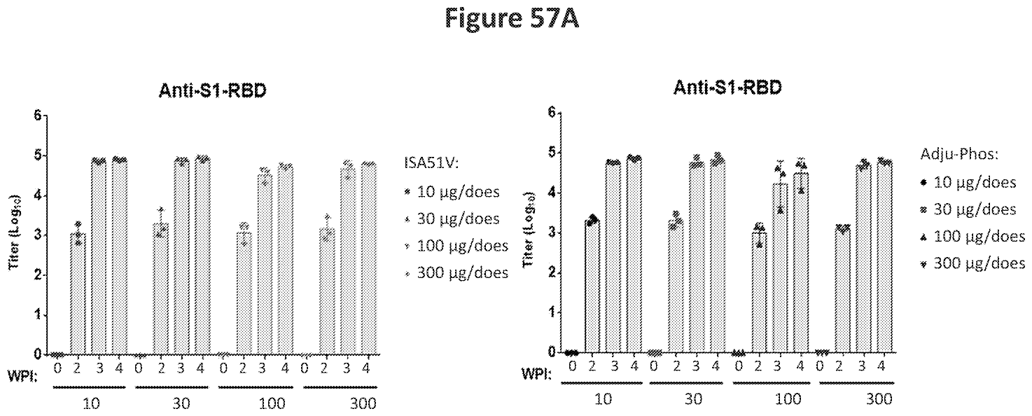

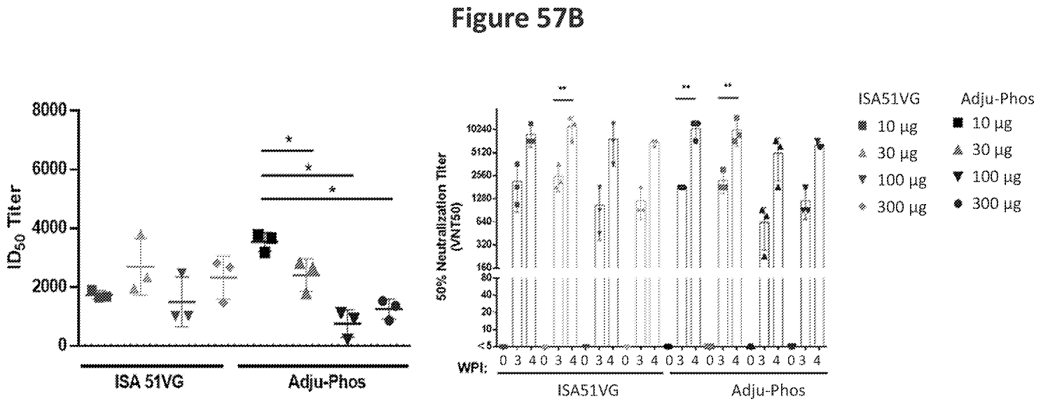

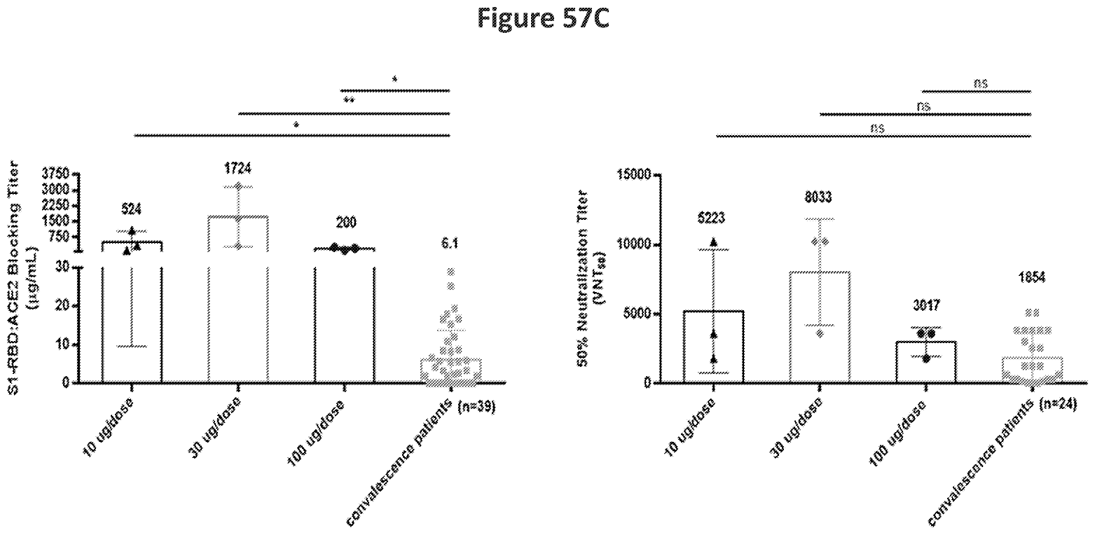

Results of binding antibody (BAb) testing at all time points demonstrated that vaccines formulated with both adjuvant systems elicited similar levels of anti S1-RBD ELISA titers across all doses ranging from 10 to 300 μg indicative of an excellent immunogenicity of the vaccine formulations even with low quantities of the primary protein immunogen (FIG. 57A). In addition, a 100-μg dose of S1-RBD-sFc without the synthetic peptide components stimulated high S1-RBD binding activity similar to previous guinea pig studies (data not shown).

In the S1-RBD:ACE2 binding inhibition ELISA test, doses of 10 and 30 μg induced as strong inhibitory activity as the high doses at 100 and 300 μg at 4 WPI (FIG. 57B, left panel). The most potent inhibitory activity was seen with the lowest dose of S1-RBD-sFc protein (10 μg) formulated with rationally designed peptides and the ADJU-PHOS®/CpG1 adjuvant. In the replicating virus neutralization assay against the Taiwanese SARS-CoV-2 isolate (as discussed above for guinea pig studies), the 4 WPI immune sera induced by the vaccine composition did not show a significant dose-dependent effect. However, low doses of adjuvanted protein, 10 and 30 μg, could neutralize viral infection at VNT50 of >10,240 dilution fold (FIG. 57B, right panel). The rat immune sera at 6 WPI from each vaccinated dose group were assayed, (a) in comparison with a set of convalescent sera of COVID-19 patients for titers in S1-RBD:ACE2 binding inhibition ELISA, expressed in blocking level of μg/mL (FIG. 57C, left panel); and (b) by a SARS-CoV-2 CPE assay in Vero-E6 cells, expressed as VNT50 (FIG. 57C, right panel). As shown in FIG. 57C, all doses of the vaccine formulations elicited neutralizing titers in rats that are significantly higher than those in convalescent patients by S1-RBD:ACE2 binding ELISA and higher (but not achieving statistical significance due to the spread in the patient data and the low number of animals) by VNT50.

b. Cellular immunogenicity Testing in Rats

To address the issue related to Th1/Th2 response balance, cellular responses in vaccinated rats were evaluated using ELISpot.

i. Procedure for Rat Th1/Th2 Balance Study

A total of 12 male Sprague Dawley rats at 8-10 weeks of age (300-350 gm/BW) were purchased from BioLASCO Taiwan Co., Ltd. After a 3-day acclimation, animals were randomly assigned to 4 groups. All procedures on animals were performed in accordance with the regulations and guidelines reviewed and approved by the Institutional Animal Care and Use Committee (IACUC) at UBIAsia. The IACUC number is AT-2028. The rats were vaccinated intramuscularly at weeks 0 (prime) and 2 (boost) with different doses ranging from 1 to 100 μg of a vaccine composition containing S1-RBD-sFc (SEQ ID NO: 235) with five Th/CTL peptides selected from S, M and N proteins of SARS-CoV-2 (SEQ ID NOs: 345₂346, 348. 348, and 361) and a proprietary universal Th peptide URITh®1a (SEQ ID NO: 66) formulated in ADJU-PHOS®/CpG1 adjuvant. The immune sera from rats (n=3 for each dose group) were collected at weeks 0, 2, 3, and 4 for assessment of antigenic activities. Splenocytes were collected at 4 WPI and restimulated in vitro at 2 μg/well either with the Th/CTL peptide pool plus S1-RBD or with the Th/CTL peptide pool alone. IFN-γ, IL-2, and II -4-secreting splenocytes were determined by ELISpot analysis. Cytokine-secreting cells (SC) per million cells was calculated by subtracting the negative control wells.

ii. ELISpot for Measurement of Cellular Responses

Spleens from vaccinated rats at 4 WPI were collected in Lymphocyte-conditioned medium (LCM; RPMI-1640 medium supplemented with 10% FBS and penicillin/streptomycin) and processed into single cell suspensions. Cell pellets were resuspended in 5 mL of RBC lysis buffer for 3 min at room temperature (RT), and RPMI-1640 medium containing penicillin/streptomycin was then added to stop the reaction. After centrifugation, cell pellets resuspended in LCM were used in ELISpot assay. ELISpot assays were performed using the Rat IFN-γ ELISpotPLUS kit (MABTECH, Cat. No.: 3220-4APW), Rat IL-4 T cell ELISpot kit (U-CyTech, Cat. No.: CT081) and Rat IL-2 ELISpot Kit (R&D Systems, Cat. No.: XEL502). ELISpot plates precoated with capture antibody were blocked with LCM for at least 30 min at RT. 250,000 rat splenocytes were plated into each well and stimulated with S1-RBD-His protein plus Th/CTL peptide pool, S1-RBD-His protein, Th/CTL peptide pool, or each single Th/CTL peptide for 18-24 hrs at 37° C. Cells were stimulated with a final concentration of 1 μg of each protein/peptide per well in LCM. The spots were developed based on manufacturer's instructions. LCM and ConA were used for negative and positive controls, respectively. Spots were scanned and quantified by AID iSpot reader. Spot-forming unit (SFU) per million cells was calculated by subtracting the negative control wells.

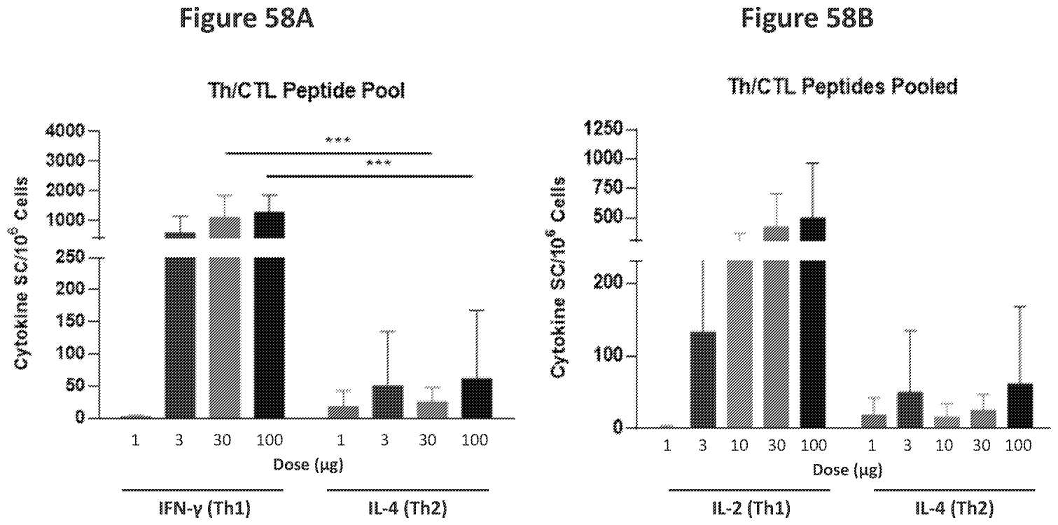

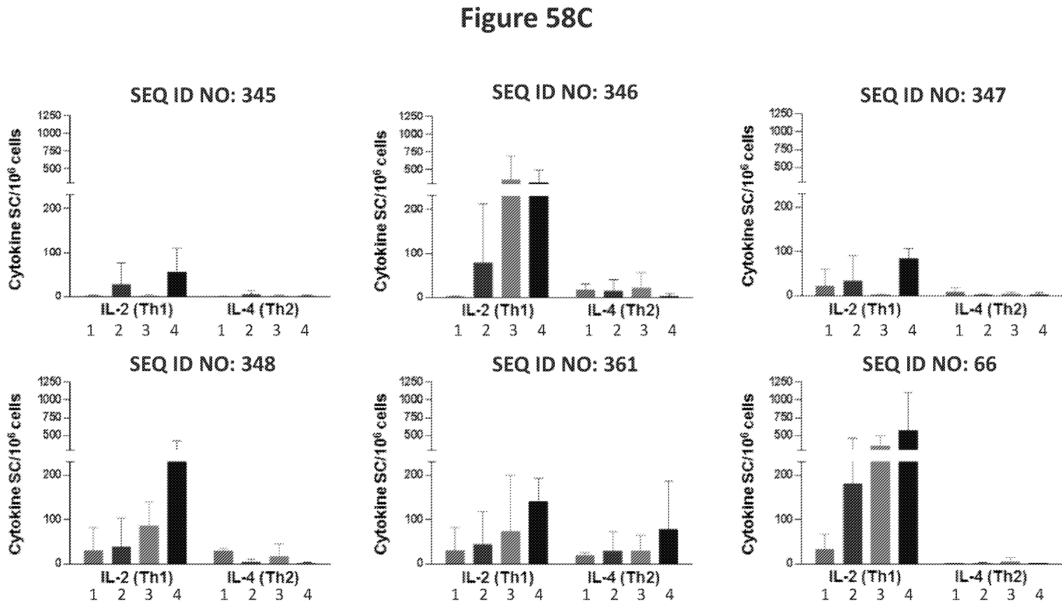

A dose-dependent trend in IFN-γ secretion was observed in splenocytes, while little secretion of IL-4 was seen (FIG. 58A). The results indicated that the vaccine composition was highly immunogenic and induced a Th1-prone cellular immune response as shown by the high ratios of IFN-γ/IL-4 or IL-2/IL-4. High ratios of IL-2/IL-4 were also observed in the presence of the Th/CTL peptide pool (FIG. 58B) and for restimulation with individual peptides, which induced little IL-4 secretion (FIG. 58C). Bars represent the mean SD (n=3). The secretion of IFN-γ or IL-2 was observed to be significantly higher than that of IL-4 in 30 and 100 μg group (*** p<0,005 using Least Square Mean and paired wise comparison) but they were not statistically different in 1 or 3 μg dose groups.

### 2. Challenge Studies in Transgenic Mice

The initial challenge study of the vaccine composition was performed in the AAV/hACE2 transduced BALB/c mouse model established by Dr. Tau, Mi-Hua at Academia Sinica in Taiwan; adaptations of this model are also reported by other investigators.

a. Animal Procedures for BALB/C Challenge Studies

A total of 12 male BALB/C at 8-10 weeks of age were purchased from BioLASCO Taiwan Co., Ltd. After a 3-day acclimation, animals were randomly assigned to 4 groups. All procedures on animals were performed in accordance with the regulations and guidelines reviewed and approved by the Institutional Animal Care and Use Committee (IACUC) at UBI Asia. The IACUC numbers are AT2032 and AT2033.

The mice were vaccinated by IM route at weeks 0 (prime) and 2 (boost) with 3, 9, or 30 ug of the vaccine composition containing S1-RBD-sFc (SEQ ID NO: 235) together with Th/CTL peptides (SEQ ID NOs: 345, 346, 348, 348, 361, and 66) formulated in ADJU-PHOS®/CpG1 adjuvant. The immune sera from mice were collected at weeks 0, 3 and 4 for assessment of immunogenic and functional activities by the assay methods described below.

AAV6/CB-hACE2 and AAV9/CB-hACE2 were produced by AAV core facility in Academia Sinica. BALB/C mice (8-10 weeks old) were anaesthetized by intraperitoneal injection of a mixture of Atropine (0.4 mg/ml)/Ketamine (20 mg/ml)/Xylazine (0.4%). The mice were then intratracheally (IT) injected with 3×1011 vg of AAV6/hACE2 in 100 μL saline. To transduce extrapulmonary organs, 1×1012 vg of AAV9/hACE2 in 100 μL saline were intraperitoneally injected into the mice.

Two weeks after AAV6/CB-hACE2 and AAV9/CB-hACE2 transduction, the mice were anesthetized and intranasally challenged with 1×104 PFU of the SARS-CoV-2 virus (hCoV-19/Taiwan/4/2020 TCDC #4 obtained from National Taiwan University, Taipei, Taiwan) in a volume of 100 μL. The mouse challenge experiments were evaluated and approved by the IACUC of Academia Sinica. Surviving mice from the experiments were sacrificed using carbon dioxide, according to the ISCIII IACUC guidelines. All animals were weighed after the SARS-CoV-2 challenge once per day.

b. RT-PCR for SARS-CoV-2 RNA Quantification

To measure the RNA levels of SARS-CoV-2, specific primers targeting 26,141 to 26,253 regions in the envelope (E) gene of the SARS-CoV-2 genome were used by Taqman real-time RT-PCR method that described in the previous study (Corman, et al. 2020). Forward primer E-Sarbeco-F1 (5′-ACAGGTACGTTAATAGTTAATAGCGT-3′; SEQ FD NO: 368) and the reverse primer E-Sarbeco-R2 (5′-ATATTGCAGCAGTACGCACACA-3′; SEQ ID NO: 369), in addition to the probe E-Sarbeco-P1 (5′-FAM-ACACTAGCCATCCTTACTGCGCTTCG-BBQ-3′; SEQ ID NO: 370) were used. A total of 30 μL RNA solution was collected from each sample using RNeasy Mini Kit (QIAGEN, Germany) according to the manufacturer's instructions. 5 μL of RNA sample was added in a total 25 μL mixture using Superscript III one-step RT-PCR system with Platinum Tag Polymerase (Thermo Fisher Scientific, USA). The final reaction mix contained 400 nM forward and reverse primers, 200 nM probe, 1.6 mM of deoxy-ribonucleoside triphosphate (dNTP), 4 mM magnesium sulphate, 50 nM ROX reference dye and 1 μL of enzyme mixture from the kit. The cycling conditions were performed with a one-step PCR protocol: 55° C. for 10 min for cDNA synthesis, followed by 3 min at 94° C. and 45 amplification cycles at 94° C. for 15 sec and 58° C. for 30 sec. Data were collected and calculated by Applied Biosystems 7500 Real-Time PCR System (Thermo Fisher Scientific, USA). A synthetic 113-bp oligonucleotide fragment was used as a qPCR standard to estimate copy numbers of viral genome. The oligonucleotides were synthesized by Genomics BioSci and Tech Co. Ltd. (Taipei, Taiwan).

c. Challenge Study

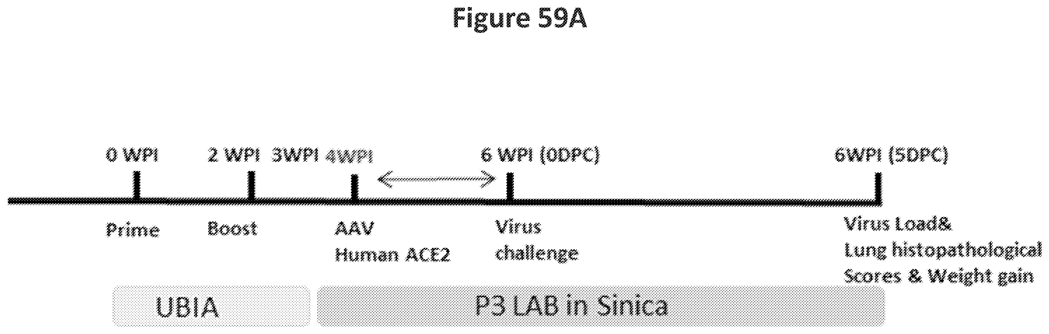

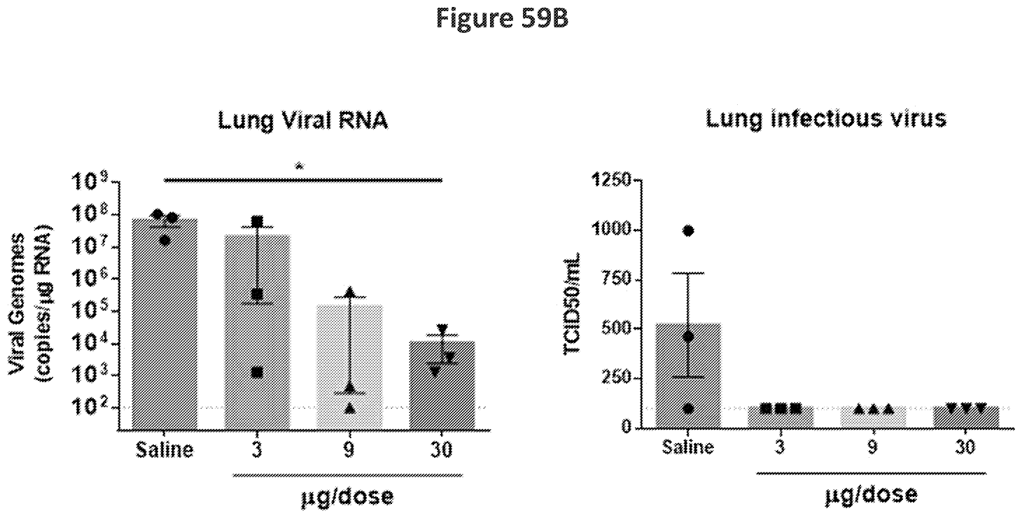

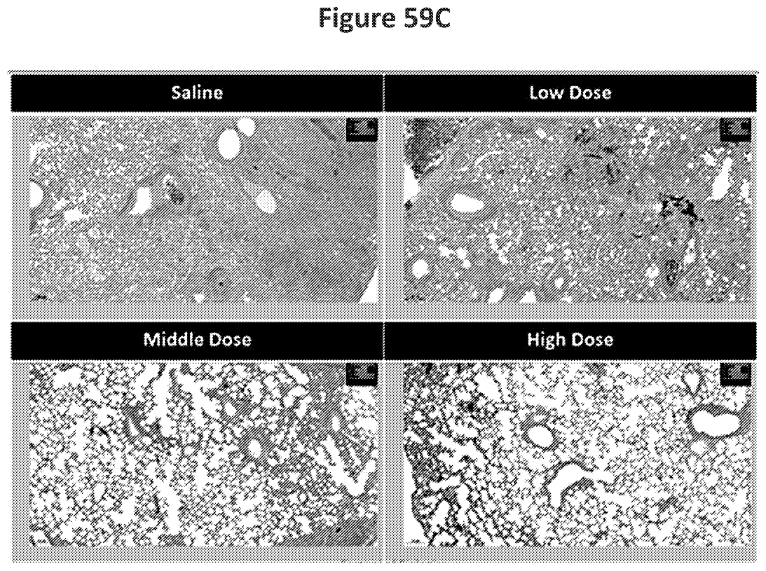

Groups of 3 mice were vaccinated at study 0 and 2 WPI with the vaccine composition described above containing 3, 9, or 30 μg of protein and formulated with ADJU-PHOS®/CpG1. The mice were infected with adeno-associated virus (AAV) expressing hACE2 at 4 WPI and challenged 2 weeks later with 106 TCID50 of SARS-CoV-2 by the intranasal (IN) route (FIG. 59A). Efficacy of the vaccine was measured using lung viral loads and body weight measurements. As shown in FIG. 59B, vaccination with 30 μg of the vaccine composition significantly reduced lung viral loads (˜3.5 log10 viral genome copies/μg RNA or ˜5-fold TCID50/mL of infectious virus) compared to saline group (p<0.05 as measured by paired t test). As shown in FIG. 59C, vaccination with middle and high doses led to clear reduction in lung pathology. Vaccination with 3 or 9 μg of the vaccine composition reduced live virus detection by cell culture method (TCID50) to below of the level of detection (LOD, FIG. 59B, right panel) but it did not appear to reduce viral loads significantly when measured by RT-PCR (FIG. 59B, left panel). Similarly, body weight measurements showed a significant difference between the high-dose group and the control group (data not shown). In sum, despite the lack of a statistical power (N=3 mice) in this study, it appears that the highest dose at 30 μg per dose could have had the maximum protective efficacy when one combines the lack of live virus detection and the lack of inflammatory cell infiltrations as well as lack of immunopathology in the lungs altogether.

3. lmmunogenicitv and Challenge Studies in Rhesus Macaques

Based on an established model using rhesus macaques (RM), an immunization study of the vaccine composition containing S1-RBD-sFc (SEQ ID NO: 235) together with Th/CTL peptides (SEQ ID NOs: 345, 346, 348, 348, 361, and 66) was performed as described below.

a. Immunogenicity Studies in Non-Human Primates

The study was conducted at JOINN Laboratories (Beijing) in rhesus macaques aged approximately 3-6 years. Animals were housed individually in stainless steel cages, an environmentally monitored, and well-ventilated room (conventional grade) maintained at a temperature of 18-26° C. and a relative humidity of 40-70%. Animals were quarantined and acclimatized for at least 14 days. The general health of the animals was evaluated and recorded by a veterinarian within three days upon arrival. Detailed clinical observations, body weight, body temperature, electrocardiogram (ECG), hematology, coagulation and clinical chemistry were performed on monkeys. The data were reviewed by a veterinarian before being transferred from the holding colony. Based on pre-experimental body weights obtained on Day −1, all animals were randomly assigned to respective dose groups using a computer-generated randomization procedure. All animals in Groups 1 to 4 were given either control or test article via intramuscular (IM) injection. Doses were administered to the quadriceps injection of one hind limbs. Monkeys were observed at least twice daily (AM and PM) during the study periods for clinical signs which included, but not limited to mortality, morbidity, feces, emesis, and the changes in water and food intake. Animals were bled at regular intervals for the immunogenicity studies described below.

Rhesus macaques (3-6 years old) were divided into four groups and injected. intramuscularly with high dose (100 μg/dose), medium dose (30 μg/dose), low dose (10 μg/dose) vaccine and physiological saline, respectively. All grouped animals were immunized at three times (days 0, 28 and 70) before challenged with 106 TCID50/ml SARS-CoV-2 virus by intratracheal routes (performed on day 82). Macaques were euthanized and lung tissues were collected at 7 days post challenge. At days 3, 5, 7 dpi, the throat swabs were collected. Blood samples were collected 0, 14, 28, 35, 42, 70, and 76 days post immunization, and 0, 3, 5, 7 days post challenge for neutralizing antibody test of SARS-CoV-2. Lung tissues were collected at 7 days post challenge and used for RT-PCR assay and histopathological assay. Analysis of lymphocyte subset percent (CD3+, CD4+and CD8+) and key cytokines (TNF-α, IFN-γ, IL-2, M-4, IL-6) were also performed in collected blood samples on days 0 and 3 post challenge, respectively.

b. Immunogenicity and Challenge Studies in Rhesus Macaques

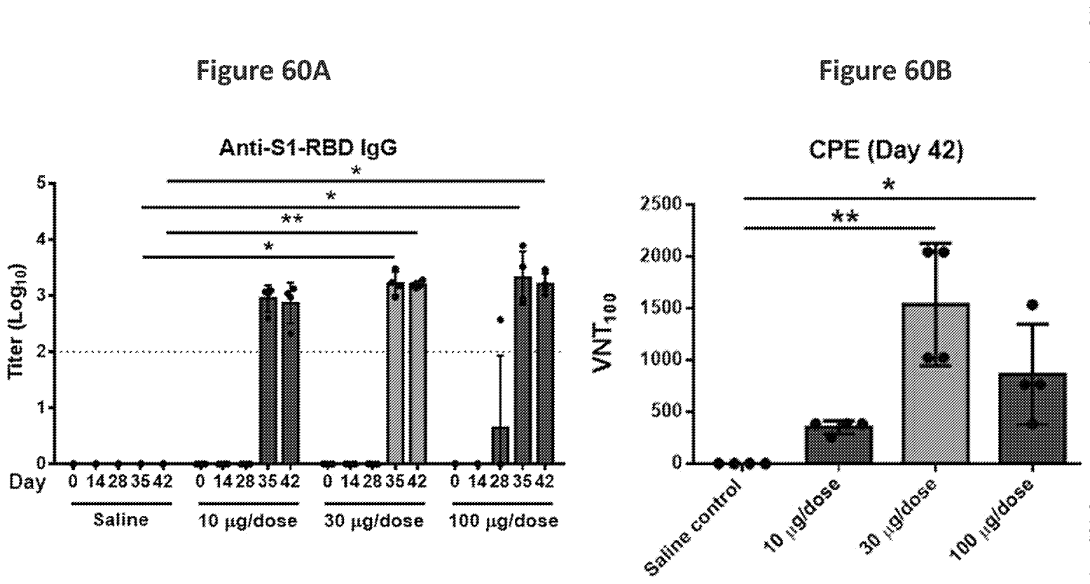

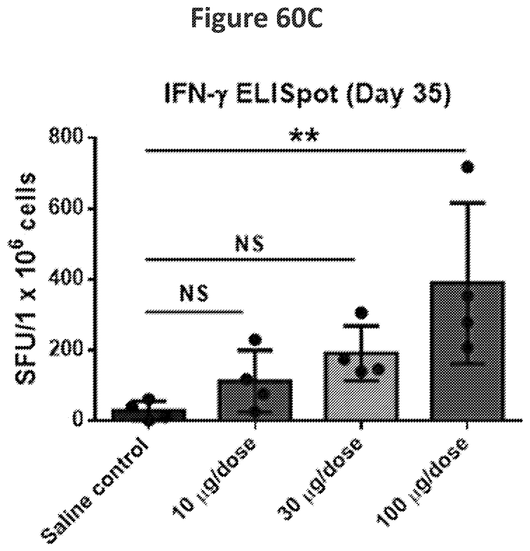

Based on an established model using rhesus macaques (RM), an immunization study of the vaccine composition by IM injection was initiated with RM (N=4/group) receiving 0, 10, 30, or 100 μg of the composition at 0 and 4 WPI. Immunogenicity measurements indicated that the serum IgG binding to S1-RBD was increased over baseline in all animals with binding titers reaching around 3 logs at 5 and 7 WPI (FIG. 60A). Strong neutralizing antibody responses were induced, with the 30 μg dose being most potent (FIG. 60B). ELISpot analysis indicated that vaccine composition activated antigen-specific IFN-γ-secreting T cells in a dose-dependent manner (FIG. 60C) with T cell responses highest at the 100 μg dose level.

### 4. Toxicity Stud in Preparation for Clinical Trials

To enable clinical trials, the vaccine composition containing S1-RBD-sFc (SEQ ID NO: 235) together with Th/CTL, peptides (SEQ ID NOs: 345, 346, 348, 348. 361, and 66) was tested in a GLP-compliant repeat-dose toxicology study in Sprague-Dawley rats as described below.

a. Protocol for Toxicology Studies

A total of 160 rats (80/sex) were randomly assigned to 8 groups based on the body weights obtained on Day −1 (1 days prior to the first dosing, the first dosing day was defined as Day 1), of which 120 rats were assigned to Groups 1, 2, 3 and 4 (15/sex/group) for the toxicity study, and 40 rats to Groups 5, 6, 7 and 8 (5/sex/group) for the satellite study. Rats were treated with saline injection for Groups 1 and 5 as negative control, vaccine composition placebo for Groups 2 and 6 as adjuvant control, and vaccine composition at doses of 100, 300 μg/animal for Groups 3 and 7 as well as Groups 4 and 8, respectively. Rats were treated via intramuscular injection into the one-side hind limbs muscle (quadriceps femoris and gastrocnemius, left side for the first dose and right side for the second dose) at multiple sites once every two weeks for 2 consecutive weeks, total 2 doses (on Days 1 and 15). The dose volume was 0.5 mL/animal. Clinical observations (including injection sites observation), body weight, food consumption, body temperature, ophthalmoscopic examinations, hematology, coagulation, clinical chemistry, urinalysis, T lymphocyte subpopulation, number of T lymphocyte spots secreting ITN-γ by peripheral blood mononuclear cells (PBMCs), cytokines, and immunogenicity, neutralizing antibody titer and IgG2b/IgG1 ratio analysis were performed during the study. The first 10 animals/sex/group in Groups 1 to 4 were designated for the terminal necropsy after 2 weeks of dosing (Day 18) and the remaining 5 animals/sex/group were designated for the 4-week recovery necropsy after the last dosing (Day 44). All animals in Groups 1 to 4 were given complete necropsy examinations, and then the organ weights, macroscopic and microscopic examinations were evaluated.

b. Toxicity Study in Preparation for Clinical Trials

To enable clinical trials, the vaccine composition was tested in a GLIA-compliant repeat-dose toxicology study in Sprague-Dawley rats. The study included a 300 μg dose, 3 times higher than that of the highest dose intended for clinical use. Although the schedule of 2 injections did not exceed that intended for clinical use, this is acceptable according to the WHO guidelines46. The study was also designed to evaluate the immunogenicity of the vaccine composition. One hundred and sixty (160) rats were randomly divided into 8 groups (80 males and 80 females) of which 40 rats were included in the satellite immunogenicity study. The low-and high dose groups were inoculated with the vaccine composition at 100 μg/animal (0.5 mL) and 300 μg/animal (0.5 mL) respectively; control groups were injected either with saline (0.9% saline) or adjuvant (vaccine composition placebo) at the same dose volume. The first ten animals/sex/group were designated for the terminal necropsy after two weeks of dosing at 2 WPI (Day 18) and the remaining 20 animals/sex/group were designated for the 4-week recovery necropsy after the last dosing at 4 WPI (Day 44). Under the experimental conditions, rats received IM injections into one hind limb muscle (quadriceps femoris and gastrocnemius, left side for the first dose and right side for the second dose) at multiple sites once every two weeks for 2 consecutive weeks, total 2 doses at 0 and 2 WPI (on Days 1 and 15).

Treatment with the vaccine composition at dose levels of up to 300 μg/animal at weeks 1 and 3 was well tolerated with no signs of systemic toxicity. Neither test article-related mortality nor moribundity was noted throughout the study. No vaccine-related abnormal findings were noted in clinical observations (including injection site observations) throughout the study. Neither erythema nor edema were noted at injection sites, and the Draize score was 0 for all observation time points. Similarly, no vaccine-related changes in body weight, food consumption, body temperature, hematology, chemistries (other than AG ratio), ophthalmoscopic examinations or urinalysis were observed, and no statistically significant changes were noted in CD3+, CD3+CD4+, CD3+CD8+, and the ratio of CD3+CD4+/CD3+CD8. Statistically significant increases were seen in fibrinogen, IFN-γ, and IL-6, while decreases in albumin/globulin ratio were observed; these results are consistent with an acute phase response to a vaccine, and all resolved by the end of the recovery period. Histopathological examinations of epididymides, skin, liver, prostate and mammary gland, revealed minimal inflammatory cell infiltrations with no visible lesions or abnormalities.

Immunogenicity of the vaccine composition measured in satellite groups showed that the vaccine was able to induce substantial levels of anti-SARS-CoV-2 S1RBD IgG in animals receiving two doses of 100 μg/animal or 300 μg/animal at 2 and 4 WPI (a 14-day interval) (data not shown). The S1-RBD binding IgG titers rose modestly over time after the boost at 2 WPI (Day 15), which reached around 2.6 log10 and 3.3 log10 in rats immunized with the vaccine composition at 100 μg/animal and 300 μg/animal, respectively, at 6 WPI (Day 44). The findings observed in this study are as expected for a vaccine designed to stimulate immune responses resulting in production of high titers of antibodies. Anti-SARS-CoV-2 S1-RBD IgG titers, subtype IgG and serum cytokine production by ELISA were performed to determine the Th1/Th2 responses. On analyses of S1-RBD-specific IgG subclasses, the patterns and induction levels of Th2-related subclass IgGI anti-SARS-CoV-2 S1-RBD were comparable to what was observed in total IgG anti-SARS-CoV-2 St-RBD. Only slight induction of Th1-related subclass IgG2b anti-SARS-CoV2 S1-RBD was detected in rats vaccinated with the vaccine composition at 6 WPI (Day 43). However, the serum cytokine pattern measured by ELISA indicated a Th1/Th2 balanced response (data not shown).

Clinical trials of the vaccine composition have begun in Taiwan. The first study, entitled “Phase I, Open-Label Study to Evaluate the Safety, Tolerability, and iimmunogenicity of UB-612 Vaccine in Healthy Adult Volunteers”, was initiated in Taiwan in September 2020. This trial includes three dose groups (N=20 per group) of UB-612 (10, 30, or 100 μg) given at days 1 and 29 (2 dose regimen). The primary endpoint is the occurrence of adverse events within seven days of vaccination; secondary endpoints include adverse events during the six-month follow-up perod, standard laboratory safety measures, antigen-specific antibody titers, seroconversion rates, T cell responses and increase of neutralizing antibody titers.

### EXAMPLE 17

### A Phase I, Open-Label Study to Evaluate the Safety, Tolerability, and Immunogenicity of the High Precision Designer Vaccine in Healthy Adult Volunteers

### 1. Objectives

The primary objective was to evaluate the safety, tolerability, and immunogenicity of the disclosed high precision designer vaccine in healthy adult volunteers.

### 2. Methodology

Open-label, two-dose intramuscular administration at Day 0 and week 4 with low and high doses of the disclosed high precision designer vaccine.

### 3. Number of Subjects

A total of 40 participants.

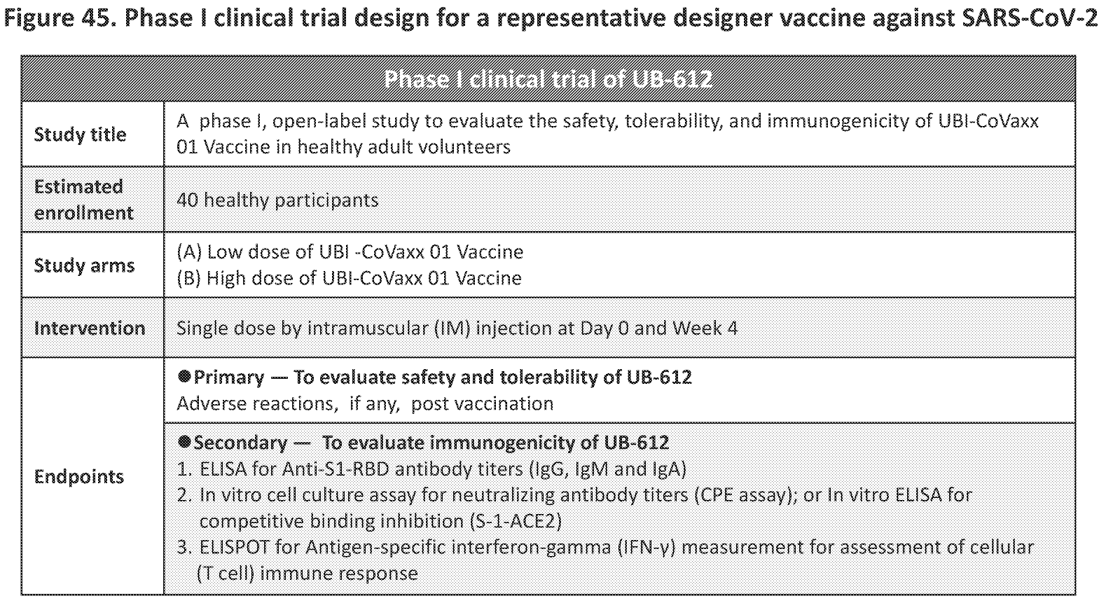

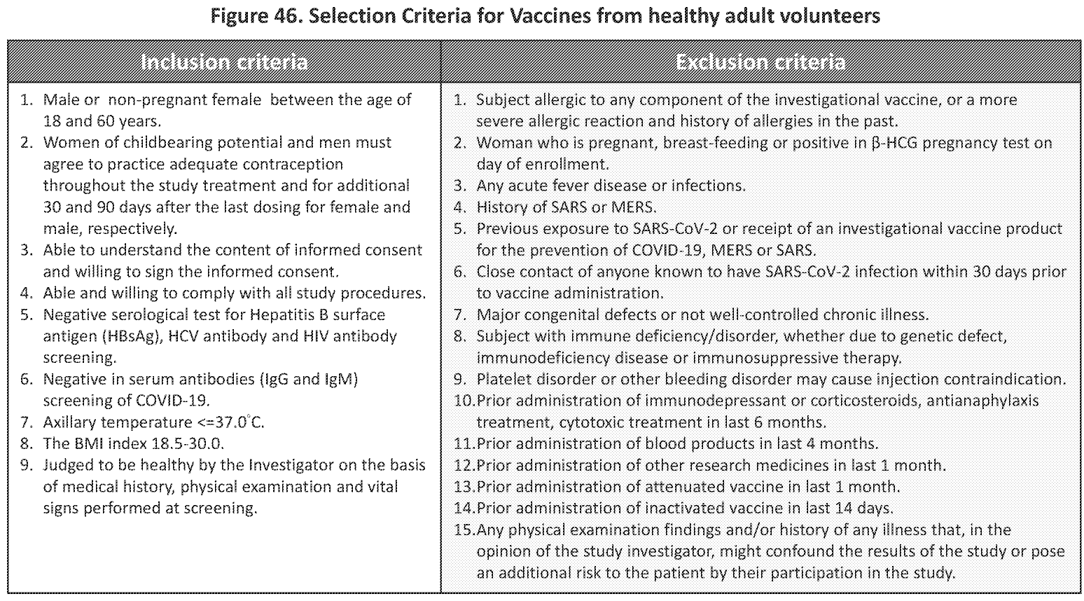

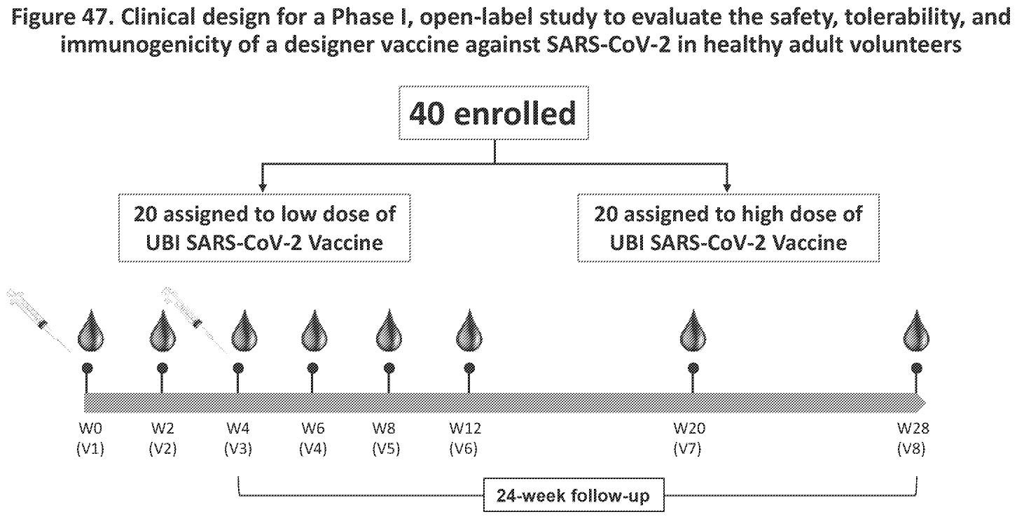

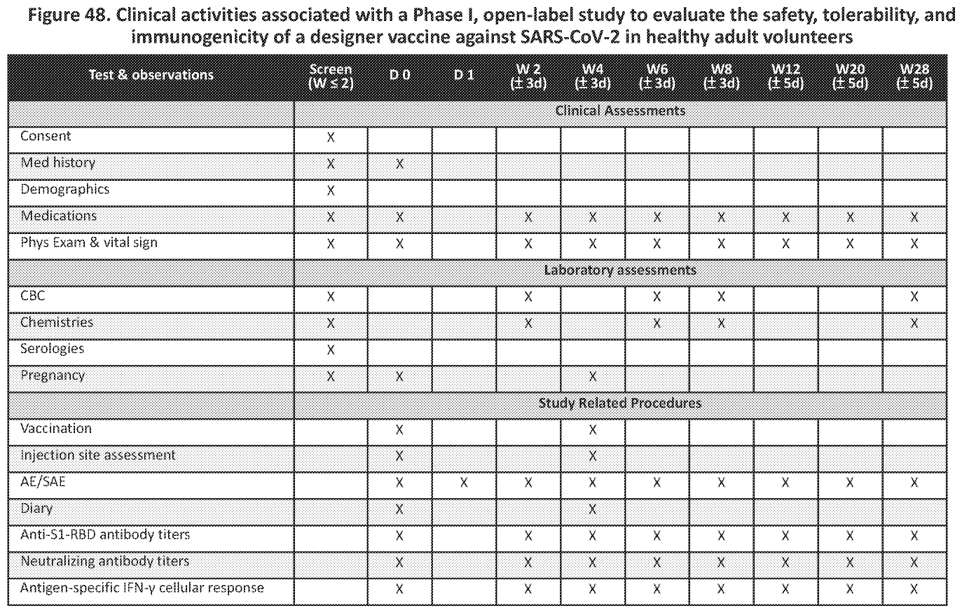

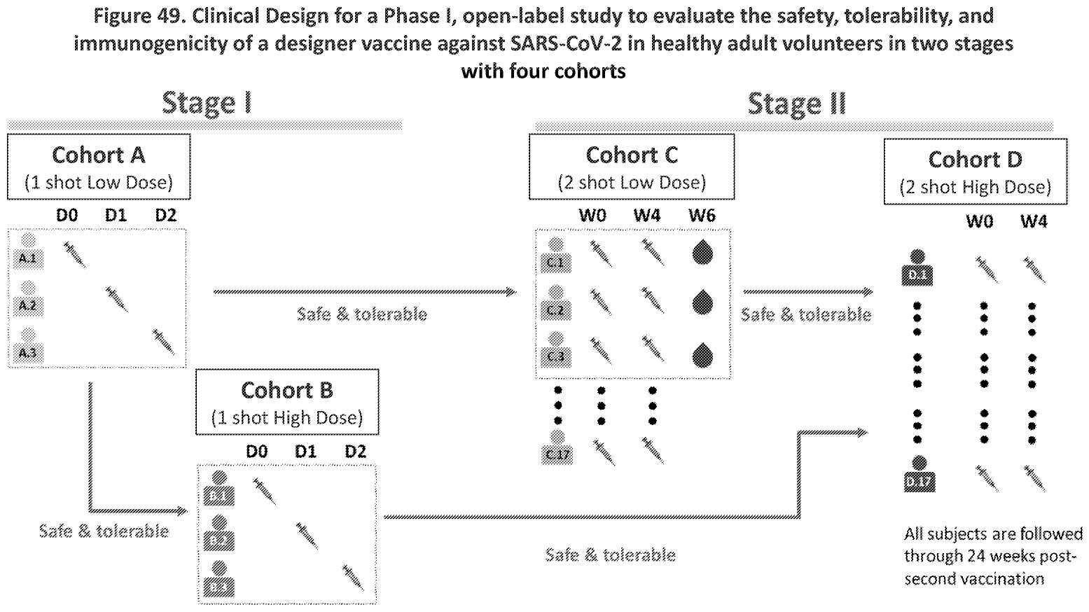

a. Study arms, intervention, primary and secondary endpoints are described in detail in FIG. 45 along with inclusion and exclusion criteria in FIG. 46.
b. Clinical design for a phase I, open-label study to evaluate the safety, tolerability, and immunogenicity of a designer vaccine against SARS-CoV-2 in healthy adults are delineated as shown in FIG. 47.
c. Clinical activities associated with a phase I, open-label study to evaluate the safety, tolerability, and immunogenicity of a designer vaccine against SARS-CoV-2 in healthy adult volunteers are delineated in detail, as shown in FIG. 48.
d. Clinical design for a phase I, open-label study to evaluate the safety, tolerability, and immunogenicity of a designer vaccine against SARS-CoV-2 in healthy adult volunteers in two stages with four cohorts are delineated in detail, as shown in FIG. 49.

### EXAMPLE 18

### Designer Long-Acting Protein Drug ACE2-ECD-sFc Generated High Antiviral Effect Measured in a Neutralizing Assay for Inhibition os SARS-CoV-2 Induced CPE In Vero Cells

The coronaviruses SARS-CoV-1 (2003) and SARS-CoV-2 (2019) enter host cells through binding of the viral envelope-anchored spike (S) protein to the receptor angiotensin-converting enzyme 2 (ACE2). Among other unique features of the S protein, SARS-CoV-2 binds to ACE2 with a higher affinity (up to 20-fold) compared to SARS-CoV-1, which corresponds to a rapid human-to-human transmissibility of new infections observed for SARS-CoV-2. As ACE2 plays a crucial role in the spread of SARS-CoV-2, an engineered soluble ACE2-like protein could potentially work as an effective interceptor to block viral invasion, thereby achieving therapeutic purpose while, at the same time, safeguarding the normal physiological function of the membrane-bound ACE2 from being further reduced and damaged.

Using a proprietary technology platform, a unique ACE receptor-based, long-acting fusion protein product of UMP grade can be used to treat COVID-19 of both symptomatic and asymptomatic patients. The technology platform integrates the plasmid construction of extracellular domain of ACE2 (ACE2-ECD) that links to a single chain immunoglobulin Fc fragment (sFc), expression and production in CHO-S cell line of ACE2-sFc fusion protein, and purification and bin-characterization of the protein species. The ACE2-sFc product is under preclinical testing and being planned for a parallel accelerated phase-1 safety study with patients confirmed having mild-to-severe SARS-CoV-2 infection upon clinical diagnosis and PCR confirmation.

A diverse array of in vitro bioassays has been performed demonstrating that the fusion protein ACE2-sFc is functionally active. These assays include a SPR-based binding affinity assay, a molecular and cellular recognition by SARS-CoV-2 spike (S) protein, and a neutralization of the S protein-ACE interaction by ACL 2-sFc. A proof-of-concept inhibition of SARS-CoV-2 infection has been confirmed on the cellular level. ACE2-sFc, either alone or in synergic combination with anti-IL6R mAb or the currently approved Remdesivir, could be of significant clinical utility for treatment of COVID-19.

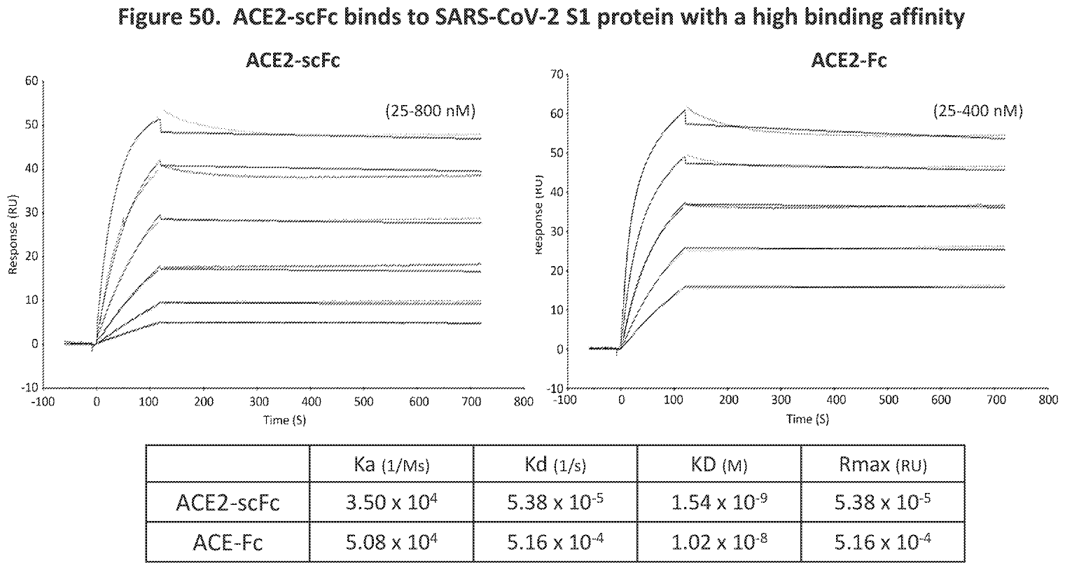

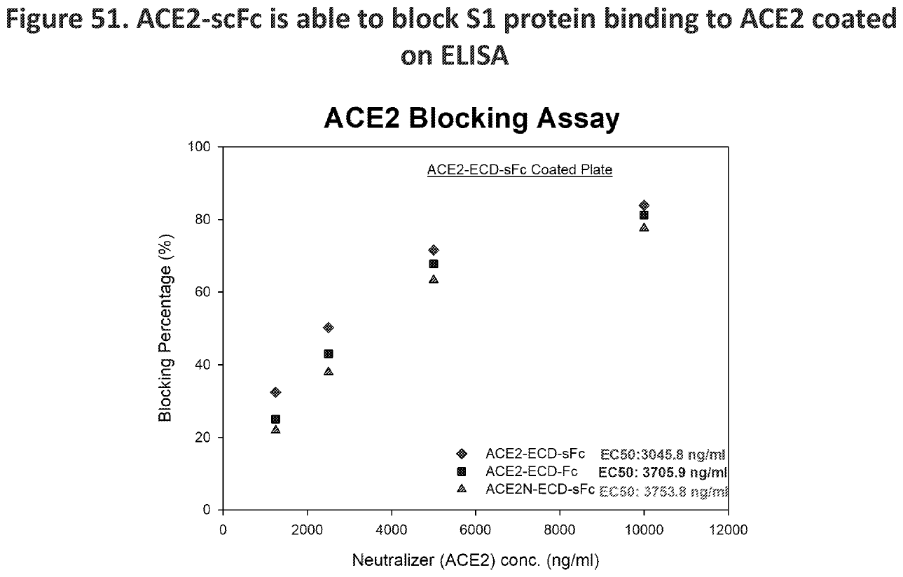

A “Single Chain Fc Platform” was employed to produce a potent, song-acting neutralizing protein product ACE2-ECD-sFc (SEQ ID NO: 237). Due to the receptor binding inhibition nature, the ACE2-ECD-sFc protein is anticipated to meet little drug resistance if the coronavirus mutates. As shown in FIG. 50, due to the bulky conformation of the bivalent Fc fusion nature, the ACE-ECD-Fc has a faster departure rate (about 10×) when binding to the S1 protein compared to the single chain (ACE ECD-sFc protein) indicating that the Fc protein has a 10× lower binding affinity when compared to that of the single chain (sFc) fusion protein. As shown in FIG. 51, although all three types of ACE-ECD fusion proteins (ACE2 ECD-sFc, ACE2 ECD-Fc, and ACE2 ECD-sFc) all have significant capability to block S1 binding to ACE-2 coated on an ELISA plate. The ACE2-ECD-sFc has a higher % of blocking inhibition when compare to the other two types. This result indicates that the relative inhibition in viral induced Cytopathic Effect (CPE) on the Vero cells, when tested in two separate laboratories (KeXin Lab in Beijing and Sinica Lab in Taipei) as shown in Table 36, where an equivalent titer of 8,192 was achieved by 2.4 mg/mL of ACE2-ECD-sFc in an assay, would offer a highly effective treatment for patients encountering acute attack by SARS-CoV-2 infection based on the observation that a full protection can be obtained in a primate challenge study with serum titer neutralizing antibodies in the range of around 50. A phase I/II trial will be conducted in mild to severe COVID-19 patients to observe the safety and efficacy of such a long-acting protein drug.

TABLE 1
Amino Acid Sequences of Membrane Glycoprotein M from
SARS-CoV-2, SARS-CoV, and MERS-CoV

| SEQ | | |
|---|---|---|
| ID NO | Description | Sequence |
| 1 | SARS-CoV-2 | MADSNGTITVEELKKLLEQWNLVIGFLFLTWICLLQFAYANRNRFLYIIKL |
| | M Protein | IFLWLLWPVTLACFVLAAVYRINWITGGIAIAMACLVGLMWLSYFIASFRL |
| | (YP_009724393.1) | FARTRSMWSFNPETNILLNVPLHGTTLTRPLLESELVIGAVILRGHLRIAG |
| | | HHLGRCDIKDLPKEITVATSRTLSYYKLGASQRVAGDSGFAAYSRYRIGNY |
| | | KLNTDHSSSSDNIALLVQ |
| 2 | SARS-CoV | MADNGTITVEELKQLLEQWNLVIGFLFLAWIMLLQFAYSNRNRFLYIIKLV |
| | M Protein | FLWLLWPVTLACFVLAAVYRINWVTGGIAIAMACIVGLMWLSYFVASFRLF |
| | (NP_828855.1) | ARTRSMWSFNPETNILLNVPLRGTIVTRPLMBSELVIGAVIIRGHLRMAGH |
| | | SLGRCDIKDLPKEITVATSRTLSYYKLGASQRVGTDSGFAAYNRYRIGNYK |
| | | LNTDHAGSNDNIALLVQ |
| 3 | MERS-CoV | MSNMTQLTEAQIIAIIKDWNFAWSLIFLLITIVLQYGYPSRSMTVYVFKMF |
| | M Protein | VLWLLWPSSMALSIFSAIYPIDLASQIISGIVAAVSAMMWISYFVQSIRLF |
| | (AGV08396.1) | MRTGSWWSFNPETNCLLNVPFGGTTVVRPLVEDSTSVTAVVTNGHLKMAGM |
| | | HFGACDYDRLPNEVTVAKPNVLIALKMVKRQSYGTNSGVAIYHRYKAGNYR |
| | | SPPITADIELALLRA |
| 4 | SARS-CoV-2 M₁₋₂₃ | MADSNGTITVEELKKLLEQWNLV |
| | (Antigenic peptide) | |
| 5 | KKK-SARS-CoV-2 M₁₋₂₃ | KKKMADSNGTITVEELKKLLEQWNLV |
| | (Antigenic peptide) | |

TABLE 2
Amino Acid Sequences of Nucleocapsid Phosphoprotein
N from SARS-CoV-2, SARS-CoV, and MERS-CoV

| SEQ | | |
|---|---|---|
| ID NO | Description | Sequence |
| 6 | SARS-CoV-2 | MSDNGPQNQRNAPRITFGGPSDSTGSNQNGERSGARSKQRRPQGLPNNT |
| | N Protein | ASWFTALTQHGKEDLKFPRGQGVPINTNSSPDDQIGYYRRATRRIRGGD |
| | (YP_009724397.2) | GKMKDLSPRWYFYYLGTGPEAGLPYGANKDGIIWVATEGALNTPKDHIG |
| | | TRNPANNAAIVLQLPQGTTLPKGFYAEGSRGGSQASSRSSSRSRNSSRN |
| | | STPGSSRGTSPARMAGNGGDAALALLLLDRLNQLESKMSGKGQQQQGQT |
| | | VTKKSAAEASKKPRQKRTATKAYNVTQAFGRRGPEQTQGNFGDQELIRQ |
| | | GTDYKHWPQIAQFAPSASAFFGMSRIGMEVTPSGTWLTYTGAIKLDDKD |
| | | PNFKDQVILLNKHIDAYKTFPPTEPKKDKKKKADETQALPQRQKKQQTV |
| | | TLLPAADLDDFSKQLQQSMSSADSTQA |
| 7 | SARS-CoV | MSDNGPQSNQRSAPRITFGGPTDSTDNNQNGGRNGARPKQRRPQGLPNN |
| | N Protein | TASWFTALTQHGKEELRFPRGQGVPINTNSGPDDQIGYYRRATRRVRGG |
| | (NP_828858.1) | DGKMKELSPRWYFYYLGTGPEASLPYGANKEGIVWVATEGALNTPKDHI |
| | | GTRNPNNNAATVLQLPQGTTLPKGFYAEGSRGGSQASSRSSSRSRGNSR |
| | | NSTPGSSRGNSPARMASGGGETALALLLLDRLNQLESKVSGKGQQQQGQ |
| | | TVTKKSAAEASKKPRQKRTATKQYNVTQAFGRRGPEQTQGNFGDQDLIR |
| | | QGTDYKHWPQIAQFAPSASAFFGMSRIGMEVTPSGTWLTYHGAIKLDDK |
| | | DPQFKDNVILLNKHIDAYKTFPPTEPKKDKKKKTDEAQPLPQRQKKQPT |
| | | VTLLPAADMDDFSRQLQNSMSGASADSTQA |
| 8 | MERS-CoV | MASPAAPRAVSFADNNDITNTNLSRGRGRNPKPRAAPNNTVSWYTGLTQ |
| | N Protein | HGKVPLTFPPGQGVPLNANSTPAQNAGYWRRQDRKINTGNGIKQLAPRW |
| | (AGN70936) | YFYYTGTGPEAALPFRAVKDGIVWVHEDGATDAPSTFGTRNPNNDSAIV |
| | | TQFAPGTKLPKNFHIEGTGGNSQSSSRASSVSRNSSRSSSQGSRSGNST |
| | | RGTSPGPSGIGAVGGDLLYLDLLNRLQALESGKVKQSQPKVITKKDAAA |
| | | AKNKMRHKRTSTKSFNMVQAFGLRGPGDLQGNFGDLQLNKLGTEDPRWP |
| | | QIAELAPTASAFMGMSQFKLTHQNNDDHGNPVYFLRYSGAIKLDPKNPN |
| | | YNKWLELLEQNIDAYKTFPKKEKKQKAPKEESTDQMSEPPKEQRVQGSI |
| | | TQRTRTRPSVQPGPMIDVNTD |
| 9 | SARS-CoV-2 N₁₃₈₋₁₄₆ | ALNTPKDHI |
| | (CTL epitope) | |
| 10 | SARS-CoV-2 N₁₅₉₋₁₆₇ | LQLLQGTTL |
| | (CTL epitope) | |
| 11 | SARS-CoV-2 N₂₁₉₋₂₂₇ | LALLLLDRL |
| | (CTL epitope) | |
| 12 | SARS-CoV-2 N₂₂₂₋₂₃₀ | LLLDRLNQL |
| | (CTL epitope) | |
| 13 | SARS-CoV-2 N₃₀₅₋₃₁₉ | AQFAPSASAFFGMSR |
| | (Th epitope) | |
| 14 | SARS-CoV-2 N₃₁₆₋₃₂₄ | GMSRIGMEV |
| | (CTL epitope) | |
| 15 | SARS-CoV-2 N₃₂₂₋₃₃₁ | MEVTPSGTWL |
| | (CTL epitope) | |
| 16 | SARS-CoV-2 N₃₅₁₋₃₅₉ | ILLNKHIDA |
| | (CTL epitope) | |
| 17 | SARS-CoV-2 N₃₅₅₋₄₁₉ | KHIDAYKTFPPTEPKKDKKKKAKETQALPQRQKKQQTVTLLPAADLDDF |
| | (Antigenic peptide) | SKQLQQSMSSADSTQA |
| 18 | KKK-SARS-CoV-2 N₃₅₅₋₄₁₉ | KKKKHIDAYKTFPPTEPKKDKKKKADETQALPQRQKKQQTVTLLPAADL |
| | (Antigenic peptide) | DDFSKQLQQSMSSADSTQA |
| 19 | SARS-CoV-2 N₃₆₁₋₃₆₉ | FTFPPTEPK |
| | (CTL epitope) | |

TABLE 3
Amino Acid Sequences of Surface Glycoprotein Sfrom SARS-CoV-2, SARS, and MERS

| SEQ ID NO | Description | Sequence |
|---|---|---|
| 20 | SARS-CoV-2 | MFVFLVLLPLVSSQCVNLTTRTQLPPAYTNSFTRGVYYPDKVFRSSVLHST |
| | S Protein | QDLFLPFFSNVTWFHAIHVSGTNGTKRFDNPVLPFNDGVYFASTEKSNIIR |
| | (YP_009724390.1) | GWIFGTTLDSKTQSLLIVNNATNVVIKVCEFQFCNDPFLGVYYHKNNKSWM |
| | | ESEFRVYSSANNCTFEYVSQPFLMDLEGKQGNFKNLREFVFKNIDGYFKIY |
| | | SKHTPINLVRDLPQGFSALEPLVDLPIGINITRFQTLLALHRSYLTPGDSS |
| | | SGWTAGAAAYYVGYLQPRTFLLKYNENGTITDAVDCALDPLSETKCTLKSF |
| | | TVEKGIYQTSNFRVQPTESIVRFPNITNLCPFGEVFNATRFASVYAWNRKR |
| | | ISNCVADYSVLYNSASFSTFKCYGVSPTKLNDLCFTNVYADSFVIRGDEVR |
| | | QIAPGQTGKIADYNYKLPDDFTGCVIAWNSNNLDSKVGGNYNYLYRLFRKS |
| | | NLKPFERDISTEIYQAGSTPCNGVEGFNCYFPLQSYGFQPTNGVGYQPYRV |
| | | VVLSFELLHAPATVCGPKKSTNLVKNKCVNFNFNGLTGTGVLTESNKKFLP |
| | | FQQFGRDIADTTDAVRDPQTLEILDITPCSFGGVSVITPGTNTSNQVAVLY |
| | | QDVNCTEVPVAIHADQLTPTWRVYSTGSNVFQTRAGCLIGAEHVNNSYECD |
| | | IPIGAGICASYQTQTNSPRRARSVASQSIIAYTMSLGAENSVAYSNNSIAI |
| | | PTNFTISVTTEILPVSMTKTSVDCTMYICGDSTECSNLLLQYGSFCTQLNR |
| | | ALTGIAVEQDKNTQEVFAQVKQIYKTPPIKDFGGFNFSQILPDPSKPSKRS |
| | | FIEDLLFNKVTLADAGFIKQYGDCLGDIAARDLICAQKFNGLTVLPPLLTD |
| | | EMIAQYTSALLAGTITSGWTFGAGAALQIPFAMQMAYRFNGIGVTQNVLYE |
| | | NQKLIANQFNSAIGKIQDSLSSTASALGKLQDVVNQNAQALNTLVKQLSSN |
| | | FGAISSVLNDILSRLDKVEAEVQIDRLITGRLQSLQTYVTQQLIRAAEIRA |
| | | SANLAATKMSECVLGQSKRVDFCGKGYHLMSFPQSAPHGVVFLHVTYVPAQ |
| | | EKNFTTAPAICHDGKAHFPREGVFVSNGTHWFVTQRNFYEPQIITTDNTFV |
| | | SGNCDVVIGIVNNTVYDPLQPELDSFKEELDKYFKNHTSPDVDLGDISGIN |
| | | ASVVNIQKEIDRLNEVAKNLNESLIDLQELGKYEQYIKWPWYIWLGFIAGL |
| | | IAIVMVTIMLCCMTSCCSCLKGCCSCGSCCKFDEDDSEPVLKGVLKHYT |
| 21 | SARS-CoV | MFIFLLFLTLTSGSDLDRCTTFDDVQAPNYTQHTSSMRGVYYPDEIFRSDT |
| | S Protein | LYLTQDLFLPFYSNVTGFHTINHTFGNPVIPFKDGIYFAATEKSNVVRGWV |
| | (NP_828851.1) | FGSTMNNKSQSVIIINNSTNVVIRACNFELCDNPFFAVSKPMGTQTHTMIF |
| | | DNAFNCTFEYISDAFSLDVSEKSGNFKHLREFVFKNKDGFLYVYKGYQPID |
| | | VVRDLPSGFNTLKPIFKLPLGINITNFRAILTAFSPAQDIWGTSAAAYFVG |
| | | YLKPTTFMLKYDENGTITDAVDCSQNPLAELKCSVKSFEIDKGIYQTSNFR |
| | | VVPSGDVVRFPNITNLCPFGEVFNATKFPSVYAWERKKISNCVADYSVLYN |
| | | STFFSTFKCYGVSATKLNDLCFSNVYADSFVVKGDDVRQIAPGQTGVIADY |
| | | NYKLPDDFMGCVLAWNTRNIDATSTGNYNYKYRYLRHGKLRPFERDISNVP |
| | | FSPDGKPCTPPALNCYWPLNDYGFYTTTGIGYQPYRVVVLSFELLNAPATV |
| | | CGPKLSTDLIKNQCVNFNFNGLTGTGVLTPSSKRFQPFQQFGRDVSDFTDS |
| | | VRDPKTSEILDISPCAFGGVSVITPGTNASSEVAVLYQDVNCTDVSTAIHA |
| | | DQLTPAWRIYSTGNNVFQTQAGCLIGAEHVDTSYECDIPIGAGICASYHTV |
| | | SLLRSTSQKSIVAYTMSLGADSSIAYSNNTIAIPTNFSISITTEVMPVSMA |
| | | KTSVDCNMYICGDSTECANLLLQYGSFCTQLNRALSGIAAEQDRNTREVFA |
| | | QVKQMYKTPTLKYFGGFNFSQILPDPLKPTKRSFIEDLLFNKVTLADAGFM |
| | | KQYGECLGDINARDLICAQKFNGLTVLPPLLTDDMIAAYTAALVSGTATAG |
| | | WTFGAGAALQIPFAMQMAYRFNGIGVTQNVLYENQKQIANQFNKAISQIQE |
| | | SLTTTSTALGKLQDVVNQNAQLANTLVKQLSSNFGAISSVLNDILSRLDKV |
| | | EAEVQIDRLITGRLQSLQTYVTQQLIRAAEIRASANLAATKMSECVLGQSK |
| | | RVDFCGKGYHLMSFPQAAPHGVVFLHVTYVPSQERNFTTAPAICHEGKAYF |
| | | PREGVFVFNGTSWFITQRNFFSPQIITTDNTFVSGNCDVVIGIINNTVYDP |
| | | LQPELDSFKEELDKYFKNHTSPDVDLGDISGINASVVNIQKEIDRLNEVAK |
| | | NLNESLIDLQELGKYQEYIKWPWYVWLGFIAGLIAIVMVTILLCCMTSCCS |
| | | CLKGACSCGSCCKFDEDDSEPVLKGVKLHYT |
| 22 | MERS-CoV | MIHSVFLLMFLLTPTESYVDVGPDSVKSACIEVDIQQTFFDKTWPRPIDVS |
| | S Protein | KADGIIYPQGRTYSNITITYQGLFPYQGDHGDMYVYSAGHATGTTPQKLFV |
| | (AHE78097.1) | ANYSQDVKQFANGFVVRIGAAANSTGTVIISPSTSATIRKIYPAFMLGSSV |
| | | GNFSDGKMGRFFNHTLVLLPDGCGTLLRAFYCILEPRSGNHCPAGNSYTSF |
| | | ATYHTPATDCSDGNYNRNASLNSFKEYFNLRNCTFMYTYNITEDEILEWFG |
| | | ITQTAQGVHLFSSRYVDLYGGNMFQFATLPVYDTIKYYSIIPHSIRSIQSD |
| | | RKAWAAFYVYKLQPLTFLLDFSVDGYIRRAIDCGFNDLSQLHCSYESFDVE |
| | | SGVYSVSSFEAKPSGSVVEQAEGVECDFSPLLSGTPPQVYNFKRLVFTNCN |
| | | YNLTKLLSLFSVNDFTCSQISPAAIASNCYSSLILDYFSYPLSMKSDLSVS |
| | | SAGPISQFNYKQSFSNPTCLILATVPHNLTTITKPLKYSYINKCSRLLSDD |
| | | RTEVPQLVNANQYSPCVSIVPSTVWEDGDYYRKQLSPLEGGGWLVASGSTV |
| | | AMTEQLQMGFGITVQYGTDTNSVCPKLEFANDTKIASQLGNCVEYSLYGVS |
| | | GRGVFQNCTAVGVRQQRFVYDAYQNLVGYYSDDGNYYCLRACVSVPVSVIY |
| | | DKETKTHATLFGSVACEHISSTMSQYSRSTRSMLKRRDSTYGPLQTPVGCV |
| | | LGLVNSSLFVEDCKLPLGQSLCALPDTPSTLTPRSVRSVPGEMRLASIAFN |
| | | HPIQVDQLNSSYFKLSIPTNFSFGVTQEYIQTTIQKVTVDCKQYVCNGFQK |
| | | CEQLLREYGQFCSKINQALHGANLRQDDSVRNLFASVKSSQSSPIIPGFGG |
| | | DFNLTLLEPVSISTGSRSARSAIEDLLFDKVTIADPGYMQGYDDCMQQGPA |
| | | SARDLICAQYVAGYKVLPPLMDVNMEAAYTSSLLGSIAGVGWTAGLSSFAA |
| | | IPFAQSIFYRLNGVGITQQVLSENQKLIANKFNQALGAMQTGFTTTNEAFH |
| | | KVQDAVNNNAQALSKLASELSNTFGAISASIGDIIQRLDVLEQDAQIDRLI |
| | | NGRLTTLNAFVAQQLVRSESAALSAQLAKDKVNECVKAQSKRSGFCGQGTH |
| | | IVSFVVNAPNGLYFMHVGYYPSNHIEVVSAYGLCDAANPTNCIAPVNGYFI |
| | | KTNNTRIVDEWSYTGSSFYAPEPITSLNTKYVAPQVTYQNISTNLPPPLLG |
| | | NSTGIDFQDELDEFFKNVSTSIPNFGSLTQINTTLLDLTYEMLSLQQVVKA |
| | | LNESYIDLKELGNYTYYNKWPWYIWLGFIAGLVALALCVFFILCCTGCGTN |
| | | CMGKLKCNRCCDRYEEYDLEPHKVHVH |
| 23 | SARS-CoV-2 S₄₄₃₋₄₄₂ | SKVGGN |
| | (B cell epitope) | |
| | (Loop C) | |
| 24 | SARS-CoV-2 S₄₄₃₋₄₄₈_mod | [symbol] KVGG [symbol] |
| | (B cell epitope) | |
| | (Loop C-cyclized) | |
| 25 | SARS-CoV S₄₃₀₋₄₃₅ | ATSTGN |
| | (Loop C) | |
| 26 | SARS-CoV-2 S₄₈₀₋₅₀₉ | CNGVEGFNCYFPLQSYGFQPTNGVGYQPYR |
| | (B cell epitope) | |
| 27 | KKK-SARS-CoV-2 S₄₈₀₋₅₀₉ | KKKCNGVEGFNCYFPLQSYGFQPTNGVGYQPYR |
| | (B cell epitope) | |
| 28 | SARS-CoV S₄₆₇₋₄₉₅ | CTPPAL-NCYWPLNDYGFYTTTGIGYQPYR |
| 29 | SARS-CoV-2 S₄₈₀₋₄₉₀ | CNGVEGFNCYF |
| | (B cell epitope) | |
| 30 | SARS-CoV-2 S₄₈₀₋₄₈₈ | CNGVEGFNC |
| | (B cell epitope) | |
| | (Loop A) | |
| 31 | SARS-CoV-2 S₄₉₆₋₅₀₈ | GFQPTNGVGYQPY |
| | (B cell epitope) | |
| | (Loop B) | |
| 32 | SARS-Cov-2 5₄₉₆₋₅₀₈_mod | [symbol] FQPTNGVGYQP [symbol] |
| | (B cell epitope) | |
| | (Loop B-cyclized) | |
| 33 | SARS-CoV-2 S₄₉₆₋₅₀₅ | GFQPTNGVGY |
| | (B cell epitope) | |
| 34 | SARS-CoV-2 S₄₉₆₋₅₀₅_mod | [symbol] FQPTNGVG [symbol] |
| | (B cell epitope) | |
| 35 | SARS-CoV-2 S₅₀₄₋₅₁₅ | GYQPYRVVVLSF |
| | (CTL epitope) | |
| 36 | SARS-CoV-2 S₅₃₉₋₅₄₆ | VNFNFNGL |
| | (CTLepitope) | |
| 37 | SARS-CoV-2 S₇₈₅₋₈₃₉ | VKQIYKTPPIKDFGGFNFSQILPDPSKPSKRSFIEDLLFNKVTLADAGFIK |
| | (Antigenic peptide) | QYGD |
| 38 | KKK-SARS-CoV-2 S₇₈₅₋₈₃₉ | KKKVKQIYKTPPIKDFGGFNFSQILPDPSKPSKRSFIEDLLFNKVTLADAG |
| | (Antigenic peptide) | FIKQYGD |
| 39 | SARS-CoV-2 S₈₉₁₋₉₀₆ | GAALQIPFAMQMAYRF |
| | (Th epitope) | |
| 40 | SARS-CoV-2 S₉₀₂₋₉₁₇ | MAYRFNGIGVTQNVLY |
| | (Th epitope) | |
| 41 | SARS-CoV-2 S₉₅₇₋₉₇₃ | QALNTLVKQLSSNFGAI |
| | (Th epitope) | |
| 42 | SARS-CoV-2 S₉₇₆₋₉₈₄ | VLNDILSRL |
| | (CTL epitope) | |
| 43 | SARS-CoV-2 S₉₉₆₋₁₀₀₄ | LITGRLQSL |
| | (CTL epitope) | |
| 44 | SARS-CoV-2 S₁₀₁₁₋₁₀₂₈ | QLIRAAEIRASANLAATK |
| | (Th epitope) | |
| 45 | SARS-CoV-2 S₁₀₆₀₋₁₀₆₈ | VVFLHVTYV |
| | (CTL epitope) | |
| 46 | SARS-CoV-2 S₁₁₈₅₋₁₁₉₃ | RLNEVAKNL |
| | (CTL epitope) | |
| 47 | SARS-CoV-2 S₁₁₉₂₋₁₂₀₀ | NLNESLIDL |
| | (CTL epitope) | |
| 48 | SARS-CoV-2 S₁₂₂₀₋₁₂₂₈ | FIAGLIAIV |
| | (CTL epitope) | |

*Peptides are cyclized by cysteine disulfide bonds with the cysteines underlined. The
Cysteines/Serines that substitute the amino acids of the SARS-CoV-2 fragments are in italics.

```
TABLE 4
SARS-CoV-2 CTL epitopes for use in vaccine
design (validated by PBMC binding and
      stimulation assay through previous
    SARS-CoV studies)
```

| SEQ | Pro- | | Position | | | MHC |
|---|---|---|---|---|---|---|
| ID | tein | Epitope | SARS- | SARS- | MHC | allele |
| NO | source | seq. | CoV | CoV-2 | allele | class |
| 45 | S | VVFLHVTYV | 1042- | 1060- | HLA- | I |
| | | | 1050 | 1068 | A*02:01 | |
| 42 | S | VLNDILSRL | 958- | 976- | HLA- | I |
| | | (Sp8) | 966 | 984 | A*02:01 | |
| 46 | S | RLNEVAKNL | 1167- | 1185- | HLA- | I |
| | | (SSp-1) | 1175 | 1193 | A*02:01 | |
| 36 | S | VNFNFNGL | 525- | 539- | Protect- | I |
| | | (S525) | 532 | 546 | ing mice | |
| | | | | | from | |
| | | | | | lethal | |
| | | | | | SARS | |
| | | | | | challenge | |
| 48 | S | FIAGLIAIV | 1202- | 1220- | HLA- | I |
| | | | 1210 | 1228 | A*02:01 | |
| 43 | S | LITGRLQSL | 978- | 996- | HLA-A2 | I |
| | | | 986 | 1004 | | |
| 47 | S | NLNESLIDL | 1174- | 1192- | HLA- | I |
| | | | 1182 | 1200 | A*02:01 | |
| 35 | S | GYQPYRVVVL | 490- | 504- | HLA- | I |
| | | SF | 501 | 515 | A*02:01 | |
| 12 | N | LLLDRLNQL | 223- | 222- | HLA- | I |
| | | | 231 | 230 | A*02:01 | |
| 11 | N | LALLLLDRL | 220- | 219- | HLA- | I |
| | | | 228 | 227 | A*02:01 | |
| 10 | N | LQLPQGTTL | 159- | 159- | HLA- | I |
| | | | 167 | 167 | A*02:01 | |
| 14 | N | GMSRIGMEV | 317- | 316- | HLA- | I |
| | | | 325 | 324 | A*02:01 | |
| 19 | N | KTFPPTEPK | 362- | 361- | HLA- | I |
| | | | 370 | 369 | A*1101 | |
| 9 | N | ALNTPKDHI | 139- | 138- | HLA- | I |
| | | | 147 | 146 | A*02:01 | |
| 16 | N | ILLNKHIDA | 352- | 351- | HLA- | I |
| | | | 360 | 359 | A*02:01 | |
| 15 | N | MEVTPSGTWL | 323- | 322- | HLA- | I |
| | | | 332 | 331 | B*40:01 | |

Adapted from Ahmed, S. F., et al, 2020

TABLE 5
SARS-CoV-2 Th epitopes for use in vaccine
design (validated by PBMC binding and
stimulation assay through previous
SARS-CoV studies)

| SEQ | Pro- | | Position | | | MHC |
|---|---|---|---|---|---|---|
| ID | tein | Epitope | SARS- | SARS | MHC | class |
| NO | source | seq. | CoV | -CoV-2 | allele | allele |
| 39 | S | GAALQIPFA | 873- | 891- | HLA- | II |
| | | MQMAYRF | 888 | 906 | DRA*01:01 | |
| | | | | | HLA- | |
| | | | | | DRB1*07:01 | |
| 40 | S | MAYRFNGIG | 884- | 902- | HLA- | II |
| | | VTQNVLY | 899 | 917 | DRB1*04:01 I | |
| 44 | S | QLIRAAEIR | 993- | 1011- | HLA- | II |
| | | ASANLAATK | 1010 | 1028 | DRB1*04:01 I | |
| 41 | S | QALNTLVKQ | 939- | 957- | HLA- | II |
| | | LSSNFGAI | 955 | 973 | DRB1*04:01 I | |
| 13 | N | AQFAPSASA | 306- | 305- | | II |
| | | FFGMSR | 320 | 319 | | |

Adapted from Ahmed, S. F., et al, 2020

TABLE 6
Amino Acid Sequences of Pathogen Protein
Derived Th Epitopes Including Idealized
Artificial Th Epitopes for Employment in
the Design of SARS-CoV-2 Peptide
Immunogen Constructs

| | | SEQ |
|---|---|---|
| | | ID |
| Description | Sequence | NO |
| MvF Th | DLSDLKGLLLHKLDGL | 49 |
| (SSAL1 Th1) | EI EIR III RIE I | 50 |
| | V V VVV V V | 51 |
| | F F FFF F F | 52 |
| | XXSXXXGXXXHXXXGX | 53 |
| MvF1 Th (UBITh ® 5) | LSEIKGVIVHRLEGV | 54 |
| MvF2 Th | ISEIKGVIVHKIEGI | 55 |
| MVF3 Th | ISISEIKGVIVHKIEGILF | 56 |
| | T RT TR T | 57 |
| | ISIXEIXXVIVXXIEXILF | 58 |
| KKKMvF3 Th | KKKISISEIKGVIVHKIEGILF | 59 |
| | T RT TR T | 60 |
| | KKKISIXEIXXVIVXXIEXILF | 61 |
| MvF4 Th (UBITh ® 3) | ISISEIKGVIVHKIETILF | 62 |
| | T RT TR | 63 |
| | ISIXEIXXVIVXXIETILF | 64 |
| MvF5 Th | ISITEIKGVIVHRIETILF | 65 |
| (UBITh ® 1) | | |
| KKKMVF5 Th | KKKISITEIKGVIVHRIETILF | 66 |
| (UBITh ® 1a) | | |
| HBsAg1 Th | KKKLFLLTKLLTLPQSLD | 67 |
| (SSAL2 Th2) | RRRIKII RII I L IR | 68 |
| | VRVV VV V I V | 69 |
| | F FF FF F V F | 70 |
| | F | 71 |
| | XXXXXXXTXXXTXPXSXX | 72 |
| HBsAg2 Th | KKKIITITRIITTPQSLD | 73 |
| | FFLL L ITTI | 74 |
| | KKKXXXXTRIXTIXXXXD | 75 |
| HBsAg3 Th | KKKIITITRIITIITTID | 76 |
| (UBITh ® 2) | | |
| HBsAg Th | FFLLTRILTIPQSLD | 77 |
| (UBITh ® 4) | | |
| KKK-HBsAg Th | KKKFFLLTRILTIPQSLD | 78 |
| HBsAg Th | FFLLTRILTIPQSL | 79 |
| Bordetella | GAYARCPNGTRALTVAELRGNAEL | 80 |
| pertussis Th | | |
| (UBITh ® 7) | | |
| Cholera | ALNIWDRFDVFCTLGATTGYLKGNS | 81 |
| Toxin Th | | |
| Clostridium | QYIKANSKFIGITEL | 82 |
| tetani TT1 Th | | |
| Clostridium | KKQYIKANSKFIGITEL | 83 |
| tetani1 Th | | |
| (UBITh ® 6) | | |
| Clostridium | FNNFTVSFWLRVPKVSASHLE | 84 |
| tetani TT2 Th | | |
| Clostridium | KFIIKRYTPNNEIDSF | 85 |
| tetani TT3 Th | | |
| Clostridium | VSIDKFRIFCKALNPK | 86 |
| tetani TT4 Th | | |
| Clostridium | WVRDIIDDFTNESSQKT | 87 |
| tetani2 Th | | |
| Diphtheria Th | DSETADMLEKTVAALSILPGHGC | 88 |
| EBV BHRF1 Th | AGLTLSLLVICSYLFISRG | 89 |
| EBV EBNA-1 Th | PGPLRESIVCYFMVFLQTHI | 90 |
| EBV CP Th | VPGLYSPCRAFFNKEELL | 91 |
| EBV GP340 Th | TGHGARTSTEPTTDY | 92 |
| EBV BPLF1 Th | KELKRQYEKKLRQ | 93 |
| EBV EBNA-2 | TVFYNIPPMPL | 94 |
| HCMVIE1 Th | DKREMWMACIKELH | 95 |
| Influenza | FVFTLTVPSER | 96 |
| MP1_1 Th | | |
| Influenza | SGPLKAEIAQRLEDV | 97 |
| MP1_2 Th | | |
| Influenza | DRLRRDQKS | 98 |
| NSP1 Th | | |
| Plasmodium | DHEKKHAKMEKASSVFNVVNS | 99 |
| falciparum Th | | |
| Schistosoma | KWFKTNAPNGVDEKHRH | 100 |
| mansoni Th | | |

TABLE 7
Examples of Optional Heterologous Spacers,
CpG Oligonucleotides, and RT-PCR
Primers/Probes

| | | SEQ |
|---|---|---|
| Descrip- | | ID |
| tion | Sequence/Composition | NO |
| Naturally- | Naturally-occurring amino | N/A |
| Occurring | acids include: | |
| Amino | alanine, arginine, asparagine, | |
| Acids | aspartic acid, cysteine, glut- | |
| | amic acid, glutamine, glycine, | |
| | histidine, isoleucine, leucine, | |
| | lysine, methionine, phenyla- | |
| | lanine, proline, serine, | |
| | threonine, tryptophan, | |
| | tyrosine and valine | |
| Non- | Non-naturally occurring amino | N/A |
| Naturally- | acids include, but are not | |
| Occurring | limited to: ϵ-N Lysine, β- | |
| Amino | alanine, ornithine, nor- | |
| Acids | leucine, norvaline, hydroxy- | |
| | proline, thyroxine, γ-amino | |
| | butyric acid, homoserine, | |
| | citrulline, aminobenzoic acid, | |
| | 6-aminocaproic acid (Aca; 6- | |
| | Aminohexanoic acid), hydroxy- | |
| | proline, mercaptopropionic | |
| | acid (MPA), 3-nitro-tyrosine, | |
| | pyroglutamic acid, and the like | |
| Chemicals | —NHCH(X)CH₂SCH₂CO—, | N/A |
| | —NHCH(X)CH₂SCH₂CO(ϵN)Lys-, | |
| | —NHCH(X)CH₂S-succinimidyl | |
| | (ϵN)Lys-, | |
| | —NHCH(X)CH₂S-(succinimidyl)- | |
| Gly-Gly | -GG- | N/A |
| Epsilon-N | ϵ-K | N/A |
| Lysine | | |
| Epsilon-N | ϵ-K-KKK | 101 |
| Lysine-KKK | | |
| KKK- | KKK-ϵ-K | 102 |
| Epsilon- | | |
| N Lysine | | |
| Hinge | Pro-Pro-Xaa-Pro-Xaa-Pro | 103 |
| Sequence | | |
| CpG1 | 5′ TCg TCg TTT TgT CgT TTT gTC | 104 |
| | gTT TTg TCg TT 3′ | |
| | (fully phosphorothioated) | |
| CpG2 | Phosphate TCg TCg TTT TgT CgT | 105 |
| | TTT gTC gTT 3′ | |
| | (fully phosphorothioated) | |
| CpG3 | 5′ TCg TCg TTT TgT CgT TTT gTC | 106 |
| | gTT 3′ | |
| | (fully phosphorothioated) | |
| E-Sarbeco- | 5′-ACAGGTACGTTAATAGTTAATAGCGT- | 368 |
| F1 Forward | 3′ | |
| Primer | | |
| E-Sarbeco- | 5′-ATATTGCAGCAGTACGCACACA-3′ | 369 |
| R2 Reverse | | |
| Primer | | |
| E-Sarbeco- | 5′-FAM- | 370 |
| P1 Probe | ACACTAGCCATCCTTACTGCGCTTCG- | |
| | BBQ-3′ | |

TABLE 8
Amino Acid Sequences of SARS-CoV-2 Peptide Immunogen Constructs

| | SEQ | |
|---|---|---|
| | ID | |
| Peptide Description | NO: | Sequence |
| SARS-CoV-2 S₄₈₀₋₄₀₀-KKK-ϵK-UBITh1 | 107 | CNGVEGFNCYF-KKK-ϵK-UBTh1 |
| UBITh1-ϵK-KKK-SARS-CoV-2 S₄₈₀₋₄₉₀ | 108 | UBTh1-ϵK-KKK-CNGVEGFNCYF |
| UBITh1-ϵK-KKK-SARS-CoV-2 S₄₉₆₋₅₀₈_mod | 109 | UBTh1-ϵK-KKK-CFQPTNGVGYQPC |
| SARS-CoV-2 S₄₉₆₋₅₀₈_mod-KKK-ϵK-UBITh1 | 110 | CFQPTNGVGYQPC-KKK-ϵK-UBTh1 |
| UBITh1-ϵK-KKK-SARS-CoV-2 S₄₉₆₋₅₀₅_mod | 111 | UBTh1-ϵK-KKK-CFQPTNGVGC |
| SARS-CoV-2 S₄₉₆₋₅₀₅_mod-KKK-ϵK-UBITh1 | 112 | CFQPTNGVGC-KKK-ϵK-UBTh1 |
| SARS-CoV-2 S₄₈₀₋₅₀₉-KKK-ϵK-UBITh1 | 113 | CNGVEGFNCYFPLQSYGFQPTNGVGYQPYR-KKK-ϵK-UBITh1 |
| UBITh1-ϵK-KKK-SARS-CoV-2 S₄₈₀₋₅₀₉_mod | 114 | UBTh1-ϵK-KKK-CNGVEGFNCYFPLQSYGFQPTNGVGYQPYR |
| UBITh1-ϵK-KKK-SARS-CoV-2 S₄₉₇₋₅₀₈_mod | 335 | UBITh1-ϵK-KKK-CQPTNGVGYQPCR |
| UBITh1-ϵK-KKK-SARS-CoV-2 S₄₈₀₋₅₀₈_mod | 336 | UBITh1-ϵK-KKK-SNGVEGFNSYFPLQSYGCQPTNGVGYQPCR |
| UBITh1-ϵK-KKK-SARS-CoV-2 S₃₆₁₋₃₉₁_mod | 337 | UBTh1-ϵK-KKK-CVADYSVLYNSASFSTFKSYGVSPTKLNDLC |
| SARS-CoV-2 S₃₆₁₋₃₉₁_mod-KKK-ϵK-UBITh1 | 338 | CVADYSVLYNSASFSTFKSYGVSPTKLNDLC-KKK-ϵK-UBTh1 |
| UBITh1-ϵK-KKK-SARS-CoV-2 S₃₆₃₋₃₈₈_mod | 339 | UBTh1-ϵK-KKK-CDYSVLYNSASFSTFKSYGVSPTKLC |
| SARS-CoV-2 S₃₆₃₋₃₈₈_mod-KKK-ϵK-UBITh1 | 340 | CDYSVLYNSASFSTFKSYGVSPTKLC-KKK-ϵK-UBTh1 |
| UBITh1-ϵK-KKK-SARS-CoV-2 S₄₄₃₋₅₀₇ | 341 | UBTh1-ϵK-KKK-SKVGGNYNYLYRLFRKSNLKPFERDISTEIY |
| | | QAGSTPCNGVEGFNCYFPLQSYGFQPTNGVGYQP |
| SARS-CoV-2 S₄₄₃₋₅₀₇-KKK-ϵK-UBITh1 | 342 | SKVGGNYNYLYRLFRKSNLKPFERDISTEIYQAGSTPCNGVEGF |
| | | NCYFPLQSYGFQPTNGVGYQP-KKK-ϵK-UBTh1 |
| UBITh1-ϵK-KKK-SARS-CoV-2 S₄₄₃₋₅₀₇_mod | 343 | UBTh1-ϵK-KKK-CKVGGNYNYLYRLFRKSNLRPFERDISTEIY |
| | | QAGSTPSNGVEGFNSYFPLQSYGFQFTNGVGYQC |
| SARS-CoV-2 S₄₄₃₋₅₀₇_mod-KKK-ϵK-UBITh1 | 344 | CKVGGNYNYLYRLFRKSNLKPFERDISTEIYQAGSTPSNGVEGF |
| | | NSYFPLQSYGFQPTNGVGYQC-KKK-ϵK-UBTh1 |
| UBITh1-ϵK-KKK-SARS-CoV-2 S₄₄₃₋₄₄₈_mod | 115 | UBTh1-ϵK-KKK-CKVGGC |
| SARS-CoV-2 S₄₄₃₋₄₄₈_mod-KKK-ϵK-UBITh1 | 116 | CKVGGC-KKK-ϵK-UBTh1 |
| Clostridium tetani1 Th-ϵK-KKK-SARS- | 117 | KKQYTKANSKFIGITEL-ϵK-KKK-CFQPTNGVGC |
| CoV-2 S₄₉₆₋₅₀₅_mod | | |
| MvF1 Th-ϵK-KKK-SARS-CoV-2 | 118 | LSEIKGVIVHRLEGV-ϵK-KKK-CFQPTNGVGC |
| S₄₉₆₋₅₀₅_mod | | |
| Bordetella pertussis Th-ϵK-KKK- | 119 | GAYARCPNGTRALTVAELRGNAEL-ϵK-KKK-CFQPTNGVGC |
| SARS-CoV-2 S₄₉₆₋₅₀₅_mod | | |
| Clostridium tetani2 Th-ϵK-KKK-SARS- | 120 | WVRDIIDDFTNESSQKT-ϵK-KKK-CFQPTNGVGC |
| CoV-2 S₄₉₆₋₅₀₅_mod | | |
| Diphtheria Th-ϵK-KKK-SARS-CoV-2 | 121 | DSETADNLEKTVAALSLLPGHGC-ϵK-KKK-CFQPTNGVGC |
| S₄₉₆₋₅₀₅_mod | | |
| Plasmodium falciparum Th-ϵK-KKK- | 122 | DHEKKHAKMEKASSVFNVVNS-ϵK-KKK-CFQPTNGVGC |
| SARS-CoV-2 S₄₉₆₋₅₀₅_mod | | |
| Schistosoma mansoni Th-ϵK-KKK- | 123 | KWFKTNAPNGVDEKHRH-ϵK-KKK-CFQPTNGVGC |
| SARS-CoV-2 S₄₉₆₋₅₀₅_mod | | |
| Cholera Toxin Th-ϵK-KKK-SARS-CoV-2 | 124 | ALNIWDRFDVFCTLGATTGYLKGNS-ϵK-KKK-CFQPTNGVGC |
| S₄₉₆₋₅₀₅_mod | | |
| MvF2 Th-ϵK-KKK-SARS-CoV-2 | 125 | ISEIKGVIVHKIEGI-ϵK-KKK-CFQPTNGVGC |
| S₄₉₆₋₅₀₅_mod | | |
| KKKMvF3 Th-ϵK-KKK-SARS-CoV-2 | 126 | KKKISISEIKGVIVHKIEGILF-ϵK-KKK-CFQPTNGVGC |
| S₄₉₆₋₅₀₅_mod | | T RT TR T |
| HBsAg1 Th-ϵK-KKK-SARS-CoV-2 | 127 | KKKLFLLTKLLTLPQSLD-ϵK-KKK-CFQPTNGVGC |
| S₄₉₆₋₅₀₅_mod | | RRRIKII RII I L IR |
| | | VRVV VV V I V |
| | | F FF FF F V F |
| | | F |
| MvF4 Th (UBITh ® 3)-ϵK-KKK-SARS- | 128 | ISISEIKGVIVHKIETILF-ϵK-KKK-CFQPTNGVGC |
| CoV-2 S₄₉₆₋₅₀₅_mod | | T RT TR |
| HBsAg2 Th-ϵK-KKK-SARS-CoV-2 | 129 | KKKIITITRIITIPQSLD-ϵK-KKK-CFQPTNGVGC |
| S₄₉₆₋₅₀₅_mod | | FFLL L ITTI |
| HBsAg3 Th (UBITh ® 2)-ϵK-KKK-SARS- | 130 | KKKIITITRIITIITTID-ϵK-KKK-CFQPTNGVGC |
| CoV-2 S₄₉₆₋₅₀₅_mod | | |
| Influenza MP1_1 Th-ϵK-KKK-SARS- | 131 | FVFTLTVPSER-ϵK-KKK-CFQPTNGVGC |
| CoV-2 S₄₉₆₋₅₀₅_mod | | |
| Influenza MP1_2 Th-ϵK-KKK-SARS- | 132 | SGPLKAEIAQRLEDV-ϵK-KKK-CFQPTNGVGC |
| CoV-2 S₄₉₆₋₅₀₅_mod | | |
| Influenza NSP1 Th-ϵK-KKK-SARS- | 133 | DRLRRDQKS-ϵK-KKK-CFQPTNGVGC |
| CoV-2 S₄₉₆₋₅₀₅_mod | | |
| EBV BHRF1 Th-ϵK-KKK-SARS-CoV-2 | 134 | AGLTLSLLVICSYLFISRG-ϵK-KKK-CFQPTNGVGC |
| S₄₉₆₋₅₀₅_mod | | |
| Clostridium tetani TT1 Th-ϵK-KKK- | 135 | QYIKANSKFIGITEL-ϵK-KKK-CFQPTNGVGC |
| SARS-CoV-2 S₄₉₆₋₅₀₅_mod | | |
| EBV EBNA-1 Th-ϵK-KKK-SARS-CoV-2 | 136 | PGPLRESIVCYFMVFLQTHI-ϵK-KKK-CFQPTNGVGC |
| S₄₉₆₋₅₀₅_mod | | |
| Clostridium tetani TT2 Th-ϵK-KKK- | 137 | FNNFTVSFWLRVPKVSASHLE-ϵK-KKK-CFQPTNGVGC |
| SARS-CoV-2 S₄₉₆₋₅₀₅_mod | | |
| Clostridium tetani TT3 Th-ϵK-KKK- | 138 | KFIIKRYTPNNEIDSF-ϵK-KKK-CFQPTNGVGC |
| SARS-CoV-2 S₄₉₆₋₅₀₅_mod | | |
| Clostridium tetani TT4 Th-ϵK-KKK- | 139 | VSIDKFRIFCKALNPK-ϵK-KKK-CFQPTNGVGC |
| SARS-CoV-2 S₄₉₆₋₅₀₅_mod | | |
| EBV CP Th-ϵK-KKK-SARS-CoV-2 | 140 | VPGLYSPCRAFFNKEELL-ϵK-KKK-CFQPTNGVGC |
| S₄₉₆₋₅₀₅_mod | | |
| HCMV IE1 Th-ϵK-KKK-SARS-CoV-2 | 141 | DKREMWMACIKELH-ϵK-KKK-CFQPTNGVGC |
| S₄₉₆₋₅₀₅_mod | | |
| EBV GP340 Th-ϵK-KKK-SARS-CoV-2 | 142 | TGHGARTSTEPTTDY-ϵK-KKK-CFQPTNGVGC |
| S₄₉₆₋₅₀₅_mod | | |
| EBV BPLF1 Th-ϵK-KKK-SARS-CoV-2 | 143 | RELKRQYEKKLRQ-ϵK-KKK-CFQPTNGVGC |
| S₄₉₆₋₅₀₅_mod | | |
| EBV EBNA-2 Th-ϵK-KKK-SARS-CoV-2 | 144 | TVFYNIPPMPL-ϵK-KKK-CFQPTNGVGC |
| S₄₉₆₋₅₀₅_mod | | |
| KKK-SARS-CoV-2 S CTL 1060-1068 | 145 | KKKVVFLHVTYV |
| KKK-SARS-CoV-2 S CTL 976-984 | 146 | KKKVLNDILSRL |
| KKK-SARS-CoV-2 S CTL 1185-1193 | 147 | KKKRLNEVAKNL |
| KKK-SARS-CoV-2 S CTL 539-546 | 148 | KKKVNFNFNGL |
| KKK-SARS-CoV-2 S CTL 1220-1228 | 149 | KKKFIAGLIAIV |
| KKK-SARS-CoV-2 S CTL 996-1004 | 150 | KKKLITGRLQSL |
| KKK-SARS-CoV-2 S CTL 1192-1200 | 151 | KKKNLNESLIDL |
| KKK-SARS-CoV-2 S CTL 504-515 | 152 | KKKGYQPYRVVVLSF |
| KKK-SARS-CoV-2 N CTL 222-230 | 153 | KKKLLLDRLNQL |
| KKK-SARS-CoV-2 N CTL 219-227 | 154 | KKKLALLLLDRL |
| KKK-SARS-CoV-2 N CTL 159-167 | 155 | KKKLQLPQGTTL |
| KKK-SARS-CoV-2 N CTL 316-324 | 156 | KKKGMSRIGMEV |
| KKK-SARS-CoV-2 N CTL 361-369 | 157 | KKKKTFPPTEPK |
| KKK-SARS-CoV-2 N CTL 138-146 | 158 | KKKALNTPKDHI |
| KKK-SARS-CoV-2 N CTL 351-359 | 159 | KKKILLNKHIDA |
| KKK-SARS-CoV-2 N CTL 322-331 | 160 | KKKMEVTPSGTWL |
| KKK-SARS-CoV-2 S Th 891-906 | 161 | KKKGAALQIPFAMQMAYRF |
| KKK-SARS-CoV-2 S Th 902-917 | 162 | KKKMAYRFNGIGVTQNVLY |
| KKK-SARS-CoV-2 S Th 1011-1028 | 163 | KKKQLIRAAEIRASANLAATK |
| KKK-SARS-CoV-2 S Th 957-973 | 164 | KKKQALNTLVKQLSSNFGAI |
| KKK-SARS-CoV-2 N Th 305-319 | 165 | KKKAQFAPSASAFFGMSR |
| KKK-SARS-CoV-2 S957-984 | 345 | KKK-QALNTLVKQLSSNFGAISSVLNDILSRL |
| (Th/CTL epitope) | | |
| KKK-SARS-CoV-2 S891-917 | 346 | KKK-GAALQIPFAMQMAYRFNGIGVTQNVLY |
| (Th epitope) | | |
| KKK-SARS-CoV-2 N305-331 | 347 | KKK-AQFAPSASAFFGMSRIGMEVTPSGTWL |
| (Th/CTL epitope) | | |
| KKK-SARS-CoV-2 S996-1028 | 348 | KKK-LITGRLQSLQTYVTQQLIRAAEIRASANLAATK |
| (Th/CTL epitope) | | |
| KKK-SARS-CoV-2 S1185-1200 | 349 | KKK-RLNEVAKNLNESLIDL |
| (CTL epitope) | | |
| KKK-SARS-CoV-2 N351-369 | 350 | KKK-ILLNKHIDAYKTFPPTEPK |
| (CTL epitope) | | |
| KKK-SARS-CoV-2 N219-230 | 351 | KKK-LALLLLDRLNQL |
| (CTL epitope) | | |
| KKK-SARS-CoV-2 M89-111 | 361 | KKK-GLMWLSYFIASFRLFARTRSMWS |
| (Th/CTL epitope) | | |

* Peptides are cyclized by cysteine disulfide bonds with the cysteines underlined. The Cysteines/Serines that substitute the amino acids of the SARS-CoV-2 fragments are in italics.

TABLE 9
Wild-Type and Mutated Hinge Regions from
IgG1, IgG2, IgG3, and IgG4

| SEQ | | |
|---|---|---|
| ID | | |
| NO | Sequence | Description |
| 166 | EPKSCDKTHTCPPCP | Wild-type IgG1 |
| 167 | EPKSXDKTHTXPPXP | Mutated IgG1 |
| 168 | EPKSXDKTHTXPP | |
| 169 | EPKSXDKTHT | |
| 170 | DKTHTXPPXP | |
| 171 | ERKCCVECPPCP | Wild-type IgG2 |
| 172 | ERKXXVEXPPXP | Mutated IgG2 |
| 173 | ERKXXVEXPP | |
| 174 | VEXPPXP | |
| 175 | ELKTPLGDTTHTCPRCP | Wild-type IgG3 |
| 176 | ELKTPLGDTTHTXPRXP | Mutated IgG3 |
| 177 | ELKTPLGDTTHTXPR | |
| 178 | ELKTPLGDTTHT | |
| 179 | EPKSCDTPPPCPRCP | Wild-type IgG3 |
| 180 | EPKSXDTPPPXPRXP | Mutated IgG3 |
| 181 | EPKSXDTPPPXPR | |
| 182 | EPKSXDTPPP | |
| 183 | DTPPPXPRXP | |
| 184 | ESKYGPPCPSCP | Wild-type IgG4 |
| 185 | EXKYGPPCPXCP | Mutated IgG4 |
| 186 | EXKYGPPCP | |
| 187 | KYGPPCPXCP | |

X: Ser, Gly, Thr, Ala, Val, Leu, Ile, Met, and/or deletion

TABLE 10
Examples of Amino Acid Sequences of Mutated
Hinge Regions Derived from IgG1

| SEQ | |
|---|---|
| ID | |
| NO | Sequence¹ |
| 188 | EPKSSDKTHTSPPSP |
| 189 | EPKSSDKTHTSPP |
| 190 | EPKSSDKTHTSPPP |
| 191 | EPKSSDKTHT |
| 192 | DKTHTSPPSP |
| 193 | DKTHTSPP |
| 194 | EPKSDKTHTPPP |
| 195 | EPKSDKTHTSPPSP |
| 196 | EPKSGDKTHTGPPGP |
| 197 | EPKSGDKTHTGPP |
| 198 | EPKSGDKTHTGPPP |
| 199 | EPKSGDKTHT |
| 200 | DKTHTGPPGP |
| 201 | DKTHTGPP |
| 202 | EPKSDRTHTGPPGP |
| 203 | EPKSGDKTHTSPPSP |
| 204 | EPKSGDKTHTGPPSP |
| 205 | EPKSSDKTHTGPPGP |
| 206 | EPKSSDKTHTGPPSP |
| 207 | EPKSSDKTHTGPP |
| 208 | EPKSGDKTHTSPP |
| 209 | EPKSTDKTHTTPPTP |
| 210 | EPKSTDKTHTTPP |
| 211 | EPKSTDKTHTTPPP |
| 212 | EPKSTDKTHT |
| 213 | DKTHTTPPTP |
| 214 | DKTHTTPP |
| 215 | EPKSDKTHTTPPTP |
| 216 | EPKSSDKTHTTPPTP |
| 217 | EPKSSDKTHTSPPTP |
| 218 | EPKSADKTHTLPPMP |
| 219 | EPKSVDKTHTLPPIP |
| 220 | EPKSLDKTHTAPPAP |
| 221 | EPKSVDKTHTAPP |
| 222 | EPKSMDKTHTVPP |
| 223 | EPKSIDKTHTLPP |
| 224 | DKTHTAPPLP |
| 225 | DKTHTVPPLP |

¹Underlined residues represent sites of mutation in relation to the sequence of wild-type IgG

TABLE 11
Amino Acid Sequences of sFC and Fc Fusion Proteins

| SEQ | | |
|---|---|---|
| ID | | |
| NO | Sequence | Type |
| 226 | NITNLCPFGEVFNATRFASVYAWNRKRISNCVADYSVLYNSASFSTFKCYGVSPTKL | S protein |
| | NDLCFTNVYADSFVIRGDEVRQIAPGQTGKIADYNYKLPDDFTGCVIAWNSNNLDSK | RBD |
| | VGGNYNYLYRLFRKSNLKPFERDISTEIYQAGSTPCNGVEGFNCYFPLQSYGFQPTN | (SARS-CoV-2) |
| | GVGYQPYRVVVLSFELLHAPATVCGPKKS | |
| 227 | NITNLCPFGEVFNATRFASVYAWNRKRISNCVADYSVLYNSASFSTFKCYGVSPTKL | S protein |
| | NDLAFTNVYADSFVIRGDEVRQIAPGQTGKIADYNYKLPDDFTGCVIAWNSNNLDSK | RBDa |
| | VGGNYNYLYRLFRKSNLKPFERDISTEIYQAGSTPCNGVEGFNCYFPLQSYGFQPTN | (SARS-CoV-2) |
| | GVGYQPYRVVVLSFELLHAPATVAGPKKS | |
| 228 | MSSSSWLLLSLVAVTAAQSTIEEQAKTFLDKFNHEAEDLFYQSSLASWNYNTNITEE | ACE2 |
| | NVQNMNNAGDKWSAFLKEQSTLAQMYPLQEIQNLTVKLQLQALQQNGSSVLSEDKSK | (Homo |
| | RLNTILNTMSTIYSTGKVCNPDNPQECLLLEPGLNEIMANSLDYNERLWAWESWRSE | sapiens) |
| | VGKQLRPLYEEYVVLKNEMARANHYEDYGDYWRGDYEVNGVDGYDYSRGQLIEDVEH | |
| | TFEEIKPLYEHLHAYVRAKLMNAYPSYISPIGCLPAHLLGDMWGRFWTNLYSLTVPF | |
| | GQKPNIDVTDAMVDQAWDAQRIFKEAEKFFVSVGLPNMTQGFWENSMLTDPGNVQKA | |
| | VCHPTAWDLGKGDFRILMCTKVTMDDFLTAHHEMGHIQYDMAYAAQPFLLRNGANEG | |
| | FHEAVGEXMSLSAATPKHLKSIGLLSPDFQEDNETEINFLLKQALTIVGTLPFTYML | |
| | EKWRWMVFKGEIPKDQWMKKWWEMKREIVGVVEPVPHDETYCDPASLFHVSNDYSFI | |
| | RYYTRTLYQFQFQEALCQAAKHEGPLHKCDISNSTEAGQKLFNMLRLGKSEPWTLAL | |
| | ENVVGAKNMNVRPLLNYFEPLFTWLKDQNKNSFVGWSTDWSPYADQSIKVRISLKSA | |
| | LGDKAYEWNDNEMYLFRSSVAYAMRQYFLKVKNQMILFGEEDVRVANLKPRISFNFF | |
| | VTAPKNVSDIIPRTEVEKAIRMSRSRINDAFRLNDNSLEFLGIQPTLGPPNQPPVSI | |
| | WLIVFGVVMGVIVVGIVILIFTGIRDRKKKNKARSGENPYASIDISKGENNPGFQNT | |
| | DDVQTSF | |
| 229 | MSSSSWLLLSLVAVTAAQSTIEEQAKTFLDKFNHEAEDLFYQSSLASWNYNTNITEE | ACE2 extra- |
| | NVQNMNNAGDKWSAFLKEQSTLAQMYPLQEIQNLTVKLQLQALQQNGSSVLSEDKSK | cellular |
| | RLNTILNTMSTIYSTGKVCNPDNPQECLLLEPGLNEIMANSLDYNERLWAWESWRSE | domain (ECD) |
| | VGKQLRPLYEEYVVLKNEMARANHYEDYGDYWRGDYEVNGVDGYDYSRGQLIEDVEH | (Homo |
| | TFEEIKPLYEHLHAYVRAKLMNAYPSYISPIGCLPAHLLGDMWGRFWTNLYSLTVPF | sapiens) |
| | GQKPNIDVTDAMVDQAWDAQRIFKEAEKFFVSVGLPNMTQGFWENSMLTDPGNVQKA | |
| | VCHPTAWDLGKGDFRILMCTKVTMDDFLTAHHEMGHIQYDMAYAAQPFLLRNGANEG | |
| | FHEAVGEIMSLSAATPKHLKSIGLLSPDFQEDNETEINFLLKQALTIVGTLPFTYML | |
| | EKWRWMVFKGEIPKDQWMKKWWEMKREIVGVVEPVPHDETYCDPASLFHVSNDYSFI | |
| | RYYTRTLYQFQFQEALCQAAKHEGPLHKCDISNSTEAGQKLFNMLRLGKSEPWTLAL | |
| | ENVVGAKNMNVRPLLNYFEPLFTWLKDQNKNSFVGWSTDWSPYADQSIKVRISLKSA | |
| | LGDKAYEWNDNEMYLFRSSVAYAMRQYFLKVKNQMILFGEEDVRVANLKPRISFNFF | |
| | VTAPKNVSDIIPRTEVEKAIRMSRSRINDAFRLNDNSLEFLGIQPTLGPPNQPPVS | |
| 230 | MSSSSWLLLSLVAVTAAQSTIEEQAKTFLDKFNHEAEDLFYQSSLASWNYNTNITEE | ACE2N |
| | NVQNMNNAGDKWSAFLKEQSTLAQMYPLQEIQNLTVKLQLQALQQNGSSVLSEDKSK | extra-cellular |
| | RLNTILNTMSTIYSTGKVCNPDNPQECLLLEPGLNEIMANSLDYNERLWAWESWRSE | domain (ECD) |
| | VGKQLRPLYEEYVVLKNEMARANHYEDYGDYWRGDYEVNGVDGYDYSRGQLIEDVEH | (Homo |
| | TFEEIKPLYEHLHAYVRAKLMNAYPSYISPIGCLPAHLLGDMWGRFWTNLYSLTVPF | sapiens) |
| | GQKPNIDVTDAMVDQAWDAQRIFKEAEKFFVSVGLPNMTQGFWENSMLTDPGNVQKA | |
| | VCHPTAWDLGKGDFRILMCTKVTMDDFLTAHNEMGNIQYDMAYAAQPFLLRNGANEG | |
| | FHEAVGEIMSLSAATPKHLKSIGLLSPDFQEDNETEINFLLKQALTIVGTLPFTYML | |
| | EKWRWMVFKGEIPKDQWMKKWWEMKREIVGVVEPVPHDETYCDPASLFHVSNDYSFI | |
| | RYYTRTLYQFQFQEALCQAAKHEGPLHKCDISNSTEAGQKLFNMLRLGKSEPWTLAL | |
| | ENVVGAKNMNVRPLLNYFEPLFTWLKDQNKNSFVGWSTDWSPYADQSIKVRISLKSA | |
| | LGDKAYEWNDNEMYLFRSSVAYAMRQYFLKVKNQMILFGEEDVRVANLKPRISFNFF | |
| | VTAPKNVSDIIPRTEVEKAIRMSRSRINDAFRLNDNSLEFLGIQPTLGPPNQPPVS | |
| 231 | APELLGGPSVFLFPPKPKDTLMISRTPEVTCVVVDVSHEDPEVKFNWYVDGVEVHNA | Fc peptide |
| | KTKPREEQYSSTYRVVSVLTVLHQDWLNGKEYKCKVSNKALPAPIEKTISKAKGQPR | (Wild-Type) |
| | EPQVYTLPPSRDELTKNQVSLTCLVKGFYPSDXAVEWESNGQPENNYKTTPPVLDSD | |
| | GSFFLYSKLTVDKSRWQQGNVFSCSVMHEALHNHYTQKSLSLSPG | |
| 232 | APELLGGPSVFLFPPKPKDTLMISRTPEVTCVVVDVSHEDPEVKFNWYVDGVEVHNA | Fc peptide |
| | KTKPREEQYHSTYRWSVLTVLHQDWLNGKEYKCKVSNKALPAPIEKTISKAKGQPR | Mut Glycos. |
| | EPQVYTLPPSRDELTKNQVSLTCLVKGFYPSDIAVEWESNGQPENNYKTTPPVLDSD | (N->H) |
| | GSFFLYSKLTVDKSRWQQGNVFSCSVMHEALHNHYTQKSLSLSPG | |
| 233 | APELLGGPSVFLFPPKPKDTLMISRTPEVTCVVVDVSHEDPEVKFNWYVDGVEVHNA | Fc peptide |
| | KTKPREEQYASTYRVVSVLTVLHQDWLNGKEYKCKVSNKALPAPIEKTISKAKGQPR | Mut. Glycos. |
| | EPQVYTLPPSRDELTKNQVSLTCLVKGFYPSDIAVEWESNGQPENNYKTTPPVLDSD | (N->A) |
| | GSFFLYSKLTVDKSRWQQGNVFSCSVMHEALHNHYTQKSLSLSPG | |
| 234 | APELLGGPSVFLFPPKPKDTLMISRTPEVTCVVVDVSHEDPEVKFNWYVDGVEVHNA | Fc peptide |
| | KTKPREEQYXSTYRVVSVLTVLHQDWLNGKEYKCKVSNKALPAPIEKTISKAKGQPR | Mut. Glycos. |
| | EPQVYTLPPSRDELTKNQVSLTCLVKGFYPSDIAVEWESNGQPENNYKTTPPVLDSD | (N->X) |
| | GSFFLYSKLTVDKSRWQQGNVFSCSVMHEALHNHYTQKSLSLSPG | X = N, H, A |
| 235 | NITNLCPFGEVFNATRFASVYAWNRKRISNCVADYSVLYNSASFSTFKCYGVSPTKL | S-RBD-sFc |
| | NDLCFTNVYADSFVIRGDEVRQIAPGQTGKIADYNYKLPDDFTGCVIAWNSNNLDSK | Fusion |
| | VGGNYNYLYRLFRKSNLKPFERDISTEIYQAGSTPCNGVEGFNCYFPLQSYGFQPTN | protein |
| | GVGYQPYRVVVLSFELLHAPATVCGPKKSEPRSSDKTATSPPSPAPELLGGPSVFLF | |
| | PPKPKDTLMISRTPEVTCVVVDVSHEDPEVKFNWYVDGVEVHNAKTKPREEQYASTY | |
| | RVVSVLTVLHQDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSFFLYSKLTVDK | |
| | SRWQQGNVFSCSVMHEALHNHYTQKSLSLSPG | |
| 236 | NITNLCPFGEVFNATRFASVYAWNRKRISNCVADYSVLYNSASFSTFKCYGVSPTKL | S-RBDa-sFc |
| | NDLAFTNVYADSFVIRGDEVRQIAPGQTGKIADYNYKLPDDFTGCVIAWNSNNLDSK | Fusion |
| | VGGNYNYLYRLFRKSNLKPFERDISTEIYQAGSTPCNGVEGFNCYFPLQSYGFQPTN | protein |
| | GVGYQPYRVVVLSFELLHAPATVAGPKKSSWRSSDRTWTSPPSPAPELLGGPSVFLF | |
| | PPKPKDTLMISRTPEVTCVVVDVSHEDPEVKFNWYVDGVEVHNAKTKPREEQYHSTY | |
| | RVVSVLTVLHQDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSFFLYSKLTVDK | |
| | SRWQQGNVFSCSVMHEALHNHYTQKSLSLSPG | |
| 237 | MSSSSWLLLSLVAVTAAQSTIEEQAKTFLDKFNHEAEDLFYQSSLASWNYNTNITEE | ACE2-ECD- |
| | NVQNMNNAGDKWSAFLKEQSTLAQMYPLQEIQNLTVKLQLQALQQNGSSVLSEDKSK | sFc Fusion |
| | RLNTILNTMSTIYSTGKVCNPDNPQECLLLEPGLNEIMANSLDYNERLWAWESWRSE | protein |
| | VGKQLRPLYEEYVVLKNEMARANHYEDYGDYWRGDYEVNGVDGYDYSRGQLIEDVEH | |
| | TFEEIKPLYEHLHAYVRAKLMNAYPSYISPIGCLPAHLLGDMWGRFWTNLYSLTVPF | |
| | GQKPNIDVTDAMVDQAWDAQRIFKEAEKFFVSVGLPNMTQGFWENSMLTDPGNVQKA | |
| | VCHPTAWDLGKGDFRILMCTKVTMDDFLTAHHEMGHIQYDMAYAAQPFLLRNGANEG | |
| | FHEAVGEIMSLSAATPKHLKSIGLLSPDFQEDNETEINFLLKQALTIVGTLPFTYML | |
| | EKWRWMVFKGEIPKDQWMKKWWEMKREIVGVVEPVPHDETYCDPASLFHVSNDYSFI | |
| | RYYTRTLYQFQFQEALCQAAKHEGPLHKCDISNSTEAGQKLFNMLRLGKSEPWTLAL | |
| | ENVVGAKNMNVRPLLNYFEPLFTWLKDQNKNSFVGWSTDWSPYADQSIKVRISLKSA | |
| | LGDKAYEWNDNEMYLFRSSVAYAMRQYFLKVKNQMILFGEEDVRVANLKPRISFNFF | |
| | VTAPKNVSDIIPRTEVEKAIRMSRSRINDAFRLNDNSLEFLGIQPTLGPPNQPPVSE | |
| | PKSSDKTHTSPPSPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSLS | |
| | PG | |
| 238 | MSSSSWLLLSLVAVTAAQSTTEEQAKTFLDKFNHEAEDLFYQSSLASWNYNTNITEE | ACE2N-ECD- |
| | NVQNMNNAGDKWSAFLKEQSTLAQMYPLQEIQNLTVKLQLQALQQNGSSVLSEDKSK | sFc Fusion |
| | RLNTILNTMSTIYSTGKVCNPDNPQECLLLEPGLNEIMANSLDYNERLWAWESWRSE | protein |
| | VGKQLRPLYEEYVVLKNEMARANHYEDYGDYWRGDYEVNGVDGYDYSRGQLIEDVEH | |
| | TFEEIKPLYEHLHAYVRAKLMNAYPSYISPIGCLPAHLLGDMWGRFWTNLYSLTVPF | |
| | GQKPNIDVTDAMVDQAWDAQRIFKEAEKFFVSVGLPNMTQGFWENSMLTDPGNVQKA | |
| | VCHPTAWDLGKGDFRILMCTKVTMDDFLTAHNEMGNIQYDMAYAAQPFLLRNGANEG | |
| | FHEAVGEIMSLSAATPKHLKSIGLLSPDFQEDNETEINFLLKQALTIVGTLPFTYML | |
| | EKWRWMVFKGEIPKDQWMKKWWEMKREIVGVVEPVPHDETYCDPASLFHVSNDYSFI | |
| | RYYTRTLYQFQFQEALCQAAKHEGPLHKCDISNSTEAGQKLFNMLRLGKSEPWTLAL | |
| | ENVVGAKNMNVRPLLNYFEPLFTWLKDQNKNSFVGWSTDWSPYADQSIKVRISLKSA | |
| | LGDKAYEWNDNEMYLFRSSVAYAMRQYFLKVKNQMILFGEEDVRVANLKPRISFNFF | |
| | VTAPKNVSDIIPRTEVEKAIRMSRSRINDAFRLNDNSLEFLGIQPTLGPPNQPPVSE | |
| | PKSSDKTHTSPPSPAPELLGGPSVFLFPPKPKDTLMISRTPEVTCVVVDVSHEDPEV | |
| | KFNWYVDGVEVHNAKTKPREEQYHSTYRVVSVLTVLHQDWLNGKEYKCKVSNKALPA | |
| | PIEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSLS | |
| | PG | |
| 355 | NITNLCPFGEVFNATRFASVYAWNRKRISNCVADYSVLYNSASFSTFKCYGVSPTKL | S-RBD-Fc |
| | NDLCFTNVYADSFVIRGDEVRQIAPGQTGKIADYNYKLPDDFTGCVIAWNSNNLDSK | Fusion |
| | VGGNYNYLYRLFRKSNLKPFERDISTEIYQAGSTPCNGVEGFNCYFPLQSYGFQPTN | protein |
| | GVGYQPYRVVVLSFELLHAPATVCGPKKSEPKSCDKTHTCPPCPAPELLGGPSVFLF | |
| | PPKPKDTLMISRTPEVTCVVVDVSHEDPEVKFNWYVDGVEVHNAKTKPREEQYHSTY | |
| | RVVSVLTVLHQDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSFFLYSKLTVDK | |
| | SRWQQGNVFSCSVMHEALHNHYTQKSLSLSPG | |
| 356 | MSSSSWLLLSLVAVTAAQSTIEEQAKTFLDKFNHEAEDLFYQSSLASWNYNTNITEE | ACE2-ECD-Fc |
| | NVQNMNNAGDKWSAFLKEQSTLAQMYPLQEIQNLTVKLQLQALQQNGSSVLSEDKSK | Fusion |
| | RLNTILNTMSTIYSTGKVCNPDNPQECLLLEPGLNEIMANSLDYNERLWAWESWRSE | protein |
| | VGKQLRPLYEEYVVLKNEMARANHYEDYGDYWRGDYEVNGVDGYDYSRGQLIEDVEH | |
| | TFEEIKPLYEHLHAYVRAKLMNAYPSYISPIGCLPAHLLGDMWGRFWTNLYSLTVPF | |
| | GQKPNIDVTDAMVDQAWDAQRIFKEAEKFFVSVGLPNMTQGFWENSMLTDPGNVQKA | |
| | VCHPTAWDLGKGDFRILMCTKVTMDDFLTAHHEMGHIQYDMAYAAQPFLLRNGANEG | |
| | FHEAVGEIMSLSAATPKHLKSIGLLSPDFQEDNETEINFLLKQALTIVGTLPFTYML | |
| | EKWRWMVFKGEIPKDQWMKKWWEMKREIVGVVEPVPHDETYCDPASLFHVSNDYSFI | |
| | RYYTRTLYQFQFQEALCQAAKHEGPLHKCDISNSTEAGQKLFNMLRLGKSEPWTLAL | |
| | ENVVGAKNMNVRPLLNYFEPLFTWLKDQNKNSFVGWSTDWSPYADQSIKVRISLKSA | |
| | LGDKAYEWNDNEMYLFRSSVAYAMRQYFLKVKNQMILFGEEDVRVANLKPRISFNFF | |
| | VTAPKNVSDIIPRTEVEKAIRMSRSRINDAFRLNDNSLEFLGIQPTLGPPNQPPVSE | |
| | PKSCDKTHTCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHYTQKSLSLS | |
| | PG | |
| 359 | HHHHHHENLYFQGNITNLCPFGEVFNATRFASVYAWNRKRISNCVADYSVLYNSASF | S-RBD-His |
| | STFKCYGVSPTKLNDLCFTNVYADSFVIRGDEVRQIAPGQTGKIADYNYKLPDDFTG | Fusion |
| | CVIAWNSNNLDSKVGGNYNYLYRLFRKSNLKPFERDISTEIYQAGSTPCNGVEGFNC | protein |
| | YFPLQSYGFQPTNGVGYQPYRVVVLSFELLHAPATVCGPKKS | |

TABLE 12
Nucleic Acid Sequences of sFc and Fc Fusion Proteins

| SEQ | | |
|---|---|---|
| ID | | |
| NO | Sequences | Type |
| 239 | ATGTTCCTGCTGACCACCAAGAGGACCATGTTCGTGTTCCTGGTGCTGCTGCCCCTGG | S protein |
| | TGTCCTCCCAGTGCGTGAACCTGACCACCAGGACCCAGCTGCCCCCCGCCTACACCAA | (SARS-CoV-2) |
| | CTCCTTCACCAGGGGCGTGTACTACCCCGACAAGGTGTTCAGGTCCTCCGTGCTGCAC | |
| | TCCACCCAGGACCTGTTCCTGCCCTTCTTCTCCAACGTGACCTGGTTCCACGCCATCC | |
| | ACGTGTCCGGCACCAACGGCACCAAGAGGTTCGACAACCCCGTGCTGCCCTTCAACGA | |
| | CGGCGTGTACTTCGCCTCCACCGAGAAGTCCAACATCATCAGGGGCTGGATCTTCGGC | |
| | ACCACCCTGGACTCCAAGACCCAGTCCCTGCTGATCGTGAACAACGCCACCAACGTGG | |
| | TGATCAAGGTGTGCGAGTTCCAGTTCTGCAACGACCCCTTCCTGGGCGTGTACTACCA | |
| | CAAGAACAACAAGTCCTGGATGGAGTCCGAGTTCAGGGTGTACTCCTCCGCCAACAAC | |
| | TGCACCTTCGAGTACGTGTCCCAGCCCTTCCTGATGGACCTGGAGGGCAAGCAGGGCA | |
| | ACTTCAAGAACCTGAGGGAGTTCGTGTTCAAGAACATCGACGGCTACTTCAAGATCTA | |
| | CTCCAAGCACACCCCCATCAACCTGGTGAGGGACCTGCCCCAGGGCTTCTCCGCCCTG | |
| | GAGCCCCTGGTGGACCTGCCCATCGGCATCAACATCACCAGGTTCCAGACCCTGCTGG | |
| | CCCTGCACAGGTCCTACCTGACCCCCGGCGACTCCTCCTCCGGCTGGACCGCCGGCGC | |
| | CGCCGCCTACTACGTGGGCTACCTGCAGCCCAGGACCTTCCTGCTGAAGTACAACGAG | |
| | AACGGCACCATCACCGACGCCGTGGACTGCGCCCTGGACCCCCTGTCCGAGACCAAGT | |
| | GCACCCTGAAGTCCTTCACCGTGGAGAAGGGCATCTACCAGACCTCCAACTTCAGGGT | |
| | GCAGCCCACCGAGTCCATCGTGAGGTTCCCCAACATCACCAACCTGTGCCCCTTCGGC | |
| | GAGGTGTTCAACGCCACCAGGTTCGCCTCCGTGTACGCCTGGAACAGGAAGAGGATCT | |
| | CCAACTGCGTGGCCGACTACTCCGTGCTGTACAACTCCGCCTCCTTCTCCACCTTCAA | |
| | GTGCTACGGCGTGTCCCCCACCAAGCTGAACGACCTGTGCTTCACCAACGTGTACGCC | |
| | GACTCCTTCGTGATCAGGGGCGACGAGGTGAGGCAGATCGCCCCCGGCCAGACCGGCA | |
| | AGATCGCCGACTACAACTACAAGCTGCCCGACGACTTCACCGGCTGCGTGATCGCCTG | |
| | GAACTCCAACAACCTGGACTCCAAGGTGGGCGGCAACTACAACTACCTGTACAGGCTG | |
| | TTCAGGAAGTCCAACCTGAAGCCCTTCGAGAGGGACATCTCCACCGAGATCTACCAGG | |
| | CCGGCTCCACCCCCTGCAACGGCGTGGAGGGCTTCAACTGCTACTTCCCCCTGCAGTC | |
| | CTACGGCTTCCAGCCCACCAACGGCGTGGGCTACCAGCCCTACAGGGTGGTGGTGCTG | |
| | TCCTTCGAGCTGCTGCACGCCCCCGCCACCGTGTGCGGCCCCAAGAAGTCCACCAACC | |
| | TGGTGAAGAACAAGTGCGTGAACTTCAACTTCAACGGCCTGACCGGCACCGGCGTGCT | |
| | GACCGAGTCCAACAAGAAGTTCCTGCCCTTCCAGCAGTTCGGCAGGGACATCGCCGAC | |
| | ACCACCGACGCCGTGAGGGACCCCCAGACCCTGGAGATCCTGGACATCACCCCCTGCT | |
| | CCTTCGGCGGCGTGTCCGTGATCACCCCCGGCACCAACACCTCCAACCAGGTGGCCGT | |
| | GCTGTACCAGGACGTGAACTGCACCGAGGTGCCCGTGGCCATCCACGCCGACCAGCTG | |
| | ACCCCCACCTGGAGGGTGTACTCCACCGGCTCCAACGTGTTCCAGACCAGGGCCGGCT | |
| | GCCTGATCGGCGCCGAGCACGTGAACAACTCCTACGAGTGCGACATCCCCATCGGCGC | |
| | CGGCATCTGCGCCTCCTACCAGACCCAGACCAACTCCCCCAGGAGGGCCAGGTCCGTG | |
| | GCCTCCCAGTCCATCATCGCCTACACCATGTCCCTGGGCGCCGAGAACTCCGTGGCCT | |
| | ACTCCAACAACTCCATCGCCATCCCCACCAACTTCACCATCTCCGTGACCACCGAGAT | |
| | CCTGCCCGTGTCCATGACCAAGACCTCCGTGGACTGCACCATGTACATCTGCGGCGAC | |
| | TCCACCGAGTGCTCCAACCTGCTGCTGCAGTACGGCTCCTTCTGCACCCAGCTGAACA | |
| | GGGCCCTGACCGGCATCGCCGTGGAGCAGGACAAGAACACCCAGGAGGTGTTCGCCCA | |
| | GGTGAAGCAGATCTACAAGACCCCCCCCATCAAGGACTTCGGCGGCTTCAACTTCTCC | |
| | CAGATCCTGCCCGACCCCTCCAAGCCCTCCAAGAGGTCCTTCATCGAGGACCTGCTGT | |
| | TCAACAAGGTGACCCTGGCCGACGCCGGCTTCATCAAGCAGTACGGCGACTGCCTGGG | |
| | CGACATCGCCGCCAGGGACCTGATCTGCGCCCAGAAGTTCAACGGCCTGACCGTGCTG | |
| | CCCCCCCTGCTGACCGACGAGATGATCGCCCAGTACACCTCCGCCCTGCTGGCCGGCA | |
| | CCATCACCTCCGGCTGGACCTTCGGCGCCGGCGCCGCCCTGCAGATCCCCTTCGCCAT | |
| | GCAGATGGCCTACAGGTTCAACGGCATCGGCGTGACCCAGAACGTGCTGTACGAGAAC | |
| | CAGAAGCTGATCGCCAACCAGTTCAACTCCGCCATCGGCAAGATCCAGGACTCCCTGT | |
| | CCTCCACCGCCTCCGCCCTGGGCAAGCTGCAGGACGTGGTGAACCAGAACGCCCAGGC | |
| | CCTGAACACCCTGGTGAAGCAGCTGTCCTCCAACTTCGGCGCCATCTCCTCCGTGCTG | |
| | AACGACATCCTGTCCAGGCTGGACAAGGTGGAGGCCGAGGTGCAGATCGACAGGCTGA | |
| | TCACCGGCAGGCTGCAGTCCCTGCAGACCTACGTGACCCAGCAGCTGATCAGGGCCGC | |
| | CGAGATCAGGGCCTCCGCCAACCTGGCCGCCACCAAGATGTCCGAGTGCGTGCTGGGC | |
| | CAGTCCAAGAGGGTGGACTTCTGCGGCAAGGGCTACCACCTGATGTCCTTCCCCCAGT | |
| | CCGCCCCCCACGGCGTGGTGTTCCTGCACGTGACCTACGTGCCCGCCCAGGAGAAGAA | |
| | CTTCACCACCGCCCCCGCCATCTGCCACGACGGCAAGGCCCACTTCCCCAGGGAGGGC | |
| | GTGTTCGTGTCCAACGGCACCCACTGGTTCGTGACCCAGAGGAACTTCTACGAGCCCC | |
| | AGATCATCACCACCGACAACACCTTCGTGTCCGGCAACTGCGACGTGGTGATCGGCAT | |
| | CGTGAACAACACCGTGTACGACCCCCTGCAGCCCGAGCTGGACTCCTTCAAGGAGGAG | |
| | CTGGACAAGTACTTCAAGAACCACACCTCCCCCGACGTGGACCTGGGCGACATCTCCG | |
| | GCATCAACGCCTCCGTGGTGAACATCCAGAAGGAGATCGACAGGCTGAACGAGGTGGC | |
| | CAAGAACCTGAACGAGTCCCTGATCGACCTGCAGGAGCTGGGCAAGTACGAGCAGTAC | |
| | ATCAAGTGGCCCTGGTACATCTGGCTGGGCTTCATCGCCGGCCTGATCGCCATCGTGA | |
| | TGGTGACCATCATGCTGTGCTGCATGACCTCCTGCTGCTCCTGCCTGAAGGGCTGCTG | |
| | CTCCTGCGGCTCCTGCTGCAAGTTCGACGAGGACGACTCCGAGCCCGTGCTGAAGGGC | |
| | GTGAAGCTGCACTACACC | |
| 240 | AACATCACCAACCTGTGCCCCTTCGGCGAGGTGTTCAACGCCACCAGGTTCGCCTCCG | S protein |
| | TGTACGCCTGGAACAGGAAGAGGATCTCCAACTGCGTGGCCGACTACTCCGTGCTGTA | RBD |
| | CAACTCCGCCTCCTTCTCCACCTTCAAGTGCTACGGCGTGTCCCCCACCAAGCTGAAC | (SARS-CoV-2) |
| | GACCTGTGCTTCACCAACGTGTACGCCGACTCCTTCGTGATCAGGGGCGACGAGGTGA | |
| | GGCAGATCGCCCCCGGCCAGACCGGCAAGATCGCCGACTACAACTACAAGCTGCCCGA | |
| | CGACTTCACCGGCTGCGTGATCGCCTGGAACTCCAACAACCTGGACTCCAAGGTGGGC | |
| | GGCAACTACAACTACCTGTACAGGCTGTTCAGGAAGTCCAACCTGAAGCCCTTCGAGA | |
| | GGGACATCTCCACCGAGATCTACCAGGCCGGCTCCACCCCCTGCAACGGCGTGGAGGG | |
| | CTTCAACTGCTACTTCCCCCTGCAGTCCTACGGCTTCCAGCCCACCAACGGCGTGGGC | |
| | TACCAGCCCTACAGGGTGGTGGTGCTGTCCTTCGAGCTGCTGCACGCCCCCGCCACCG | |
| | TGTGCGGCCCCAAGAAGTCC | |
| 241 | AACATCACCAACCTGTGCCCCTTCGGCGAGGTGTTCAACGCCACCAGGTTCGCCTCCG | S protein |
| | TGTACGCCTGGAACAGGAAGAGGATCTCCAACTGCGTGGCCGACTACTCCGTGCTGTA | RBDa |
| | CAACTCCGCCTCCTTCTCCACCTTCAAGTGCTACGGCGTGTCCCCCACCAAGCTGAAC | (SARS-CoV-2) |
| | GACCTGGCCTTCACCAACGTGTACGCCGACTCCTTCGTGATCAGGGGCGACGAGGTGA | |
| | GGCAGATCGCCCCCGGCCAGACAGACCAAGATCGCCGACTACAACTACAAGCTGCCGA | |
| | CGACTTCACCGGCTGCGTGATCGCCTGGAACTCCAACAACCTGGACTCCAAGGTGGGC | |
| | GGCAACTACAACTACCTGTACAGGCTGTTCAGGAAGTCCAACCTGAAGCCCTTCGAGA | |
| | GGGACATCTCCACCGAGATCTACCAGGCCGGCTCCACCCCCTGCAACGGCGTGGAGGG | |
| | CTTCAACTGCTACTTCCCCCTGCAGTCCTACGGCTTCCAGCCCACCAACGGCGTGGGC | |
| | TACCAGCCCTACAGGGTGGTGGTGCTGTCCTTCGAGCTGCTGCACGCCCCCGCCACCG | |
| | TGGCCGGCCCCAAGAAGTCC | |
| 242 | ATGTCCTCCTCCTCCTGGCTGCTGCTGTCCCTGGTGGCCGTGACCGCCGCCCAGTCCA | ACE2 |
| | CCATCGAGGAGCAGGCCAAGACCTTCCTGGACAAGTTCAACCACGAGGCCGAGGACCT | (Homo |
| | GTTCTACCAGTCCTCCCTGGCCTCCTGGAACTACAACACCAACATCACCGAGGAGAAC | sapiens) |
| | GTGCAGAACATGAACAACGCCGGCGACAAGTGGTCCGCCTTCCTGAAGGAGCAGTCCA | |
| | CCCTGGCCCAGATGTACCCCCTGCAGGAGATCCAGAACCTGACCGTGAAGCTGCAGCT | |
| | GCAGGCCCTGCAGCAGAACGGCTCCTCCGTGCTGTCCGAGGACAAGTCCAAGAGGCTG | |
| | AACACCATCCTGAACACCATGTCCACCATCTACTCCACCGGCAAGGTGTGCAACCCCG | |
| | ACAACCCCCAGGAGTGCCTGCTGCTGGAGCCCGGCCTGAACGAGATCATGGCCAACTC | |
| | CCTGGACTACAACGAGAGGCTGTGGGCCTGGGAGTCCTGGAGGTCCGAGGTGGGCAAG | |
| | CAGCTGAGGCCCCTGTACGAGGAGTACGTGGTGCTGAAGAACGAGATGGCCAGGGCCA | |
| | ACCACTACGAGGACTACGGCGACTACTGGAGGGGCGACTACGAGGTGAACGGCGTGGA | |
| | CGGCTACGACTACTCCAGGGGCCAGCTGATCGAGGACGTGGAGCACACCTTCGAGGAG | |
| | ATCAAGCCCCTGTACGAGCACCTGCACGCCTACGTGAGGGCCAAGCTGATGAACGCCT | |
| | ACCCCTCCTACATCTCCCCCATCGGCTGCCTGCCCGCCCACCTGCTGGGCGACATGTG | |
| | GGGCAGGTTCTGGACCAACCTGTACTCCCTGACCGTGCCCTTCGGCCAGAAGCCCAAC | |
| | ATCGACGTGACCGACGCCATGGTGGACCAGGCCTGGGACGCCCAGAGGATCTTCAAGG | |
| | AGGCCGAGAAGTTCTTCGTGTCCGTGGGCCTGCCCAACATGACCCAGGGCTTCTGGGA | |
| | GAACTCCATGCTGACCGACCCCGGCAACGTGCAGAAGGCCGTGTGCCACCCCACCGCC | |
| | TGGGACCTGGGCAAGGGCGACTTCAGGATCCTGATGTGCACCAAGGTGACCATGGACG | |
| | ACTTCCTGACCGCCCACGACGAGATGGGCCACATCCAGTACGACATGGCCTACGCCGC | |
| | CCAGCCCTTCCTGCTGAGGAACGGCGCCAACGAGGGCTTCCACGAGGCCGTGGGCGAG | |
| | ATCATGTCCCTGTCCGCCGCCACCCCCAAGCACCTGAAGTCCATCGGCCTGCTGTCCC | |
| | CCGACTTCCAGGAGGACAACGAGACCGAGATCAACTTCCTGCTGAAGCAGGCCCTGAC | |
| | CATCGTGGGCACCCTGCCCTTCACCTACATGCTGGAGAAGTGGAGGTGGATGGTGTTC | |
| | AAGGGCGAGATCCCCAAGGACCAGTGGATGAAGAAGTGGTGGGAGATGAAGAGGGAGA | |
| | TCGTGGGCGTGGTGGAGCCCGTGCCCCACGACGAGACCTACTGCGACCCCGCCTCCCT | |
| | GTTCCACGTGTCCAACGACTACTCCTTCATGAGGTACTAGACCAGGAGCCTGTACGAG | |
| | TTCCAGTTCCAGGAGGCCCTGTGCCAGGCCGCCAAGCACGAGGGCCCCCTGCACAAGT | |
| | GCGACATCTCCAACTCCACCGAGGCCGGCCAGAAGCTGTTCAACATGCTGAGGCTGGG | |
| | CAAGTCCGAGCCCTGGACCCTGGCCCTGGAGAACGTGGTGGGCGCCAAGAACATGAAC | |
| | GTGAGGCCCCTGCTGAACTACTTCGAGCCCCTGTTCACCTGGCTGAAGGACCAGAACA | |
| | AGAACTCCTTCGTGGGCTGGTCCACCGACTGGTCCCCCTACGCCGACCAGTCCATCAA | |
| | GGTGAGGATCTCCCTGAAGTCCGCCCTGGGCGACAAGGCCTACGAGTGGAACGACAAC | |
| | GAGATGTACCTGTTCAGGTCCTCCGTGGCCTACGCCATGAGGCAGTACTTCCTGAAGG | |
| | TGAAGAACCAGATGATCCTGTTCGGCGAGGAGGACGTGAGGGTGGCCAACCTGAAGCC | |
| | CAGGATCTCCTTCAACTTCTTCGTGACCGCCCCCAAGAACGTGTCCGACATCATCCCC | |
| | AGGACCGAGGTGGAGAAGGCCATCAGGATGTCCAGGTCCAGGATCAACGACGCCTTCA | |
| | GGCTGAACGACAACTCCCTGGAGTTCCTGGGCATCCAGCCCACCCTGGGCCCCCCCAA | |
| | CCAGCCCCCCGTGTCCATCTGGCTGATCGTGTTCGGCGTGGTGATGGGCGTGATCGTG | |
| | GTGGGCATCGTGATCCTGATCTTCACCGGCATCAGGGACAGGAAGAAGAAGAACAAGG | |
| | CCAGGTCCGGCGAGAACCCCTACGCCTCCATCGACATCTCCAAGGGCGAGAACAACCC | |
| | CGGCTTCCAGAACACCGACGACGTGCAGACCTCCTTC | |
| 243 | ATGTCCTCCTCCTCCTGGCTGCTGCTGTCCCTGGTGGCCGTGACCGCCGCCCAGTCCA | ACE2 extra- |
| | CCATCGAGGAGCAGGCCAAGACCTTCCTGGACAAGTTCAACCACGAGGCCGAGGACCT | cellular |
| | GTTCTACCAGTCCTCCCTGGCCTCCTGGAACTACAACACCAACATCACCGAGGAGAAC | domain |
| | GTGCAGAACATGAACAACGCCGGCGACAAGTGGTCCGCCTTCCTGAAGGAGCAGTCCA | (ECD) |
| | CCCTGGCCCAGATGTACCCCCTGCAGGAGATCCAGAACCTGACCGTGAAGCTGCAGCT | (Homo |
| | GCAGGCCCTGCAGCAGAACGGCTCCTCCGTGCTGTCCGAGGACAAGTCCAAGAGGCTG | sapiens) |
| | AACACCATCCTGAACACCATGTCCACCATCTACTCCACCGGCAAGGTGTGCAACCCCG | |
| | ACAACCCCCAGGAGTGCCTGCTGCTGGAGCCCGGCCTGAACGAGATCATGGCCAACTC | |
| | CCTGGACTACAACGAGAGGCTGTGGGCCTGGGAGTCCTGGAGGTCCGAGGTGGGCAAG | |
| | CAGCTGAGGCCCCTGTACGAGGAGTACGTGGTGCTGAAGAACGAGATGGCCAGGGCCA | |
| | ACCACTACGAGGACTACGGCGACTACTGGAGGGGCGACTACGAGGTGAACGGCGTGGA | |
| | CGGCTACGACTACTCCAGGGGCCAGCTGATCGAGGACGTGGAGCACACCTTCGAGGAG | |
| | ATCAAGCCCCTGTACGAGCACCTGCACGCCTACGTGAGGGCCAAGCTGATGAACGCCT | |
| | ACCCCTCCTACATCTCCCCCATCGGCTGCCTGCCCGCCCACCTGCTGGGCGACATGTG | |
| | GGGCAGGTTCTGGACCAACCTGTACTCCCTGACCGTGCCCTTCGGCCAGAAGCCCAAC | |
| | ATCGACGTGACCGACGCCATGGTGGACCAGGCCTGGGACGCCCAGAGGATCTTCAAGG | |
| | AGGCCGAGAAGTTCTTCGTGTCCGTGGGCCTGCCCAACATGACCCAGGGCTTCTGGGA | |
| | GAACTCCATGCTGACCGACCCCGGCAACGTGCAGAAGGCCGTGTGCCACCCCACCGCC | |
| | TGGGACCTGGGCAAGGGCGACTTCAGGATCCTGATGTGCACCAAGGTGACCATGGACG | |
| | ACTTCCTGACCGCCCACCACGAGATGGGCCACATCCAGTACGACATGGCCTACGCCGC | |
| | CCAGCCCTTCCTGCTGAGGAACGGCGCCAACGAGGGCTTCCACGAGGCCGTGGGCGAG | |
| | ATCATGTCCCTGTCCGCCGCCACCCCCAAGCACCTGAAGTCCATCGGCCTGCTGTCCC | |
| | CCGACTTCCAGGAGGACAACGAGACCGAGATCAACTTCCTGCTGAAGCAGGCCCTGAC | |
| | CATCGTGGGCACCCTGCCCTTCACCTACATGCTGGAGAAGTGGAGGTGGATGGTGTTC | |
| | AAGGGCGAGATCCCCAAGGACCAGTGGATGAAGAAGTGGTGGGAGATGAAGAGGGAGA | |
| | TCGTGGGCGTGGTGGAGCCCGTGCCCCACGACGAGACCTACTGCGACCCCGCCTCCCT | |
| | GTTCCACGTGTCCAACGACTACTCCTTCATCAGGTACTACACCAGGACCCTGTACCAG | |
| | TTCCAGTTCCAGGAGGCCCTGTGCCAGGCCGCCAAGCACGAGGGCCCCCTGCACAAGT | |
| | GCGACATCTCCAACTCCACCGAGGCCGGCCAGAAGCTGTTCAACATGCTGAGGCTGGG | |
| | CAAGTCCGAGCCCTGGACCCTGGCCCTGGAGAACGTGGTGGGCGCCAAGAACATGAAC | |
| | GTGAGGCCCCTGCTGAACTACTTCGAGCCCCTGTTCACCTGGCTGAAGGACCAGAACA | |
| | AGAACTCCTTCGTGGGCTGGTCCACCGACTGGTCCCCCTACGCCGACCAGTCCATCAA | |
| | GGTGAGGATCTCCCTGAAGTCCGCCCTGGGCGACAAGGCCTACGAGTGGAACGACAAC | |
| | GAGATGTACCTGTTCAGGTCCTCCGTGGCCTACGCCATGAGGCAGTACTTCCTGAAGG | |
| | TGAAGAACCAGATGATCCTGTTCGGCGAGGAGGACGTGAGGGTGGCCAACCTGAAGCC | |
| | CAGGATCTCCTTCAACTTCTTCGTGACCGCCCCCAAGAACGTGTCCGACATCATCCCC | |
| | AGGACCGAGGTGGAGAAGGCCATCAGGATGTCCAGGTCCAGGATCAACGACGCCTTCA | |
| | GGCTGAACGACAACTCCCTGGAGTTCCTGGGCATCCAGCCCACCCTGGGCCCCCCCAA | |
| | CCAGCCCCCCGTGTCC | |
| 244 | ATGTCCTCCTCCTCCTGGCTGCTGCTGTCCCTGGTGGCCGTGACCGCCGCCCAGTCCA | ACE2N extra- |
| | CCATCGAGGAGCAGGCCAAGACCTTCCTGGACAAGTTCAACCACGAGGCCGAGGACCT | cellular |
| | GTTCTACCAGTCCTCCCTGGCCTCCTGGAACTACAACACCAACATCACCGAGGAGAAC | domain |
| | GTGCAGAACATGAACAACGCCGGCGACAAGTGGTCCGCCTTCCTGAAGGAGCAGTCCA | (ECD) |
| | CCCTGGCCCAGATGTACCCCCTGCAGGAGATCCAGAACCTGACCGTGAAGCTGCAGCT | (Homo |
| | GCAGGCCCTGCAGCAGAACGGCTCCTCCGTGCTGTCCGAGGACAAGTCCAAGAGGCTG | sapiens) |
| | AACACCATCCTGAACACCATGTCCACCATCTACTCCACCGGCAAGGTGTGCAACCCCG | |
| | ACAACCCCCAGGAGTGCCTGCTGCTGGAGCCCGGCCTGAACGAGATCATGGCCAACTC | |
| | CCTGGACTACAACGAGAGGCTGTGGGCCTGGGAGTCCTGGAGGTCCGAGGTGGGCAAG | |
| | CAGCTGAGGCCCCTGTACGAGGAGTACGTGGTGCTGAAGAACGAGATGGCCAGGGCCA | |
| | ACCACTACGAGGACTACGGCGACTACTGGAGGGGCGACTACGAGGTGAACGGCGTGGA | |
| | CGGCTACGACTACTCCAGGGGCCAGCTGATCGAGGACGTGGAGCACACCTTCGAGGAG | |
| | ATCAAGCCCCTGTACGAGCACCTGCACGCCTACGTGAGGGCCAAGCTGATGAACGCCT | |
| | ACCCCTCCTACATCTCCCCCATCGGCTGCCTGCCCGCCCACCTGCTGGGCGACATGTG | |
| | GGGCAGGTTCTGGACCAACCTGTACTCCCTGACCGTGCCCTTCGGCCAGAAGCCCAAC | |
| | ATCGACGTGACCGACGCCATGGTGGACCAGGCCTGGGACGCCCAGAGGATCTTCAAGG | |
| | AGGCCGAGAAGTTCTTCGTGTCCGTGGGCCTGCCCAACATGACCCAGGGCTTCTGGGA | |
| | GAACTCCATGCTGACCGACCCCGGCAACGTGCAGAAGGCCGTGTGCCACCCCACCGCC | |
| | TGGGACCTGGGCAAGGGCGACITCAGGATCCTGATGTGCACCAAGGTGACLATGGACG | |
| | ACTTCCTGACCGCCCACAAOGAGATGGGCAACATCCAGTACGACATGGCCTACGCCGC | |
| | CCAGCCCTTCCTGCTGAGGAACGGCGCCAACGAGGGCTTCCACGAGGCCGTGGGCGAG | |
| | ATCATGTCCCTGTCCGCCGCCACCCCCAAGCACCTGAAGTCCATCGGCCTGCTGTCCC | |
| | CCGACTTCCAGGAGGACAACGAGACCGAGATCAACTTCCTGCTGAAGCAGGCCCTGAC | |
| | CATCGTGGGCACCCTGCCCTTCACCTACATGCTGGAGAAGTGGAGGTGGATGGTGTTC | |
| | AAGGGCGAGATCCCCAAGGACCAGTGGATGAAGAAGTGGTGGGAGATGAAGAGGGAGA | |
| | TCGTGGGCGTGGTGGAGCCCGTGCCCCACGACGAGACCTACTGCGACCCCGCCTCCCT | |
| | GTTCCACGTGTCCAACGACTACTCCTTCATCAGGTACTACACCAGGACCCTGTACCAG | |
| | TTCCAGTTCCAGGAGGCCCTGTGCCAGGCCGCCAAGCACGAGGGCCCCCTGCACAAGT | |
| | GCGACATCTCCAACTCCACCGAGUUCGGCCAGAAGCTGTTCAACATGCTGAGGCTGGG | |
| | CAAGTCCGAGCCCTGGACCCTGGCCCTGGAGAACGTGGTGGGCGCCAAGAACATGAAC | |
| | GTGAGGCCCCTGCTGAACTACTTCGAGCCCCTGTTCACCTGGCTGAAGGACCAGAACA | |
| | AGAACTCCTTCGTGGGCTGGTCCACCGACTGGTCCCCCTACGCCGACCAGTCCATCAA | |
| | GGTGAGGATCTCCCTGAAGTCCGCCCTGGGCGACAAGGCCTACGAGTGGAACGACAAC | |
| | GAGATGTACCTGTTCAGGTCCTCCGTGGCCTACGCCATGAGGCAGTACTTCCTGAAGG | |
| | TGAAGAACCAGATGATCCTGTTCGGCGAGGAGGACGTGAGGGTGGCCAACCTGAAGCC | |
| | CAGGATCTCCTTCAACTTCTTCGTGACCGCCCCCAAGAACGTGTCCGACATCATCCCC | |
| | AGGACCGAGGTGGAGAAGGCCATCAGGATGTCCAGGTCCAGGATCAACGACGCCTTCA | |
| | GGCTGAACGACAACTCCCTGGAGTTCCTGGGCATCCAGCCCACCCTGGGCCCCCCCAA | |
| | CCAGCCCCCCGTGTCC | |
| 245 | GCCCCCGAGCTGCTGGGuGGCCCCTCCGTGTTCCTGTTCCCCCCCAAGCCCAAGGACA | Fc peptide |
| | CCCTGATGATCTCCAGGACCCCCGAGGTGACCTGCGTGGTGGTGGACGTGTCCCACGA | Mut. Glyco. |
| | GGACCCCGAGGTGAAGTTCAACTGGTACGTGGACGGCGTGGAGGTGCACAACGCCAAG | (N->H) |
| | ACCAAGCCCAGGGAGGAGCAGTACCACTCCACCTACAGGGTGGTGTCCGTGCTGACCG | |
| | TGCTGCACCAGGACTGGCTGAACGGCAAGGAGTACAAGTGCAAGGTGTCCAACAAGGC | |
| | CCTGCCCGCCCCCATCGAGAAGACCATCTCCAAGGCCAAGGGCCAGCCCAGGGAGCCC | |
| | CAGGTGTACACCCTGCCCCCCTCCAGGGACGAGCTGACCAAGAACCAGGTGTCCCTGA | |
| | CCTGCCTGGTGAAGGGCTTCTACCCCTCCGACATCGCCGTGGAGTGGGAGTCCAACGG | |
| | CCAGCCCGAGAACAACTACAAGACCACCCCCCCCGTGCTGGACTCCGACGGCTCCTTC | |
| | TTCCTGTACTCCAAGCTGACCGTGGACAAGTCCAGGTGGCAGCAGGGCAACGTGTTCT | |
| | CCTGCTCCGTGATGCACGAGGCCCTGCACAACCACTACACCCAGAAGTCCCTGTCCCT | |
| | GTCCCCCGGC | |
| 246 | AACATCACCAACCTGTGCCCCTTCGGCGAGGTGTTCAACGCCACCAGGTTCGCCTCCG | S-RBD-sFc |
| | TGTACGCCTGGAACAGGAAGAGGATCTCCAACTGCGTGGCCGACTACTCCGTGCTGTA | Fusion |
| | CAACTCCGCCTCCTTCTCCACCTTCAAGTGCTACGGCGTGTCCCCCACCAAGCTGAAC | protein |
| | GACCTGTGCTTCACCAACGTGTACGCCGACTCCTTCGTGATCAGGGGCGACGAGGTGA | |
| | GGCAGATCGCCCCCGGCCAGACCGGCAAGATCGCCGACTACAACTACAAGCTGCCCGA | |
| | CGACTTCACCGGCTGCGTGATCGCCTGGAACTCCAACAACCTGGACTCCAAGGTGGGC | |
| | GGCAACTACAACTACCTGTACAGGCTGTTCAGGAAGTCCAACCTGAAGCCCTTCGAGA | |
| | GGGACATCTCCACCGAGATCTACCAGGCCGGCTCCACCCCCTGCAACGGCGTGGAGGG | |
| | CTTCAACTGCTACTTCCCCCTGCAGTCCTACGGCTTCCAGCCCACCAACGGCGTGGGC | |
| | TACCAGCCCTACAGGGTGGTGGTGCTGTCCTTCGAGCTGCTGCACGCCCCCGCCACCG | |
| | TGTGCGGCCCCAAGAAGTCCGAGCCCAAGTCCTCCGACAAGACCCACACCTCCCCCCC | |
| | CTCCCCCGCCCCCGAGCTGCTGGGCGGCCCCTCCGTGTTCCTGTTCCCCCCCAAGCCC | |
| | AAGGACACCCTGATGATCTCCAGGACCCCCGAGGTGACCTGCGTGGTGGTGGACGTGT | |
| | CCCACGAGGACCCCGAGGTGAAGTTCAACTGGTACGTGGACGGCGTGGAGGTGCACAA | |
| | CGCCAAGACCAAGCCCAGGGAGGAGCAGTACCACTCCACCTACAGGGTGGTGTCCGTG | |
| | CTGACCGTGCTGCACCAGGACTGGCTGAACGGCAAGGAGTACAAGTGCAAGGTGTCCA | |
| | ACAAGGCCCTGCCCGCCCCCATCGAGAAGACCATCTCCAAGGCCAAGGGCCAGCCCAG | |
| | GGAGCCCCAGGTGTACACCCTGCCCCCCTCCAGGGACGAGCTGACCAAGAACCAGGTG | |
| | TCCCTGACCTGCCTGGTGAAGGGCTTCTACCCCTCCGACATCGCCGTGGAGTGGGAGT | |
| | CCAACGGCCAGCCCGAGAACAACTACAAGACCACCCCCCCCGTGCTGGACTCCGACGG | |
| | CTCCTTCTTCCTGTACTCCAAGCTGACCGTGGACAAGTCCAGGTGGCAGCAGGGCAAC | |
| | GTGTTCTCCTGCTCCGTGATGCACGAGGCCCTGCACAACCACTACACCCAGAAGTCCC | |
| | TGTCCCTGTCCCCCGGC | |
| 247 | AACATCACCAACCTGTGCCCCTTCGGCGAGGTGTTCAACGCCACCAGGTTCGCCTCCG | S-RBDa-sFc |
| | TGTACGCCTGGAACAGGAAGAGGATCTCCAACTGCGTGGCCGACTACTCCGTGCTGTA | Fusion |
| | CAACTCCGCCTCCTTCTCCACCTTCAAGTGCTACGGCGTGTCCCCCACCAAGCTGAAC | protein |
| | GACCTGGCCTTCACCAACGTGTACGCCGACTCCTTCGTGATCAGGGGCGACGAGGTGA | |
| | GGCAGATCGCCCCCGGCCAGACCGGCAAGATCGCCGACTACAACTACAAGCTGCCCGA | |
| | CGACTTCACCGGCTGCGTGATCGCCTGGAACTCCAACAACCTGGACTCCAAGGTGGGC | |
| | GGCAACTACAACTACCTGTACAGGCTGTTCAGGAAGTCCAACCTGAAGCCCTTCGAGA | |
| | GGGACATCTCCACCGAGATCTACCAGGCCGGCTCCACCCCCTGCAACGGCGTGGAGGG | |
| | CTTCAACTGCTACTTCCCCCTGCAGTCCTACGGCTTCCAGCCCACCAACGGCGTGGGC | |
| | TACCAGCCCTACAGGGTGGTGGTGCTGTCCTTCGAGCTGCTGCACGCCCCCGCCACCG | |
| | TGGCCGGCCCCAAGAAGTCCGAGCCCAAGTCCTCCGACAAGACCCACACCTCCCCCCC | |
| | CTCCCCCGCCCCCGAGCTGCTGGGCGGCCCCTCCGTGTTCCTGTTCCCCCCCAAGCCC | |
| | AAGGACACCCTGATGATCTCCAGGACCCCCGAGGTGACCTGCGTGGTGGTGGACGTGT | |
| | CCCACGAGGACCCCGAGGTGAAGTTCAACTGGTACGTGGACGGCGTGGAGGTGCACAA | |
| | CGCCAAGACCAAGCCCAGGGAGGAGCAGTACCACTCCACCTACAGGGTGGTGTCCGTG | |
| | CTGACCGTGCTGCACCAGGACTGGCTGAACGGCAAGGAGTACAAGTGCAAGGTGTCCA | |
| | ACAAGGCCCTGCCCGCCCCCATCGAGAAGACCATCTCCAAGGCCAAGGGCCAGCCCAG | |
| | GGAGCCCCAGGTGTACACCCTGCCCCCCTCCAGGGACGAGCTGACCAAGAACCAGGTG | |
| | TCCCTGACCTGCCTGGTGAAGGGCTTCTACCCCTCCGACATCGCCGTGGAGTGGGAGT | |
| | CCAACGGCCAGCCCGAGAACAACTACAAGACCACCCCCCCCGTGCTGGACTCCGACGG | |
| | CTCCTTCTTCCTGTACTCCAAGCTGACCGTGGACAAGTCCAGGTGGCAGCAGGGCAAC | |
| | GTGTTCTCCTGCTCCGTGATGCACGAGGCCCTGCACAACCACTACACCCAGAAGTCCC | |
| | TGTCCCTGTCCCCCGGC | |
| 248 | ATGTCCTCCTCCTCCTGGCTGCTGCTGTCCCTGGTGGCCGTGACCGCCGCCCAGTCCA | ACE2-ECD- |
| | CCATCGAGGAGCAGGCCAAGACCTTCCTGGACAAGTTCAACCACGAGGCCGAGGACCT | sFc Fusion |
| | GTTCTACCAGTCCTCCCTGGCCTCCTGGAACTACAACACCAACATCACCGAGGAGAAC | protein |
| | GTGCAGAACATGAACAACGCCGGCGACAAGTGGTCCGCCTTCCTGAAGGAGCAGTCCA | |
| | CCCTGGCCCAGATGTACCCCCTGCAGGAGATCCAGAACCTGACCGTGAAGCTGCAGCT | |
| | GCAGGCCCTGCAGCAGAACGGCTCCTCCGTGCTGTCCGAGGACAAGTCCAAGAGGCTG | |
| | AACACCATCCTGAACACCATGTCCACCATCTACTCCACCGGCAAGGTGTGCAACCCCG | |
| | ACAACCCCCAGGAGTGCCTGCTGCTGGAGCCCGGCCTGAACGAGATCATGGCCAACTC | |
| | CCTGGACTACAACGAGAGGCTGTGGGCCTGGGAGTCCTGGAGGTCCGAGGTGGGCAAG | |
| | CAGCTGAGGCCCCTGTACGAGGAGTACGTGGTGCTGAAGAACGAGATGGCGAGGGCCA | |
| | ACCACTACGAGGACTACGGCGACTACTGGAGGGGCGACTACGAGGTGAACGGCGTGGA | |
| | CGGCTACGACTACTCCAGGGGCCAGCTGATCGAGGACGTGGAGCACACCTTCGAGGAG | |
| | ATCAAGCCCCTGTACGAGCACCTGCACGCCTACGTGAGGGCCAAGCTGATGAACGCCT | |
| | ACCCCTCCTACATCTCCCCCATCGGCTGCCTGCCCGCCCACCTGCTGGGCGACATGTG | |
| | GGGCAGGTTCTGGACCAACCTGTACTCCCTGACCGTGCCCTTCGGCCAGAAGCCCAAC | |
| | ATCGACGTGACCGACGCCATGGTGGACCAGGCCTGGGACGCCCAGAGGATCTTCAAGG | |
| | AGGCCGAGAAGTTCTTCGTGTCCGTGGGCCTGCCCAACATGACCCAGGGCTTCTGGGA | |
| | GAACTCCATGCTGACCGACCCCGGCAACGTGCAGAAGGCCGTGTGCCACCCCACCGCC | |
| | TGGGACCTGGGCAAGGGCGACTTCAGGATCCTGATGTGCACCAAGGTGACCATGGACG | |
| | ACTTCCTGACCGCCCACCACGAGATGGGCCACATCCAGTACGACATGGCCTACGCCGC | |
| | CCAGCCCTTCCTGCTGAGGAACGGCGCCAACGAGGGCTTCCACGAGGCCGTGGGCGAG | |
| | ATCATGTCCCTGTCCGCCGCCACCCCCAAGCACCTGAAGTCCATCGGCCTGCTGTCCC | |
| | CCGACTTCCAGGAGGACAACGAGACCGAGATCAACTTCCTGCTGAAGCAGGCCCTGAC | |
| | CATCGTGGGCACCCTGCCCTTCACCTACATGCTGGAGAAGTGGAGGTGGATGGTGTTC | |
| | AAGGGCGAGATCCCCAAGGACCAGTGGATGAAGAAGTGGTGGGAGATGAAGAGGGAGA | |
| | TCGTGGGCGTGGTGGAGCCCGTGCCCCACGACGAGACCTACTGCGACCCCGCCTCCCT | |
| | GTTCCACGTGTCCAACGACTACTCCTTCATCAGGTACTACACCAGGACCCTGTACCAG | |
| | TTCCAGTTCCAGGAGGCCCTGTGCCAGGCCGCCAAGCACGAGGGCCCCCTGCACAAGT | |
| | GCGACATCTCCAACTCCACCGAGGCCGGCCAGAAGCTGTTCAACATGCTGAGGCTGGG | |
| | CAAGTCCGAGCCCTGGACCCTGGCCCTGGAGAACGTGGTGGGCGCCAAGAACATGAAC | |
| | GTGAGGCCCCTGCTGAACTACTTCGAGCCCCTGTTCACCTGGCTGAAGGACCAGAACA | |
| | AGAACTCCTTCGTGGGCTGGTCCACCGACTGGTCCCCCTACGCCGACCAGTCCATCAA | |
| | GGTGAGGATCTCCCTGAAGTCCGCCCTGGGCGACAAGGCCTACGAGTGGAACGACAAC | |
| | GAGATGTACCTGTTCAGGTCCTCCGTGGCCTACGCCATGAGGCAGTACTTCCTGAAGG | |
| | TGAAGAACCAGATGATCCTGTTCGGCGAGGAGGACGTGAGGGTGGCCAACCTGAAGCC | |
| | CAGGATCTCCTTCAACTTCTTCGTGACCGCCCCCAAGAACGTGTCCGACATCATCCCC | |
| | AGGACCGAGGTGGAGAAGGCCATCAGGATGTCCAGGTCCAGGATCAACGACGCCTTCA | |
| | GGCTGAACGACAACTCCCTGGAGTTCCTGGGCATCCAGCCCACCCTGGGCCCCCCCAA | |
| | CCAGCCCCCCGTGTCCGAGCCCAAGTCCTCCGACAAGACCCACACCTCCCCCCCCTCC | |
| | CCCGCCCCCGAGCTGCTGGGCGGCCCCTCCGTGTTCCTGTTCCCCCCCAAGCCCAAGG | |
| | ACACCCTGATGATCTCCAGGACCCCCGAGGTGACCTGCGTGGTGGTGGACGTGTCCCA | |
| | CGAGGACCCCGAGGTGAAGTTCAACTGGTACGTGGACGGCGTGGAGGTGCACAACGCC | |
| | AAGACCAAGCCCAGGGAGGAGCAGTACCACTCCACCTACAGGGTGGTGTCCGTGCTGA | |
| | CCGTGCTGCACCAGGACTGGCTGAACGGCAAGGAGTACAAGTGCAAGGTGTCCAACAA | |
| | GGCCCTGCCCGCCCCCATCGAGAAGACCATCTCCAAGGCCAAGGGCCAGCCCAGGGAG | |
| | CCCCAGGTGTACACCCTGCCCCCCTCCAGGGACGAGCTGACCAAGAACCAGGTGTCCC | |
| | TGACCTGCCTGGTGAAGGGCTTCTACCCCTCCGACATCGCCGTGGAGTGGGAGTCCAA | |
| | CGGCCAGCCCGAGAACAACTACAAGACCACCCCCCCCGTGCTGGACTCCGACGGCTCC | |
| | TTCTTCCTGTACTCCAAGCTGACCGTGGACAAGTCCAGGTGGCAGCAGGGCAACGTGT | |
| | TCTCCTGCTCCGTGATGCACGAGGCCCTGCACAACCACTACACCCAGAAGTCCCTGTC | |
| | CCTGTCCCCCGGC | |
| 249 | ATGTCCTCCTCCTCCTGGCTGCTGCTGTCCCTGGTGGCCGTGACCGCCGCCCAGTCCA | ACE2N-ECD- |
| | CCATCGAGGAGCAGGCCAAGACCTTCCTGGACAAGTTCAACCACGAGGCCGAGGACCT | sFc Fusion |
| | GTTCTACCAGTCCTCCCTGGCCTCCTGGAACTACAACACCAACATCACCGAGGAGAAC | protein |
| | GTGCAGAACATGAACAACGCCGGCGACAAGTGGTCCGCCTTCCTGAAGGAGCAGTCCA | |
| | CCCTGGCCCAGATGTACCCCCTGCAGGAGATCCAGAACCTGACCGTGAAGCTGCAGCT | |
| | GCAGGCCCTGCAGCAGAACGGCTCCTCCGTGCTGTCCGAGGACAAGTCCAAGAGGCTG | |
| | AACACCATCCTGAACACCATGTCCACCATCTACTCCACCGGCAAGGTGTGCAACCCCG | |
| | ACAACCCCCAGGAGTGCCTGCTGCTGGAGCCCGGCCTGAACGAGATCATGGCCAACTC | |
| | CCTGGACTACAACGAGAGGCTGTGGGCCTGGGAGTCCTGGAGGTCCGAGGTGGGCAAG | |
| | CAGCTGAGGCCCCTGTACGAGGAGTACGTGGTGCTGAAGAACGAGATGGCCAGGGCCA | |
| | ACCACTACGAGGACTACGGCGACTACTGGAGGGGCGACTACGAGGTGAACGGCGTGGA | |
| | CGGCTACGACTACTCCAGGGGCCAGCTGATCGAGGACGTGGAGCACACCTTCGAGGAG | |
| | ATCAAGCCCCTGTACGAGCACCTGCACGCCTACGTGAGGGCCAAGCTGATGAACGCCT | |
| | ACCCCTCCTACATCTCCCCCATCGGCTGCCTGCCCGCCCACCTGCTGGGCGACATGTG | |
| | GGGCAGGTTCTGGACCAACCTGTACTCCCTGACCGTGCCCTTCGGCCAGAAGCCCAAC | |
| | ATCGACGTGACCGACGCCATGGTGGACCAGGCCTGGGACGCCCAGAGGATCTTCAAGG | |
| | AGGCCGAGAAGTTCTTCGTGTCCGTGGGCCTGCCCAACATGACCCAGGGCTTCTGGGA | |
| | GAACTCCATGCTGACCGACCCCGGCAACGTGCAGAAGGCCGTGTGCCACCCCACCGCC | |
| | TGGGACCTGGGCAAGGGCGACTTCAGGATCCTGATGTGCACCAAGGTGACCATGGACG | |
| | ACTTCCTGACCGCCCACAACGAGATGGGCAACATCCAGTACGACATGGCCTACGCCGC | |
| | CCAGCCCTTCCTGCTGAGGAACGGCGCCAACGAGGGCTTCCACGAGGCCGTGGGCGAG | |
| | ATCATGTCCCTGTCCGCCGCCACCCCCAAGCACCTGAAGTCCATCGGCCTGCTGTCCC | |
| | CCGACTTCCAGGAGGACAACGAGACCGAGATCAACTTCCTGCTGAAGCAGGCCCTGAC | |
| | CATCGTGGGCACCCTGCCCTTCACCTACATGCTGGAGAAGTGGAGGTGGATGGTGTTC | |
| | AAGGGCGAGATCCCCAAGGACCAGTGGATGAAGAAGTGGTGGGAGATGAAGAGGGAGA | |
| | TCGTGGGCGTGGTGGAGCCCGTGCCCCACGACGAGACCTACTGCGACCCCGCCTCCCT | |
| | GTTCCACGTGTCCAACGACTACTCCTTCATCAGGTACTACACCAGGACCCTGTACCAG | |
| | TTCCAGTTCCAGGAGGCCCTGTGCCAGGCCGCCAAGCACGAGGGCCCCCTGCACAAGT | |
| | GCGACATCTCCAACTCCACCGAGGCCGGCCAGAAGCTGTTCAACATGCTGAGGCTGGG | |
| | CAAGTCCGAGCCCTGGACCCTGGCCCTGGAGAACGTGGTGGGCGCCAAGAACATGAAC | |
| | GTGAGGCCCCTGCTGAACTACTTCGAGCCCCTGTTCACCTGGCTGAAGGACCAGAACA | |
| | AGAACTCCTTCGTGGGCTGGTCCACCGACTGGTCCCCCTACGCCGACCAGTCCATCAA | |
| | GGTGAGGATCTCCCTGAAGTCCGCCCTGGGCGACAAGGCCTACGAGTGGAACGACAAC | |
| | GAGATGTACCTGTTCAGGTCCTCCGTGGCCTACGCCATGAGGCAGTACTTCCTGAAGG | |
| | TGAAGAACCAGATGATCCTGTTCGGCGAGGAGGACGTGAGGGTGGCCAACCTGAAGCC | |
| | CAGGATCTCCTTCAACTTCTTCGTGACCGCCCCCAAGAACGTGTCCGACATCATCCCC | |
| | AGGACCGAGGTGGAGAAGGCCATCAGGATGTCCAGGTCCAGGATCAACGACGCCTTCA | |
| | GGCTGAACGACAACTCCCTGGAGTTCCTGGGCATCCAGCCCACCCTGGGCCCCCCCAA | |
| | CCAGCCCCCCGTGTCCGAGCCCAAGTCCTCCGACAAGACCCACACCTCCCCCCCCTCC | |
| | CCCGCCCCCGAGCTGCTGGGCGGCCCCTCCGTGTTCCTGTTCCCCCCCAAGCCCAAGG | |
| | ACACCCTGATGATCTCCAGGACCCCCGAGGTGACCTGCGTGGTGGTGGACGTGTCCCA | |
| | CGAGGACCCCGAGGTGAAGTTCAACTGGTACGTGGACGGCGTGGAGGTGCACAACGCC | |
| | AAGACCAAGCCCAGGGAGGAGCAGTACCACTCCACCTACAGGGTGGTGTCCGTGCTGA | |
| | CCGTGCTGCACCAGGACTGGCTGAACGGCAAGGAGTACAAGTGCAAGGTGTCCAACAA | |
| | GGCCCTGCCCGCCCCCATCGAGAAGACCATCTCCAAGGCCAAGGGCCAGCCCAGGGAG | |
| | CCCCAGGTGTACACCCTGCCCCCCTCCAGGGACGAGCTGACCAAGAACCAGGTGTCCC | |
| | TGACCTGCCTGGTGAAGGGCTTCTACCCCTCCGACATCGCCGTGGAGTGGGAGTCCAA | |
| | CGGCCAGCCCGAGAACAACTACAAGACCACCCCCCCCGTGCTGGACTCCGACGGCTCC | |
| | TTCTTCCTGTACTCCAAGCTGACCGTGGACAAGTCCAGGTGGCAGCAGGGCAACGTGT | |
| | TCTCCTGCTCCGTGATGCACGAGGCCCTGCACAACCACTACACCCAGAAGTCCCTGTC | |
| | CCTGTCCCCCGGC | |
| 357 | AACATCACCAACCTGTGCCCCTTCGGCGAGGTGTTCAACGCCACCAGGTTCGCCTCCG | S-RBD-Fc |
| | TGTACGCCTGGAACAGGAAGAGGATCTCCAACTGCGTGGCCGACTACTCCGTGCTGTA | Fusion |
| | CAACTCCGCCTCCTTCTCCACCTTCAAGTGCTACGGCGTGTCCCCCACCAAGCTGAAC | protein |
| | GACCTGTGCTTCACCAACGTGTACGCCGACTCCTTCGTGATCAGGGGCGACGAGGTGA | |
| | GGCAGATCGCCCCCGGCCAGACCGGCAAGATCGCCGACTACAACTACAAGCTGCCCGA | |
| | CGACTTCACCGGCTGCGTGATCGCCTGGAACTCCAACAACCTGGACTCCAAGGTGGGC | |
| | GGCAACTACAACTACCTGTACAGGCTGTTCAGGAAGTCCAACCTGAAGCCCTTCGAGA | |
| | GGGACATCTCCACCGAGATCTACCAGGCCGGCTCCACCCCCTGCAACGGCGTGGAGGG | |
| | CTTCAACTGCTACTTCCCCCTGCAGTCCTACGGCTTCCAGCCCACCAACGGCGTGGGC | |
| | TACCAGCCCTACAGGGTGGTGGTGCTGTCCTTCGAGCTGCTGCACGCCCCCGCCACCG | |
| | TGTGCGGCCCCAAGAAGTCCGAGCCCAAGTCCTGCGACAAGACCCACACCTGCCCCCC | |
| | CTGCCCCGCCCCCGAGCTGCTGGGCGGCCCCTCCGTGTTCCTGTTCCCCCCCAAGCCC | |
| | AAGGACACCCTGATGATCTCCAGGACCCCCGAGGTGACCTGCGTGGTGGTGGACGTGT | |
| | CCCACGAGGACCCCGAGGTGAAGTTCAACTGGTACGTGGACGGCGTGGAGGTGCACAA | |
| | CGCCAAGACCAAGCCCAGGGAGGAGCAGTACCACTCCACCTACAGGGTGGTGTCCGTG | |
| | CTGACCGTGCTGCACCAGGACTGGCTGAACGGCAAGGAGTACAAGTGCAAGGTGTCCA | |
| | ACAAGGCCCTGCCCGCCCCCATCGAGAAGACCATCTCCAAGGCCAAGGGCCAGCCCAG | |
| | GGAGCCCCAGGTGTACACCCTGCCCCCCTCCAGGGACGAGCTGACCAAGAACCAGGTG | |
| | TCCCTGACCTGCCTGGTGAAGGGCTTCTACCCCTCCGACATCGCCGTGGAGTGGGAGT | |
| | CCAACGGCCAGCCCGAGAACAACTACAAGACCACCCCCCCCGTGCTGGACTCCGACGG | |
| | CTCCTTCTTCCTGTACTCCAAGCTGACCGTGGACAAGTCCAGGTGGCAGCAGGGCAAC | |
| | GTGTTCTCCTGCTCCGTGATGCACGAGGCCCTGCACAACCACTACACCCAGAAGTCCC | |
| | TGTCCCTGTCCCCCGGC | |
| 358 | ATGTCCTCCTCCTCCTGGCTGCTGCTGTCCCTGGTGGCCGTGACCGCCGCCCAGTCCA | ACE2-ECD-Fc |
| | CCATCGAGGAGCAGGCCAAGACCTTCCTGGACAAGTTCAACCACGAGGCCGAGGACCT | Fusion |
| | GTTCTACCAGTCCTCCCTGGCCTCCTGGAACTACAACACCAACATCACCGAGGAGAAC | protein |
| | GTGCAGAACATGAACAACGCCGGCGACAAGTGGTCCGCCTTCCTGAAGGAGCAGTCCA | |
| | CCCTGGCCCAGATGTACCCCCTGCAGGAGATCCAGAACCTGACCGTGAAGCTGCAGCT | |
| | GCAGGCCCTGCAGCAGAACGGCTCCTCCGTGCTGTCCGAGGACAAGTCCAAGAGGCTG | |
| | AACACCATCCTGAACACCATGTCCACCATCTACTCCACCGGCAAGGTGTGCAACCCCG | |
| | ACAACCCCCAGGAGTGCCTGCTGCTGGAGCCCGGCCTGAACGAGATCATGGCCAACTC | |
| | CCTGGACTACAACGAGAGGCTGTGGGCCTGGGAGTCCTGGAGGTCCGAGGTGGGCAAG | |
| | CAGCTGAGGCCCCTGTACGAGGAGTACGTGGTGCTGAAGAACGAGATGGCCAGGGCCA | |
| | ACCACTACGAGGACTACGGCGACTACTGGAGGGGCGACTACGAGGTGAACGGCGTGGA | |
| | CGGCTACGACTACTCCAGGGGCCAGCTGATCGAGGACGTGGAGCACACCTTCGAGGAG | |
| | ATCAAGCCCCTGTACGAGCACCTGCACGCCTACGTGAGGGCCAAGCTGATGAACGCCT | |
| | ACCCCTCCTACATCTCCCCCATCGGCTGCCTGCCCGCCCACCTGCTGGGCGACATGTG | |
| | GGGCAGGTTCTGGACCAACCTGTACTCCCTGACCGTGCCCTTCGGCCAGAAGCCCAAC | |
| | ATCGACGTGACCGACGCCATGGTGGACCAGGCCTGGGACGCCCAGAGGATCTTCAAGG | |
| | AGGCCGAGAAGTTCTTCGTGTCCGTGGGCCTGCCCAACATGACCCAGGGCTTCTGGGA | |
| | GAACTCCATGCTGACCGACCCCGGCAACGTGCAGAAGGCCGTGTGCCACCCCACCGCC | |
| | TGGGACCTGGGCAAGGGCGACTTCAGGATCCTGATGTGCACCAAGGTGACCATGGACG | |
| | ACTTCCTGACCGCCCACCACGAGATGGGCCACATCCAGTACGACATGGCCTACGCCGC | |
| | CCAGCCCTTCCTGCTGAGGAACGGCGCCAACGAGGGCTTCCACGAGGCCGTGGGCGAG | |
| | ATCATGTCCCTGTCCGCCGCCACCCCCAAGCACCTGAAGTCCATCGGCCTGCTGTCCC | |
| | CCGACTTCCAGGAGGACAACGAGACCGAGATCAACTTCCTGCTGAAGCAGGCCCTGAC | |
| | CATCGTGGGCACCCTGCCCTTCACCTACATGCTGGAGAAGTGGAGGTGGATGGTGTTC | |
| | AAGGGCGAGATCCCCAAGGACCAGTGGATGAAGAAGTGGTGGGAGATGAAGAGGGAGA | |
| | TCGTGGGCGTGGTGGAGCCCGTGCCCCACGACGAGACCTACTGCGACCCCGCCTCCCT | |
| | GTTCCACGTGTCCAACGACTACTCCTTCATCAGGTACTACACCAGGACCCTGTACCAG | |
| | TTCCAGTTCCAGGAGGCCCTGTGCCAGGCCGCCAAGCACGAGGGCCCCCTGCACAAGT | |
| | GCGACATCTCCAACTCCACCGAGGCCGGCCAGAAGCTGTTCAACATGCTGAGGCTGGG | |
| | CAAGTCCGAGCCCTGGACCCTGGCCCTGGAGAACGTGGTGGGCGCCAAGAACATGAAC | |
| | GTGAGGCCCCTGCTGAACTACTTCGAGCCCCTGTTCACCTGGCTGAAGGACCAGAACA | |
| | AGAACTCCTTCGTGGGCTGGTCCACCGACTGGTCCCCCTACGCCGACCAGTCCATCAA | |
| | GGTGAGGATCTCCCTGAAGTCCGCCCTGGGCGACAAGGCCTACGAGTGGAACGACAAC | |
| | GAGATGTACCTGTTCAGGTCCTCCGTGGCCTACGCCATGAGGCAGTACTTCCTGAAGG | |
| | TGAAGAACCAGATGATCCTGTTCGGCGAGGAGGACGTGAGGGTGGCCAACCTGAAGCC | |
| | CAGGATCTCCTTCAACTTCTTCGTGACCGCCCCCAAGAACGTGTCCGACATCATCCCC | |
| | AGGACCGAGGTGGAGAAGGCCATCAGGATGTCCAGGTCCAGGATCAACGACGCCTTCA | |
| | GGCTGAACGACAACTCCCTGGAGTTCCTGGGCATCCAGCCCACCCTGGGCCCCCCCAA | |
| | CCAGCCCCCCGTGTCCGAGCCCAAGTCCTGCGACAAGACCCACACCTGCCCCCCCTGG | |
| | CCCGCCCCCGAGCTGCTGGGCGGCCCCTCCGTGTTCCTGTTCCCCCCCAAGCCCAAGG | |
| | ACACCCTGATGATCTCCAGGACCCCCGAGGTGACCTGCGTGGTGGTGGACGTGTCCCA | |
| | CGAGGACCCCGAGGTGAAGTTCAACTGGTACGTGGACGGCGTGGAGGTGCACAACGCC | |
| | AAGACCAAGCCCAGGGAGGAGCAGTACCACTCCACCTACAGGGTGGTGTCCGTGCTGA | |
| | CCGTGCTGCACCAGGACTGGCTGAACGGCAAGGAGTACAAGTGCAAGGTGTCCAACAA | |
| | GGCCCTGCCCGCCCCCATCGAGAAGACCATCTCCAAGGCCAAGGGCCAGCCCAGGGAG | |
| | CCCCAGGTGTACACCCTGCCCCCCTCCAGGGACGAGCTGACCAAGAACCAGGTGTCCC | |
| | TGACCTGCCTGGTGAAGGGCTTCTACCCCTCCGACATCGCCGTGGAGTGGGAGTCCAA | |
| | CGGCCAGCCCGAGAACAACTACAAGACCACCCCCCCCGTGCTGGACTCCGACGGCTCC | |
| | TTCTTCCTGTACTCCAAGCTGACCGTGGACAAGTCCAGGTGGCAGCAGGGCAACGTGT | |
| | TCTCCTGCTCCGTGATGCACGAGGCCCTGCACAACCACTACACCCAGAAGTCCCTGTC | |
| | CCTGTCCCCCGGC | |
| 360 | CACCACCACCACCACCACGAGAACCTGTACTTCCAGGGCAACATCACCAACCTGTGCC | S-RBD-His |
| | CCTTCGGCGAGGTGTTCAACGCCACCAGGTTCGCCTCCGTGTACGCCTGGAACAGGAA | Fusion |
| | GAGGATCTCCAACTGCGTGGCCGACTACTCCGTGCTGTACAACTCCGCCTCCTTCTCC | protein |
| | ACCTTCAAGTGCTACGGCGTGTCCCCCACCAAGCTGAACGACCTGTGCTTCACCAACG | |
| | TGTACGCCGACTCCTTCGTGATCAGGGGCGACGAGGTGAGGCAGATCGCCCCCGGCCA | |
| | GACCGGCAAGATCGCCGACTACAACTACAAGCTGCCCGACGACTTCACCGGCTGCGTG | |
| | ATCGCCTGGAACTCCAACAACCTGGACTCCAAGGTGGGCGGCAACTACAACTACCTGT | |
| | ACAGGCTGTTCAGGAAGTCCAACCTGAAGCCCTTCGAGAGGGACATCTCCACCGAGAT | |
| | CTACCAGGCCGGCTCCACCCCCTGCAACGGCGTGGAGGGCTTCAACTGCTACTTCCCC | |
| | CTGCAGTCCTACGGCTTCCAGCCCACCAACGGCGTGGGCTACCAGCCCTACAGGGTGG | |
| | TGGTGCTGTCCTTCGAGCTGCTGCACGCCCCCGCCACCGTGTGCGGCCCCAAGAAGTC | |
| | C | |

TABLE 13
SARS-CoV-2 antigenic peptides

| SEQ | | | |
|---|---|---|---|
| ID | Protein | | |
| NO | source | Position | Amino acid sequence |
| 250 | M | KKK-(64-86) | KKK-CFVLAAVYRINWITGGIAIAMAC |
| 251 | M | KKK-(69-83) | KKK-AVYRINWITGGIAIA |
| 252 | E | KKK-(1-18) | KKK-MYSFVSEETGTLIVNSVL |
| 253 | N | 73-90 | PINTNSSPDDQIGYYRRA |
| 254 | N | 55-90 | ALTQHGKEDLKFPRGQGVPINTNSSPDDQIGYYRRA |
| 255 | N | 37-90 | SKQRRPQGLPNNTASWFTALTQHGKEDLKFPRGQGVPINTNSSPDDQIGYYRRA |
| 256 | N | 19-90 | GPSDSTGSNQNGERSGARSKQRRPQGLPNNTASWFTALTQHGKEDLKFPRGQGV |
| | | | PINTNSSPDDQIGYYRRA |
| 257 | N | 1-90 | MSDNGPQNQRNAPRITFGGPSDSTGSNQNGARSGARSKQRRPQGLPNNTASWFT |
| | | | ALTQHGKEDLKFPRGQGVPINTNSSPDDQIGYYRRA |
| 258 | N | 73-108 | PINTNSSPDDQIGYYRRATRRIRGGDGKMKDLSPRW |
| 259 | N | 160-178 | QLPQGTTLPKGFYAEGSRG |
| 260 | N | 142-178 | PKDHTGTRNPANNAAIVLQLPQGTTLPKGFYAEGSRG |
| 261 | N | 125-178 | ANKDGLIWVATEGALNTPKDHIGTRNPANNAAIVLQLPQGTTLPKGFYAEGSRG |
| 262 | N | KKK-125-178 | KKK-ANKDGIIWVATEGALNTPKDHIGTRNPANNAAIVLQLPQGTTLPKGFYAE |
| | | | GSRG |
| 263 | N | 109-178 | YFYYLGTGPEAGLPYGANKDGIIWVATEGALNTPKDHIGTRNPANNAAIVLQLP |
| | | | QGTTLPKGFYAEGSRG |
| 264 | N | 91-178 | TRRIRGGDGKMKDLSPRWYFYYLGTGPEAGLPYGANKDGIIWVATEGALNTPKD |
| | | | HIGTRNPANNAAIVLQLPQGTTLPKGFYAEGSRG |
| 265 | N | 160-195 | QLPQGTTLPKGFYAEGSRGGSQASSRSSSRSRNSSR |
| 266 | N | KKK-160-195 | KKK-QLPQGTTLPKGFYAEGSRGGSQASSRSSSRSRNSSR |
| 267 | N | 249-266 | KSAAEASKKPRQKRTATK |
| 268 | N | 231-266 | ESKMSGKGQQQQGQTVTKKSAAEASKKPRQKRTATK |
| 269 | N | 213-266 | NGGDAALALLLLDRLNQLESKMSGKGQQQQGQTVTKKSAAEASKKPRQKRTATK |
| 270 | N | KKK-213-266 | KKK-NGGDAALALLLLDRLNQLESKMSGKGQQQQGQTVTKKSAAEASKKPRQKR |
| | | | TATK |
| 271 | N | 196-266 | NSTPGSSRGTSPARMAGNGGDAALALLLLDRLSQLESKMSGKGQQQQGQTVTKK |
| | | | SAAEASKKPRQKRTATK |
| 272 | N | 179-266 | GSQASSRSSSRSRNSSRNSTPGSSRGTSPARMAGNGGDAALALLLLDRLNQLES |
| | | | KMSGKGQQQQGQTVTKKSAAEASKKPRQKRTATK |
| 273 | N | 249-283 | KSAAEASKKPRQKRTATKAYNVTQAFGRRGPEQTQ |
| 274 | N | 337-354 | IKLDDKDPNFKDQVILLN |
| 275 | N | 319-354 | RIGMEVTPSGTWLTYTGAIKLDDKDPNFKDQVILLN |
| 276 | N | 301-354 | WPQIAQFAPSASAFFGMSRIGMEVTPSGTWLTYTGAIKLDDKDPNFKDQVILLN |
| 277 | N | 284-354 | GNFGDQELIRQGTDYKHWPQIAQFAPSASAFFGMSRIGMEVTPSGTWLTYTGAI |
| | | | KLDDKDPNFKDQVILLN |
| 278 | N | 267-354 | AYNVTQAFGRRGPEQTQGNFGDQELIRQGTDYKHWPQIAQFAPSASAFFGMSRI |
| | | | GMEVTPSGTWLTYTGAIKLDDKDPNFKDQVILLN |
| 279 | S | 570-588 | ADTTDAVRDPQTLEILDIT |
| 280 | S | 552-588 | LTESNKKFLPFQQFGRDIADTTDAVRDPQTLEILDIT |
| 281 | S | KKK-534-588 | KKK-VKNKSVNFNFNGLTGTGVLTESNKKFLPFQQFGRDIADTTDAVRDPQTLE |
| | | | ILDIT |
| 282 | S | KKK-518-588 | KKK-LHAPATVSGPKKSTNLVKNKSVNFNFNGLTGTGVLTESNKKFLPFQQFGR |
| | | | DIADTTDAVRDPQTLEILDIT |
| 283 | S | KKK-500-588 | KKK-TNGVGYQPYRVVVLSFELLHAPATVSGPKKSTNLVKNKSVNFNFNGLTGT |
| | | | GVLTESNKKFLPFQQFGRDIADTTDAVRDPQTLEILDIT |
| 284 | S | 573-604 | TDAVRDPQTLEILDITPSSFGGVSVITPGTNT |
| 285 | S | 659-678 | SYESDIPIGAGISASYQTQT |
| 286 | S | 641-678 | NVFQTRAGSLIGAEHVNNSYESDIPIGAGISASYQTQT |
| 287 | S | KKK-623-678 | KKK-AIHADQLTPTWRVYSTGSNVFQTRAGSLIGAEHVNNSYESDIPIGAGISA |
| | | | SYQTQT |
| 288 | S | 607-678 | QVAVLYQDVNSTEVPVAIHADQLTPTWRVYSTGSNVPQTRAGSLIGAEHVNNSY |
| | | | ESDIPIGAGISASYQTQT |
| 289 | S | KKK-589-678 | KKK-PSSFGGVSVITPGTNTSNQVAVLYQDVNSTEVPVAIHADQLTPTWRVYST |
| | | | GSNVFQTRAGSLIGAEHVNNSYESDIPIGAGISASYQTQT |
| 290 | S | 661-696 | ESDIPIGAGISASYQTQTNSPRRARSVASQSLIAYT |
| 291 | S | 750-766 | SSNLLLQYGSFSTQLNRA |
| 292 | S | 731-766 | MTKTSVDSTMYISGDSTESSNLLLQYGSFSTQLNRA |
| 293 | S | KKK-713-766 | KKK-AIPTNFTISVTTEILPVSMTKTSVDSTMYISGDSTESSNLLLQYGSFSTQ |
| | | | LNRA |
| 294 | S | 697-766 | MSLGAENSVAYSNNSIAIPTNFTISVTTEILPVSMTKTSVDSTMYISGDSTESS |
| | | | NLLLQYGSFSTQLNRA |
| 295 | S | KKK-679-766 | KKK-NSPRRARSVASQSIIAYTMSLGAENSVAYSNNSIAIPTNFTISVTTEILP |
| | | | VSMTKTSVDSTMYISGDSTESSNLLLQYGSFSTQLNRA |
| 296 | S | 767-804 | LTGIAVEQDKNTQEVFAQVKQIYKTPPIKDFGGFNFSQ |
| 297 | S | 821-858 | LLFNKVTLADAGFIKQYGDSLGDIAARDLISAQKFNGL |
| 298 | S | 910-927 | GVTQNVLYENQKLIANQF |
| 299 | S | 892-927 | AALQIPFAMQMAYRFNGIGVTQNVLYENQKLIANQF |
| 300 | S | KKK-875-927 | KKK-TSALLAGTITSGWTFGAGAALQIPFAMQMAYRFNGIGVTQNVLYENQKLI |
| | | | ANQF |
| 301 | S | 858-927 | LTVLPPLLTDEMIAQYTSALLAGTITSGWTIGAGAALQIPFAMQMAYRFNGIGV |
| | | | TQNVLYENQKLIANQF |
| 302 | S | KKK-840-927 | KKK-SLGDIAARDLISAQKFNGLTVLPPLLTDEMIAQYTSALLAGTITSGWTFG |
| | | | AGAALQIPFAMQMAYRFNGIGVTQNVLYENQKLIANQF |
| 303 | S | 910-945 | GVTQNVLYENQKLIANQFNSAIGKIQDSLSSTASAL |
| 304 | S | 998-1015 | TGRLQSLQTYVTQQLIRA |
| 305 | S | 980-1015 | ILSRLDKVEAEVQIDRLITGRLQSLQTYVTQQLIRA |
| 306 | S | KKK-962-1015 | KKK-LVKQLSSNFGAISSVLNDILSRLDKVEAEVQIDRLITGRLQSLQTYVTQQ |
| | | | LIRA |
| 307 | S | 946-1015 | GKLQDVVNQNAQALNTLVKQLSSNFGAISSVLNDILSRLDKVEAEVQIDRLITG |
| | | | RLQSLQTYVTQQLIRA |
| 308 | S | KKK-928-1015 | KKK-NSAIGKIQDSLSSTASALGKLQDVVNQNAQALNTLVKQLSSNFGAISSVL |
| | | | NDILSRLDKVEAEVQIDRLITGRLQSLQTYVTQQLIRA |
| 309 | S | 998-1033 | TGRLQSLQTYVTQQLIRAAEIRASANLAATKMSESV |
| 310 | S | 1086-1103 | KAHFPREGVFVSNGTHWF |
| 311 | S | 1068-1103 | VPAQEKNFTTAPAISHDGKAHFPREGVFVSNGTHWF |
| 312 | S | KKK-1050-1103 | KKK-MSFPQSAPHGVVFLHVTYVPAQEKNFTTAPAISHDGKAHFPREGVFVSNG |
| | | | THWF |
| 313 | S | 1134-1103 | LGQSKRVDFSGKGYHLMSFPQSAPHGVVFLHVTYVPAQEKNFTTAPAISHDGKA |
| | | | HFPREGVFVSNGTHWF |
| 314 | S | 1016-1103 | AEIRASANLAATKMSESVLGQSKRVDFSGKGYHLMSFPQSAPHGVVFLHVTYVP |
| | | | AQEKNFTTAPAISHDGKAHFPREGVFVSNGTHWF |
| 315 | S | 498-514 | QPTNGVGYQPYRVVVLS |
| 316 | S | KKK-480-514 | KKK-SNGVEGFNSYFPLQSYGFQPTNGVGYQPYRVVVLS |
| 317 | S | KKK-464-514 | KKK-FERDISTEIYQAGSTPSNGVEGFNSYFPLQSYGFQPTNGVGYQPYRVVVL |
| | | | S |
| 318 | S | KKK-448-514 | KKK-NYNYLYRLFRKSNLKPFERDISTEIYQAGSTPSNGVEGTNSYFPLQSYGF |
| | | | QPTNGVGYQPYRVVVLS |
| 319 | S | KKK-434-514 | KKK-IAWNSNNLDSKVGGNYNYLYRLFRKSNLKPFERDISTETYQAGSTPSNGV |
| | | | EGFNSYFPLQSYGFQPTNGVGYQPYRVVVLS |
| 320 | S | 1086-1121 | KAHFPREGVFVSNGTHWFVTQRNFYEPQIITTDNTF |
| 321 | S | 1164-1183 | VDLGDISGINASVVNIQKEI |
| 322 | S | KKK-1144-1183 | KKK-ELDSFKEELDKYFKNHTSPDVDLGDISGINASVVNIQKEI |
| 323 | S | KKK-1124-1183 | KKK-GNSDVVIGIVNNTVYDPLQPELDSFKEELDKYFKNHTSPDVDLGDISGIN |
| | | | ASVVNIQKEI |
| 324 | S | KKK-1104-1183 | KKK-VTQRNFYEPQLITTDNTFVSGNSDVVIGIVNNTVYDPLQPELDSFKEELD |
| | | | KYFKNHTSPDVDLGDISGINASVVNIWKEI |
| 325 | S | 1166-1203 | LGDISGINASVVNIQKEIDRLNEVAKNLNESLIDLQEL |
| 326 | S | 1213-1233 | PWYIWLGFIAGLIAIVMVTIM |
| 327 | S | 1195-1233 | ESLIDLQELGKYEQYIKWPWYIWLGFIAGLIAIVMVTIM |
| 352 | S | KK-1191-1234 | KK-KNLNESLIDLQRLGKYEQYIKWPWYIWLGFIAGLIAIVMVTIML |
| 353 | S | KKK-1251-1273 | KKK-GSSSKFDEDDSEPVLKGVKLHYT |
| 354 | S | KKK-1181-1223 | KKK-KEIDRLNEVAKNLNESLIDLQELGKYEQYIKWPWYIWLGFIAG |
| 328 | Orf9b | 34-50 | QNNVGPKVYPIILRLGS |
| 329 | Orf9b | 17-50 | PQIQLAVTRMENAVGRDQNNVGPKVYPIILRLGS |
| 330 | Orf9b | KKK-1-50 | KKK-MDPKISEMHPALRLVDPQIQLAVTRMENAVGRDQNNVGPKVYPIILRLGS |
| 331 | Orf9b | KKK-34-68 | KKK-QNNVGPKVYPIILRLGSPLSLNMARKTLNSLEDKA |
| 332 | Orf9b | 82-97 | ATTEELPDEFVVVTVK |
| 333 | OrF9b | 66-97 | DKAFQLTPIAVQMTKLATTEELPDEFVVVTVK |
| 334 | Orf9b | KKK-51-97 | KKK-PLSLNMARKTLNSLEDKAFQLTPIAVQMTKLATTEELPDEFVVVTVK |

* The cysteine residues were replaced by serine that are underlined.

TABLE 14
N-linked glycan structures of S-RBD-sFc

| N-linked | | N-linked | |
|---|---|---|---|
| glycan | Symbol | glycan | Symbol |
| G0F-N | [symbol] | G1F | [symbol] |
| G0F | [symbol] | G2F | [symbol] |
| Man5 | [symbol] | G1F + N | [symbol] |
| G0F + N | [symbol] | G2F + N | [symbol] |
| A4G0F | [symbol] | A3G3F | [symbol] |

TABLE 15
O-linked glycan structures of S-RBD-sFc

| O-linked | | O-linked | |
|---|---|---|---|
| glycan | Symbol | glycan | Symbol |
| GalNAC | [symbol] | GalNAc-3GnG | [symbol] |
| GalNAc-3G | [symbol] | GalNAc-6Gn | [symbol] |
| GalNAC-3SG | [symbol] | GalNAC-6S-3SG | [symbol] |

TABLE 16
N-linked glycan structures of ACE2-ECD-sFc

| N-linked | | N-linked | | N-linked | |
|---|---|---|---|---|---|
| glycan | Symbol | glycan | Symbol | glycan | Symbol |
| G0-N | [symbol] | G0F − N | [symbol] | AAG0F | [symbol] |
| G0 | [symbol] | G0F | [symbol] | A4G1F | [symbol] |
| ManS | [symbol] | G0F + N | [symbol] | A4G2F | [symbol] |
| A4G4 | [symbol] | G1F | [symbol] | A4G3F | [symbol] |
| G2F | [symbol] | G1F + N | [symbol] | A4G4F | [symbol] |
| G2F + N | [symbol] | A3G3F | [symbol] | | |

TABLE 17
O-linked glycan structures of ACE2-ECD-sFc

| O-linked | | O-linked | |
|---|---|---|---|
| glycan | Symbol | glycan | Symbol |
| GalNAC | [symbol] | GalNAc-6Gn | [symbol] |
| GalNAC-3G | [symbol] | GalNAc-3GnG | [symbol] |
| GalNAc-3SG | [symbol] | GalNAc-6S-3GnG | [symbol] |
| GalNAC-6S-3SG | [symbol] | GalNAC-6GnG-3SG | [symbol] |

TABLE 18
Specificity assessment of UBI SARS-CoV-2 ELISA Performance Characteristics:
Lack of cross-reactivities to other viral infections

| UBI ® SARS-CoV-2 ELiSA | HL63(+) | HKU1(+) | HIV(+) | HCV(+) | HBV(+) | NHP | Total |
|---|---|---|---|---|---|---|---|
| Positive | 0 | 0 | 0 | 0 | 0 | 0 | 0 |
| Negative | 2 | 1 | 51 | 92 | 360 | 416 | 922 |
| Total Results | 2 | 1 | 51 | 92 | 360 | 416 | 922 |

TABLE 19
Specificity Assessment based on data collected from ″Non-COVID-
19″ individuals in the US, Taiwan, and China

| | | |
|---|---|---|
| Normal human with testing | Plate 1 | Mean NRC 0.068 |
| conducted at UBI Hauppauge, | | Cut-off 0.268 |
| NY (plasma) | Plate 2 | Mean NRC 0.066 |
| n = 197 | | Cut-off 0.266 |
| (Mean ± SD = 0.066 ± 0.016) | Plate 3 | Mean NRC 0.068 |
| | | Cut-off 0.268 |
| | Plate 4 | Mean NRC 0.064 |
| | | Cut-off 0.264 |
| Employee health checkup | Plate 1 | Mean NRC 0.055 |
| samples with testing conducted | | Cut-off 0.255 |
| at ChuBei, Taiwan (plasma) | | |
| n = 90 | | |
| (Mean ± SD = 0.057 ± 0.014) | | |
| Normal human samples with | Plate 1 | Mean NRC 0.063 |
| testing conducted at NTU | | Cut-off 0.263 |
| Hospital, Taiwan (serum) | Plate 2 | Mean NRC 0.062 |
| n = 17 | | Cut-off 0.262 |
| (Mean ± SD = 0.053 ± 0.006) | | |
| Healthy Control samples with | Plate 1 | Mean NRC 0.087 |
| testing conducted in China | | Cut-off 0.287 |
| (plasma) | Plate 2 | Mean NRC 0.092 |
| n = 12 | | Cut-off 0.292 |
| (Mean ± SD = 0.083 ± 0.010) | | |
| Employee health-check samples | Plate 1 | Mean NRC 0.055 |
| with testing conducted at | | Cut-off 0.255 |
| CurieMed Laboratory at | | |
| HsinChu, Taiwan (serum) | | |
| n = 92 | | |
| (Mean ± SD = 0.052 ± 0.007) | | |
| Health Check-up samples with | Plate 1 | Mean NRC 0.058 |
| testing conducted at CurieMed | | Cut-off 0.2S8 |
| Laboratory at HsinChu, Taiwan | | |
| (serum) | | |
| n = 8 | | |
| (Mean ± SD = 0.067 ± 0.007) | | |
| Individuals with other viral | Plate 1 | Mean NRC 0.055 |
| infection (plasma): | | Cut-off 0.255 |
| HIV(+) samples with testing | | |
| conducted at UBIA (UBIA), | | |
| ChuBei, Taiwan | | |
| n = 40 | | |
| (Mean ± SD = 0.058 ± 0.008) | | |
| Individuals with other viral | Plate 1 | Mean NRC 0.087 |
| infection (plasma): | | Cut-off 0.287 |
| HIV(+) samples with testing | Plate 2 | Mean NRC 0.092 |
| conducted in China | | Cut-off 0.292 |
| n = 11 | | |
| (Mean SD 0.079 ± 0.012) | | |
| Individuals with other viral | Plate 1 | Mean NRC 0.060 |
| infection (serum): | | Cut-off 0.260 |
| HCV(+) samples with testing | | |
| conducted at CurieMed | | |
| Laboratory at HsinChu, Taiwan | | |
| n = 92 | | |
| (Mean ± SD = 0.050 ± 0.006) | | |
| Individuals with other viral | Plate 1 | Mean NRC 0.063 |
| infection (serum): | | Cut-off 0.263 |
| HBV(+) samples conducted at | Plate 2 | Mean NRC 0.061 |
| CurieMed Laboratory at Hsin | | Cut-off 0.261 |
| Chu, Taiwan | Plate 3 | Mean NRC 0.059 |
| n = 360 | | Cut-off 0.259 |
| (Mean ± SD = 0.058 ± 0.020) | Plate 4 | Mean NRC 0.046 |
| | | Cut-off 0.246 |

| | |
|---|---|
| All studies for known | Mean NRC ± SD from |
| NON-COVID-19 individuals | All Test Plate Runs = |
| Total: n = 919 | 0.065 ± 0.014 |
| (Mean ± SD = 0.063 ± 0.012) | |

TABLE 20
Sensitivity assessment of anti-SARS-CoV-2 IgG detection with UBI SARS-CoV-2 EUSA

| | UBI ® SARS-CoV-2 ELISA | | PCR | | Total |
|---|---|---|---|---|---|
| Blood sample collection | Positive | Negative | Positive | Negative | Results |
| <10 days post onset of symptoms | 0 | 10 | 10 | 0 | 10 |
| >10 days Hospitalization | 23 | 0 | 23 | 0 | 23 |
| Patients on day of discharge | 5 | 0 | 5 | 0 | 5 |
| >14 days upon return visit to | 8 | 0 | 8 | 0 | 8 |
| hospital after discharge | | | | | |
| Total | 36 | 10 | 36 | 0 | 46 |

Performance Characteristics: Sensitivity in PCR-confirmed COVID-19 hospitalized patients:
Relative Sensitivity (<10 days post onset of symptoms) = 0/10 = 0%
Relative Sensitivity (>10 days post onset of symptoms) = 23/23 = 100%
Relative Sensitivity (day of discharge from the hospital) = 5/5 = 100%
Overall Sensitivity (All 46 samples) = 36/46 = 78.2%
Accuracy for positive predictive value (for those >10 days post onset of symptoms) = 36/36 = 100%

TABLE 21
Study 1: Performance Characteristics: Sensitivity and Specificity based on COVID-19
samples collected 10 days after onset of symptoms

| | UBI ® SARS-CoV-2 ELISA | | PCR | | Total |
|---|---|---|---|---|---|
| Time of Blood sample collection | Positive | Negative | Positive | Negative | Results |
| >10 days onset of symptoms | 23 | 0 | 23 | 0 | 23 |
| Patients on day of hospital | 5 | 0 | 5 | 0 | 5 |
| discharge | | | | | |
| 14 days return visit after hospital | 8 | 0 | 8 | 0 | 8 |
| discharge | | | | | |
| Donors not exposed to SARS-CoV-2 | 0 | 922 | 0 | 0 | 922 |
| Total | 36 | 922 | 36 | 0 | 958 |

Relative Sensitivity (>10 days onset of symptoms): 100%
Overall Sensitivity including those at the onset of symptoms (from 46 different individuals): 78.2%
Relative Specificity: 100%
Accuracy for positive predictive value for patients enrolled in the hospital and 10 days post symptom onset = 36/(36 + 0) = 100%
Accuracy for negative predictive value = 922/(0 + 922) = 100%

TABLE 22
Study 2: Anti-SARS-CoV-2 IgG detection using UBI ® SARS-CoV-2
ELISA with serum/plasma samples from COVID-19 patients in Taiwan

| | | Run | | | | | | |
|---|---|---|---|---|---|---|---|---|
| | | Cut-off | Test | | | Retest | | |
| | | Days Post | 0.264 | | | 0.259 | | |
| | Patient ID | symptom | OD450 | S/C | | OD450 | S/C | |
| ID | Detailed | onset | nm | ratio | Result | nm | ratio | Result |
| 1 | 23437830 | Day 07 | 0.074 | 0.281 | Negative | 0.075 | 0.289 | Negative |
| | | Day 20 | 0.555 | 2.103 | Positive | 0.604 | 2.332 | Positive |
| | | Day 23 | 1.579 | 5.979 | Positive | 1.551 | 5.986 | Positive |
| 2 | 23438469 | Day 02 | 0.103 | 0.388 | Negative | 0.096 | 0.372 | Negative |
| | | Day 10 | 2.743 | 10.392 | Positive | 2.588 | 9.993 | Positive |
| | | Day 14 | 2.436 | 9.228 | Positive | 2.474 | 9.554 | Positive |
| | | Day 18 | 2.557 | 9.684 | Positive | 2.516 | 9.712 | Positive |
| | | Day 29 | 2.668 | 10.105 | Positive | 2.665 | 10.288 | Positive |
| 3 | 23441330 | Day 16 | 0.695 | 2.634 | Positive | 0.728 | 2.811 | Positive |
| | | Day 20 | 1.067 | 4.041 | Positive | 1.210 | 4.671 | Positive |
| | | Day 24 | 1.305 | 4.943 | Positive | 1.575 | 6.082 | Positive |
| | | Day 34 | 1.330 | 5.038 | Positive | 1.526 | 5.891 | Positive |
| 4 | 23442118 | Day 08 | 0.091 | 0.345 | Negative | 0.081 | 0.314 | Negative |
| | | Day 17 | 0.695 | 2.634 | Positive | 0.728 | 2.811 | Positive |
| 5 | 23445222 | Day 12 | 1.357 | 5.138 | Positive | 1.548 | 5.975 | Positive |
| | | Day 16 | 2.306 | 8.736 | Positive | 2.295 | 8.861 | Positive |
| | | Day 20 | 2.686 | 10.173 | Positive | 2.669 | 10.306 | Positive |
| | | Day 28 | 2.547 | 9.647 | Positive | 2.566 | 9.906 | Positive |
| 6 | 23445031 | Day 06 | 0.056 | 0.212 | Negative | 0.057 | 0.220 | Negative |
| | | Day 22 | 2.725 | 10.323 | Positive | 2.558 | 9.876 | Positive |
| 7 | 23448543 | Day 09 | 0.063 | 0.238 | Negative | 0.067 | 0.258 | Negative |
| | | Day 17 | 0.523 | 1.983 | Positive | 0.547 | 2.113 | Positive |
| 8 | 23447053 | Day 18 | 1.953 | 7.397 | Positive | 1.976 | 7.629 | Positive |
| | | Day 27 | 0.509 | 1.927 | Positive | 0.528 | 2.037 | Positive |
| 9 | 23454829 | Day 07 | 0.098 | 0.372 | Negative | 0.108 | 0.416 | Negative |
| | | Day 15 | 0.748 | 2.834 | Positive | 0.715 | 2.762 | Positive |
| 10 | 23454816 | Day 08 | 0.905 | 3.428 | Positive | 0.996 | 3.844 | Positive |
| | | Day 16 | 2.350 | 8.900 | Positive | 2.293 | 8.854 | Positive |
| 11 | 23457072 | Day 02 | 0.447 | 1.695 | Positive | 0.398 | 1.535 | Positive |
| 12 | 23454638 | Day 14 | 2.906 | 11.009 | Positive | 2.951 | 11.395 | Positive |
| 13 | 23462088 | Day 03 | 0.069 | 0.263 | Negative | 0.076 | 0.295 | Negative |
| | | Day 08 | 2.481 | 9.397 | Positive | 2.455 | 9.478 | Positive |
| 14 | 19766019 | Day 15 | 2.977 | 11.277 | Positive | 2.883 | 11.132 | Positive |
| | | Day 19 | 0.300 | 1.137 | Positive | 0.298 | 1.150 | Positive |
| 15 | 23461598 | Day 14 | 0.607 | 2.300 | Positive | 0.768 | 2.964 | Positive |
| 16 | 23453229 | Day 18 | 2.650 | 10.036 | Positive | 2.683 | 10.359 | Positive |
| 17 | 23451993 | Day 22 | 1.429 | 5.412 | Positive | 1.285 | 4.961 | Positive |

TABLE 23
Study 2: Sensitivity assessment with UBI ® SARS-CoV-2 ELISA

| | UBI ® SARS-CoV-2 ELISA | | PCR | | Total |
|---|---|---|---|---|---|
| Blood Sample Collection | Positive | Negative | Positive | Negative | Results |
| <7 days post onset of symptoms | 1 | 3 | 4 | 0 | 4 |
| 7-14 days post onset of symptoms | 7 | 4 | 11 | 0 | 11 |
| >14 days post onset of symptoms | 22 | 0 | 22 | 0 | 22 |
| Total | 30 | 7 | 37 | 0 | 37 |

Performance Characteristics: Sensitivity in PCR-confirmed COVID-19 hospitalized patients:
Relative Sensitivity (<7 days post onset of symptoms) = 1/4 = 25%
Relative Sensitivity (7-14 days post onset of symptoms) = 7/11 = 63.6%
Relative Sensitivity (>14 days post onset of symptoms) = 22/22 = 100%
Overall Sensitivity (All 37 samples) = 30/37 = 81.1%
Accuracy for positive predictive value (>14 days post onset of symptoms) = 22/22 = 100%

TABLE 24
Positive Agreement by Days Post-Symptom Onset

| | | UBI ® SARS-CoV-2 ELISA | | | |
|---|---|---|---|---|---|
| Days Post | | Number | Number | Positive | |
| Symptom | Number | IgG | IgG | Percent | 95% Confidence |
| Onset | Tested | Positive | Negative | Agreement | Interval |
| 0-7 days | 13 | 5 | 8 | 38.5% | (17.7%, 64.5%) |
| 8-14 days | 39 | 31 | 8 | 79.5% | (64.5%, 89.2%) |
| >15 days | 53 | 53 | 0 | 100.0% | (93.2%, 100%) |

TABLE 25
Negative Percent Agreement

| | UBI ® SARS-CoV-2 ELISA | | | |
|---|---|---|---|---|
| | | | Negative | 95% |
| Number | | | Percent | Confidence |
| Tested | Negative | Positive | Agreement | interval |
| 154 | 154 | 0 | 100% | 97.6%-100.0% |

TABLE 26
Summary results of Independent evaluation

| | | Comparator Method | | | |
|---|---|---|---|---|---|
| UBI SARS-CoV-2 | | Positive | Negative | Negative, | |
| ELISA | | (IgG+) | (IgG−) | HIV+ | Total |
| Positive | IgG+ | 52 | 0 | 0 | 52 |
| Negative | IgG− | 6 | 87 | 10 | 103 |

| | | | | |
|---|---|---|---|---|
| Total | 58 | 87 | 10 | 155 |

TABLE 27
Summary statistics of independent evaluation

| Measure | Estimate | 95% Cl |
|---|---|---|
| IgG Sensitivity | 89.7% (52/58) | (79.2%; 95.2%) |
| IgG Specificity | 100% (97/97) | (96.2%; 100%) |
| Combined PPV for prevalence = | 100% | (52.3%; 100%) |
| 5.0% | | |
| Combined NPV for prevalence = | 99.5% | (98.9%; 99.7%) |
| 5.0% | | |
| Cross-reactivity with HIV+ | 0.0% (0/10), | |
| | not detected | |

TABLE 28
Immunization schedule of the RBD-sFc designer proteins
into Guinea pigs

| | | # of | Immunization dose | |
|---|---|---|---|---|
| Group | Immunogen | Animals/ | volume/Immunization | |
| # | in ISA50V | group | site | Schedule |
| G1 | S-RBD-sFc | 5 | Prime: 100 μg | 0, 3, 5 wpi |
| | | | Boost: 50 μg | |
| G2 | S-RBDa-sFc | 5 | Prime: 100 μg | 0, 3, 5 wpi |
| | | | Boost: 50 μg | |
| G3 | S-RBD-Fc | 5 | Prime: 100 μg | 0, 3, 5 wpi |
| | | | Boost: 50 μg | |

TABLE 29
Titers of Neutralizing Antibodies in Immune Sera A ssessed by CPE Assay

| | | | | Titers of Neutralizing Antibodies in immune | | | | |
|---|---|---|---|---|---|---|---|---|
| | | # of | | sera by CPE assay at 0, 3, and 5 wpi | | | | |
| Group | Immunogen | Animals/ | Immunization | 0 wpi | | 3 wpi | | 5 wpi |
| # | in ISA50V | group | dose, IM | Kexin* | Sinica* | Kexin* | Sinica* | Sinica* |
| G1 | S-RBD-sFc | 5 | Prime: 100 μg | <8 | <4 | >256 | 512-1024 | >8192 |
| | (SEQ ID NO: 235) | | Boost: 50 μg | | | >256 | | |
| G2 | S-RBDa-sFc | 5 | Prime: 100 μg | <8 | <4 | 192 | 128 | 4096 |
| | (SEQ ID NO: 236) | | Boost: 50 μg | | | 128 | | |
| G3 | S-RBD-Fc | 5 | Prime: 100 μg | <8 | <4 | 192 | 256 | 4096 |
| | (SEQ ID NO: 355) | | Boost: 50 μg | | | 256 | | |

*CPE assay conducted at Kexin Laboratory in Beijing and Sinica Lab in Taipei independently

TABLE 30
Size Exclusion Chromatography of S-RBD-sFc (pH from 5.7 to 7.0) at 37 °C for 24 hours

| | | HMW-1 | HMW-1’ | HMW-2 | S-RBD-sFc |
|---|---|---|---|---|---|
| Sample | Condition | (%) | (%) | (%) | (%) |
| Control | 35° C. for 24 hrs | 5.0 | — | 1.0 | 94.0 |
| pH 5.7 | 35° C. for 24 hrs | 0.2 | — | 0.2 | 99.6 |
| pH 5.9 | 35° C. for 24 hrs | 0.1 | — | 0.0 | 99.9 |
| pH 6.1 | 35° C. for 24 hrs | 0.1 | — | 0.0 | 99.9 |
| pH 6.2. | 35° C. for 24 hrs | 0.1 | — | 0.0 | 99.9 |
| pH 6.4 | 35° C. for 24 hrs | 0.0 | — | 0.0 | 99.9 |
| pH 6.7 | 35° C. for 24 hrs | 0.0 | — | 0.0 | 100 |
| pH 7.0 | 35° C. for 24 hrs | 0.0 | — | 0.0 | 100 |
| Control | UV for 24 hrs | 2.6 | — | 8.7 | 88.7 |
| PH 5.7 | UV for 24 hrs | 0.0 | — | 1.4 | 98.6 |
| pH 5.9 | UV for 24 hrs | 0.0 | — | 1.3 | 98.7 |
| pH 6.1 | UV for 24 hrs | 0.0 | — | 1.4 | 98.6 |
| pH 6.2 | UV for 24 hrs | 0.0 | — | 1.4 | 98.6 |
| pH 6.4 | UV for 24 hrs | 0.0 | — | 1.6 | 98.5 |
| pH 6.7 | UV for 24 hrs | 0.0 | — | 1.8 | 98.2 |
| pH 7.0 | UV for 24 hrs | 0.0 | — | 2 2 | 97.8 |
| Control | 50° C. for 1 hr | 11.7 | 46.7 | 12.9 | 28.8 |
| 25 mM Arg | 50° C. for 1 hr | 9.3 | 46.1 | 13.3 | 31.4 |
| 50 mM Arg | 50° C. for 1 hr | 7.7 | 45.1 | 13.8 | 33.4 |
| 100 mM Arg | 50° C. for 1 hr | 5.2 | 44.0 | 14.5 | 36.3 |
| 25 mM Sucrose | 50° C. for 1 hr | 9.9 | 45.7 | 13.0 | 31.4 |
| 50 mM Sucrose | 50° C. for 1 hr | 9.2 | 46.2 | 13.2 | 31.4 |
| 100 mM Sucrose | 50° C. for 1 hr | 7.3 | 45.5 | 13.7 | 33.6 |
| 5% Glycerol | 50° C. for 1 hr | 6.7 | 44.8 | 13.6 | 34.9 |
| 10% Glycerol | 50° C. for 1 hr | 3.7 | 42.2 | 14.4 | 39.8 |
| 15% Glycerol | 50° C. for 1 hr | 0.0 | 33.4 | 15.1 | 51.5 |
| pH 5.9 | UV for 72 hrs | 0.0 | — | 2.7 | 97.3 |
| pH 5.9 + 25 mM Arg | UV for 72 hrs | 0.1 | — | 5.1 | 94.8 |
| pH 5.9 + 50 mM Arg | UV for 72 hrs | 0.4 | — | 6.1 | 93.6 |
| pH 5.9 + 100 mM Arg | UV for 72 hrs | 1.4 | — | 5.8 | 92.9 |
| pH 5.9 | UV for 72 hrs | 0.0 | — | 2.7 | 37.3 |
| pH 5.9 + 25 mM Sucrose | UV for 72 hrs | 0.0 | — | 3.0 | 97.0 |
| pH 5.9 + 50 mM Sucrose | UV for 72 hrs | 0.0 | — | 2.8 | 97.2 |
| pH 5.9 + 100 mM Sucrose | UV for 72 hrs | 0.0 | — | 2.8 | 97.2 |
| pH 5.9 | UV for 72 hrs | 0.0 | — | 2.7 | 97.3 |
| pH 5.9 + 5% Glycerol | UV for 72 hrs | 0.0 | — | 2.8 | 97.2 |
| pH 5.9 + 10% Glycerol | UV for 72 hrs | 0.0 | — | 2.6 | 97.4 |
| pH 5.9 + 15% Glycerol | UV for 72 hrs | 0.1 | — | 2.8 | 97.0 |

TABLE 31
Summary of pH and excipient selection of Sl-RBD-sFc

| Parameter | Condition | Result |
|---|---|---|
| pH | Histidine buffer | 10 mM histidine reduced thermal and UV induced protein |
| | (pH 5.7~7.0) | aggregation. |
| | | Between pH 5.7~7.0, the lower pH, the higher protein solubility. |
| | | According to the size exclusion chromatography result, pH |
| | | 5.7~6,4 is preferable for storing S1-RBD-sFc. |
| Amino acid | Arginine | Adding arginine mitigated heat induced protein aggregation but |
| | | induce UV induced aggregation instead. |
| | | Arginine effect is concentration dependent. |
| Sugar | Sucrose | Sucrose mitigated heat induced protein aggregation but has no |
| | | impact on UV induced protein aggregation. |
| | | Sucrose effect on heat stress is concentration dependent. |
| Polyl | Glycerol | Glycerol mitigated heat induced protein aggregation but has no |
| | | impact on UV induced protein aggregation. |
| | | Glycerol effect on heat stress is concentration dependent. |
| Surfactant | Polysorbate 80 | Adding polysorbate 80 prevented protein precipitation during |
| | | agitation. |

TABLE 32
Selection of Peptides comprising SARS-CoV-2
Th/CTL epitopes with known MHC I/II binding
for high precision SARS-CoV-2 designer
vaccine

| | Type | | | | |
|---|---|---|---|---|---|
| | of | | SEQ | | |
| Posi- | epi- | Amino acid | ID | | |
| tion | tope | sequence | NO | MHC I | MHC II |
| S₉₅₇₋₉₈₄ | Th/ | KKK- | 345 | HLA- | HLA- |
| | CTL | QALNTLVKQLSS | | A | DRB1 |
| | | NFGAI | | *11:01 | *04:01 |
| | | SSVLNDILSRL | | | |
| | | QALNTLVKQLSS | 41 | | |
| | | NFGAI | | | |
| | | (HLA DRB1*04:01) | | | |
| | | SVLNDILSR | 362 | | |
| | | (HLA-A*11:01; | | | |
| | | 43.38% coverage) | | | |
| S₈₉₁₋₉₁₇ | Th | KKK- | 346 | | HLA- |
| | | GAALQIPFAMQMAYRFN | | | DRA |
| | | GIGVTQNVLY | | | *01:01 |
| | | GAALQIPFAMQMAYRF | 39 | | HLA- |
| | | (HLA-DRA*01:01; | | | DRB1 |
| | | HLA-DRB1*07:01) | | | *07:01 |
| | | MAYRFNGIGVTQNVLY | 40 | | HLA- |
| | | (HLA-DRB1*04:01) | | | DRB1 |
| | | | | | *04:01 |
| N₃₀₅₋₃₃₁ | Th/ | KKK- | 347 | HLA- | II |
| | CTL | AQFAPSASAFFGMS | | B | |
| | | RIGMEVTPSGTWL | | *40:01 | |
| | | AQFAPSASAFFGMS | 363 | | |
| | | RIGM | | | |
| | | (HLA-B*40:01) | | | |
| | | MEVTPSGTWL | 15 | | |
| | | (HLA-B*40:01; | | | |
| | | 77.23% coverage) | | | |
| S₉₉₆₋₁₀₂₈ | Th/ | KKK- | 348 | | |
| | CTL | LITGRLQSLQTYVTQ | | | |
| | | QLIRAAEIRASANLAATK | | | |
| | | LITGRLQSL | 43 | HLA- | |
| | | (HLA-A2) | | A2 | |
| | | QLIRAAEIRASANLAATK | 44 | HLA- | HLA- |
| | | (HLA-DRB1*04:01) | | A | DRB1 |
| | | | | *02:01 | *04:01 |
| | | RLQSLQTYV | 364 | HLA- | |
| | | (HLA-A*02:01, | | A* | |
| | | 69.63%; coverage) | | 31:01 | |
| | | VQIDRLITGR | 365 | | |
| | | (HLA-A*31:01; | | | |
| | | 80.62% coverage) | | | |
| M₈₉₋₁₁₁ | Th/ | KKK- | 361 | HLA- | II |
| | CTL | GLMWLSYFI | | A | |
| | | ASFRLFARTRSMWS | | *02:01 | |
| | | GLMWLSYFI | 366 | | |
| | | (HLA-A*02:01) | | | |
| | | 100% coverage to | | | |
| | | MHC I | | | |
| | | IASFRLFARTRSMWS | 367 | | |
| | | (MHC II) 65%; | | | |
| | | coverage to MHC II | | | |

Bold: MHC I
Underlined: MHC II

TABLE 33
Composition of UB-612 20 μg/mL

| Description | SEQ ID NO | Unit Q′ty/mL | | Function | Quality Grade |
|---|---|---|---|---|---|
| S1-RBD-sFc | 235 | 17.6 | μg | Immunogen | (GMP)¹ |
| Th/CTL peptides | 345 | 0.4 | μg | Immunogen | (GMP)¹ |
| | 346 | 0.4 | μg | | |
| | 347 | 0.4 | μg | | |
| | 348 | 0.4 | μg | | |
| | 361 | 0.4 | μg | | |
| | 66 | 0.4 | μg | | |
| CpG1 | 104 | 0.4 | μg | Adjuvant | GMP |
| ADJU-PHOS | — | 1.6 | mg | Adjuvant | GMP |
| Histidine | — | 4.0 | mM | protein buffer | Ph Eur, JP, USP |
| Histidine HCl•H₂O | — | 6.0 | mM | protein buffer | Ph Eur, BP, JP |
| Arginine HCl | — | 50.0 | mM | protein buffer | Ph Eur, BP, JP, USP |
| TWEEN 80 | — | 0.06% (v/v) | | surfactant/emulsifier | Ph Eur, JP, NF |
| Hydrochloric acid | — | q.s. to pH 5.9-6.0 | | pH adjuster | Ph Eur, BP, JP, NF |
| Sodium chloride | — | 9 | mg | osmotic pressure keeper | Ph Eur, BP, USP |
| 2-Phenoxyethanol | — | 0.5% (v/v) | | preservative | USP-NF |
| Water for injection (q.s.to) | — | 1.0 | mL | solvent | Ph USP |

¹Materials to be used for the Phase 2 and 2/3 clinical trials will be manufactured to cGMP

TABLE 34
Composition of UB-612 60 μg/mL

| Description | SEQ ID NO | Unit Q′ty/mL | | Function | Grade |
|---|---|---|---|---|---|
| S1-RBD-sFc | 235 | 52.8 | μg | Immunogen | (GMP)¹ |
| Th/CTL peptides | 345 | 1.2 | μg | Immunogen | (GMP)¹ |
| | 346 | 1.2 | μg | | |
| | 347 | 1.2 | μg | | |
| | 348 | 1.2 | μg | | |
| | 361 | 1.2 | μg | | |
| | 66 | 1.2 | μg | | |
| CpG1 | 104 | 1.2 | μg | Adjuvant | (GMP)¹ |
| ADJU-PHOS | — | 1.6 | mg | Adjuvant | GMP |
| Histidine | — | 4.0 | mM | protein buffer | Ph Eur, JP, USP |
| Histidine HCl•H₂O | — | 6.0 | mM | protein buffer | Ph Eur, BP, JP |
| Arginine HCl | — | 50.0 | mM | protein buffer | Ph Eur, BP, JP, USP |
| TWEEN 80 | — | 0.06% (v/v) | | surfactant/emulsifier | Ph Eur, JP, NF |
| Hydrochloric acid | — | q.s. to pH 5.9-6.0 | | pH adjuster | Ph Eur, BP, JP, NF |
| Sodium chloride | — | 9 | mg | osmotic pressure keeper | Ph Eur, BP, USP |
| 2-Phenoxy ethanol | — | 0.5% (v/v) | | preservative | USP-NF |
| Water for injection (q.s.to) | — | 1.0 | mL | solvent | Ph USP |

¹Materials to be used for the Phase 2 and 2/3 clinical trials will be manufactured to cGMP

TABLE 35
Composition of UB-612 200 μg/mL

| Description | SEQ ID NO | Unit Q’ty/mL | | Function | Grade |
|---|---|---|---|---|---|
| S1-RBD-sFc | 235 | 176.0 | μg | Immunogen | (GMP)¹ |
| Th/CTL peptides | 345 | 4.0 | μg | Immunogen | (GMP)¹ |
| | 346 | 4.0 | μg | | |
| | 347 | 4.0 | μg | | |
| | 348 | 4.0 | μg | | |
| | 361 | 4.0 | μg | | |
| | 66 | 4.0 | μg | | |
| CpG1 | 104 | 4.0 | μg | Adjuvant | GMP |
| ADJU-PHOS | — | 1.6 | mg | Adjuvant | GMP |
| Histidine | — | 4.0 | mM | protein buffer | Ph Eur, JP, USP |
| Histidine HCl•H₂O | — | 6.0 | mM | protein buffer | Ph Eur, BR, JP |
| Arginine HCl | — | 50.0 | mM | protein buffer | Ph Eur, BP, JP, USP |
| TWEEN 80 | — | 0.06% (v/v) | | surfactant/emulsifier | Ph Eur, JP, NF |
| Hydrochloric acid | — | q.s. to pH 5.9-6.0 | | pH adjuster | Ph Eur, BP, JP, NF |
| Sodium chloride | — | 9 | mg | osmotic pressure keeper | Ph Eur, BP, USP |
| 2-Phenoxyethanol | — | 0.5% (v/v) | | preservative | USP-NF |
| Water for injection (q.s.to) | — | 1.0 | mL | solvent | Ph USP |

¹Materials to be used for the Phase 2 and 2/3 clinical trials will be manufactured to cGMP

TABLE 36
Equivalent to Titers of Neutralizing Antibodies in purified
ACE2-ECD-sFc by CPE Assay

| | | | Titers of Neutralizing |
|---|---|---|---|
| Group | Type of | Dose | Antibodies by CPE assay |
| # | ACE2 | mg/mL | Tested by Sinica |
| G1 | ACE2-ECD | 2.4 | 1024 |
| | (SEQ ID NO: 229) | | |
| G2 | ACE2-ECD-sFc | 2.4 | >8192 |
| | (SEQ ID NO: 237) | | |
| G3 | ACE2-ECD-Fc | 2.4 | 4096 |
| | (SEQ ID NO: 356) | | |
| G3 | ACE2n-ECD-sFc | 2.4 | 8192 |
| | (SEQ ID NO: 238) | | |
| G3 | PBS | 0 | <4, <4, <4 |

1. A fusion protein comprising:
a) an amino acid sequence derived from the receptor binding domain (RBD) of the Spike (S) protein from SARS-CoV-2 selected from the group consisting of SEQ ID NO: 225 and SEQ ID NO: 226;
b) an optional hinge region from an IgG molecule selected from the group consisting of SEQ ID NO: 166-225; and
c) an Fc fragment of an IgG molecule selected from the group consisting of SEQ ID NOs: 231-234,
wherein the amino acid sequence in (a) is covalently linked to the Fc fragment in (c) through directly or through the optional hinge region in (b).

2. The fusion protein according to claim 1, wherein
the Fc fragment in (c) has an amino acid sequence of SEQ ID NO: 232 and
the optional hinge region has an amino acid sequence of SEQ ID NO: 166 or SEQ ID NO: 188.

3. The fusion protein according to claim 1, wherein the fusion protein is selected from the group consisting of: S1-RBD-sFc of SEQ ID NOs: 235, S1-RBDa-sFc of SEQ ID NO: 236, and S1-RBD-Fc of SEQ ID NO: 355.

4. A COVID-19 vaccine composition comprising:
a) the fusion protein according to claim 3; and
b) a pharmaceutically acceptable excipient.

5. The COVID-19 vaccine composition according to claim 4, wherein the fusion protein is S1-RBD-sFc of SEQ ID NO: 235.

6. The COVID-19 vaccine composition according to claim 4 further comprising a Th/CTL peptide.

7. The COVID-19 vaccine composition according to claim 6, wherein the Th/CTL peptide is derived from the SARS-CoV-2 M protein of SEQ ID NO: 1, the SARS-CoV-2 N protein of SEQ ID NO: 6, the SARS-CoV-2 S protein of SEQ ID NO: 20, a pathogen protein, or any combination thereof.

8. The COVID-19 vaccine composition according to claim 7, wherein
a. the Th/CTL peptide derived from the SARS-CoV-2 M protein is SEQ ID NO: 361;
b. the Th/CTL peptide derived from the SARS-CoV-2 N protein is selected from the group consisting of SEQ ID NOs: 9-16, 19, 153-160, 165, 347, 350, 351, and 363;
c. the Th/CTL peptide derived from the SARS-CoV-2 S protein is selected from the group consisting of SEQ ID NOs: 35-36, 39-48, 145-152, 161-164, 345-346, 348, 362, 364, and 365;
d. the Th/CTL peptide derived from a pathogen protein is selected from the group consisting of SEQ ID NOs: 49-100.

9. The COVID-19 vaccine composition according to claim 4, further comprising a mixture of Th/CTL peptides of SEQ ID NOs: 345, 346, 347, 348, 361, and 66.

10. The COVID-19 vaccine composition according to claim 9, wherein each of the Th/CTL peptides are present in the mixture in equal-weight amounts.

11. The COVID-19 vaccine composition according to claim 10, wherein the ration (w:w) of the S1-RBD-sFc protein to the total weight of the mixture of Th/CTL peptides is 88:12.

12. The COVID-19 vaccine composition according to claim 4, wherein the pharmaceutically acceptable excipient is an adjuvant, buffer, surfactant, emulsifier, pH adjuster, saline solution, preservative, solvent, or any combination thereof.

13. The COVID-19 vaccine composition according to claim 4, wherein the pharmaceutically acceptable excipient is selected from the group consisting of a CpG oligonucleotide, aluminum phosphate, histidine, histidine HCl.H₂O, arginine HCl, polyoxyethylene (20) sorbitan monooleate, hydrochloric acid, sodium chloride, 2-phenoxyethanol, water, and any combination thereof.

14. A COVID-19 vaccine composition comprising:
a. a S1-RBD-sFc protein of SEQ ID NO: 235;
b. a Th/CTL peptide selected from the group consisting of SEQ ID NOs: 9-16, 19, 35-36, 39-100, 145-165, 345-348, 350, 351, 362-365, and any combination thereof;
c. a pharmaceutically acceptable excipient.

15. The COVID-19 vaccine composition according to claim 14, wherein the Th/CTL peptides in (b) is a mixture of SEQ ID NOs: 345, 346, 347, 348, 361, and 66.

16. The COVID-19 vaccine composition according to claim 15, wherein each of the Th/CTL peptides are present in the mixture in equal-weight amounts.

17. The COVID-19 vaccine composition according to claim 16, wherein the ration (w:w) of the S1-RBD-sFc protein to the total weight of the mixture of Th/CTL peptides is 88:12.

18. The COVID-19 vaccine composition according to claim 14, wherein the pharmaceutically acceptable excipient is an adjuvant, buffer, surfactant, emulsifier, pH adjuster, saline solution, preservative, solvent, or any combination thereof.

19. The COVID-19 vaccine composition according to claim 14, wherein the pharmaceutically acceptable excipient is selected from the group consisting of a CpG oligonucleotide, aluminum phosphate, histidine, histidine HCl.H₂O, arginine HCl, polyoxyethylene (20) sorbitan monooleate, hydrochloric acid, sodium chloride, 2-phenoxyethanol, water, and any combination thereof.

20. The COVID-19 vaccine composition according to claim 14, wherein
the Th/CTL peptide is a mixture of SEQ ID NOs: 345, 346, 347, 348, 361, and 66, wherein each peptide is present in the mixture in equal-weight amounts;
the pharmaceutically acceptable excipient is a combination of a CpG1 oligonucleotide, aluminum phosphate, histidine, histidine HCl.H₂O, arginine HCl, polyoxyethylene (20) sorbitan monooleate, hydrochloric acid, sodium chloride, and 2-phenoxyethanol in water.

21. The COVID-19 vaccine composition according to claim 20, wherein the total amount of the S1-RBD-sFc protein of SEQ ID NO: 235 is between about 10 μg to about 200 μg; and
the total amount of the Th/CTL peptides is between about 2μg to about 25 μg.

22. (canceled)

23. (canceled)

24. (canceled)

25. A method for preventing COVID-19 in a subject comprising administering a pharmaceutically effective amount of the vaccine composition according to claim 12 to a subject.

26. The method according to claim 25, wherein the pharmaceutically effective amount of the vaccine composition is administered to the subject in two doses.

27. (canceled)

28. A method for generating antibodies against SARS-CoV-2 comprising administering a pharmaceutically effective amount of the vaccine composition according to claim 14 to a subject.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A method for detecting infection by SARS-CoV-2 comprising:
a) attaching an antigenic peptide selected from the group consisting of SEQ ID NOs: 4-5, 17-18, 23-24, 26, 29-34, 37-38, 259, 261, 263, 265, 266, 270, 281, 308, 321, 322, 323, and 324 and any combination thereof to a solid support,
b) exposing the antigenic peptide attached to the solid support in (a) to a biological sample containing antibodies from a patient, under conditions conducive to binding of the antibody to the peptide, and
c) detecting the presence of antibodies bound to the peptide attached to the solid support in (b).

41. The method according to claim 40, wherein the antigenic peptide of (a) is selected from the group consisting of SEQ ID NOs: 5, 18, 38, 261, 266, 281, 322, and any combination thereof.

42. (canceled)

43. An S-RBD peptide immunogen construct having about 20 or more amino acids, represented by the formulae:
(Th)ₘ-(A)ₙ-(S-RBD B cell epitope peptide)-X
or
(S-RBD B cell epitope peptide)-(A)ₙ-(Th)ₘ-(Th)ₘ-X
or
(Th)ₘ-(A)ₙ-(S-RBD B cell epitope peptide)-(A)ₙ-(Th)ₘ-X
wherein
Th is a heterologous T helper epitope;
A is a heterologous spacer;
(S1-RBD B cell epitope peptide) is a B cell epitope peptide having from 6 to about 35 amino acid residues from S1-RBD (SEQ ID NO: 226) or variants thereof;
X is an α-COOH or α-CONH2 of an amino acid;
m is from 1 to about 4; and
n is from 0 to about 10.

44. The S1-RBD peptide immunogen construct according to claim 43, wherein the S1-RBD B cell epitope peptide forms intra-disulfide bond to allow local constraint of the epitope selected from the group consisting of SEQ ID NOs: 23-24, 26-27, and 29-34.

45. The S1-RBD peptide immunogen construct according to claim 43, wherein the heterologous T helper is selected from the group consisting of SEQ ID NOs: 49-100.

46. The S1-RBD peptide immunogen construct according to 43, wherein the S1-RBD B cell epitope peptide is selected from the group consisting of SEQ ID NOs: 23-24, 26-27, 29-34, and 315-319 and the Th epitope is selected from the group consisting of SEQ ID NOs: 49-100.

47. The S1-RBD peptide immunogen construct according to 43, wherein the peptide immunogen construct is selected from the group consisting of SEQ ID NOs: 107-144.

48. An S1-RBD peptide immunogen construct comprising:
a. a B cell epitope comprising from about 6 to about 35 amino acid residues from the S1-RBD sequence of SEQ ID NO: 226;
b. a heterologous T helper epitope comprising an amino acid sequence selected from the group consisting of SEQ ID NOs: 49-100 and any combination thereof; and
c. an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 101), Lys-Lys-Lys-ε-N-Lys (SEQ ID NO: 102), and Pro-Pro-Xaa-Pro-Xaa-Pro (SEQ ID NO: 103), and any combination thereof,
wherein the B cell epitope is covalently linked to the T helper epitope directly or through the optional heterologous spacer.

49. The S1-RBD peptide immunogen construct according to claim 48, wherein the B cell epitope is selected from the group consisting of SEQ ID NOs: 23-24, 26-27, 29-34, and 315-319.

50. The S1-RBD peptide immunogen construct according to claim 48, wherein the optional heterologous spacer is (α, ε-N)Lys, ε-N-Lys-Lys-Lys-Lys (SEQ ID NO: 101), Lys-Lys-Lys-ε-N-Lys (SEQ ID NO: 102), or Pro-Pro-Xaa-Pro-Xaa-Pro (SEQ ID NO:103), where Xaa is any amino acid.

51. The S1-RBD peptide immunogen construct according to claim 48, wherein the T helper epitope is covalently linked to the amino- or carboxyl-terminus of the B cell epitope.

52. The S1-RBD peptide immunogen construct according to claim 48, wherein the T helper epitope is covalently linked to the amino- or carboxyl- of the B cell epitope through the optional heterologous spacer.

53. A composition comprising the S1-RBD peptide immunogen construct according to claim 43.

54. A pharmaceutical composition comprising:
a. a peptide immunogen construct according to claim 43; and
b. a pharmaceutically acceptable delivery vehicle and/or adjuvant.

55. The pharmaceutical composition according to claim 54, wherein
a. the S1-RBD B cell epitope peptide is selected from the group consisting of SEQ ID NOs: 23-24, 26-27, 29-34, and 315-319;
b. the heterologous T helper epitope is selected from the group consisting of SEQ ID NOs: 49-100; and
c. the heterologous spacer is selected from the group consisting of an amino acid, Lys-, Gly-, Lys-Lys-Lys-, (α, ε-N)Lys, ε-N-Lys-Lys-Lys-Lys (SEQ ID NO: 101), Lys-Lys-Lys-ε-N-Lys (SEQ ID NO: 102), and Pro-Pro-Xaa-Pro-Xaa-Pro (SEQ ID NO: 103), and any combination thereof; and
wherein the S1-RBD peptide immunogen construct is mixed with an CpG oligodeoxynucleotide (ODN) to form a stabilized immunostimulatory complex.

56. The pharmaceutical composition according to claim 54, wherein
a. the S1-RBD peptide immunogen construct is selected from the group consisting of SEQ ID NOs: 107-144; and
wherein the S1-RBD peptide immunogen construct is mixed with an CpG oligodeoxynucleotide (ODN) to form a stabilized immunostimulatory complex.

57. The pharmaceutical composition according to claim 56, wherein the pharmaceutical composition further contains a separate peptide containing an endogenous SARS-CoV-2 Th epitope sequence of SEQ ID NOs: 13, 39-41, 44, 161-165, or any combination thereof.

58. The pharmaceutical composition of 56, wherein the pharmaceutical composition further contains a separate peptide containing an endogenous SARS-CoV-2 CTL epitope sequence of SEQ ID NOs: 9-12, 14-16, 19, 35-36, 42-43, 45-48, 145-160, or any combination thereof.

59. The pharmaceutical composition according to claim 56, wherein the pharmaceutical composition further contains
a. a separate peptide containing an endogenous SARS-CoV-2 Th epitope sequence of SEQ ID NOs: 13, 39-41, 44, 161-165, or any combination thereof; and
b. a separate peptide containing an endogenous SARS-CoV-2 CTL epitope sequence of SEQ ID NOs: 9-12, 14-16, 19, 35-36, 42-43, 45-48, 145-160, or any combination thereof.

60. (canceled)

61. (canceled)

62. (canceled)

63. (canceled)

64. (canceled)

65. (canceled)

66. (canceled)

67. A method of preventing and/or treating COVID-19 in an animal comprising administering the pharmaceutical composition according to claim 54 to the animal.

68. A method of preventing and/or treating COVID-19 in an animal comprising administering the pharmaceutical composition of according to claim 57 to the animal.

69. A method of preventing and/or treating COVID-19 in an animal comprising administering the pharmaceutical composition of according to claim 58 to the animal.

70. A method of preventing and/or treating COVID-19 in an animal comprising administering the pharmaceutical composition of according to claim 59 to the animal.

71. A fusion protein comprising:
a) an amino acid sequence derived from the extracellular domain (ECD) of human receptor ACE2 (ECD-hACE2) selected from the group consisting of SEQ ID NO: 228 and SEQ ID NO: 229
b) an optional hinge region from an IgG molecule selected from the group consisting of SEQ ID NO: 166-225; and
c) an Fc fragment of an IgG molecule selected from the group consisting of SEQ ID NOs: 231-234,
wherein the amino acid sequence in (a) is covalently linked to the Fc fragment in (c) through directly or through the optional hinge region in (b).

72. The fusion protein according to claim 71, wherein
the Fc fragment in (c) has an amino acid sequence of SEQ ID NO: 232 and
the optional hinge region has an amino acid sequence of SEQ ID NO: 166 or SEQ ID NO: 188.

73. The fusion protein according to claim 71, wherein the fusion protein is selected from the group consisting of: ACE2-ECD-sFc of SEQ ID NOs: 237, ACE2-ECDN-sFc of SEQ ID NO: 238, and ACE2-ECD-Fc of SEQ ID NO: 356.


SEQUENCE LISTING (370 sequences)

SEQ ID NO: 1 - protein, 222 aa, Artificial Sequence
  SARS-CoV-2
  PEPTIDE (1)..(222): SARS-CoV-2 membrane glycoprotein (M)
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 2 - protein, 221 aa, Artificial Sequence
  Severe acute respiratory syndrome-related coronavirus
  PEPTIDE (1)..(221): SARS-CoV membrane glycoprotein M
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 3 - protein, 219 aa, Artificial Sequence
  Middle East respiratory syndrome-related coronavirus
  PEPTIDE (1)..(219): MERS-CoV M protein
MSNMTQLTEAQIIAIIKDWNFAWSLIFLLITIVLQYGYPSRSMTVYVFKMFVLWLLWPSSMALSIFSAIYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 4 - protein, 23 aa, Artificial Sequence
  SARS-CoV-2
  PEPTIDE (1)..(23): 2019-nCoV membrane glycoprotein (M) 1-23
MADSNGTITVEELKKLLEQWNLV

SEQ ID NO: 5 - protein, 26 aa, Artificial Sequence
  synthetic peptide
  PEPTIDE (1)..(26): KKK-SARS-CoV-2 membrane glycoprotein (M) 1-23
KKKMADSNGTITVEELKKLLEQWNLV

SEQ ID NO: 6 - protein, 419 aa, Artificial Sequence
  SARS-CoV-2
  PEPTIDE (1)..(419): SARS-CoV-2 nucleocapsid phosphoprotein (N)
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 7 - protein, 422 aa, Artificial Sequence
  Severe acute respiratory syndrome-related coronavirus
  PEPTIDE (1)..(422): SARS-CoV nucleocapsid protein (N)
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 8 - protein, 413 aa, Artificial Sequence
  Middle East respiratory syndrome-related coronavirus
  PEPTIDE (1)..(413): MERS-CoV N protein
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSVSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 9 - protein, 9 aa, Artificial Sequence
  SARS-CoV-2
  PEPTIDE (1)..(9): SARS-CoV-2 N protein CTL epitope 138-146
ALNTPKDHI

SEQ ID NO: 10 - protein, 9 aa, Artificial Sequence
  SARS-CoV-2
  PEPTIDE (1)..(9): SARS-CoV-2 N protein CTL epitope 159-167
LQLPQGTTL

SEQ ID NO: 11 - protein, 9 aa, Artificial Sequence
  SARS-CoV-2
  PEPTIDE (1)..(9): SARS-CoV-2 N protein CTL epitope 219-227
LALLLLDRL

SEQ ID NO: 12 - protein, 9 aa, Artificial Sequence
  SARS-CoV-2
  PEPTIDE (1)..(9): SARS-CoV-2 N protein CTL epitope 222-230
LLLDRLNQL

SEQ ID NO: 13 - protein, 15 aa, Artificial Sequence
  SARS-CoV-2
  PEPTIDE (1)..(15): SARS-CoV-2 N protein Th epitope 305-319
AQFAPSASAFFGMSR

SEQ ID NO: 14 - protein, 9 aa, Artificial Sequence
  SARS-CoV-2
  PEPTIDE (1)..(9): SARS-CoV-2 N protein CTL epitope 316-324
GMSRIGMEV

SEQ ID NO: 15 - protein, 10 aa, Artificial Sequence
  SARS-CoV-2
  PEPTIDE (1)..(10): SARS-CoV-2 N protein CTL epitope 322-331
MEVTPSGTWL

SEQ ID NO: 16 - protein, 9 aa, Artificial Sequence
  SARS-CoV-2
  PEPTIDE (1)..(9): SARS-CoV-2 N protein CTL epitope 351-359
ILLNKHIDA

SEQ ID NO: 17 - protein, 65 aa, Artificial Sequence
  SARS-CoV-2
  PEPTIDE (1)..(65): SARS-CoV-2 nucleocapsid phosphoprotein (N) 355-419
KHIDAYKTFPPTEPKKDKKKKADETQALPQRQKKQQTVTLLPAADLDDFSKQLQQSMSSADSTQA

SEQ ID NO: 18 - protein, 68 aa, Artificial Sequence
  Synthetic peptide
  PEPTIDE (1)..(68): KKK-SARS-CoV-2 nucleocapsid phosphoprotein (N) 355-419
KKKKHIDAYKTFPPTEPKKDKKKKADETQALPQRQKKQQTVTLLPAADLDDFSKQLQQSMSSADSTQA

SEQ ID NO: 19 - protein, 9 aa, Artificial Sequence
  SARS-CoV-2
  PEPTIDE (1)..(9): SARS-CoV-2 N protein CTL epitope 361-369
KTFPPTEPK

SEQ ID NO: 20 - protein, 1273 aa, Artificial Sequence
  SARS-CoV-2
  PEPTIDE (1)..(1273): SARS-CoV-2 surface glycoprotein (S)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 1255 aa, Artificial Sequence
  Severe acute respiratory syndrome-related coronavirus
  PEPTIDE (1)..(1255): SARS-CoV spike glycoprotein (S)
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 22 - protein, 1353 aa, Artificial Sequence
  Middle East respiratory syndrome-related coronavirus
  PEPTIDE (1)..(1353): MERS-CoV S Protein
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 23 - protein, 6 aa, Artificial Sequence
  SARS-CoV-2
  PEPTIDE (1)..(6): SARS-CoV-2 S443-448
SKVGGN

SEQ ID NO: 24 - protein, 6 aa, Artificial Sequence
  Synthetic peptide
  PEPTIDE (1)..(6): SARS-CoV-2 S443-448_mod (S443 to C443, N448 to C448)
CKVGGC

SEQ ID NO: 25 - protein, 6 aa, Artificial Sequence
  Severe acute respiratory syndrome-related coronavirus
  PEPTIDE (1)..(6): SARS-CoV S430-435
ATSTGN

SEQ ID NO: 26 - protein, 30 aa, Artificial Sequence
  SARS-CoV-2
  PEPTIDE (1)..(30): SARS-CoV-2  S480-509
CNGVEGFNCYFPLQSYGFQPTNGVGYQPYR

SEQ ID NO: 27 - protein, 33 aa, Artificial Sequence
  Synthetic peptide
  PEPTIDE (1)..(33): KKK-SARS-CoV-2 S480-509
KKKCNGVEGFNCYFPLQSYGFQPTNGVGYQPYR

SEQ ID NO: 28 - protein, 29 aa, Artificial Sequence
  Severe acute respiratory syndrome-related coronavirus
  PEPTIDE (1)..(29): SARS S468-495
CTPPALNCYWPLNDYGFYTTTGIGYQPYR

SEQ ID NO: 29 - protein, 11 aa, Artificial Sequence
  SARS-CoV-2
  PEPTIDE (1)..(11): SARS-CoV-2 S480-490
CNGVEGFNCYF

SEQ ID NO: 30 - protein, 9 aa, Artificial Sequence
  SARS-CoV-2
  PEPTIDE (1)..(9): SARS-CoV-2 S480-488
CNGVEGFNC

SEQ ID NO: 31 - protein, 13 aa, Artificial Sequence
  SARS-CoV-2
  PEPTIDE (1)..(13): SARS-CoV-2 S496-508
GFQPTNGVGYQPY

SEQ ID NO: 32 - protein, 13 aa, Artificial Sequence
  Synthetic peptide
  SITE (1)..(13): SARS-CoV-2 S496-508_mod (G496 to C496, Y508 to C508)
CFQPTNGVGYQPC

SEQ ID NO: 33 - protein, 10 aa, Artificial Sequence
  SARS-CoV-2
  PEPTIDE (1)..(10): SARS-CoV-2 S496-505
GFQPTNGVGY

SEQ ID NO: 34 - protein, 10 aa, Artificial Sequence
  Synthetic peptide
  PEPTIDE (1)..(10): 2019-nCoV S496-505_mod (G496 to C496, Y505 to C505)
CFQPTNGVGC

SEQ ID NO: 35 - protein, 12 aa, Artificial Sequence
  SARS-CoV-2
  PEPTIDE (1)..(12): SARS-CoV-2 S protein CTL epitope 504-515
GYQPYRVVVLSF

SEQ ID NO: 36 - protein, 8 aa, Artificial Sequence
  SARS-CoV-2
  PEPTIDE (1)..(8): SARS-CoV-2 S protein CTL epitope 539-546
VNFNFNGL

SEQ ID NO: 37 - protein, 55 aa, Artificial Sequence
  SARS-CoV-2
  PEPTIDE (1)..(55): SARS-CoV-2 surface glycoprotein (S) 785-839
VKQIYKTPPIKDFGGFNFSQILPDPSKPSKRSFIEDLLFNKVTLADAGFIKQYGD

SEQ ID NO: 38 - protein, 58 aa, Artificial Sequence
  synthetic peptide
  PEPTIDE (1)..(58): KKK-SARS-CoV-2 surface glycoprotein (S) 785-839
KKKVKQIYKTPPIKDFGGFNFSQILPDPSKPSKRSFIEDLLFNKVTLADAGFIKQYGD

SEQ ID NO: 39 - protein, 16 aa, Artificial Sequence
  SARS-CoV-2
  PEPTIDE (1)..(16): SARS-CoV-2 S protein Th epitope 891-906
GAALQIPFAMQMAYRF

SEQ ID NO: 40 - protein, 16 aa, Artificial Sequence
  SARS-CoV-2
  PEPTIDE (1)..(16): SARS-CoV-2 S protein Th epitope 902-917
MAYRFNGIGVTQNVLY

SEQ ID NO: 41 - protein, 17 aa, Artificial Sequence
  SARS-CoV-2
  PEPTIDE (1)..(17): SARS-CoV-2 S protein Th epitope 957-973
QALNTLVKQLSSNFGAI

SEQ ID NO: 42 - protein, 9 aa, Artificial Sequence
  SARS-CoV-2
  PEPTIDE (1)..(9): SARS-CoV-2  S protein CTL epitope 976-984
VLNDILSRL

SEQ ID NO: 43 - protein, 9 aa, Artificial Sequence
  SARS-CoV-2
  PEPTIDE (1)..(9): SARS-CoV-2 S protein CTL epitope 996-1004
LITGRLQSL

SEQ ID NO: 44 - protein, 18 aa, Artificial Sequence
  SARS-CoV-2
  PEPTIDE (1)..(18): SARS-CoV-2 S protein Th epitope 1011-1028
QLIRAAEIRASANLAATK

SEQ ID NO: 45 - protein, 9 aa, Artificial Sequence
  SARS-CoV-2
  PEPTIDE (1)..(9): SARS-CoV-2 S protein CTL epitope 1060-1068
VVFLHVTYV

SEQ ID NO: 46 - protein, 9 aa, Artificial Sequence
  SARS-CoV-2
  PEPTIDE (1)..(9): SARS-CoV-2 S protein CTL epitope 1185-1193
RLNEVAKNL

SEQ ID NO: 47 - protein, 9 aa, Artificial Sequence
  SARS-CoV-2
  PEPTIDE (1)..(9): SARS-CoV-2 S protein CTL epitope 1192-1200
NLNESLIDL

SEQ ID NO: 48 - protein, 9 aa, Artificial Sequence
  SARS-CoV-2
  PEPTIDE (1)..(9): SARS-CoV-2 S protein CTL epitope 1220-1228
FIAGLIAIV

SEQ ID NO: 49 - protein, 16 aa, Measles virus
  PEPTIDE (1)..(16): MvF Th
DLSDLKGLLLHKLDGL

SEQ ID NO: 50 - protein, 16 aa, Measles virus
  PEPTIDE (1)..(16): MvF Th
EISDEIRLIIIKRIEI

SEQ ID NO: 51 - protein, 16 aa, Measles virus
  PEPTIDE (1)..(16): MvF Th
DVSDVKGVVVHKVDGV

SEQ ID NO: 52 - protein, 16 aa, Measles virus
  PEPTIDE (1)..(16): MvF Th
DFSDFKGFFFHKFDGF

SEQ ID NO: 53 - protein, 16 aa, Measles virus
  PEPTIDE (1)..(16): MvF Th
  SITE (1)..(1): D or E
  SITE (2)..(2): L or I or V or F
  SITE (4)..(4): D or E
  SITE (5)..(5): L or I or V or F
  SITE (6)..(6): K or R
  SITE (8)..(8): L or I or V or F
  SITE (9)..(9): L or I or V or F
  SITE (10)..(10): L or I or V or F
  SITE (12)..(12): K or R
  SITE (13)..(13): L or I or V or F
  SITE (14)..(14): D or E
  SITE (16)..(16): L or I or V or F
XXSXXXGXXXHXXXGX

SEQ ID NO: 54 - protein, 15 aa, Measles virus
  PEPTIDE (1)..(15): MvF1 Th
LSEIKGVIVHRLEGV

SEQ ID NO: 55 - protein, 15 aa, Measles virus
  PEPTIDE (1)..(15): MvF 2 Th
ISEIKGVIVHKIEGI

SEQ ID NO: 56 - protein, 19 aa, Measles virus
  PEPTIDE (1)..(19): MvF3 Th
ISISEIKGVIVHKIEGILF

SEQ ID NO: 57 - protein, 19 aa, Measles virus
  PEPTIDE (1)..(19): MvF 3Th
ISITEIRTVIVTRIETILF

SEQ ID NO: 58 - protein, 19 aa, Measles virus
  PEPTIDE (1)..(19): MvF3 Th
  SITE (4)..(4): S or T
  SITE (7)..(7): K or R
  SITE (8)..(8): G or T
  SITE (12)..(12): H or T
  SITE (13)..(13): K or R
  SITE (16)..(16): G or T
ISIXEIXXVIVXXIETILF

SEQ ID NO: 59 - protein, 22 aa, Measles virus
  PEPTIDE (1)..(22): KKKMvF3 Th
KKKISISEIKGVIVHKIEGILF

SEQ ID NO: 60 - protein, 22 aa, Measles virus
  PEPTIDE (1)..(22): KKKMvF3 Th
KKKISITEIRTVIVTRIETILF

SEQ ID NO: 61 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 62 - protein, 19 aa, Measles virus
  PEPTIDE (1)..(19): MvF4 Th
ISISEIKGVIVHKIETILF

SEQ ID NO: 63 - protein, 19 aa, Measles virus
  PEPTIDE (1)..(19): MvF4 Th
ISITEIRTVIVTRIETILF

SEQ ID NO: 64 - protein, 19 aa, Measles virus
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 65 - protein, 19 aa, Measles virus
  PEPTIDE (1)..(19): MvF5 Th (UBITh1)
ISITEIKGVIVHRIETILF

SEQ ID NO: 66 - protein, 22 aa, Measles virus
  PEPTIDE (1)..(22): KKKMvF5 Th (UBITh1a)
KKKISITEIKGVIVHRIETILF

SEQ ID NO: 67 - protein, 18 aa, Hepatitis B virus
  PEPTIDE (1)..(18): HBsAg1 Th
KKKLFLLTKLLTLPQSLD

SEQ ID NO: 68 - protein, 18 aa, Hepatitis B virus
  PEPTIDE (1)..(18): HBsAg1 Th
RRRIKIITRIITIPLSIR

SEQ ID NO: 69 - protein, 18 aa, Hepatitis B virus
  PEPTIDE (1)..(18): HBsAg1 Th
KKKVRVVTKVVTVPISVD

SEQ ID NO: 70 - protein, 18 aa, Hepatitis B virus
  PEPTIDE (1)..(18): HBsAg1 Th
KKKFFFFTKFFTFPVSFD

SEQ ID NO: 71 - protein, 18 aa, Hepatitis B virus
  PEPTIDE (1)..(18): HBsAg1 Th
KKKLFLLTKLLTLPFSLD

SEQ ID NO: 72 - protein, 18 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 73 - protein, 18 aa, Hepatitis B virus
  PEPTIDE (1)..(18): HBsAg2 Th
KKKIITITRIITIPQSLD

SEQ ID NO: 74 - protein, 18 aa, Hepatitis B virus
  PEPTIDE (1)..(18): HBsAg2 Th
KKKFFLLTRILTIITTID

SEQ ID NO: 75 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 76 - protein, 18 aa, Hepatitis B virus
  PEPTIDE (1)..(18): HBsAg 3 Th
KKKIITITRIITIITTID

SEQ ID NO: 77 - protein, 15 aa, Hepatitis B virus
  PEPTIDE (1)..(15): HBsAg4 Th (UBITh4)
FFLLTRILTIPQSLD

SEQ ID NO: 78 - protein, 18 aa, Hepatitis B virus
  PEPTIDE (1)..(18): KKK-HBsAg Th
KKKFFLLTRILTIPQSLD

SEQ ID NO: 79 - protein, 14 aa, Hepatitis B virus
  PEPTIDE (1)..(14): HBsAg Th
FFLLTRILTIPQSL

SEQ ID NO: 80 - protein, 24 aa, Bordetella pertussis
  PEPTIDE (1)..(24): Bordetella pertussis Th
GAYARCPNGTRALTVAELRGNAEL

SEQ ID NO: 81 - protein, 25 aa, Artificial Sequence
  Cholera Toxin
  PEPTIDE (1)..(25): Cholera Toxin Th
ALNIWDRFDVFCTLGATTGYLKGNS

SEQ ID NO: 82 - protein, 15 aa, Clostridium tetani
  PEPTIDE (1)..(15): Clostridium tetani TT1 Th
QYIKANSKFIGITEL

SEQ ID NO: 83 - protein, 17 aa, Clostridium tetani
  PEPTIDE (1)..(17): Clostridium tetani 1 Th
KKQYIKANSKFIGITEL

SEQ ID NO: 84 - protein, 21 aa, Clostridium tetani
  PEPTIDE (1)..(21): Clostridium tetani  TT2 Th
FNNFTVSFWLRVPKVSASHLE

SEQ ID NO: 85 - protein, 16 aa, Clostridium tetani
  PEPTIDE (1)..(16): Clostridium tetani TT3 Th
KFIIKRYTPNNEIDSF

SEQ ID NO: 86 - protein, 16 aa, Clostridium tetani
  PEPTIDE (1)..(16): Clostridium tetani  TT4 Th
VSIDKFRIFCKALNPK

SEQ ID NO: 87 - protein, 17 aa, Clostridium tetani
  PEPTIDE (1)..(17): Clostridium tetani 2 Th
WVRDIIDDFTNESSQKT

SEQ ID NO: 88 - protein, 23 aa, Artificial Sequence
  Diphtheria bacilli
  PEPTIDE (1)..(23): Diphtheria Th
DSETADNLEKTVAALSILPGHGC

SEQ ID NO: 89 - protein, 19 aa, Artificial Sequence
  Epstein-Barr virus
  PEPTIDE (1)..(19): EBV BHRF1 Th
AGLTLSLLVICSYLFISRG

SEQ ID NO: 90 - protein, 20 aa, Artificial Sequence
  Epstein-Barr virus
  PEPTIDE (1)..(20): EBV EBNA-1 Th
PGPLRESIVCYFMVFLQTHI

SEQ ID NO: 91 - protein, 18 aa, Artificial Sequence
  Epstein-Barr virus
  PEPTIDE (1)..(18): EBV CP Th
VPGLYSPCRAFFNKEELL

SEQ ID NO: 92 - protein, 15 aa, Artificial Sequence
  Epstein-Barr virus
  PEPTIDE (1)..(15): EBV GP340 Th
TGHGARTSTEPTTDY

SEQ ID NO: 93 - protein, 13 aa, Artificial Sequence
  Epstein-Barr virus
  PEPTIDE (1)..(13): EBV BPLF1 Th
KELKRQYEKKLRQ

SEQ ID NO: 94 - protein, 11 aa, Artificial Sequence
  Epstein-Barr virus
  PEPTIDE (1)..(11): EBV EBNA-2 Th
TVFYNIPPMPL

SEQ ID NO: 95 - protein, 14 aa, Human cytomegalovirus
  PEPTIDE (1)..(14): HCMV IE1 Th
DKREMWMACIKELH

SEQ ID NO: 96 - protein, 11 aa, Influenza virus
  PEPTIDE (1)..(11): Influenza Matrix protein 1_1 Th
FVFTLTVPSER

SEQ ID NO: 97 - protein, 15 aa, Influenza virus
  PEPTIDE (1)..(15): Influenza Matrix protein 1_2 Th
SGPLKAEIAQRLEDV

SEQ ID NO: 98 - protein, 9 aa, Influenza virus
  PEPTIDE (1)..(9): Influenza Non-structural protein 1 Th
DRLRRDQKS

SEQ ID NO: 99 - protein, 21 aa, Plasmodium falciparum
  PEPTIDE (1)..(21): Plasmodium falciparum Th
DHEKKHAKMEKASSVFNVVNS

SEQ ID NO: 100 - protein, 17 aa, Schistosoma mansoni
  PEPTIDE (1)..(17): Schistosoma mansoni Th
KWFKTNAPNGVDEKHRH

SEQ ID NO: 101 - protein, 4 aa, Artificial Sequence
  Synthetic peptide
  SITE (1)..(1): epsilon-K
  PEPTIDE (1)..(4): epsilon-K-KKK as a spacer
KKKK

SEQ ID NO: 102 - protein, 4 aa, Artificial Sequence
  Synthetic peptide
  PEPTIDE (1)..(4): KKK-epsilon-K as a spacer
  SITE (4)..(4): epsilon-K
KKKK

SEQ ID NO: 103 - protein, 6 aa, Artificial Sequence
  Synthetic peptide
  PEPTIDE (1)..(6): Flexible Hinge Spacer
PPXPXP

SEQ ID NO: 104 - DNA, 32 bp, Artificial Sequence
  CpG1 oligonucleotide ODN
  misc_feature (1)..(32): CpG1
  misc_feature (1)..(32)
tcgtcgttttgtcgttttgtcgttttgtcgtt

SEQ ID NO: 105 - DNA, 24 bp, Artificial Sequence
  CpG2 oligonucleotide ODN
  misc_feature (1)..(24): CpG2
tcgtcgttttgtcgttttgtcgtt

SEQ ID NO: 106 - DNA, 24 bp, Artificial Sequence
  CpG3 Oligonucleotide ODN
  misc_feature (1)..(24): CpG3
tcgtcgttttgtcgttttgtcgtt

SEQ ID NO: 107 - protein, 34 aa, Artificial Sequence
  Synthetic peptide
  PEPTIDE (1)..(11): SARS-CoV-2 S480-490
  PEPTIDE (12)..(15): KKK-epsilon K as a spacer
  SITE (15)..(15): epsilon K
  PEPTIDE (16)..(34): MvF5 Th (UBITh1)
CNGVEGFNCYFKKKKISITEIKGVIVHRIETILF

SEQ ID NO: 108 - protein, 34 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(34): SARS-CoV-2 S480-490
ISITEIKGVIVHRIETILFKKKKCNGVEGFNCYF

SEQ ID NO: 109 - protein, 36 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(36): SARS-CoV-2 S496-508_mod
ISITEIKGVIVHRIETILFKKKKCFQPTNGVGYQPC

SEQ ID NO: 110 - protein, 36 aa, Artificial Sequence
  Synthetic peptide
  PEPTIDE (1)..(13): SARS-CoV-2 S496-508_mod
  PEPTIDE (14)..(17): KKK-epsilon K as a spacer
  SITE (17)..(17): epsilon K
  PEPTIDE (18)..(36): MvF5 Th (UBITh1)
CFQPTNGVGYQPCKKKKISITEIKGVIVHRIETILF

SEQ ID NO: 111 - protein, 33 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(33): SARS-CoV-2 S496-505_mod
ISITEIKGVIVHRIETILFKKKKCFQPTNGVGC

SEQ ID NO: 112 - protein, 33 aa, Artificial Sequence
  Synthetic peptide
  PEPTIDE (1)..(10): SARS-CoV-2 S496-505_mod
  PEPTIDE (11)..(14): KKK-epsilon K as a spacer
  SITE (14)..(14): epsilon K
  PEPTIDE (15)..(33): MvF5 Th (UBITh1)
CFQPTNGVGCKKKKISITEIKGVIVHRIETILF

SEQ ID NO: 113 - protein, 53 aa, Artificial Sequence
  Synthetic peptide
  PEPTIDE (1)..(30): SARS-CoV-2 S480-509
  PEPTIDE (31)..(34): KKK-epsilon K as a spacer
  SITE (34)..(34): epsilon K
  PEPTIDE (35)..(53): MvF5 Th (UBITh1)
CNGVEGFNCYFPLQSYGFQPTNGVGYQPYRKKKKISITEIKGVIVHRIETILF

SEQ ID NO: 114 - protein, 53 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): SARS-CoV-2 S480-509_mod
ISITEIKGVIVHRIETILFKKKKCNGVEGFNCYFPLQSYGFQPTNGVGYQPYR

SEQ ID NO: 115 - protein, 29 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(29): SARS-CoV-2 S443-448_mod
ISITEIKGVIVHRIETILFKKKKCKVGGC

SEQ ID NO: 116 - protein, 29 aa, Artificial Sequence
  Synthetic peptide
  PEPTIDE (1)..(6): SARS-CoV-2 S443-448_mod
  PEPTIDE (7)..(10): KKK-epsilon K as a spacer
  SITE (10)..(10): epsilon K
  PEPTIDE (11)..(29): MvF5 Th (UBITh1)
CKVGGCKKKKISITEIKGVIVHRIETILF

SEQ ID NO: 117 - protein, 31 aa, Artificial Sequence
  Alpha-Synuclein peptide immunogen construct
  PEPTIDE (1)..(17): Clostridium tetani1 Th
  PEPTIDE (18)..(21): epsilon-K-KKK spacer
  SITE (18)..(18): epsilon-K
  PEPTIDE (22)..(31): SARS-CoV-2 S496-505_mod
KKQYIKANSKFIGITELKKKKCFQPTNGVGC

SEQ ID NO: 118 - protein, 29 aa, Artificial Sequence
  alpha-Synuclein (G111-G132)
  PEPTIDE (1)..(15): MvF1 Th
  PEPTIDE (16)..(19): epsilon-K-KKK spacer
  SITE (16)..(16): epsilon-K
  PEPTIDE (20)..(29): SARS-CoV-2 S496-505_mod
LSEIKGVIVHRLEGVKKKKCFQPTNGVGC

SEQ ID NO: 119 - protein, 38 aa, Artificial Sequence
  Synthetic peptide
  PEPTIDE (1)..(24): Bordetella pertussis Th
  PEPTIDE (25)..(28): epsilon-K-KKK spacer
  SITE (25)..(25): epsilon-K
  PEPTIDE (29)..(38): SARS-CoV-2 S496-505_mod
GAYARCPNGTRALTVAELRGNAELKKKKCFQPTNGVGC

SEQ ID NO: 120 - protein, 31 aa, Artificial Sequence
  Alpha-Synuclein peptide immunogen construct
  PEPTIDE (1)..(17): Clostridium tetani2 Th
  PEPTIDE (18)..(21): epsilon-K-KKK spacer
  SITE (18)..(18): epsilon-K
  PEPTIDE (22)..(31): SARS-CoV-2 S496-505_mod
WVRDIIDDFTNESSQKTKKKKCFQPTNGVGC

SEQ ID NO: 121 - protein, 37 aa, Artificial Sequence
  Alpha-Synuclein peptide immunogen construct
  PEPTIDE (1)..(23): Diphtheria Th
  PEPTIDE (24)..(27): epsilon-K-KKK spacer
  SITE (24)..(24): epsilon-K
  PEPTIDE (28)..(37): SARS-CoV-2 S496-505_mod
DSETADNLEKTVAALSILPGHGCKKKKCFQPTNGVGC

SEQ ID NO: 122 - protein, 35 aa, Artificial Sequence
  Alpha-Synuclein peptide immunogen construct
  PEPTIDE (1)..(21): Plasmodium falciparum Th
  PEPTIDE (22)..(25): epsilon-K-KKK spacer
  SITE (22)..(22): epsilon-K
  PEPTIDE (26)..(35): SARS-CoV-2 S496-505_mod
DHEKKHAKMEKASSVFNVVNSKKKKCFQPTNGVGC

SEQ ID NO: 123 - protein, 31 aa, Artificial Sequence
  Alpha-Synuclein peptide immunogen construct
  PEPTIDE (1)..(17): Schistosoma mansoni Th
  PEPTIDE (18)..(21): epsilon-K-KKK spacer
  SITE (18)..(18): epsilon-K
  PEPTIDE (22)..(31): SARS-CoV-2 S496-505_mod
KWFKTNAPNGVDEKHRHKKKKCFQPTNGVGC

SEQ ID NO: 124 - protein, 39 aa, Artificial Sequence
  Alpha-Synuclein peptide immunogen construct
  PEPTIDE (1)..(25): Cholera Toxin Th
  PEPTIDE (26)..(29): epsilon-K-KKK spacer
  SITE (26)..(26): epsilon-K
  PEPTIDE (30)..(39): SARS-CoV-2 S496-505_mod
ALNIWDRFDVFCTLGATTGYLKGNSKKKKCFQPTNGVGC

SEQ ID NO: 125 - protein, 29 aa, Artificial Sequence
  Alpha-Synuclein peptide immunogen construct
  PEPTIDE (1)..(15): MvF2 Th
  PEPTIDE (16)..(19): epsilon-K-KKK spacer
  SITE (16)..(16): epsilon-K
  PEPTIDE (20)..(29): SARS-CoV-2 S496-505_mod
ISEIKGVIVHKIEGIKKKKCFQPTNGVGC

SEQ ID NO: 126 - protein, 36 aa, Artificial Sequence
  Synthetic peptide
  PEPTIDE (1)..(22): KKKMvF3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
  SITE (23)..(23): epsilon K as a spacer
  PEPTIDE (23)..(26): epsilon-K-KKK spacer
  PEPTIDE (27)..(36): SARS-CoV-2 S496-505_mod
KKKISIXEIXXVIVXXIEXILFKKKKCFQPTNGVGC

SEQ ID NO: 127 - protein, 32 aa, Artificial Sequence
  Synthetic peptide
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
  SITE (19)..(19): epsilon K as a spacer
  PEPTIDE (19)..(22): epsilon-K-KKK spacer
  PEPTIDE (23)..(32): SARS-CoV-2 S496-505_mod
XXXXXXXTXXXTXPXSXXKKKKCFQPTNGVGC

SEQ ID NO: 128 - protein, 33 aa, Artificial Sequence
  Alpha-Synuclein peptide immunogen construct
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
  SITE (20)..(20): epsilon-K
  PEPTIDE (20)..(23): epsilon-K-KKK spacer
  PEPTIDE (24)..(33): SARS-CoV-2 S496-505_mod
ISIXEIXXVIVXXIETILFKKKKCFQPTNGVGC

SEQ ID NO: 129 - protein, 32 aa, Artificial Sequence
  Synthetic peptide
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
  SITE (19)..(19): epsilon K as a spacer
  PEPTIDE (19)..(22): epsilon-K-KKK spacer
  PEPTIDE (23)..(31): SARS-CoV-2 S496-505_mod
KKKXXXXTRIXTIXXXXDKKKKCFQPTNGVGC

SEQ ID NO: 130 - protein, 32 aa, Artificial Sequence
  Alpha-Synuclein peptide immunogen construct
  PEPTIDE (1)..(18): HBsAg3 Th (UBITh2)
  SITE (19)..(19): epsilon-K
  PEPTIDE (19)..(22): epsilon-K-KKK spacer
  PEPTIDE (23)..(32): SARS-CoV-2 S496-505_mod
KKKIITITRIITIITTIDKKKKCFQPTNGVGC

SEQ ID NO: 131 - protein, 25 aa, Artificial Sequence
  Alpha-Synuclein peptide immunogen construct
  PEPTIDE (1)..(11): Influenza MP1_1 Th
  PEPTIDE (12)..(15): epsilon-K-KKK spacer
  SITE (12)..(12): epsilon-K
  PEPTIDE (16)..(25): SARS-CoV-2 S496-505_mod
FVFTLTVPSERKKKKCFQPTNGVGC

SEQ ID NO: 132 - protein, 29 aa, Artificial Sequence
  Alpha-Synuclein peptide immunogen construct
  PEPTIDE (1)..(15): Influenza MP1_2 Th
  PEPTIDE (16)..(19): epsilon-K-KKK spacer
  SITE (16)..(16): epsilon-K
  PEPTIDE (20)..(29): SARS-CoV-2 S496-505_mod
SGPLKAEIAQRLEDVKKKKCFQPTNGVGC

SEQ ID NO: 133 - protein, 23 aa, Artificial Sequence
  Alpha-Synuclein peptide immunogen construct
  PEPTIDE (1)..(9): Influenza NSP1 Th
  PEPTIDE (10)..(13): epsilon-K-KKK spacer
  SITE (10)..(10): epsilon-K
  PEPTIDE (14)..(23): SARS-CoV-2 S496-505_mod
DRLRRDQKSKKKKCFQPTNGVGC

SEQ ID NO: 134 - protein, 33 aa, Artificial Sequence
  Alpha-Synuclein peptide immunogen construct
  PEPTIDE (1)..(19): EBV BHRF1 Th
  PEPTIDE (20)..(23): epsilon-K-KKK spacer
  SITE (20)..(20): epsilon-K
  PEPTIDE (24)..(33): SARS-CoV-2 S496-505_mod
AGLTLSLLVICSYLFISRGKKKKCFQPTNGVGC

SEQ ID NO: 135 - protein, 29 aa, Artificial Sequence
  Alpha-Synuclein peptide immunogen construct
  PEPTIDE (1)..(15): Clostridium tetani TT1 Th
  PEPTIDE (16)..(19): epsilon-K-KKK spacer
  SITE (16)..(16): epsilon-K
  PEPTIDE (20)..(29): SARS-CoV-2 S496-505_mod
QYIKANSKFIGITELKKKKCFQPTNGVGC

SEQ ID NO: 136 - protein, 34 aa, Artificial Sequence
  Alpha-Synuclein peptide immunogen construct
  PEPTIDE (1)..(20): EBV EBNA-1 Th
  PEPTIDE (21)..(24): epsilon-K-KKK spacer
  SITE (21)..(21): epsilon-K
  PEPTIDE (25)..(34): SARS-CoV-2 S496-505_mod
PGPLRESIVCYFMVFLQTHIKKKKCFQPTNGVGC

SEQ ID NO: 137 - protein, 35 aa, Artificial Sequence
  Alpha-Synuclein peptide immunogen construct
  PEPTIDE (1)..(21): Clostridium tetani TT2 Th
  PEPTIDE (22)..(25): epsilon-K-KKK spacer
  SITE (22)..(22): epsilon-K
  PEPTIDE (26)..(35): SARS-CoV-2 S496-505_mod
FNNFTVSFWLRVPKVSASHLEKKKKCFQPTNGVGC

SEQ ID NO: 138 - protein, 30 aa, Artificial Sequence
  Alpha-Synuclein peptide immunogen construct
  PEPTIDE (1)..(16): Clostridium tetani TT3 Th
  PEPTIDE (17)..(20): epsilon-K-KKK spacer
  SITE (17)..(17): epsilon-K
  PEPTIDE (21)..(30): SARS-CoV-2 S496-505_mod
KFIIKRYTPNNEIDSFKKKKCFQPTNGVGC

SEQ ID NO: 139 - protein, 30 aa, Artificial Sequence
  Alpha-Synuclein peptide immunogen construct
  PEPTIDE (1)..(16): Clostridium tetani TT4 Th
  PEPTIDE (17)..(20): epsilon-K-KKK spacer
  SITE (17)..(17): epsilon-K
  PEPTIDE (21)..(30): SARS-CoV-2 S496-505_mod
VSIDKFRIFCKALNPKKKKKCFQPTNGVGC

SEQ ID NO: 140 - protein, 32 aa, Artificial Sequence
  Alpha-Synuclein peptide immunogen construct
  PEPTIDE (1)..(18): EBV CP Th
  PEPTIDE (19)..(22): epsilon-K-KKK spacer
  SITE (19)..(19): epsilon-K
  PEPTIDE (23)..(32): SARS-CoV-2 S496-505_mod
VPGLYSPCRAFFNKEELLKKKKCFQPTNGVGC

SEQ ID NO: 141 - protein, 28 aa, Artificial Sequence
  Alpha-Synuclein peptide immunogen construct
  PEPTIDE (1)..(14): HCMV IE1 Th
  PEPTIDE (15)..(18): epsilon-K-KKK spacer
  SITE (15)..(15): epsilon-K
  PEPTIDE (19)..(28): SARS-CoV-2 S496-505_mod
DKREMWMACIKELHKKKKCFQPTNGVGC

SEQ ID NO: 142 - protein, 29 aa, Artificial Sequence
  Alpha-Synuclein peptide immunogen construct
  PEPTIDE (1)..(15): EBV GP340 Th
  PEPTIDE (16)..(19): epsilon-K-KKK spacer
  SITE (16)..(16): epsilon-K
  PEPTIDE (20)..(29): SARS-CoV-2 S496-505_mod
TGHGARTSTEPTTDYKKKKCFQPTNGVGC

SEQ ID NO: 143 - protein, 27 aa, Artificial Sequence
  Alpha-Synuclein peptide immunogen construct
  PEPTIDE (1)..(13): EBV BPLF1 Th
  PEPTIDE (14)..(17): epsilon-K-KKK spacer
  SITE (14)..(14): epsilon-K
  PEPTIDE (18)..(27): SARS-CoV-2 S496-505_mod
KELKRQYEKKLRQKKKKCFQPTNGVGC

SEQ ID NO: 144 - protein, 25 aa, Artificial Sequence
  Alpha-Synuclein peptide immunogen construct
  PEPTIDE (1)..(11): EBV EBNA-2 Th
  PEPTIDE (12)..(15): epsilon-K-KKK spacer
  SITE (12)..(12): epsilon-K
  PEPTIDE (16)..(25): SARS-CoV-2 S496-505_mod
TVFYNIPPMPLKKKKCFQPTNGVGC

SEQ ID NO: 145 - protein, 12 aa, Artificial Sequence
  synthetic peptide
  PEPTIDE (1)..(3): KKK- linker
  PEPTIDE (4)..(12): SARS-CoV-2 S protein CTL epitope 1060-1068
KKKVVFLHVTYV

SEQ ID NO: 146 - protein, 12 aa, Artificial Sequence
  synthetic peptide
  PEPTIDE (1)..(3): KKK- linker
  PEPTIDE (4)..(12): 2019-nCoV S protein CTL epitope 976-984
KKKVLNDILSRL

SEQ ID NO: 147 - protein, 12 aa, Artificial Sequence
  SARS-CoV-2
  PEPTIDE (1)..(3): KKK- linker
  PEPTIDE (4)..(12): 2019-nCoV S protein CTL epitope 1185-1193
KKKRLNEVAKNL

SEQ ID NO: 148 - protein, 11 aa, Artificial Sequence
  SARS-CoV-2
  PEPTIDE (1)..(3): KKK- linker
  PEPTIDE (4)..(11): 2019-nCoV S protein CTL epitope 539-546
KKKVNFNFNGL

SEQ ID NO: 149 - protein, 12 aa, Artificial Sequence
  synthetic peptide
  PEPTIDE (1)..(3): KKK- linker
  PEPTIDE (4)..(12): 2019-nCoV S protein CTL epitope 1220-1228
KKKFIAGLIAIV

SEQ ID NO: 150 - protein, 12 aa, Artificial Sequence
  synthetic peptide
  PEPTIDE (1)..(3): KKK- linker
  PEPTIDE (4)..(12): 2019-nCoV S protein CTL epitope 996-1004
KKKLITGRLQSL

SEQ ID NO: 151 - protein, 12 aa, Artificial Sequence
  synthetic peptide
  PEPTIDE (1)..(3): KKK- linker
  PEPTIDE (4)..(12): 2019-nCoV S protein CTL epitope 1192-1200
KKKNLNESLIDL

SEQ ID NO: 152 - protein, 15 aa, Artificial Sequence
  synthetic peptide
  PEPTIDE (1)..(3): KKK- linker
  PEPTIDE (4)..(15): 2019-nCoV S protein CTL epitope 504-515
KKKGYQPYRVVVLSF

SEQ ID NO: 153 - protein, 12 aa, Artificial Sequence
  synthetic peptide
  PEPTIDE (1)..(3): KKK- linker
  PEPTIDE (4)..(12): 2019-nCoV N protein CTL epitope 222-230
KKKLLLDRLNQL

SEQ ID NO: 154 - protein, 12 aa, Artificial Sequence
  synthetic peptide
  PEPTIDE (1)..(3): KKK- linker
  PEPTIDE (4)..(12): 2019-nCoV N protein CTL epitope 219-227
KKKLALLLLDRL

SEQ ID NO: 155 - protein, 12 aa, Artificial Sequence
  synthetic peptide
  PEPTIDE (1)..(3): KKK- linker
  PEPTIDE (4)..(12): 2019-nCoV N protein CTL epitope 159-167
KKKLQLPQGTTL

SEQ ID NO: 156 - protein, 12 aa, Artificial Sequence
  synthetic peptide
  PEPTIDE (1)..(3): KKK- linker
  PEPTIDE (4)..(12): 2019-nCoV N protein CTL epitope 316-324
KKKGMSRIGMEV

SEQ ID NO: 157 - protein, 12 aa, Artificial Sequence
  synthetic peptide
  PEPTIDE (1)..(3): KKK- linker
  PEPTIDE (4)..(12): 2019-nCoV N protein CTL epitope 361-369
KKKKTFPPTEPK

SEQ ID NO: 158 - protein, 12 aa, Artificial Sequence
  synthetic peptide
  PEPTIDE (1)..(3): KKK- linker
  PEPTIDE (4)..(12): 2019-nCoV N protein CTL epitope 138-146
KKKALNTPKDHI

SEQ ID NO: 159 - protein, 12 aa, Artificial Sequence
  synthetic peptide
  PEPTIDE (1)..(3): KKK- linker
  PEPTIDE (4)..(12): 2019-nCoV N protein CTL epitope 351-359
KKKILLNKHIDA

SEQ ID NO: 160 - protein, 13 aa, Artificial Sequence
  synthetic peptide
  PEPTIDE (1)..(3): KKK- linker
  PEPTIDE (4)..(13): 2019-nCoV N protein CTL epitope 322-331
KKKMEVTPSGTWL

SEQ ID NO: 161 - protein, 19 aa, Artificial Sequence
  synthetic peptide
  PEPTIDE (1)..(3): KKK- linker
  PEPTIDE (4)..(19): 2019-nCoV S protein Th epitope 891-906
KKKGAALQIPFAMQMAYRF

SEQ ID NO: 162 - protein, 19 aa, Artificial Sequence
  synthetic peptide
  PEPTIDE (1)..(3): KKK- linker
  PEPTIDE (4)..(19): 2019-nCoV S protein Th epitope 902-917
KKKMAYRFNGIGVTQNVLY

SEQ ID NO: 163 - protein, 21 aa, Artificial Sequence
  synthetic peptide
  PEPTIDE (1)..(3): KKK- linker
  PEPTIDE (4)..(21): 2019-nCoV S protein Th epitope 1011-1028
KKKQLIRAAEIRASANLAATK

SEQ ID NO: 164 - protein, 20 aa, Artificial Sequence
  synthetic peptide
  PEPTIDE (1)..(3): KKK- linker
  PEPTIDE (4)..(20): 2019-nCoV S protein Th epitope 957-973
KKKQALNTLVKQLSSNFGAI

SEQ ID NO: 165 - protein, 18 aa, Artificial Sequence
  synthetic peptide
  PEPTIDE (1)..(3): KKK- linker
  PEPTIDE (4)..(18): 2019-nCoV N protein Th epitope 305-319
KKKAQFAPSASAFFGMSR

SEQ ID NO: 166 - protein, 15 aa, Homo sapiens
  PEPTIDE (1)..(15): Wild-type IgG1
EPKSCDKTHTCPPCP

SEQ ID NO: 167 - protein, 15 aa, Artificial Sequence
  Artificial peptide
  PEPTIDE (1)..(15): Mutated IgG1
EPKSXDKTHTXPPXP

SEQ ID NO: 168 - protein, 13 aa, Artificial Sequence
  Artificial peptide
  PEPTIDE (1)..(13): Mutated IgG1
EPKSXDKTHTXPP

SEQ ID NO: 169 - protein, 10 aa, Artificial Sequence
  Artificial peptide
  PEPTIDE (1)..(10): Mutated IgG1
EPKSXDKTHT

SEQ ID NO: 170 - protein, 10 aa, Artificial Sequence
  Artificial peptide
  PEPTIDE (1)..(10): Mutated IgG1
DKTHTXPPXP

SEQ ID NO: 171 - protein, 12 aa, Homo sapiens
  PEPTIDE (1)..(12): Wild-type IgG2
ERKCCVECPPCP

SEQ ID NO: 172 - protein, 12 aa, Artificial Sequence
  Artificial peptide
  PEPTIDE (1)..(12): Mutated IgG2
ERKXXVEXPPXP

SEQ ID NO: 173 - protein, 10 aa, Artificial Sequence
  Artificial peptide
  PEPTIDE (1)..(10): Mutated IgG2
ERKXXVEXPP

SEQ ID NO: 174 - protein, 7 aa, Artificial Sequence
  Artificial peptide
  PEPTIDE (1)..(7): Mutated IgG2
VEXPPXP

SEQ ID NO: 175 - protein, 17 aa, Homo sapiens
  PEPTIDE (1)..(17): Wild-type IgG3
ELKTPLGDTTHTCPRCP

SEQ ID NO: 176 - protein, 17 aa, Artificial Sequence
  Artificial peptide
  PEPTIDE (1)..(17): Mutated IgG3
ELKTPLGDTTHTXPRXP

SEQ ID NO: 177 - protein, 15 aa, Artificial Sequence
  Artificial peptide
  PEPTIDE (1)..(15): Mutated IgG3
ELKTPLGDTTHTXPR

SEQ ID NO: 178 - protein, 12 aa, Artificial Sequence
  Artificial peptide
  PEPTIDE (1)..(12): Mutated IgG3
ELKTPLGDTTHT

SEQ ID NO: 179 - protein, 15 aa, Homo sapiens
  PEPTIDE (1)..(15): Wild-type IgG3
EPKSCDTPPPCPRCP

SEQ ID NO: 180 - protein, 15 aa, Artificial Sequence
  Artificial peptide
  PEPTIDE (1)..(15): Mutated IgG3
EPKSXDTPPPXPRXP

SEQ ID NO: 181 - protein, 13 aa, Artificial Sequence
  Artificial peptide
  PEPTIDE (1)..(13): Mutated IgG3
EPKSXDTPPPXPR

SEQ ID NO: 182 - protein, 10 aa, Artificial Sequence
  Artificial peptide
  PEPTIDE (1)..(10): Mutated IgG3
EPKSXDTPPP

SEQ ID NO: 183 - protein, 10 aa, Artificial Sequence
  Artificial peptide
  PEPTIDE (1)..(10): Mutated IgG3
DTPPPXPRXP

SEQ ID NO: 184 - protein, 12 aa, Homo sapiens
  PEPTIDE (1)..(12): Wild-type IgG4
ESKYGPPCPSCP

SEQ ID NO: 185 - protein, 12 aa, Artificial Sequence
  Artificial peptide
  PEPTIDE (1)..(12): Mutated IgG4
EXKYGPPCPXCP

SEQ ID NO: 186 - protein, 9 aa, Artificial Sequence
  Artificial peptide
  PEPTIDE (1)..(9): Mutated IgG4
EXKYGPPCP

SEQ ID NO: 187 - protein, 10 aa, Artificial Sequence
  Artificial peptide
  PEPTIDE (1)..(10): Mutated IgG4
KYGPPCPXCP

SEQ ID NO: 188 - protein, 15 aa, Artificial Sequence
  Artificial peptide
  PEPTIDE (1)..(15): Mutated Hinge Regions Derived from IgG1
EPKSSDKTHTSPPSP

SEQ ID NO: 189 - protein, 13 aa, Artificial Sequence
  Artificial peptide
  PEPTIDE (1)..(13): Mutated Hinge Regions Derived from IgG1
EPKSSDKTHTSPP

SEQ ID NO: 190 - protein, 14 aa, Artificial Sequence
  Artificial peptide
  PEPTIDE (1)..(14): Mutated Hinge Regions Derived from IgG1
EPKSSDKTHTSPPP

SEQ ID NO: 191 - protein, 10 aa, Artificial Sequence
  Artificial peptide
  PEPTIDE (1)..(10): Mutated Hinge Regions Derived from IgG1
EPKSSDKTHT

SEQ ID NO: 192 - protein, 10 aa, Artificial Sequence
  Artificial peptide
  PEPTIDE (1)..(10): Mutated Hinge Regions Derived from IgG1
DKTHTSPPSP

SEQ ID NO: 193 - protein, 8 aa, Artificial Sequence
  Artificial peptide
  PEPTIDE (1)..(8): Mutated Hinge Regions Derived from IgG1
DKTHTSPP

SEQ ID NO: 194 - protein, 12 aa, Artificial Sequence
  Artificial peptide
  PEPTIDE (1)..(12): Mutated Hinge Regions Derived from IgG1
EPKSDKTHTPPP

SEQ ID NO: 195 - protein, 14 aa, Artificial Sequence
  Artificial peptide
  PEPTIDE (1)..(14): Mutated Hinge Regions Derived from IgG1
EPKSDKTHTSPPSP

SEQ ID NO: 196 - protein, 15 aa, Artificial Sequence
  Artificial peptide
  PEPTIDE (1)..(15): Mutated Hinge Regions Derived from IgG1
EPKSGDKTHTGPPGP

SEQ ID NO: 197 - protein, 13 aa, Artificial Sequence
  Artificial peptide
  PEPTIDE (1)..(13): Mutated Hinge Regions Derived from IgG1
EPKSGDKTHTGPP

SEQ ID NO: 198 - protein, 14 aa, Artificial Sequence
  Artificial peptide
  PEPTIDE (1)..(14): Mutated Hinge Regions Derived from IgG1
EPKSGDKTHTGPPP

SEQ ID NO: 199 - protein, 10 aa, Artificial Sequence
  Artificial peptide
  PEPTIDE (1)..(10): Mutated Hinge Regions Derived from IgG1
EPKSGDKTHT

SEQ ID NO: 200 - protein, 10 aa, Artificial Sequence
  Artificial peptide
  PEPTIDE (1)..(10): Mutated Hinge Regions Derived from IgG1
DKTHTGPPGP

SEQ ID NO: 201 - protein, 8 aa, Artificial Sequence
  Artificial peptide
  PEPTIDE (1)..(8): Mutated Hinge Regions Derived from IgG1
DKTHTGPP

SEQ ID NO: 202 - protein, 14 aa, Artificial Sequence
  Artificial peptide
  PEPTIDE (1)..(14): Mutated Hinge Regions Derived from IgG1
EPKSDKTHTGPPGP

SEQ ID NO: 203 - protein, 15 aa, Artificial Sequence
  Artificial peptide
  PEPTIDE (1)..(15): Mutated Hinge Regions Derived from IgG1
EPKSGDKTHTSPPSP

SEQ ID NO: 204 - protein, 15 aa, Artificial Sequence
  Artificial peptide
  PEPTIDE (1)..(15): Mutated Hinge Regions Derived from IgG1
EPKSGDKTHTGPPSP

SEQ ID NO: 205 - protein, 15 aa, Artificial Sequence
  Artificial peptide
  PEPTIDE (1)..(15): Mutated Hinge Regions Derived from IgG1
EPKSSDKTHTGPPGP

SEQ ID NO: 206 - protein, 15 aa, Artificial Sequence
  Artificial peptide
  PEPTIDE (1)..(15): Mutated Hinge Regions Derived from IgG1
EPKSSDKTHTGPPSP

SEQ ID NO: 207 - protein, 13 aa, Artificial Sequence
  Artificial peptide
  PEPTIDE (1)..(13): Mutated Hinge Regions Derived from IgG1
EPKSSDKTHTGPP

SEQ ID NO: 208 - protein, 13 aa, Artificial Sequence
  Artificial peptide
  PEPTIDE (1)..(13): Mutated Hinge Regions Derived from IgG1
EPKSGDKTHTSPP

SEQ ID NO: 209 - protein, 15 aa, Artificial Sequence
  Artificial peptide
  PEPTIDE (1)..(15): Mutated Hinge Regions Derived from IgG1
EPKSTDKTHTTPPTP

SEQ ID NO: 210 - protein, 13 aa, Artificial Sequence
  Artificial peptide
  PEPTIDE (1)..(13): Mutated Hinge Regions Derived from IgG1
EPKSTDKTHTTPP

SEQ ID NO: 211 - protein, 14 aa, Artificial Sequence
  Artificial peptide
  PEPTIDE (1)..(14): Mutated Hinge Regions Derived from IgG1
EPKSTDKTHTTPPP

SEQ ID NO: 212 - protein, 10 aa, Artificial Sequence
  Artificial peptide
  PEPTIDE (1)..(10): Mutated Hinge Regions Derived from IgG1
EPKSTDKTHT

SEQ ID NO: 213 - protein, 10 aa, Artificial Sequence
  Artificial peptide
  PEPTIDE (1)..(10): Mutated Hinge Regions Derived from IgG1
DKTHTTPPTP

SEQ ID NO: 214 - protein, 8 aa, Artificial Sequence
  Artificial peptide
  PEPTIDE (1)..(8): Mutated Hinge Regions Derived from IgG1
DKTHTTPP

SEQ ID NO: 215 - protein, 14 aa, Artificial Sequence
  Artificial peptide
  PEPTIDE (1)..(14): Mutated Hinge Regions Derived from IgG1
EPKSDKTHTTPPTP

SEQ ID NO: 216 - protein, 15 aa, Artificial Sequence
  Artificial peptide
  PEPTIDE (1)..(15): Mutated Hinge Regions Derived from IgG1
EPKSSDKTHTTPPTP

SEQ ID NO: 217 - protein, 15 aa, Artificial Sequence
  Artificial peptide
  PEPTIDE (1)..(15): Mutated Hinge Regions Derived from IgG1
EPKSSDKTHTSPPTP

SEQ ID NO: 218 - protein, 15 aa, Artificial Sequence
  Artificial peptide
  PEPTIDE (1)..(15): Mutated Hinge Regions Derived from IgG1
EPKSADKTHTLPPMP

SEQ ID NO: 219 - protein, 15 aa, Artificial Sequence
  Artificial peptide
  PEPTIDE (1)..(15): Mutated Hinge Regions Derived from IgG1
EPKSVDKTHTLPPIP

SEQ ID NO: 220 - protein, 15 aa, Artificial Sequence
  Artificial peptide
  PEPTIDE (1)..(15): Mutated Hinge Regions Derived from IgG1
EPKSLDKTHTAPPAP

SEQ ID NO: 221 - protein, 13 aa, Artificial Sequence
  Artificial peptide
  PEPTIDE (1)..(13): Mutated Hinge Regions Derived from IgG1
EPKSVDKTHTAPP

SEQ ID NO: 222 - protein, 13 aa, Artificial Sequence
  Artificial peptide
  PEPTIDE (1)..(13): Mutated Hinge Regions Derived from IgG1
EPKSMDKTHTVPP

SEQ ID NO: 223 - protein, 13 aa, Artificial Sequence
  Artificial peptide
  PEPTIDE (1)..(13): Mutated Hinge Regions Derived from IgG1
EPKSIDKTHTLPP

SEQ ID NO: 224 - protein, 10 aa, Artificial Sequence
  Artificial peptide
  PEPTIDE (1)..(10): Mutated Hinge Regions Derived from IgG1
DKTHTAPPLP

SEQ ID NO: 225 - protein, 10 aa, Artificial Sequence
  Artificial peptide
  PEPTIDE (1)..(10): Mutated Hinge Regions Derived from IgG1
DKTHTVPPLP

SEQ ID NO: 226 - protein, 200 aa, Artificial Sequence
  SARS-CoV-2
  PEPTIDE (1)..(200): S protein RBD (SARS-CoV-2)
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 227 - protein, 200 aa, Artificial Sequence
  SARS-CoV-2
  PEPTIDE (1)..(200): S protein RBDa (SARS-CoV-2)
NITNLCPFGEVFNATRFASVYAWNRKRISNCVADYSVLYNSASFSTFKCYGVSPTKLNDLAFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVAGPKKS

SEQ ID NO: 228 - protein, 805 aa, Homo sapiens [sequence omitted]

SEQ ID NO: 229 - protein, 740 aa, Homo sapiens
  PEPTIDE (1)..(740): ACE2 extra-cellular domain (ECD) (Homo sapiens)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 230 - protein, 740 aa, Homo sapiens
  PEPTIDE (1)..(740): ACE2N extra-cellular domain (ECD) (Homo sapiens)
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 231 - protein, 216 aa, Homo sapiens
  PEPTIDE (1)..(216): Fc peptide (Wild-Type)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 232 - protein, 216 aa, Artificial Sequence
  Artificial peptide
  PEPTIDE (1)..(216): Fc peptide Mut. Glycos. (N->H)
APELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 233 - protein, 216 aa, Artificial Sequence
  Artificial peptide
  PEPTIDE (1)..(216): Fc peptide Mut. Glycos. (N->A)
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 234 - protein, 216 aa, Artificial Sequence
  Artificial peptide
  PEPTIDE (1)..(216): Fc peptide Mut. Glycos. (N->X) X = N,H,A
APELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 235 - protein, 431 aa, Artificial Sequence
  Fusion protein
  PEPTIDE (1)..(431): S-RBD-sFc Fusion protein
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSEPKSSDKTHTSPPSPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 236 - protein, 431 aa, Artificial Sequence
  Fusion protein
  PEPTIDE (1)..(431): S-RBDa-sFc Fusion protein
NITNLCPFGEVFNATRFASVYAWNRKRISNCVADYSVLYNSASFSTFKCYGVSPTKLNDLAFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVAGPKKSEPKSSDKTHTSPPSPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 237 - protein, 971 aa, Artificial Sequence
  Fusion protein
  PEPTIDE (1)..(971): ACE2-ECD-sFc Fusion protein
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTSPPSPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 238 - protein, 971 aa, Artificial Sequence
  Fusion protein
  PEPTIDE (1)..(971): ACE2N-ECD-sFc Fusion protein
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTSPPSPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 239 - DNA, 3846 bp, Artificial Sequence
  SARS-CoV-2
  misc_feature (1)..(3846): S protein (SARS-CoV-2)
atgttcctgctgaccaccaagaggaccatgttcgtgttcctggtgctgctgcccctggtgtcctcccagtgcgtgaacctgaccaccaggacccagctgccccccgcctacaccaactccttcaccaggggcgtgtactaccccgacaaggtgttcaggtcctccgtgctgcactccacccaggacctgttcctgcccttcttctccaacgtgacctggttccacgccatccacgtgtccggcaccaacggcaccaagaggttcgacaaccccgtgctgcccttcaacgacggcgtgtacttcgcctccaccgagaagtccaacatcatcaggggctggatcttcggcaccaccctggactccaagacccagtccctgctgatcgtgaacaacgccaccaacgtggtgatcaaggtgtgcgagttccagttctgcaacgaccccttcctgggcgtgtactaccacaagaacaacaagtcctggatggagtccgagttcagggtgtactcctccgccaacaactgcaccttcgagtacgtgtcccagcccttcctgatggacctggagggcaagcagggcaacttcaagaacctgagggagttcgtgttcaagaacatcgacggctacttcaagatctactccaagcacacccccatcaacctggtgagggacctgccccagggcttctccgccctggagcccctggtggacctgcccatcggcatcaacatcaccaggttccagaccctgctggccctgcacaggtcctacctgacccccggcgactcctcctccggctggaccgccggcgccgccgcctactacgtgggctacctgcagcccaggaccttcctgctgaagtacaacgagaacggcaccatcaccgacgccgtggactgcgccctggaccccctgtccgagaccaagtgcaccctgaagtccttcaccgtggagaagggcatctaccagacctccaacttcagggtgcagcccaccgagtccatcgtgaggttccccaacatcaccaacctgtgccccttcggcgaggtgttcaacgccaccaggttcgcctccgtgtacgcctggaacaggaagaggatctccaactgcgtggccgactactccgtgctgtacaactccgcctccttctccaccttcaagtgctacggcgtgtcccccaccaagctgaacgacctgtgcttcaccaacgtgtacgccgactccttcgtgatcaggggcgacgaggtgaggcagatcgcccccggccagaccggcaagatcgccgactacaactacaagctgcccgacgacttcaccggctgcgtgatcgcctggaactccaacaacctggactccaaggtgggcggcaactacaactacctgtacaggctgttcaggaagtccaacctgaagcccttcgagagggacatctccaccgagatctaccaggccggctccaccccctgcaacggcgtggagggcttcaactgctacttccccctgcagtcctacggcttccagcccaccaacggcgtgggctaccagccctacagggtggtggtgctgtccttcgagctgctgcacgcccccgccaccgtgtgcggccccaagaagtccaccaacctggtgaagaacaagtgcgtgaacttcaacttcaacggcctgaccggcaccggcgtgctgaccgagtccaacaagaagttcctgcccttccagcagttcggcagggacatcgccgacaccaccgacgccgtgagggacccccagaccctggagatcctggacatcaccccctgctccttcggcggcgtgtccgtgatcacccccggcaccaacacctccaaccaggtggccgtgctgtaccaggacgtgaactgcaccgaggtgcccgtggccatccacgccgaccagctgacccccacctggagggtgtactccaccggctccaacgtgttccagaccagggccggctgcctgatcggcgccgagcacgtgaacaactcctacgagtgcgacatccccatcggcgccggcatctgcgcctcctaccagacccagaccaactcccccaggagggccaggtccgtggcctcccagtccatcatcgcctacaccatgtccctgggcgccgagaactccgtggcctactccaacaactccatcgccatccccaccaacttcaccatctccgtgaccaccgagatcctgcccgtgtccatgaccaagacctccgtggactgcaccatgtacatctgcggcgactccaccgagtgctccaacctgctgctgcagtacggctccttctgcacccagctgaacagggccctgaccggcatcgccgtggagcaggacaagaacacccaggaggtgttcgcccaggtgaagcagatctacaagaccccccccatcaaggacttcggcggcttcaacttctcccagatcctgcccgacccctccaagccctccaagaggtccttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacacctccgccctgctggccggcaccatcacctccggctggaccttcggcgccggcgccgccctgcagatccccttcgccatgcagatggcctacaggttcaacggcatcggcgtgacccagaacgtgctgtacgagaaccagaagctgatcgccaaccagttcaactccgccatcggcaagatccaggactccctgtcctccaccgcctccgccctgggcaagctgcaggacgtggtgaaccagaacgcccaggccctgaacaccctggtgaagcagctgtcctccaacttcggcgccatctcctccgtgctgaacgacatcctgtccaggctggacaaggtggaggccgaggtgcagatcgacaggctgatcaccggcaggctgcagtccctgcagacctacgtgacccagcagctgatcagggccgccgagatcagggcctccgccaacctggccgccaccaagatgtccgagtgcgtgctgggccagtccaagagggtggacttctgcggcaagggctaccacctgatgtccttcccccagtccgccccccacggcgtggtgttcctgcacgtgacctacgtgcccgcccaggagaagaacttcaccaccgcccccgccatctgccacgacggcaaggcccacttccccagggagggcgtgttcgtgtccaacggcacccactggttcgtgacccagaggaacttctacgagccccagatcatcaccaccgacaacaccttcgtgtccggcaactgcgacgtggtgatcggcatcgtgaacaacaccgtgtacgaccccctgcagcccgagctggactccttcaaggaggagctggacaagtacttcaagaaccacacctcccccgacgtggacctgggcgacatctccggcatcaacgcctccgtggtgaacatccagaaggagatcgacaggctgaacgaggtggccaagaacctgaacgagtccctgatcgacctgcaggagctgggcaagtacgagcagtacatcaagtggccctggtacatctggctgggcttcatcgccggcctgatcgccatcgtgatggtgaccatcatgctgtgctgcatgacctcctgctgctcctgcctgaagggctgctgctcctgcggctcctgctgcaagttcgacgaggacgactccgagcccgtgctgaagggcgtgaagctgcactacacc

SEQ ID NO: 240 - DNA, 600 bp, Artificial Sequence
  SARS-CoV-2
  misc_feature (1)..(600): S protein RBD (SARS-CoV-2)
aacatcaccaacctgtgccccttcggcgaggtgttcaacgccaccaggttcgcctccgtgtacgcctggaacaggaagaggatctccaactgcgtggccgactactccgtgctgtacaactccgcctccttctccaccttcaagtgctacggcgtgtcccccaccaagctgaacgacctgtgcttcaccaacgtgtacgccgactccttcgtgatcaggggcgacgaggtgaggcagatcgcccccggccagaccggcaagatcgccgactacaactacaagctgcccgacgacttcaccggctgcgtgatcgcctggaactccaacaacctggactccaaggtgggcggcaactacaactacctgtacaggctgttcaggaagtccaacctgaagcccttcgagagggacatctccaccgagatctaccaggccggctccaccccctgcaacggcgtggagggcttcaactgctacttccccctgcagtcctacggcttccagcccaccaacggcgtgggctaccagccctacagggtggtggtgctgtccttcgagctgctgcacgcccccgccaccgtgtgcggccccaagaagtcc

SEQ ID NO: 241 - DNA, 600 bp, Artificial Sequence
  SARS-CoV-2
  misc_feature (1)..(600): S protein RBDa (SARS-CoV-2)
aacatcaccaacctgtgccccttcggcgaggtgttcaacgccaccaggttcgcctccgtgtacgcctggaacaggaagaggatctccaactgcgtggccgactactccgtgctgtacaactccgcctccttctccaccttcaagtgctacggcgtgtcccccaccaagctgaacgacctggccttcaccaacgtgtacgccgactccttcgtgatcaggggcgacgaggtgaggcagatcgcccccggccagaccggcaagatcgccgactacaactacaagctgcccgacgacttcaccggctgcgtgatcgcctggaactccaacaacctggactccaaggtgggcggcaactacaactacctgtacaggctgttcaggaagtccaacctgaagcccttcgagagggacatctccaccgagatctaccaggccggctccaccccctgcaacggcgtggagggcttcaactgctacttccccctgcagtcctacggcttccagcccaccaacggcgtgggctaccagccctacagggtggtggtgctgtccttcgagctgctgcacgcccccgccaccgtggccggccccaagaagtcc

SEQ ID NO: 242 - DNA, 2415 bp, Homo sapiens
  misc_feature (1)..(2415): ACE2 (Homo sapiens)
atgtcctcctcctcctggctgctgctgtccctggtggccgtgaccgccgcccagtccaccatcgaggagcaggccaagaccttcctggacaagttcaaccacgaggccgaggacctgttctaccagtcctccctggcctcctggaactacaacaccaacatcaccgaggagaacgtgcagaacatgaacaacgccggcgacaagtggtccgccttcctgaaggagcagtccaccctggcccagatgtaccccctgcaggagatccagaacctgaccgtgaagctgcagctgcaggccctgcagcagaacggctcctccgtgctgtccgaggacaagtccaagaggctgaacaccatcctgaacaccatgtccaccatctactccaccggcaaggtgtgcaaccccgacaacccccaggagtgcctgctgctggagcccggcctgaacgagatcatggccaactccctggactacaacgagaggctgtgggcctgggagtcctggaggtccgaggtgggcaagcagctgaggcccctgtacgaggagtacgtggtgctgaagaacgagatggccagggccaaccactacgaggactacggcgactactggaggggcgactacgaggtgaacggcgtggacggctacgactactccaggggccagctgatcgaggacgtggagcacaccttcgaggagatcaagcccctgtacgagcacctgcacgcctacgtgagggccaagctgatgaacgcctacccctcctacatctcccccatcggctgcctgcccgcccacctgctgggcgacatgtggggcaggttctggaccaacctgtactccctgaccgtgcccttcggccagaagcccaacatcgacgtgaccgacgccatggtggaccaggcctgggacgcccagaggatcttcaaggaggccgagaagttcttcgtgtccgtgggcctgcccaacatgacccagggcttctgggagaactccatgctgaccgaccccggcaacgtgcagaaggccgtgtgccaccccaccgcctgggacctgggcaagggcgacttcaggatcctgatgtgcaccaaggtgaccatggacgacttcctgaccgcccaccacgagatgggccacatccagtacgacatggcctacgccgcccagcccttcctgctgaggaacggcgccaacgagggcttccacgaggccgtgggcgagatcatgtccctgtccgccgccacccccaagcacctgaagtccatcggcctgctgtcccccgacttccaggaggacaacgagaccgagatcaacttcctgctgaagcaggccctgaccatcgtgggcaccctgcccttcacctacatgctggagaagtggaggtggatggtgttcaagggcgagatccccaaggaccagtggatgaagaagtggtgggagatgaagagggagatcgtgggcgtggtggagcccgtgccccacgacgagacctactgcgaccccgcctccctgttccacgtgtccaacgactactccttcatcaggtactacaccaggaccctgtaccagttccagttccaggaggccctgtgccaggccgccaagcacgagggccccctgcacaagtgcgacatctccaactccaccgaggccggccagaagctgttcaacatgctgaggctgggcaagtccgagccctggaccctggccctggagaacgtggtgggcgccaagaacatgaacgtgaggcccctgctgaactacttcgagcccctgttcacctggctgaaggaccagaacaagaactccttcgtgggctggtccaccgactggtccccctacgccgaccagtccatcaaggtgaggatctccctgaagtccgccctgggcgacaaggcctacgagtggaacgacaacgagatgtacctgttcaggtcctccgtggcctacgccatgaggcagtacttcctgaaggtgaagaaccagatgatcctgttcggcgaggaggacgtgagggtggccaacctgaagcccaggatctccttcaacttcttcgtgaccgcccccaagaacgtgtccgacatcatccccaggaccgaggtggagaaggccatcaggatgtccaggtccaggatcaacgacgccttcaggctgaacgacaactccctggagttcctgggcatccagcccaccctgggcccccccaaccagccccccgtgtccatctggctgatcgtgttcggcgtggtgatgggcgtgatcgtggtgggcatcgtgatcctgatcttcaccggcatcagggacaggaagaagaagaacaaggccaggtccggcgagaacccctacgcctccatcgacatctccaagggcgagaacaaccccggcttccagaacaccgacgacgtgcagacctccttc

SEQ ID NO: 243 - DNA, 2220 bp, Homo sapiens
  misc_feature (1)..(2220): ACE2 extra-cellular domain (ECD)  (Homo sapiens)
atgtcctcctcctcctggctgctgctgtccctggtggccgtgaccgccgcccagtccaccatcgaggagcaggccaagaccttcctggacaagttcaaccacgaggccgaggacctgttctaccagtcctccctggcctcctggaactacaacaccaacatcaccgaggagaacgtgcagaacatgaacaacgccggcgacaagtggtccgccttcctgaaggagcagtccaccctggcccagatgtaccccctgcaggagatccagaacctgaccgtgaagctgcagctgcaggccctgcagcagaacggctcctccgtgctgtccgaggacaagtccaagaggctgaacaccatcctgaacaccatgtccaccatctactccaccggcaaggtgtgcaaccccgacaacccccaggagtgcctgctgctggagcccggcctgaacgagatcatggccaactccctggactacaacgagaggctgtgggcctgggagtcctggaggtccgaggtgggcaagcagctgaggcccctgtacgaggagtacgtggtgctgaagaacgagatggccagggccaaccactacgaggactacggcgactactggaggggcgactacgaggtgaacggcgtggacggctacgactactccaggggccagctgatcgaggacgtggagcacaccttcgaggagatcaagcccctgtacgagcacctgcacgcctacgtgagggccaagctgatgaacgcctacccctcctacatctcccccatcggctgcctgcccgcccacctgctgggcgacatgtggggcaggttctggaccaacctgtactccctgaccgtgcccttcggccagaagcccaacatcgacgtgaccgacgccatggtggaccaggcctgggacgcccagaggatcttcaaggaggccgagaagttcttcgtgtccgtgggcctgcccaacatgacccagggcttctgggagaactccatgctgaccgaccccggcaacgtgcagaaggccgtgtgccaccccaccgcctgggacctgggcaagggcgacttcaggatcctgatgtgcaccaaggtgaccatggacgacttcctgaccgcccaccacgagatgggccacatccagtacgacatggcctacgccgcccagcccttcctgctgaggaacggcgccaacgagggcttccacgaggccgtgggcgagatcatgtccctgtccgccgccacccccaagcacctgaagtccatcggcctgctgtcccccgacttccaggaggacaacgagaccgagatcaacttcctgctgaagcaggccctgaccatcgtgggcaccctgcccttcacctacatgctggagaagtggaggtggatggtgttcaagggcgagatccccaaggaccagtggatgaagaagtggtgggagatgaagagggagatcgtgggcgtggtggagcccgtgccccacgacgagacctactgcgaccccgcctccctgttccacgtgtccaacgactactccttcatcaggtactacaccaggaccctgtaccagttccagttccaggaggccctgtgccaggccgccaagcacgagggccccctgcacaagtgcgacatctccaactccaccgaggccggccagaagctgttcaacatgctgaggctgggcaagtccgagccctggaccctggccctggagaacgtggtgggcgccaagaacatgaacgtgaggcccctgctgaactacttcgagcccctgttcacctggctgaaggaccagaacaagaactccttcgtgggctggtccaccgactggtccccctacgccgaccagtccatcaaggtgaggatctccctgaagtccgccctgggcgacaaggcctacgagtggaacgacaacgagatgtacctgttcaggtcctccgtggcctacgccatgaggcagtacttcctgaaggtgaagaaccagatgatcctgttcggcgaggaggacgtgagggtggccaacctgaagcccaggatctccttcaacttcttcgtgaccgcccccaagaacgtgtccgacatcatccccaggaccgaggtggagaaggccatcaggatgtccaggtccaggatcaacgacgccttcaggctgaacgacaactccctggagttcctgggcatccagcccaccctgggcccccccaaccagccccccgtgtcc

SEQ ID NO: 244 - DNA, 2220 bp, Homo sapiens
  misc_feature (1)..(2220): ACE2N extra-cellular domain (ECD) (Homo sapiens)
atgtcctcctcctcctggctgctgctgtccctggtggccgtgaccgccgcccagtccaccatcgaggagcaggccaagaccttcctggacaagttcaaccacgaggccgaggacctgttctaccagtcctccctggcctcctggaactacaacaccaacatcaccgaggagaacgtgcagaacatgaacaacgccggcgacaagtggtccgccttcctgaaggagcagtccaccctggcccagatgtaccccctgcaggagatccagaacctgaccgtgaagctgcagctgcaggccctgcagcagaacggctcctccgtgctgtccgaggacaagtccaagaggctgaacaccatcctgaacaccatgtccaccatctactccaccggcaaggtgtgcaaccccgacaacccccaggagtgcctgctgctggagcccggcctgaacgagatcatggccaactccctggactacaacgagaggctgtgggcctgggagtcctggaggtccgaggtgggcaagcagctgaggcccctgtacgaggagtacgtggtgctgaagaacgagatggccagggccaaccactacgaggactacggcgactactggaggggcgactacgaggtgaacggcgtggacggctacgactactccaggggccagctgatcgaggacgtggagcacaccttcgaggagatcaagcccctgtacgagcacctgcacgcctacgtgagggccaagctgatgaacgcctacccctcctacatctcccccatcggctgcctgcccgcccacctgctgggcgacatgtggggcaggttctggaccaacctgtactccctgaccgtgcccttcggccagaagcccaacatcgacgtgaccgacgccatggtggaccaggcctgggacgcccagaggatcttcaaggaggccgagaagttcttcgtgtccgtgggcctgcccaacatgacccagggcttctgggagaactccatgctgaccgaccccggcaacgtgcagaaggccgtgtgccaccccaccgcctgggacctgggcaagggcgacttcaggatcctgatgtgcaccaaggtgaccatggacgacttcctgaccgcccacaacgagatgggcaacatccagtacgacatggcctacgccgcccagcccttcctgctgaggaacggcgccaacgagggcttccacgaggccgtgggcgagatcatgtccctgtccgccgccacccccaagcacctgaagtccatcggcctgctgtcccccgacttccaggaggacaacgagaccgagatcaacttcctgctgaagcaggccctgaccatcgtgggcaccctgcccttcacctacatgctggagaagtggaggtggatggtgttcaagggcgagatccccaaggaccagtggatgaagaagtggtgggagatgaagagggagatcgtgggcgtggtggagcccgtgccccacgacgagacctactgcgaccccgcctccctgttccacgtgtccaacgactactccttcatcaggtactacaccaggaccctgtaccagttccagttccaggaggccctgtgccaggccgccaagcacgagggccccctgcacaagtgcgacatctccaactccaccgaggccggccagaagctgttcaacatgctgaggctgggcaagtccgagccctggaccctggccctggagaacgtggtgggcgccaagaacatgaacgtgaggcccctgctgaactacttcgagcccctgttcacctggctgaaggaccagaacaagaactccttcgtgggctggtccaccgactggtccccctacgccgaccagtccatcaaggtgaggatctccctgaagtccgccctgggcgacaaggcctacgagtggaacgacaacgagatgtacctgttcaggtcctccgtggcctacgccatgaggcagtacttcctgaaggtgaagaaccagatgatcctgttcggcgaggaggacgtgagggtggccaacctgaagcccaggatctccttcaacttcttcgtgaccgcccccaagaacgtgtccgacatcatccccaggaccgaggtggagaaggccatcaggatgtccaggtccaggatcaacgacgccttcaggctgaacgacaactccctggagttcctgggcatccagcccaccctgggcccccccaaccagccccccgtgtcc

SEQ ID NO: 245 - DNA, 648 bp, Artificial Sequence
  Fc peptide Mut. Glyco. (N->H)
  misc_feature (1)..(648): Fc peptide Mut. Glyco. (N->H)
gcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtaccactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggc

SEQ ID NO: 246 - DNA, 1293 bp, Artificial Sequence
  S-RBD-sFc Fusion protein
  misc_feature (1)..(1293): S-RBD-sFc Fusion protein
aacatcaccaacctgtgccccttcggcgaggtgttcaacgccaccaggttcgcctccgtgtacgcctggaacaggaagaggatctccaactgcgtggccgactactccgtgctgtacaactccgcctccttctccaccttcaagtgctacggcgtgtcccccaccaagctgaacgacctgtgcttcaccaacgtgtacgccgactccttcgtgatcaggggcgacgaggtgaggcagatcgcccccggccagaccggcaagatcgccgactacaactacaagctgcccgacgacttcaccggctgcgtgatcgcctggaactccaacaacctggactccaaggtgggcggcaactacaactacctgtacaggctgttcaggaagtccaacctgaagcccttcgagagggacatctccaccgagatctaccaggccggctccaccccctgcaacggcgtggagggcttcaactgctacttccccctgcagtcctacggcttccagcccaccaacggcgtgggctaccagccctacagggtggtggtgctgtccttcgagctgctgcacgcccccgccaccgtgtgcggccccaagaagtccgagcccaagtcctccgacaagacccacacctcccccccctcccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtaccactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggc

SEQ ID NO: 247 - DNA, 1293 bp, Artificial Sequence
  S-RBDa-sFc Fusion protein
  misc_feature (1)..(1293): S-RBDa-sFc Fusion protein
aacatcaccaacctgtgccccttcggcgaggtgttcaacgccaccaggttcgcctccgtgtacgcctggaacaggaagaggatctccaactgcgtggccgactactccgtgctgtacaactccgcctccttctccaccttcaagtgctacggcgtgtcccccaccaagctgaacgacctggccttcaccaacgtgtacgccgactccttcgtgatcaggggcgacgaggtgaggcagatcgcccccggccagaccggcaagatcgccgactacaactacaagctgcccgacgacttcaccggctgcgtgatcgcctggaactccaacaacctggactccaaggtgggcggcaactacaactacctgtacaggctgttcaggaagtccaacctgaagcccttcgagagggacatctccaccgagatctaccaggccggctccaccccctgcaacggcgtggagggcttcaactgctacttccccctgcagtcctacggcttccagcccaccaacggcgtgggctaccagccctacagggtggtggtgctgtccttcgagctgctgcacgcccccgccaccgtggccggccccaagaagtccgagcccaagtcctccgacaagacccacacctcccccccctcccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtaccactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggc

SEQ ID NO: 248 - DNA, 2913 bp, Artificial Sequence
  ACE2-ECD-sFc Fusion protein
  misc_feature (1)..(2913): ACE2-ECD-sFc Fusion protein
atgtcctcctcctcctggctgctgctgtccctggtggccgtgaccgccgcccagtccaccatcgaggagcaggccaagaccttcctggacaagttcaaccacgaggccgaggacctgttctaccagtcctccctggcctcctggaactacaacaccaacatcaccgaggagaacgtgcagaacatgaacaacgccggcgacaagtggtccgccttcctgaaggagcagtccaccctggcccagatgtaccccctgcaggagatccagaacctgaccgtgaagctgcagctgcaggccctgcagcagaacggctcctccgtgctgtccgaggacaagtccaagaggctgaacaccatcctgaacaccatgtccaccatctactccaccggcaaggtgtgcaaccccgacaacccccaggagtgcctgctgctggagcccggcctgaacgagatcatggccaactccctggactacaacgagaggctgtgggcctgggagtcctggaggtccgaggtgggcaagcagctgaggcccctgtacgaggagtacgtggtgctgaagaacgagatggccagggccaaccactacgaggactacggcgactactggaggggcgactacgaggtgaacggcgtggacggctacgactactccaggggccagctgatcgaggacgtggagcacaccttcgaggagatcaagcccctgtacgagcacctgcacgcctacgtgagggccaagctgatgaacgcctacccctcctacatctcccccatcggctgcctgcccgcccacctgctgggcgacatgtggggcaggttctggaccaacctgtactccctgaccgtgcccttcggccagaagcccaacatcgacgtgaccgacgccatggtggaccaggcctgggacgcccagaggatcttcaaggaggccgagaagttcttcgtgtccgtgggcctgcccaacatgacccagggcttctgggagaactccatgctgaccgaccccggcaacgtgcagaaggccgtgtgccaccccaccgcctgggacctgggcaagggcgacttcaggatcctgatgtgcaccaaggtgaccatggacgacttcctgaccgcccaccacgagatgggccacatccagtacgacatggcctacgccgcccagcccttcctgctgaggaacggcgccaacgagggcttccacgaggccgtgggcgagatcatgtccctgtccgccgccacccccaagcacctgaagtccatcggcctgctgtcccccgacttccaggaggacaacgagaccgagatcaacttcctgctgaagcaggccctgaccatcgtgggcaccctgcccttcacctacatgctggagaagtggaggtggatggtgttcaagggcgagatccccaaggaccagtggatgaagaagtggtgggagatgaagagggagatcgtgggcgtggtggagcccgtgccccacgacgagacctactgcgaccccgcctccctgttccacgtgtccaacgactactccttcatcaggtactacaccaggaccctgtaccagttccagttccaggaggccctgtgccaggccgccaagcacgagggccccctgcacaagtgcgacatctccaactccaccgaggccggccagaagctgttcaacatgctgaggctgggcaagtccgagccctggaccctggccctggagaacgtggtgggcgccaagaacatgaacgtgaggcccctgctgaactacttcgagcccctgttcacctggctgaaggaccagaacaagaactccttcgtgggctggtccaccgactggtccccctacgccgaccagtccatcaaggtgaggatctccctgaagtccgccctgggcgacaaggcctacgagtggaacgacaacgagatgtacctgttcaggtcctccgtggcctacgccatgaggcagtacttcctgaaggtgaagaaccagatgatcctgttcggcgaggaggacgtgagggtggccaacctgaagcccaggatctccttcaacttcttcgtgaccgcccccaagaacgtgtccgacatcatccccaggaccgaggtggagaaggccatcaggatgtccaggtccaggatcaacgacgccttcaggctgaacgacaactccctggagttcctgggcatccagcccaccctgggcccccccaaccagccccccgtgtccgagcccaagtcctccgacaagacccacacctcccccccctcccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtaccactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggc

SEQ ID NO: 249 - DNA, 2913 bp, Artificial Sequence
  ACE2N-ECD-sFc Fusion protein
  misc_feature (1)..(2913): ACE2N-ECD-sFc Fusion protein
atgtcctcctcctcctggctgctgctgtccctggtggccgtgaccgccgcccagtccaccatcgaggagcaggccaagaccttcctggacaagttcaaccacgaggccgaggacctgttctaccagtcctccctggcctcctggaactacaacaccaacatcaccgaggagaacgtgcagaacatgaacaacgccggcgacaagtggtccgccttcctgaaggagcagtccaccctggcccagatgtaccccctgcaggagatccagaacctgaccgtgaagctgcagctgcaggccctgcagcagaacggctcctccgtgctgtccgaggacaagtccaagaggctgaacaccatcctgaacaccatgtccaccatctactccaccggcaaggtgtgcaaccccgacaacccccaggagtgcctgctgctggagcccggcctgaacgagatcatggccaactccctggactacaacgagaggctgtgggcctgggagtcctggaggtccgaggtgggcaagcagctgaggcccctgtacgaggagtacgtggtgctgaagaacgagatggccagggccaaccactacgaggactacggcgactactggaggggcgactacgaggtgaacggcgtggacggctacgactactccaggggccagctgatcgaggacgtggagcacaccttcgaggagatcaagcccctgtacgagcacctgcacgcctacgtgagggccaagctgatgaacgcctacccctcctacatctcccccatcggctgcctgcccgcccacctgctgggcgacatgtggggcaggttctggaccaacctgtactccctgaccgtgcccttcggccagaagcccaacatcgacgtgaccgacgccatggtggaccaggcctgggacgcccagaggatcttcaaggaggccgagaagttcttcgtgtccgtgggcctgcccaacatgacccagggcttctgggagaactccatgctgaccgaccccggcaacgtgcagaaggccgtgtgccaccccaccgcctgggacctgggcaagggcgacttcaggatcctgatgtgcaccaaggtgaccatggacgacttcctgaccgcccacaacgagatgggcaacatccagtacgacatggcctacgccgcccagcccttcctgctgaggaacggcgccaacgagggcttccacgaggccgtgggcgagatcatgtccctgtccgccgccacccccaagcacctgaagtccatcggcctgctgtcccccgacttccaggaggacaacgagaccgagatcaacttcctgctgaagcaggccctgaccatcgtgggcaccctgcccttcacctacatgctggagaagtggaggtggatggtgttcaagggcgagatccccaaggaccagtggatgaagaagtggtgggagatgaagagggagatcgtgggcgtggtggagcccgtgccccacgacgagacctactgcgaccccgcctccctgttccacgtgtccaacgactactccttcatcaggtactacaccaggaccctgtaccagttccagttccaggaggccctgtgccaggccgccaagcacgagggccccctgcacaagtgcgacatctccaactccaccgaggccggccagaagctgttcaacatgctgaggctgggcaagtccgagccctggaccctggccctggagaacgtggtgggcgccaagaacatgaacgtgaggcccctgctgaactacttcgagcccctgttcacctggctgaaggaccagaacaagaactccttcgtgggctggtccaccgactggtccccctacgccgaccagtccatcaaggtgaggatctccctgaagtccgccctgggcgacaaggcctacgagtggaacgacaacgagatgtacctgttcaggtcctccgtggcctacgccatgaggcagtacttcctgaaggtgaagaaccagatgatcctgttcggcgaggaggacgtgagggtggccaacctgaagcccaggatctccttcaacttcttcgtgaccgcccccaagaacgtgtccgacatcatccccaggaccgaggtggagaaggccatcaggatgtccaggtccaggatcaacgacgccttcaggctgaacgacaactccctggagttcctgggcatccagcccaccctgggcccccccaaccagccccccgtgtccgagcccaagtcctccgacaagacccacacctcccccccctcccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtaccactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggc

SEQ ID NO: 250 - protein, 26 aa, Artificial Sequence
  synthetic peptide
  PEPTIDE (1)..(3): KKK- linker
  PEPTIDE (4)..(26): SARS-CoV-2 M protein (64-86)
KKKCFVLAAVYRINWITGGIAIAMAC

SEQ ID NO: 251 - protein, 18 aa, Artificial Sequence
  synthetic peptide
  PEPTIDE (1)..(3): KKK- linker
  PEPTIDE (4)..(18): SARS-CoV-2 M protein (69-83)
KKKAVYRINWITGGIAIA

SEQ ID NO: 252 - protein, 21 aa, Artificial Sequence
  synthetic peptide
  PEPTIDE (1)..(3): KKK- linker
  PEPTIDE (4)..(21): SARS-CoV-2 E (1-18)
KKKMYSFVSEETGTLIVNSVL

SEQ ID NO: 253 - protein, 18 aa, Artificial Sequence
  SARS-CoV-2
  PEPTIDE (1)..(18): SARS-CoV-2 N 73-90
PINTNSSPDDQIGYYRRA

SEQ ID NO: 254 - protein, 36 aa, Artificial Sequence
  SARS-CoV-2
  PEPTIDE (1)..(36): SARS-CoV-2 N 55-90
ALTQHGKEDLKFPRGQGVPINTNSSPDDQIGYYRRA

SEQ ID NO: 255 - protein, 54 aa, Artificial Sequence
  SARS-CoV-2
  PEPTIDE (1)..(54): SARS-CoV-2 N 37-90
SKQRRPQGLPNNTASWFTALTQHGKEDLKFPRGQGVPINTNSSPDDQIGYYRRA

SEQ ID NO: 256 - protein, 72 aa, Artificial Sequence
  SARS-CoV-2
  PEPTIDE (1)..(72): SARS-CoV-2 N 19-90
GPSDSTGSNQNGERSGARSKQRRPQGLPNNTASWFTALTQHGKEDLKFPRGQGVPINTNSSPDDQIGYYRRA

SEQ ID NO: 257 - protein, 90 aa, Artificial Sequence
  SARS-CoV-2
  PEPTIDE (1)..(90): SARS-CoV-2 N 1-90
MSDNGPQNQRNAPRITFGGPSDSTGSNQNGERSGARSKQRRPQGLPNNTASWFTALTQHGKEDLKFPRGQGVPINTNSSPDDQIGYYRRA

SEQ ID NO: 258 - protein, 36 aa, Artificial Sequence
  SARS-CoV-2
  PEPTIDE (1)..(36): SARS-CoV-2 N 73-108
PINTNSSPDDQIGYYRRATRRIRGGDGKMKDLSPRW

SEQ ID NO: 259 - protein, 19 aa, Artificial Sequence
  SARS-CoV-2
  PEPTIDE (1)..(19): SARS-CoV-2 N 160-178
QLPQGTTLPKGFYAEGSRG

SEQ ID NO: 260 - protein, 37 aa, Artificial Sequence
  SARS-CoV-2
  PEPTIDE (1)..(37): SARS-CoV-2 N 142-178
PKDHIGTRNPANNAAIVLQLPQGTTLPKGFYAEGSRG

SEQ ID NO: 261 - protein, 54 aa, Artificial Sequence
  SARS-CoV-2
  PEPTIDE (1)..(54): SARS-CoV-2 N 125-178
ANKDGIIWVATEGALNTPKDHIGTRNPANNAAIVLQLPQGTTLPKGFYAEGSRG

SEQ ID NO: 262 - protein, 57 aa, Artificial Sequence
  synthetic peptide
  PEPTIDE (1)..(3): KKK- linker
  PEPTIDE (4)..(57): SARS-CoV-2 N 125-178
KKKANKDGIIWVATEGALNTPKDHIGTRNPANNAAIVLQLPQGTTLPKGFYAEGSRG

SEQ ID NO: 263 - protein, 70 aa, Artificial Sequence
  SARS-CoV-2
  PEPTIDE (1)..(70): SARS-CoV-2 N 109-178
YFYYLGTGPEAGLPYGANKDGIIWVATEGALNTPKDHIGTRNPANNAAIVLQLPQGTTLPKGFYAEGSRG

SEQ ID NO: 264 - protein, 88 aa, Artificial Sequence
  SARS-CoV-2
  PEPTIDE (1)..(88): SARS-CoV-2 N 91-178
TRRIRGGDGKMKDLSPRWYFYYLGTGPEAGLPYGANKDGIIWVATEGALNTPKDHIGTRNPANNAAIVLQLPQGTTLPKGFYAEGSRG

SEQ ID NO: 265 - protein, 36 aa, Artificial Sequence
  SARS-CoV-2
  PEPTIDE (1)..(36): SARS-CoV-2 N 160-195
QLPQGTTLPKGFYAEGSRGGSQASSRSSSRSRNSSR

SEQ ID NO: 266 - protein, 39 aa, Artificial Sequence
  synthetic peptide
  PEPTIDE (1)..(3): KKK- linker
  PEPTIDE (4)..(39): SARS-CoV-2 N 160-195
KKKQLPQGTTLPKGFYAEGSRGGSQASSRSSSRSRNSSR

SEQ ID NO: 267 - protein, 18 aa, Artificial Sequence
  SARS-CoV-2
  PEPTIDE (1)..(18): SARS-CoV-2 N 249-266
KSAAEASKKPRQKRTATK

SEQ ID NO: 268 - protein, 36 aa, Artificial Sequence
  SARS-CoV-2
  PEPTIDE (1)..(36): SARS-CoV-2 N 231-266
ESKMSGKGQQQQGQTVTKKSAAEASKKPRQKRTATK

SEQ ID NO: 269 - protein, 54 aa, Artificial Sequence
  SARS-CoV-2
  PEPTIDE (1)..(54): SARS-CoV-2 N 213-266
NGGDAALALLLLDRLNQLESKMSGKGQQQQGQTVTKKSAAEASKKPRQKRTATK

SEQ ID NO: 270 - protein, 57 aa, Artificial Sequence
  synthetic peptide
  PEPTIDE (1)..(3): KKK- linker
  PEPTIDE (4)..(57): SARS-CoV-2 N 213-266
KKKNGGDAALALLLLDRLNQLESKMSGKGQQQQGQTVTKKSAAEASKKPRQKRTATK

SEQ ID NO: 271 - protein, 71 aa, Artificial Sequence
  SARS-CoV-2
  PEPTIDE (1)..(71): SARS-CoV-2 N 196-266
NSTPGSSRGTSPARMAGNGGDAALALLLLDRLNQLESKMSGKGQQQQGQTVTKKSAAEASKKPRQKRTATK

SEQ ID NO: 272 - protein, 88 aa, Artificial Sequence
  SARS-CoV-2
  PEPTIDE (1)..(88): SARS-CoV-2 N 179-266
GSQASSRSSSRSRNSSRNSTPGSSRGTSPARMAGNGGDAALALLLLDRLNQLESKMSGKGQQQQGQTVTKKSAAEASKKPRQKRTATK

SEQ ID NO: 273 - protein, 35 aa, Artificial Sequence
  SARS-CoV-2
  PEPTIDE (1)..(35): SARS-CoV-2 N 249-283
KSAAEASKKPRQKRTATKAYNVTQAFGRRGPEQTQ

SEQ ID NO: 274 - protein, 18 aa, Artificial Sequence
  SARS-CoV-2
  PEPTIDE (1)..(18): SARS-CoV-2 N 337-354
IKLDDKDPNFKDQVILLN

SEQ ID NO: 275 - protein, 36 aa, Artificial Sequence
  SARS-CoV-2
  PEPTIDE (1)..(36): SARS-CoV-2 N 319-354
RIGMEVTPSGTWLTYTGAIKLDDKDPNFKDQVILLN

SEQ ID NO: 276 - protein, 54 aa, Artificial Sequence
  SARS-CoV-2
  PEPTIDE (1)..(54): SARS-CoV-2 N 301-354
WPQIAQFAPSASAFFGMSRIGMEVTPSGTWLTYTGAIKLDDKDPNFKDQVILLN

SEQ ID NO: 277 - protein, 71 aa, Artificial Sequence
  SARS-CoV-2
  PEPTIDE (1)..(71): SARS-CoV-2 N 284-354
GNFGDQELIRQGTDYKHWPQIAQFAPSASAFFGMSRIGMEVTPSGTWLTYTGAIKLDDKDPNFKDQVILLN

SEQ ID NO: 278 - protein, 88 aa, Artificial Sequence
  SARS-CoV-2
  PEPTIDE (1)..(88): SARS-CoV-2 N 267-354
AYNVTQAFGRRGPEQTQGNFGDQELIRQGTDYKHWPQIAQFAPSASAFFGMSRIGMEVTPSGTWLTYTGAIKLDDKDPNFKDQVILLN

SEQ ID NO: 279 - protein, 19 aa, Artificial Sequence
  SARS-CoV-2
  PEPTIDE (1)..(19): SARS-CoV-2 S 570-588
ADTTDAVRDPQTLEILDIT

SEQ ID NO: 280 - protein, 37 aa, Artificial Sequence
  SARS-CoV-2
  PEPTIDE (1)..(37): SARS-CoV-2 S 552-588
LTESNKKFLPFQQFGRDIADTTDAVRDPQTLEILDIT

SEQ ID NO: 281 - protein, 58 aa, Artificial Sequence
  synthetic peptide
  PEPTIDE (1)..(3): KKK- linker
  PEPTIDE (4)..(58): SARS-CoV-2 S 534-588
KKKVKNKSVNFNFNGLTGTGVLTESNKKFLPFQQFGRDIADTTDAVRDPQTLEILDIT

SEQ ID NO: 282 - protein, 74 aa, Artificial Sequence
  synthetic peptide
  PEPTIDE (1)..(3): KKK- linker
  PEPTIDE (4)..(74): SARS-CoV-2 S 518-588
KKKLHAPATVSGPKKSTNLVKNKSVNFNFNGLTGTGVLTESNKKFLPFQQFGRDIADTTDAVRDPQTLEILDIT

SEQ ID NO: 283 - protein, 92 aa, Artificial Sequence
  synthetic peptide
  PEPTIDE (1)..(3): KKK- linker
  PEPTIDE (4)..(92): SARS-CoV-2 S 500-588
KKKTNGVGYQPYRVVVLSFELLHAPATVSGPKKSTNLVKNKSVNFNFNGLTGTGVLTESNKKFLPFQQFGRDIADTTDAVRDPQTLEILDIT

SEQ ID NO: 284 - protein, 32 aa, Artificial Sequence
  SARS-CoV-2
  PEPTIDE (1)..(32): SARS-CoV-2 S 573-604
TDAVRDPQTLEILDITPSSFGGVSVITPGTNT

SEQ ID NO: 285 - protein, 20 aa, Artificial Sequence
  SARS-CoV-2
  PEPTIDE (1)..(20): SARS-CoV-2 S 659-678
SYESDIPIGAGISASYQTQT

SEQ ID NO: 286 - protein, 38 aa, Artificial Sequence
  SARS-CoV-2
  PEPTIDE (1)..(38): SARS-CoV-2 S 641-678
NVFQTRAGSLIGAEHVNNSYESDIPIGAGISASYQTQT

SEQ ID NO: 287 - protein, 59 aa, Artificial Sequence
  synthetic peptide
  PEPTIDE (1)..(3): KKK- linker
  PEPTIDE (4)..(59): SARS-CoV-2 S 623-678
KKKAIHADQLTPTWRVYSTGSNVFQTRAGSLIGAEHVNNSYESDIPIGAGISASYQTQT

SEQ ID NO: 288 - protein, 72 aa, Artificial Sequence
  SARS-CoV-2
  PEPTIDE (1)..(72): SARS-CoV-2 S 607-678
QVAVLYQDVNSTEVPVAIHADQLTPTWRVYSTGSNVFQTRAGSLIGAEHVNNSYESDIPIGAGISASYQTQT

SEQ ID NO: 289 - protein, 93 aa, Artificial Sequence
  synthetic peptide
  PEPTIDE (1)..(3): KKK- linker
  PEPTIDE (4)..(20): SARS-CoV-2 S 589-678
KKKPSSFGGVSVITPGTNTSNQVAVLYQDVNSTEVPVAIHADQLTPTWRVYSTGSNVFQTRAGSLIGAEHVNNSYESDIPIGAGISASYQTQT

SEQ ID NO: 290 - protein, 36 aa, Artificial Sequence
  SARS-CoV-2
  PEPTIDE (1)..(36): SARS-CoV-2 S 661-696
ESDIPIGAGISASYQTQTNSPRRARSVASQSIIAYT

SEQ ID NO: 291 - protein, 18 aa, Artificial Sequence
  SARS-CoV-2
  PEPTIDE (1)..(18): SARS-CoV-2 S 750-766
SSNLLLQYGSFSTQLNRA

SEQ ID NO: 292 - protein, 36 aa, Artificial Sequence
  SARS-CoV-2
  PEPTIDE (1)..(36): SARS-CoV-2 S 731-766
MTKTSVDSTMYISGDSTESSNLLLQYGSFSTQLNRA

SEQ ID NO: 293 - protein, 57 aa, Artificial Sequence
  synthetic peptide
  PEPTIDE (1)..(3): KKK- linker
  PEPTIDE (4)..(57): SARS-CoV-2 S 713-766
KKKAIPTNFTISVTTEILPVSMTKTSVDSTMYISGDSTESSNLLLQYGSFSTQLNRA

SEQ ID NO: 294 - protein, 70 aa, Artificial Sequence
  SARS-CoV-2
  PEPTIDE (1)..(70): SARS-CoV-2 S 697-766
MSLGAENSVAYSNNSIAIPTNFTISVTTEILPVSMTKTSVDSTMYISGDSTESSNLLLQYGSFSTQLNRA

SEQ ID NO: 295 - protein, 91 aa, Artificial Sequence
  synthetic peptide
  PEPTIDE (1)..(3): KKK- linker
  PEPTIDE (4)..(91): SARS-CoV-2 S 679-766
KKKNSPRRARSVASQSIIAYTMSLGAENSVAYSNNSIAIPTNFTISVTTEILPVSMTKTSVDSTMYISGDSTESSNLLLQYGSFSTQLNRA

SEQ ID NO: 296 - protein, 38 aa, Artificial Sequence
  SARS-CoV-2
  PEPTIDE (1)..(38): SARS-CoV-2 S 767-804
LTGIAVEQDKNTQEVFAQVKQIYKTPPIKDFGGFNFSQ

SEQ ID NO: 297 - protein, 38 aa, Artificial Sequence
  SARS-CoV-2
  PEPTIDE (1)..(38): SARS-CoV-2 S 821-858
LLFNKVTLADAGFIKQYGDSLGDIAARDLISAQKFNGL

SEQ ID NO: 298 - protein, 18 aa, Artificial Sequence
  SARS-CoV-2
  PEPTIDE (1)..(18): SARS-CoV-2 S 910-927
GVTQNVLYENQKLIANQF

SEQ ID NO: 299 - protein, 36 aa, Artificial Sequence
  SARS-CoV-2
  PEPTIDE (1)..(36): SARS-CoV-2 S 892-927
AALQIPFAMQMAYRFNGIGVTQNVLYENQKLIANQF

SEQ ID NO: 300 - protein, 57 aa, Artificial Sequence
  synthetic peptide
  PEPTIDE (1)..(3): KKK- linker
  PEPTIDE (4)..(57): SARS-CoV-2 S 875-927
KKKTSALLAGTITSGWTFGAGAALQIPFAMQMAYRFNGIGVTQNVLYENQKLIANQF

SEQ ID NO: 301 - protein, 70 aa, Artificial Sequence
  SARS-CoV-2
  PEPTIDE (1)..(70): SARS-CoV-2 S 858-927
LTVLPPLLTDEMIAQYTSALLAGTITSGWTFGAGAALQIPFAMQMAYRFNGIGVTQNVLYENQKLIANQF

SEQ ID NO: 302 - protein, 91 aa, Artificial Sequence
  synthetic peptide
  PEPTIDE (1)..(3): KKK- linker
  PEPTIDE (4)..(91): SARS-CoV-2 S 840-927
KKKSLGDIAARDLISAQKFNGLTVLPPLLTDEMIAQYTSALLAGTITSGWTFGAGAALQIPFAMQMAYRFNGIGVTQNVLYENQKLIANQF

SEQ ID NO: 303 - protein, 36 aa, Artificial Sequence
  SARS-CoV-2
  PEPTIDE (1)..(36): SARS-CoV-2 S 910-945
GVTQNVLYENQKLIANQFNSAIGKIQDSLSSTASAL

SEQ ID NO: 304 - protein, 18 aa, Artificial Sequence
  SARS-CoV-2
  PEPTIDE (1)..(18): SARS-CoV-2 S 998-1015
TGRLQSLQTYVTQQLIRA

SEQ ID NO: 305 - protein, 36 aa, Artificial Sequence
  SARS-CoV-2
  PEPTIDE (1)..(36): SARS-CoV-2 S 980-1015
ILSRLDKVEAEVQIDRLITGRLQSLQTYVTQQLIRA

SEQ ID NO: 306 - protein, 57 aa, Artificial Sequence
  synthetic peptide
  PEPTIDE (1)..(3): KKK- linker
  PEPTIDE (4)..(57): SARS-CoV-2 S 962-1015
KKKLVKQLSSNFGAISSVLNDILSRLDKVEAEVQIDRLITGRLQSLQTYVTQQLIRA

SEQ ID NO: 307 - protein, 70 aa, Artificial Sequence
  SARS-CoV-2
  PEPTIDE (1)..(70): SARS-CoV-2 S 946-1015
GKLQDVVNQNAQALNTLVKQLSSNFGAISSVLNDILSRLDKVEAEVQIDRLITGRLQSLQTYVTQQLIRA

SEQ ID NO: 308 - protein, 91 aa, Artificial Sequence
  synthetic peptide
  PEPTIDE (1)..(3): KKK- linker
  PEPTIDE (4)..(91): SARS-CoV-2 S 928-1015
KKKNSAIGKIQDSLSSTASALGKLQDVVNQNAQALNTLVKQLSSNFGAISSVLNDILSRLDKVEAEVQIDRLITGRLQSLQTYVTQQLIRA

SEQ ID NO: 309 - protein, 36 aa, Artificial Sequence
  SARS-CoV-2
  PEPTIDE (1)..(36): SARS-CoV-2 S 998-1033
TGRLQSLQTYVTQQLIRAAEIRASANLAATKMSESV

SEQ ID NO: 310 - protein, 18 aa, Artificial Sequence
  SARS-CoV-2
  PEPTIDE (1)..(18): SARS-CoV-2 S 1086-1103
KAHFPREGVFVSNGTHWF

SEQ ID NO: 311 - protein, 36 aa, Artificial Sequence
  SARS-CoV-2
  PEPTIDE (1)..(36): SARS-CoV-2 S 1068-1103
VPAQEKNFTTAPAISHDGKAHFPREGVFVSNGTHWF

SEQ ID NO: 312 - protein, 57 aa, Artificial Sequence
  synthetic peptide
  PEPTIDE (1)..(3): KKK- linker
  PEPTIDE (4)..(57): SARS-CoV-2 S 1050-1103
KKKMSFPQSAPHGVVFLHVTYVPAQEKNFTTAPAISHDGKAHFPREGVFVSNGTHWF

SEQ ID NO: 313 - protein, 70 aa, Artificial Sequence
  SARS-CoV-2
  PEPTIDE (1)..(70): SARS-CoV-2 S 1134-1103
LGQSKRVDFSGKGYHLMSFPQSAPHGVVFLHVTYVPAQEKNFTTAPAISHDGKAHFPREGVFVSNGTHWF

SEQ ID NO: 314 - protein, 88 aa, Artificial Sequence
  SARS-CoV-2
  PEPTIDE (1)..(88): SARS-CoV-2 S 1016-1103
AEIRASANLAATKMSESVLGQSKRVDFSGKGYHLMSFPQSAPHGVVFLHVTYVPAQEKNFTTAPAISHDGKAHFPREGVFVSNGTHWF

SEQ ID NO: 315 - protein, 17 aa, Artificial Sequence
  SARS-CoV-2
  PEPTIDE (1)..(17): SARS-CoV-2 S 498-514
QPTNGVGYQPYRVVVLS

SEQ ID NO: 316 - protein, 38 aa, Artificial Sequence
  synthetic peptide
  PEPTIDE (1)..(3): KKK- linker
  PEPTIDE (4)..(38): SARS-CoV-2 S 480-514
KKKSNGVEGFNSYFPLQSYGFQPTNGVGYQPYRVVVLS

SEQ ID NO: 317 - protein, 54 aa, Artificial Sequence
  synthetic peptide
  PEPTIDE (1)..(3): KKK- linker
  PEPTIDE (4)..(54): SARS-CoV-2 S 464-514
KKKFERDISTEIYQAGSTPSNGVEGFNSYFPLQSYGFQPTNGVGYQPYRVVVLS

SEQ ID NO: 318 - protein, 70 aa, Artificial Sequence
  synthetic peptide
  PEPTIDE (1)..(3): KKK- linker
  PEPTIDE (4)..(70): SARS-CoV-2 S 448-514
KKKNYNYLYRLFRKSNLKPFERDISTEIYQAGSTPSNGVEGFNSYFPLQSYGFQPTNGVGYQPYRVVVLS

SEQ ID NO: 319 - protein, 84 aa, Artificial Sequence
  synthetic peptide
  PEPTIDE (1)..(3): KKK- linker
  PEPTIDE (4)..(84): SARS-CoV-2 S 434-514
KKKIAWNSNNLDSKVGGNYNYLYRLFRKSNLKPFERDISTEIYQAGSTPSNGVEGFNSYFPLQSYGFQPTNGVGYQPYRVVVLS

SEQ ID NO: 320 - protein, 36 aa, Artificial Sequence
  SARS-CoV-2
  PEPTIDE (1)..(36): SARS-CoV-2 S 1086-1121
KAHFPREGVFVSNGTHWFVTQRNFYEPQIITTDNTF

SEQ ID NO: 321 - protein, 20 aa, Artificial Sequence
  SARS-CoV-2
  PEPTIDE (1)..(20): SARS-CoV-2 S 1164-1183
VDLGDISGINASVVNIQKEI

SEQ ID NO: 322 - protein, 43 aa, Artificial Sequence
  synthetic peptide
  PEPTIDE (1)..(3): KKK- linker
  PEPTIDE (4)..(43): SARS-CoV-2 S 1144-1183
KKKELDSFKEELDKYFKNHTSPDVDLGDISGINASVVNIQKEI

SEQ ID NO: 323 - protein, 63 aa, Artificial Sequence
  synthetic peptide
  PEPTIDE (1)..(3): KKK- linker
  PEPTIDE (4)..(63): SARS-CoV-2 S 1124-1183
KKKGNSDVVIGIVNNTVYDPLQPELDSFKEELDKYFKNHTSPDVDLGDISGINASVVNIQKEI

SEQ ID NO: 324 - protein, 83 aa, Artificial Sequence
  synthetic peptide
  PEPTIDE (1)..(3): KKK- linker
  PEPTIDE (4)..(83): SARS-CoV-2 S 1104-1183
KKKVTQRNFYEPQIITTDNTFVSGNSDVVIGIVNNTVYDPLQPELDSFKEELDKYFKNHTSPDVDLGDISGINASVVNIQKEI

SEQ ID NO: 325 - protein, 38 aa, Artificial Sequence
  SARS-CoV-2
  PEPTIDE (1)..(38): SARS-CoV-2 S 1166-1203
LGDISGINASVVNIQKEIDRLNEVAKNLNESLIDLQEL

SEQ ID NO: 326 - protein, 21 aa, Artificial Sequence
  SARS-CoV-2
  PEPTIDE (1)..(21): SARS-CoV-2 S 1213-1233
PWYIWLGFIAGLIAIVMVTIM

SEQ ID NO: 327 - protein, 39 aa, Artificial Sequence
  SARS-CoV-2
  PEPTIDE (1)..(39): SARS-CoV-2 S 1195-1233
ESLIDLQELGKYEQYIKWPWYIWLGFIAGLIAIVMVTIM

SEQ ID NO: 328 - protein, 17 aa, Artificial Sequence
  SARS-CoV-2
  PEPTIDE (1)..(17): SARS-CoV-2 Orf9b 34-50
QNNVGPKVYPIILRLGS

SEQ ID NO: 329 - protein, 34 aa, Artificial Sequence
  SARS-CoV-2
  PEPTIDE (1)..(34): SARS-CoV-2 Orf9b 17-50
PQIQLAVTRMENAVGRDQNNVGPKVYPIILRLGS

SEQ ID NO: 330 - protein, 53 aa, Artificial Sequence
  SARS-CoV-2
  PEPTIDE (1)..(3): KKK- linker
  PEPTIDE (4)..(53): SARS-CoV-2 Orf9b 1-50
KKKMDPKISEMHPALRLVDPQIQLAVTRMENAVGRDQNNVGPKVYPIILRLGS

SEQ ID NO: 331 - protein, 38 aa, Artificial Sequence
  SARS-CoV-2
  PEPTIDE (1)..(3): KKK- linker
  PEPTIDE (4)..(38): SARS-CoV-2 Orf9b 34-68
KKKQNNVGPKVYPIILRLGSPLSLNMARKTLNSLEDKA

SEQ ID NO: 332 - protein, 16 aa, Artificial Sequence
  SARS-CoV-2
  PEPTIDE (1)..(16): SARS-CoV-2 Orf9b 82-97
ATTEELPDEFVVVTVK

SEQ ID NO: 333 - protein, 32 aa, Artificial Sequence
  SARS-CoV-2
  PEPTIDE (1)..(32): SARS-CoV-2 Orf9b 66-97
DKAFQLTPIAVQMTKLATTEELPDEFVVVTVK

SEQ ID NO: 334 - protein, 50 aa, Artificial Sequence
  synthetic peptide
  PEPTIDE (1)..(3): KKK- linker
  PEPTIDE (4)..(50): SARS-CoV-2 Orf9b 51-97
KKKPLSLNMARKTLNSLEDKAFQLTPIAVQMTKLATTEELPDEFVVVTVK

SEQ ID NO: 335 - protein, 36 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(36): SARS-CoV-2 S497-508_mod
  PEPTIDE (24)..(36): SARS-CoV-2 S497-509_mod
ISITEIKGVIVHRIETILFKKKKCQPTNGVGYQPCR

SEQ ID NO: 336 - protein, 53 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(53): SARS-CoV-2 S480-508_mod
  PEPTIDE (24)..(53): SARS-CoV-2 S480-509_mod
ISITEIKGVIVHRIETILFKKKKSNGVEGFNSYFPLQSYGCQPTNGVGYQPCR

SEQ ID NO: 337 - protein, 54 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(54): SARS-CoV-2 S361-391_mod
ISITEIKGVIVHRIETILFKKKKCVADYSVLYNSASFSTFKSYGVSPTKLNDLC

SEQ ID NO: 338 - protein, 54 aa, Artificial Sequence
  Synthetic peptide
  PEPTIDE (1)..(31): SARS-CoV-2 S361-391_mod
  PEPTIDE (32)..(35): KKK-epsilon K as a spacer
  SITE (35)..(35): epsilon K
  PEPTIDE (36)..(54): MvF5 Th (UBITh1)
CVADYSVLYNSASFSTFKSYGVSPTKLNDLCKKKKISITEIKGVIVHRIETILF

SEQ ID NO: 339 - protein, 49 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(49): SARS-CoV-2 S363-388_mod
ISITEIKGVIVHRIETILFKKKKCDYSVLYNSASFSTFKSYGVSPTKLC

SEQ ID NO: 340 - protein, 49 aa, Artificial Sequence
  Synthetic peptide
  PEPTIDE (1)..(26): SARS-CoV-2 S363-388_mod
  PEPTIDE (27)..(30): KKK-epsilon K as a spacer
  SITE (30)..(30): epsilon K
  PEPTIDE (31)..(49): MvF5 Th (UBITh1)
CDYSVLYNSASFSTFKSYGVSPTKLCKKKKISITEIKGVIVHRIETILF

SEQ ID NO: 341 - protein, 88 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(88): SARS-CoV-2 S443-507
ISITEIKGVIVHRIETILFKKKKSKVGGNYNYLYRLFRKSNLKPFERDISTEIYQAGSTPCNGVEGFNCYFPLQSYGFQPTNGVGYQP

SEQ ID NO: 342 - protein, 88 aa, Artificial Sequence
  Synthetic peptide
  PEPTIDE (1)..(65): SARS-CoV-2 S443-507
  PEPTIDE (66)..(69): KKK-epsilon K as a spacer
  SITE (69)..(69): epsilon K
  PEPTIDE (70)..(88): MvF5 Th (UBITh1)
SKVGGNYNYLYRLFRKSNLKPFERDISTEIYQAGSTPCNGVEGFNCYFPLQSYGFQPTNGVGYQPKKKKISITEIKGVIVHRIETILF

SEQ ID NO: 343 - protein, 88 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(88): SARS-CoV-2 S443-507_mod
ISITEIKGVIVHRIETILFKKKKCKVGGNYNYLYRLFRKSNLKPFERDISTEIYQAGSTPSNGVEGFNSYFPLQSYGFQPTNGVGYQC

SEQ ID NO: 344 - protein, 88 aa, Artificial Sequence
  Synthetic peptide
  PEPTIDE (1)..(65): SARS-CoV-2 S443-507_mod
  PEPTIDE (66)..(69): KKK-epsilon K as a spacer
  SITE (69)..(69): epsilon K
  PEPTIDE (70)..(88): MvF5 Th (UBITh1)
CKVGGNYNYLYRLFRKSNLKPFERDISTEIYQAGSTPSNGVEGFNSYFPLQSYGFQPTNGVGYQCKKKKISITEIKGVIVHRIETILF

SEQ ID NO: 345 - protein, 31 aa, Artificial Sequence
  synthetic peptide
  PEPTIDE (1)..(3): KKK- linker
  PEPTIDE (4)..(31): SARS-CoV-2 S957-984 (Th/CTL epitope)
KKKQALNTLVKQLSSNFGAISSVLNDILSRL

SEQ ID NO: 346 - protein, 30 aa, Artificial Sequence
  synthetic peptide
  PEPTIDE (1)..(3): KKK- linker
  PEPTIDE (4)..(30): SARS-CoV-2 S891-917 (Th epitope)
KKKGAALQIPFAMQMAYRFNGIGVTQNVLY

SEQ ID NO: 347 - protein, 30 aa, Artificial Sequence
  synthetic peptide
  PEPTIDE (1)..(3): KKK- linker
  PEPTIDE (4)..(30): SARS-CoV-2 N305-331 (Th/CTL epitope)
KKKAQFAPSASAFFGMSRIGMEVTPSGTWL

SEQ ID NO: 348 - protein, 36 aa, Artificial Sequence
  synthetic peptide
  PEPTIDE (1)..(3): KKK- linker
  PEPTIDE (4)..(36): SARS-CoV-2 S996-1028 (Th/CTL epitope)
KKKLITGRLQSLQTYVTQQLIRAAEIRASANLAATK

SEQ ID NO: 349 - protein, 19 aa, Artificial Sequence
  synthetic peptide
  PEPTIDE (1)..(3): KKK- linker
  PEPTIDE (4)..(19): SARS-CoV-2 S1185-1200 (CTL epitope)
KKKRLNEVAKNLNESLIDL

SEQ ID NO: 350 - protein, 22 aa, Artificial Sequence
  synthetic peptide
  PEPTIDE (1)..(3): KKK- linker
  PEPTIDE (4)..(22): SARS-CoV-2 N351-369 (CTL epitope)
KKKILLNKHIDAYKTFPPTEPK

SEQ ID NO: 351 - protein, 15 aa, Artificial Sequence
  synthetic peptide
  PEPTIDE (1)..(3): KKK- linker
  PEPTIDE (4)..(15): SARS-CoV-2 N219-230 (CTL epitope)
KKKLALLLLDRLNQL

SEQ ID NO: 352 - protein, 46 aa, Artificial Sequence
  synthetic peptide
  PEPTIDE (1)..(2): KK- linker
  PEPTIDE (3)..(46): SARS-CoV-2 S 1191-1234
KKKNLNESLIDLQELGKYEQYIKWPWYIWLGFIAGLIAIVMVTIML

SEQ ID NO: 353 - protein, 26 aa, Artificial Sequence
  synthetic peptide
  PEPTIDE (1)..(3): KKK- linker
  PEPTIDE (4)..(26): SARS-CoV-2 S 1251-1273
KKKGSSSKFDEDDSEPVLKGVKLHYT

SEQ ID NO: 354 - protein, 46 aa, Artificial Sequence
  synthetic peptide
  PEPTIDE (1)..(3): KKK- linker
  PEPTIDE (4)..(46): SARS-CoV-2 S 1181-1223
KKKKEIDRLNEVAKNLNESLIDLQELGKYEQYIKWPWYIWLGFIAG

SEQ ID NO: 355 - protein, 431 aa, Artificial Sequence
  Fusion protein
  PEPTIDE (1)..(431): S-RBD-Fc Fusion protein
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSEPKSCDKTHTCPPCPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 356 - protein, 971 aa, Artificial Sequence
  Fusion protein
  PEPTIDE (1)..(971): ACE2-ECD-Fc Fusion protein
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 357 - DNA, 1293 bp, Artificial Sequence
  Fusion protein
  misc_feature (1)..(1293): S-RBD-Fc Fusion protein
aacatcaccaacctgtgccccttcggcgaggtgttcaacgccaccaggttcgcctccgtgtacgcctggaacaggaagaggatctccaactgcgtggccgactactccgtgctgtacaactccgcctccttctccaccttcaagtgctacggcgtgtcccccaccaagctgaacgacctgtgcttcaccaacgtgtacgccgactccttcgtgatcaggggcgacgaggtgaggcagatcgcccccggccagaccggcaagatcgccgactacaactacaagctgcccgacgacttcaccggctgcgtgatcgcctggaactccaacaacctggactccaaggtgggcggcaactacaactacctgtacaggctgttcaggaagtccaacctgaagcccttcgagagggacatctccaccgagatctaccaggccggctccaccccctgcaacggcgtggagggcttcaactgctacttccccctgcagtcctacggcttccagcccaccaacggcgtgggctaccagccctacagggtggtggtgctgtccttcgagctgctgcacgcccccgccaccgtgtgcggccccaagaagtccgagcccaagtcctgcgacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtaccactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggc

SEQ ID NO: 358 - DNA, 2913 bp, Artificial Sequence
  Fusion protein
  misc_feature (1)..(2913): ACE2-ECD-Fc Fusion protein
atgtcctcctcctcctggctgctgctgtccctggtggccgtgaccgccgcccagtccaccatcgaggagcaggccaagaccttcctggacaagttcaaccacgaggccgaggacctgttctaccagtcctccctggcctcctggaactacaacaccaacatcaccgaggagaacgtgcagaacatgaacaacgccggcgacaagtggtccgccttcctgaaggagcagtccaccctggcccagatgtaccccctgcaggagatccagaacctgaccgtgaagctgcagctgcaggccctgcagcagaacggctcctccgtgctgtccgaggacaagtccaagaggctgaacaccatcctgaacaccatgtccaccatctactccaccggcaaggtgtgcaaccccgacaacccccaggagtgcctgctgctggagcccggcctgaacgagatcatggccaactccctggactacaacgagaggctgtgggcctgggagtcctggaggtccgaggtgggcaagcagctgaggcccctgtacgaggagtacgtggtgctgaagaacgagatggccagggccaaccactacgaggactacggcgactactggaggggcgactacgaggtgaacggcgtggacggctacgactactccaggggccagctgatcgaggacgtggagcacaccttcgaggagatcaagcccctgtacgagcacctgcacgcctacgtgagggccaagctgatgaacgcctacccctcctacatctcccccatcggctgcctgcccgcccacctgctgggcgacatgtggggcaggttctggaccaacctgtactccctgaccgtgcccttcggccagaagcccaacatcgacgtgaccgacgccatggtggaccaggcctgggacgcccagaggatcttcaaggaggccgagaagttcttcgtgtccgtgggcctgcccaacatgacccagggcttctgggagaactccatgctgaccgaccccggcaacgtgcagaaggccgtgtgccaccccaccgcctgggacctgggcaagggcgacttcaggatcctgatgtgcaccaaggtgaccatggacgacttcctgaccgcccaccacgagatgggccacatccagtacgacatggcctacgccgcccagcccttcctgctgaggaacggcgccaacgagggcttccacgaggccgtgggcgagatcatgtccctgtccgccgccacccccaagcacctgaagtccatcggcctgctgtcccccgacttccaggaggacaacgagaccgagatcaacttcctgctgaagcaggccctgaccatcgtgggcaccctgcccttcacctacatgctggagaagtggaggtggatggtgttcaagggcgagatccccaaggaccagtggatgaagaagtggtgggagatgaagagggagatcgtgggcgtggtggagcccgtgccccacgacgagacctactgcgaccccgcctccctgttccacgtgtccaacgactactccttcatcaggtactacaccaggaccctgtaccagttccagttccaggaggccctgtgccaggccgccaagcacgagggccccctgcacaagtgcgacatctccaactccaccgaggccggccagaagctgttcaacatgctgaggctgggcaagtccgagccctggaccctggccctggagaacgtggtgggcgccaagaacatgaacgtgaggcccctgctgaactacttcgagcccctgttcacctggctgaaggaccagaacaagaactccttcgtgggctggtccaccgactggtccccctacgccgaccagtccatcaaggtgaggatctccctgaagtccgccctgggcgacaaggcctacgagtggaacgacaacgagatgtacctgttcaggtcctccgtggcctacgccatgaggcagtacttcctgaaggtgaagaaccagatgatcctgttcggcgaggaggacgtgagggtggccaacctgaagcccaggatctccttcaacttcttcgtgaccgcccccaagaacgtgtccgacatcatccccaggaccgaggtggagaaggccatcaggatgtccaggtccaggatcaacgacgccttcaggctgaacgacaactccctggagttcctgggcatccagcccaccctgggcccccccaaccagccccccgtgtccgagcccaagtcctgcgacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtaccactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggc

SEQ ID NO: 359 - protein, 213 aa, Artificial Sequence
  Fusion protein
  PEPTIDE (1)..(213): S-RBD-His Fusion protein
HHHHHHENLYFQGNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 360 - DNA, 639 bp, Artificial Sequence
  Fusion protein
  misc_feature (1)..(639): S-RBD-His Fusion protein
caccaccaccaccaccacgagaacctgtacttccagggcaacatcaccaacctgtgccccttcggcgaggtgttcaacgccaccaggttcgcctccgtgtacgcctggaacaggaagaggatctccaactgcgtggccgactactccgtgctgtacaactccgcctccttctccaccttcaagtgctacggcgtgtcccccaccaagctgaacgacctgtgcttcaccaacgtgtacgccgactccttcgtgatcaggggcgacgaggtgaggcagatcgcccccggccagaccggcaagatcgccgactacaactacaagctgcccgacgacttcaccggctgcgtgatcgcctggaactccaacaacctggactccaaggtgggcggcaactacaactacctgtacaggctgttcaggaagtccaacctgaagcccttcgagagggacatctccaccgagatctaccaggccggctccaccccctgcaacggcgtggagggcttcaactgctacttccccctgcagtcctacggcttccagcccaccaacggcgtgggctaccagccctacagggtggtggtgctgtccttcgagctgctgcacgcccccgccaccgtgtgcggccccaagaagtcc

SEQ ID NO: 361 - protein, 26 aa, Artificial Sequence
  synthetic peptide
  PEPTIDE (1)..(3): KKK- linker
  PEPTIDE (4)..(26): SARS-CoV-2 M 89-111 (Th/CTL epitope)
KKKGLMWLSYFIASFRLFARTRSMWS

SEQ ID NO: 362 - protein, 9 aa, Artificial Sequence
  SARS-CoV-2
  PEPTIDE (1)..(9): SARS-CoV-2 S 975-983
SVLNDILSR

SEQ ID NO: 363 - protein, 18 aa, Artificial Sequence
  SARS-CoV-2
  PEPTIDE (1)..(18): SARS-CoV-2 N 305-322
AQFAPSASAFFGMSRIGM

SEQ ID NO: 364 - protein, 9 aa, Artificial Sequence
  SARS-CoV-2
  SITE (1)..(9): SARS-CoV-2 S 1000-1008
RLQSLQTYV

SEQ ID NO: 365 - protein, 10 aa, Artificial Sequence
  SARS-CoV-2
  PEPTIDE (1)..(10): SARS-CoV-2 S 991-1000
VQIDRLITGR

SEQ ID NO: 366 - protein, 9 aa, Artificial Sequence
  SARS-CoV-2
  PEPTIDE (1)..(9): SARS-CoV-2 M 89-97
GLMWLSYFI

SEQ ID NO: 367 - protein, 15 aa, Artificial Sequence
  SARS-CoV-2
  PEPTIDE (1)..(15): SARS-CoV-2 M 97-111
IASFRLFARTRSMWS

SEQ ID NO: 368 - DNA, 26 bp, Artificial Sequence
  Forward primer E-Sarbeco-F1
acaggtacgttaatagttaatagcgt

SEQ ID NO: 369 - DNA, 22 bp, Artificial Sequence
  Reverse primer E-Sarbeco-R2
atattgcagcagtacgcacaca

SEQ ID NO: 370 - DNA, 26 bp, Artificial Sequence
  Probe E-Sarbeco-P1
  misc_feature (1)..(1): FAM, 6-carboxyfluorescein
  misc_feature (26)..(26): BBQ, blackberry quencher
acactagccatccttactgcgcttcg